SEQUENCE LISTING (36 sequences)

SEQ ID NO: 1 - protein, 24 aa, Artificial sequence
  Chemically synthesized
ARTKGTARKSTGGKAPRKQLAGGK

SEQ ID NO: 2 - protein, 24 aa, Artificial sequence
  Chemically synthesized
ARTKGTARKSTGGKAPRKQLAGGK

SEQ ID NO: 3 - protein, 24 aa, Artificial sequence
  Chemically synthesized
ARTKQTARKSTGGKAPRKQLAGGK

SEQ ID NO: 4 - protein, 26 aa, Artificial sequence
  Chemically synthesized
ATKAARKSAPATGGVKKPHRYRPGGK

SEQ ID NO: 5 - protein, 25 aa, Artificial sequence
  Chemically synthesized
ATKAARKSAPATGGVKKPHRYPGGK

SEQ ID NO: 6 - protein, 26 aa, Artificial sequence
  Chemically synthesized
ATKAARKSAPATGGVKKPHRYRPGGK

SEQ ID NO: 7 - protein, 8 aa, Artificial sequence
  Chemically synthesized
DYKDDDDK

SEQ ID NO: 8 - DNA, 21 bp, Artificial sequence
  Chemically synthesized
cgagatggaattaacagtctt

SEQ ID NO: 9 - DNA, 21 bp, Artificial sequence
  Chemically synthesized
agggagatgcacttcgatata

SEQ ID NO: 10 - DNA, 19 bp, Artificial sequence
  Chemically synthesized
ccatagccgagcagactgg

SEQ ID NO: 11 - DNA, 23 bp, Artificial sequence
  Chemically synthesized
ggatacgtggcgtaaaatgaagt

SEQ ID NO: 12 - DNA, 20 bp, Artificial sequence
  Chemically synthesized
cgagatccctccaaaatcaa

SEQ ID NO: 13 - DNA, 20 bp, Artificial sequence
  Chemically synthesized
gtcttctgggtggcagtgat

SEQ ID NO: 14 - DNA, 21 bp, Artificial sequence
  Chemically synthesized
ccagacttacagggacactta

SEQ ID NO: 15 - DNA, 21 bp, Artificial sequence
  Chemically synthesized
ccttgaaagaagccttacaaa

SEQ ID NO: 16 - DNA, 19 bp, Artificial sequence
  Chemically synthesized
gctgtacgagagtatacac

SEQ ID NO: 17 - DNA, 19 bp, Artificial sequence
  Chemically synthesized
cttctgtactgctgactgg

SEQ ID NO: 18 - DNA, 19 bp, Artificial sequence
  Chemically synthesized
gccaagaacattccagcct

SEQ ID NO: 19 - DNA, 20 bp, Artificial sequence
  Chemically synthesized
tgcaccaccaactgcttagc

SEQ ID NO: 20 - DNA, 21 bp, Artificial sequence
  Chemically synthesized
ggcatggactgtggtcatgag

SEQ ID NO: 21 - DNA, 19 bp, Artificial sequence
  Chemically synthesized
ccgtctttgagccgagttg

SEQ ID NO: 22 - DNA, 22 bp, Artificial sequence
  Chemically synthesized
ggactcttggagtgaaacgaaa

SEQ ID NO: 23 - DNA, 20 bp, Artificial sequence
  Chemically synthesized
gtcaccgtgtcaacctgatg

SEQ ID NO: 24 - DNA, 20 bp, Artificial sequence
  Chemically synthesized
gcctgccttcaagatttctg

SEQ ID NO: 25 - DNA, 20 bp, Artificial sequence
  Chemically synthesized
aagcttcagcaaccagcatt

SEQ ID NO: 26 - DNA, 20 bp, Artificial sequence
  Chemically synthesized
ccctctctctcgctctctca

SEQ ID NO: 27 - DNA, 20 bp, Artificial sequence
  Chemically synthesized
aggggactcagagctcttcc

SEQ ID NO: 28 - DNA, 20 bp, Artificial sequence
  Chemically synthesized
tgcctgtggagtctttgatg

SEQ ID NO: 29 - DNA, 20 bp, Artificial sequence
  Chemically synthesized
cctggatgccatcaaagtct

SEQ ID NO: 30 - DNA, 20 bp, Artificial sequence
  Chemically synthesized
aatccatcggtcatgctctc

SEQ ID NO: 31 - DNA, 20 bp, Artificial sequence
  Chemically synthesized
caagaccaggacggtcattt

SEQ ID NO: 32 - DNA, 20 bp, Artificial sequence
  Chemically synthesized
cctgacgtattccaccttgg

SEQ ID NO: 33 - DNA, 20 bp, Artificial sequence
  Chemically synthesized
ctttgaggatccagcctctg

SEQ ID NO: 34 - DNA, 20 bp, Artificial sequence
  Chemically synthesized
tgcgtttctttgtgttctcg

SEQ ID NO: 35 - DNA, 20 bp, Artificial sequence
  Chemically synthesized
cgtggctttccaagaagaag

SEQ ID NO: 36 - DNA, 20 bp, Artificial sequence
  Chemically synthesized
gcttccgatctgctgaaaac

(19) United States
(12) Patent Application Publication
(10) Pub. No.: US 2015/0272939 A1
(43) Pub. Date: Oct. 1, 2015

(54) Identification of Small Molecule Inhibitors of Jumonji AT-Rich Interactive Domain 1A (JARID1A) and 1B (JARID1B) Histone Demethylase

(71) Applicant: Yale University, New Haven, CT (US)

(72) Inventors: Qin Yan, Stratford, CT (US); Joyce Sayegh, Altadena, CA (US)

(73) Assignee: Yale University, New Haven, CT (US)

(21) Appl. No.: 14/432,311

(22) PCT Filed: Oct. 2, 2013
(86) PCT No.: PCT/US13/63043

§ 371 Date: Mar. 30, 2015

Related U.S. Application Data
(60) Provisional application No. 61/708,979, filed on Oct. 2, 2012.
(60) Provisional application No. 61/776,198, filed on Mar. 11, 2013.
(60) Provisional application No. 61/839,639, filed on Jun. 26, 2013.

(51) Int. Cl. A61K 31/4439; A61K 45/06; A61K 31/41; A61K 31/37; A61K 31/4196; A61K 31/428; C12Q 1/26; A61K 31/192

(52) U.S. Cl. CPC A61K 31/4439; C12Q 1/26; A61K 45/06; A61K 31/192; A61K 31/37; A61K 31/4196; A61K 31/428; A61K 31/41; G01N 2333/90245

(57) ABSTRACT

The present invention includes a novel high-throughput screen capable of identifying compounds that inhibit JARID1B demethylase activity or JARID1A demethylase activity. The present invention further includes novel inhibitors of JARID1B demethylase activity and/or JARID1A demethylase activity, and methods using the same.

### CROSS-REFERENCE TO RELATED APPLICATIONS

The present application claims priority under 35 U.S.C. §119(e) to U.S. Provisional Applications No. 61/708,979, filed Oct. 2, 2012, No. 61/776,198, filed Mar. 11, 2013, and No. 61/839,639, filed Jun. 26, 2013, all of which applications are hereby incorporated by reference in their entireties herein.

### STATEMENT REGARDING FEDERALLY SPONSORED RESEARCH OR DEVELOPMENT

This invention was made with government support under contract numbers UL1 RR024139, P50 CA121974 and P30 CA16359 awarded by the National Institutes of Health. The government has certain rights in the invention.

### BACKGROUND OF THE INVENTION

Covalent posttranslational modification of histones on lysine tails is essential for gene regulation and DNA repair (Blair & Yan, 2012, DNA Cell Biol. 31(Suppl 1):549-61). Histone lysine methylations are now widely accepted modifications for activating or silencing gene transcription, depending on the site and degree of methylation (Blair et al., 2011, Cancers 3:1383-1404). For example, trimethylated lysine 4 in histone H3 (H3K4me3) is associated with active transcription, while trimethylated lysine 27 in histone H3 (H3K27me3) is associated with gene silencing.

The enzymes responsible for the demethylation of H3K4me3 are the Jumonji AT-Rich Interactive Domain 1 (JARID1) or Lysine Demethylase 5 (KDMS) family of lysine demethylases (Klose et al., 2007, Cell 128:889-900; Lee et al., 2007, Cell 128:877-887; Christensen et al., 2007, Cell 128:1063-1076; Iwase et al., 2007, Cell 128:1077-1088; Secombe et al., 2007, Genes Dev. 21:537-551; Yamane et al., 2007, Mol. Cell 25:801-812). This family comprises JARID1A (also known as KDMSA or RBP2), JARID1B (also known as KDMSB or PLU1), JARID1C (also known as KDMSC or SMCX), and JARID1D (also known as KDMSD or SMCY) in mammals (Blair et al., 2011, Cancers 3:1383-1404). Similar to other Jumonji C (JmjC) domain containing demethylases, the JARID1 enzymes catalyze the demethylation of histones in an iron (II) and alpha-ketoglutarate (α-KG) dependent reaction (Klose & Zhang, 2007, Nat. Rev. Mol. Cell Biol. 8:307-318). In this reaction, the oxidative decarboxylation of α-KG results in a hydroxylated methyl-lysine intermediate, which is thermodynamically unstable. The release of the hydroxyl and methyl groups as formaldehyde from this intermediate results in a demethylated lysine residue. Although all the JmjC domain histone demethylases catalyze the reaction via a similar mechanism, they clearly demonstrate specificity toward particular lysine residue(s) (Hou & Yu, 2010, Curr. Opin. Struct. Biol. 20:739-748).

The JARID1 demethylases have been linked to human diseases such as cancer and X-linked mental retardation (Blair et al., 2011, Cancers 3:1383-1404). Both JARID1A and JARID1B are potential oncoproteins, and are overexpressed in a variety of cancers (Blair et al., 2011, Cancers 3:1383-1404). Increased expression of JARID1A promotes a more stem-like phenotype and enhanced resistance to anticancer agents (Sharma et al., 2010, Cell 141:69-80). Moreover, loss of JARID1A inhibits tumorigenesis in two genetically engineered mouse cancer models (Lin et al., 2011, Proc. Natl. Acad. Sci. U.S.A. 108:13379-13386). JARID1A can also promote proliferation, migration, invasion and metastasis of lung cancer cells. (www.ncbi.nlm.nih.gov/pubmed/23722541).

JARID1B is highly expressed in human mammary tumors and breast cancer cell lines, but not in normal adult breast tissue (Lu et al., 1999, J. Biol. Chem. 274:15633-15645). Knockdown of JARID1B leads to upregulation of tumor suppressor genes including BRCA1 (Yamane et al., 2007, Mol. Cell 25:801-812). Downregulation of JARID1B in breast cancer cells decreased tumor formation potential of these cells in a mouse syngeneic or xenograft models (Yamane et al., 2007, Mol. Cell 25:801-812; Catchpole et al., 2011, Int. J. Oncol. 38:1267-1277). JARID1B is also upregulated in advanced and metastatic prostate tumors (Xiang et al., 2007, Proc. Natl. Acad. Sci. U.S.A. 104:19226-19231), and is required for continuous growth of melanoma cells (Roesch et al., 2010, Cell 141:583-594). Taken together, both JARID1A and JARID1B enzymes are very attractive targets for cancer therapy (Blair et al., 2011, Cancers 3:1383-1404). Furthermore, JARID1B promotes multidrug resistance of melanoma cells (www.ncbi.nlm.nih.gov/pubmed/23722541). Even so, no specific inhibitor of these two epigenetic regulators is currently available, and the development of small molecule inhibitors is in demand.

Small molecule inhibitor screens of other JmjC-domain containing demethylases employed methods including detection of the reaction byproduct formaldehyde (Sakurai et al., 2010, Molecular bioSystems 6:357-364; King et al., 2010, PloS one 5:e15535), mass spectrometry (Rose et al., 2010, J. Med. Chem. 53:1810-1818), AlphaScreen (Kawamura et al., 2010, Anal. Biochem. 404:86-93), and LANCE Ultra and AlphaLISA assays (Gauthier et al., 2012, J. Biomol. Screen. 17:49-58).

Several types of JmjC demethylase inhibitors have been identified previously, including α-KG analogues (Suzuki & Miyata, 2011, J. Med. Chem. 54:8236-8250), methyl-lysine analogues, 2,4-PDCA, 8-hydroxyquinoline, catechols, Ni(II), bipyridine, NCDM-32, disulfiram analogues, and hydroxamic acids (Suzuki & Miyata, 2011, J. Med. Chem. 54:8236-8250). One such analogue, 2,4-pyridine dicarboxylic acid (2,4-PDCA), inhibits the catalytic core of JARID1B (Kristensen et al., 2012, FEBS J. 279:1905-1914). However, the specificity is likely compromised as these analogues may inhibit other Fe (II) and α-KG dependent enzymes, such as prolyl hydroxylases (Suzuki & Miyata, 2011, J. Med. Chem. 54:8236-8250). Until now, no high throughput screen has been reported for the JARID1 family of histone lysine demethylases.

There is a need in the art for novel small molecule inhibitors of JARID1 demethylases. These inhibitors would prove useful in treating diseases related to the overactivity and/or overexpression of JARID1, such as cancers and X-linked mental retardation. The present invention addresses and meets these needs.

### BRIEF SUMMARY OF THE INVENTION

The present invention includes a pharmaceutical composition comprising a compound, or a salt or solvate thereof, selected from the group consisting of:
caffeic acid;
esculetin;
a compound of formula (I):

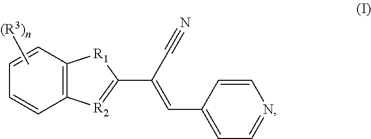

wherein in formula (I):
R¹ is S, O, NH or N(C₁-C₆ alkyl);
R² is N, CH or C—(C₁-C₆ alkyl); and
n is 0, 1, 2, 3 or 4, wherein each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy and carboxy; and,
a compound of formula (II):

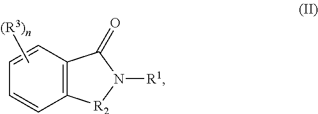

wherein in formula (II):
R¹ is C₁-C₆ alkyl, substituted C₁-C₆ alkyl, C₃-C₇ cycloalkyl, substituted C₃-C₇ cycloalkyl, aryl, substituted aryl, heteroaryl, substituted heteroaryl, heterocyclyl, substituted heretocyclyl, acyl, benzoyl, substituted benzoyl or phenylacetyl;
R² is C(R₄)₂, O, S, C(O), S(O), S(O)₂ or Se;
n is 0, 1, 2, 3 or 4, wherein:
   each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy, cyano and carboxy; and
   each occurrence of R⁴ is independently H, C₁-C₆ alkyl, or substituted C₁-C₆ alkyl.

In one embodiment, in formula (I) R¹ is S, NH or N(CH₃). In another embodiment, in formula (I) R² is N. In yet another embodiment, in formula (I) each occurrence of R³ is independently selected from the group consisting of C₁-C₆ alkyl, substituted C₁-C₆ alkyl, C₁-C₆ haloalkyl, halogen, C₁-C₆ alkoxy, nitro, amino, acetamido, hydroxy and carboxy. In yet another embodiment, in formula (I) R³ is CF₃ and n is 1. In yet another embodiment, the compound of formula (I) is selected from the group consisting of (E)-3-(pyridin-4-yl)-2-(5-(trifluoromethyl)benzo[d]thiazol-2-yl)acrylonitrile; (E)-2-(1-methyl-1H-benzo[d]imidazol-2-yl)-3-(pyridin-4-yl)acrylonitrile; and any combinations thereof. In yet another embodiment, in formula (II) R¹ is C₁-C₆ alkyl, phenylacetyl, aryl or substituted aryl. In yet another embodiment, in formula (II) R¹ is phenyl, o-tolyl, m-tolyl, p-tolyl, o-fluorophenyl, m-fluorophenyl, p-fluorophenyl, o-chlorophenyl, m-chlorophenyl, p-chlorophenyl, o-isopropylphenyl, m-isopropylphenyl, p-isopropylphenyl or isopropyl. In yet another embodiment, in formula (II) R² is C(O), S, SO₂, CH₂ or Se. In yet another embodiment, in formula (II) each occurrence of R³ is independently selected from the group consisting of C₁-C₆ alkyl, substituted C₁-C₆ alkyl, C₁-C₆ haloalkyl, halogen, C₁-C₆ alkoxy, nitro, amino, acetamido, hydroxy, cyano and carboxy. In yet another embodiment, in formula (II) n is 0, 1 or 2. In yet another embodiment, the compound of formula (II) is selected from the group consisting of 2-(4-methylphenyl)-1,2-benzisothiazol-3(2H)-one; 2-phenylbenzo[d][1,2]selenazol-3(2H)-one, 2-(4-chlorophenyl)-5,6-difluorobenzo[d]isothiazol-3(2H)-one, 2-(4-chlorophenyl)-5-(trifluoromethyl)benzo[d]isothiazol-3(2H)-one, 2-(4-chlorophenyl)-6-isocyanobenzo[d]isothiazol-3(2H)-one, and any combinations thereof. In yet another embodiment, the pharmaceutical composition further comprises a pharmaceutically acceptable carrier.

The present invention also includes a method of treating or preventing cancer in a subject in need thereof. The method comprises administering to the subject a therapeutically effective amount of a pharmaceutical composition comprising a compound selected from the group consisting of:

caffeic acid;
esculetin;
a compound of formula (I):

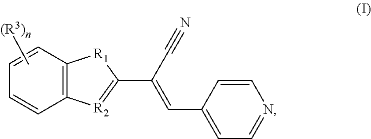

wherein in formula (I):
   R¹ is S, O, NH or N(C₁-C₆ alkyl);
   R² is N, CH or C—(C₁-C₆ alkyl); and
   n is 0, 1, 2, 3 or 4, wherein each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy and carboxy; and,
      a compound of formula (II):

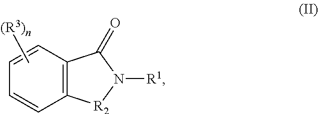

wherein in formula (II):
   R¹ is C₁-C₆ alkyl, substituted C₁-C₆ alkyl, C₃-C₇ cycloalkyl, substituted C₃-C₇ cycloalkyl, aryl, substituted aryl, heteroaryl, substituted heteroaryl, heterocyclyl, substituted heretocyclyl, acyl, benzoyl, substituted benzoyl or phenylacetyl;
   R² is C(R₄)₂, O, S, C(O), S(O), S(O)₂ or Se;
   n is 0, 1, 2, 3 or 4, wherein:
      each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy, cyano and carboxy; and
      each occurrence of R⁴ is independently H, C₁-C₆ alkyl, or substituted C₁-C₆ alkyl.

In one embodiment, administration of the pharmaceutical composition to the subject inhibits the activity of at least one JARID1 demethylase in the subject. In another embodiment, the at least one JARID1 demethylase comprises JARID1B. In yet another embodiment, the at least one JARID1 demethylase comprises JARID1A and JARID1B. In yet another embodiment, the cancer comprises a solid cancer. In yet another embodiment, the solid cancer is selected from the group consisting of breast cancer, prostate cancer, melanoma, lung cancer, and any combinations thereof. In yet another embodiment, the breast cancer comprises at least one HER2-positive breast cancer cell. In yet another embodiment, the at least one HER2-positive breast cancer cell is resistant to trastuzumab. In yet another embodiment, the subject is further administered an additional compound selected from the group consisting of a chemotherapeutic agent, an anti-cell proliferation agent, and any combinations thereof. In yet another embodiment, the chemotherapeutic agent comprises an alkylating agent, nitrosourea, antimetabolite, antitumor antibiotic, plant alkyloid, taxane, hormonal agent, bleomycin, hydroxyurea, L-asparaginase, or procarbazine. In yet another embodiment, the anti-cell proliferation agent comprises granzyme, a Bcl-2 family member, cytochrome C, or a caspase. In yet another embodiment, the pharmaceutical composition and the additional compound are co-administered to the subject. In yet another embodiment, the pharmaceutical composition and the additional compound are co-formulated and co-administered to the subject. In yet another embodiment, the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combinations thereof. In yet another embodiment, the subject is a mammal. In yet another embodiment, the mammal is a human.

The present invention also includes a kit comprising an applicator, an instructional material for use thereof, and a compound selected from the group consisting of:

caffeic acid (also known as (E)-3-(3,4-dihydroxyphenyl)acrylic acid);
esculetin (also known as 6,7-dihydroxy-2H-chromen-2-one);
a compound of formula (I):

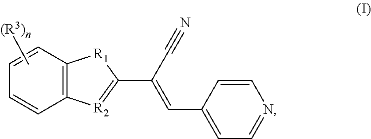

wherein in formula (I):
   R¹ is S, O, NH or N(C₁-C₆ alkyl);
   R² is N, CH or C—(C₁-C₆ alkyl); and
   n is 0, 1, 2, 3 or 4, wherein each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy and carboxy; and,
      a compound of formula (II):

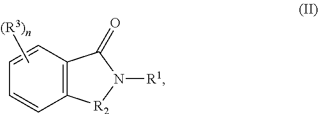

wherein in formula (II):
   R¹ is C₁-C₆ alkyl, substituted C₁-C₆ alkyl, C₃-C₇ cycloalkyl, substituted C₃-C₇ cycloalkyl, aryl, substituted aryl, heteroaryl, substituted heteroaryl, heterocyclyl, substituted heretocyclyl, acyl, benzoyl, substituted benzoyl or phenylacetyl;
   R² is C(R₄)₂, O, S, C(O), S(O), S(O)₂ or Se;
   n is 0, 1, 2, 3 or 4, wherein:
      each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy, cyano and carboxy; and
      each occurrence of R⁴ is independently H, C₁-C₆ alkyl, or substituted C₁-C₆ alkyl,
         wherein the instructional material comprises instructions for preventing or treating cancer in a subject, wherein the instructional material recites that the subject is administered a therapeutically effective amount of a pharmaceutical composition comprising the compound contained in the kit, whereby the cancer in the subject is treated or prevented.

In one embodiment, the cancer comprises breast cancer, prostate cancer, melanoma, lung cancer and any combinations thereof. In another embodiment, the breast cancer comprises at least one HER2-positive breast cancer cell. In yet another embodiment, the at least one HER2-positive breast cancer cell is resistant to trastuzumab.

The present invention also includes a high-throughput method of determining whether a compound inhibits JARID1B demethylase activity. The method comprises the steps of providing tagged full length JARID1B enzyme, incubating the tagged full length JARID1B enzyme with the compound and tagged H3K4Me3 peptide in a system at a determined temperature for a determined period of time, and determining whether any H3K4me2/1 peptide is formed in the system, whereby, if any H3K4me2/1 peptide is formed in the system, the compound is determined to inhibit JARID1B demethylase activity.

In one embodiment, the tagged full length JARID1B enzyme comprises FLAG-tagged full length JARID1B enzyme. In another embodiment, the tagged H3K4Me3 peptide comprises biotinylated H3K4Me3 peptide. In yet another embodiment, the system further comprises alpha-ketoglutarate, an iron (II) salt and ascorbate. In yet another embodiment, determining whether any H3K4me2/1 peptide is formed in the system comprises incubating an H3K4me2 antibody or an H3K4me1 antibody with at least a portion of the system. In yet another embodiment, the system is heterogeneous. In yet another embodiment, the tagged H3K4Me3 peptide is immobilized on a solid support.

The present invention also includes a high-throughput method of determining whether a compound inhibits JARID1A demethylase activity. The method comprises the steps of providing tagged full length JARID1A enzyme, incubating the tagged full length JARID1A enzyme with the compound and tagged H3K4Me3 peptide in a system at a determined temperature for a determined period of time, and determining whether any H3K4me2/1 peptide is formed in the system, whereby, if any H3K4me2/1 peptide is formed in the system, the compound is determined to inhibit JARID1A demethylase activity.

In one embodiment, the tagged full length JARID1B enzyme comprises FLAG-tagged full length JARID1B enzyme. In another embodiment, the tagged H3K4Me3 peptide comprises biotinylated H3K4Me3 peptide. In yet another embodiment, the system further comprises alpha-ketoglutarate, an iron (II) salt and ascorbate. In yet another embodiment, determining whether any H3K4me2/1 peptide is formed in the system comprises incubating an H3K4me2 antibody or an H3K4me1 antibody with at least a portion of the system. In yet another embodiment, the system is heterogeneous. In yet another embodiment, the tagged H3K4Me3 peptide is immobilized on a solid support.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1C, is a non-limiting illustration of an assay of the invention. FIG. 1A: Schematic of the demethylase assay used for detection of JARID1B demethylase activity. Upon laser excitation, energy was transferred from the streptavidin-coated donor beads to the protein A coated acceptor beads. A luminescence signal was detected at 520-620 nm. FIG. 1B: AlphaScreen optimization for antibody specificity. Bio-H3K4me3/2/1 peptides were titrated in the absence of enzyme and detected by the H3K4me1 antibody/bead mix. FIG. 1C: Overview of the high throughput screen and validation for JARID1B inhibitors.

FIG. 2, comprising FIG. 2A-2B, illustrates the analysis of recombinant FLAG-JARID1B by coomassie staining (FIG. 2A) and western blot analysis (FIG. 2B). FT, flow-through. FLAG-JARID1B appears as a ˜170 kDa band.

FIG. 3, comprising FIGS. 3A-3D, illustrates the characterization of JARID1B. FIG. 3A: Enzymatic activity of FLAG-JARID1B (4 nM) as monitored by AlphaScreen signal in the presence and absence of bio-H3K4me3 peptide substrate (64 nM). Bio-H3K4me2 peptide (64 nM) in the absence of enzyme serves as a positive control for the AlphaScreen assay. FIG. 3B: Titration and time course of the FLAG-JARID1B. All assays were carried out in triplicate using 64 nM bio-H3K4me3 peptide and 2 nM, 5 nM, or 7.5 nM FLAG-JARID1B. Reactions were quenched with EDTA at various time points. No signal was seen for bio-H3K4me3 peptide assayed in the absence of FLAG-JARID1B, and bio-H3K4me2 (64 nM) assayed in the absence of enzyme represents a positive control for the AlphaScreen assay. FIG. 3C: Demethylase activity of FLAG-JARID1B upon titration of the bio-H3K4me3 peptide for Kₘ determination. FIG. 3D: Demethylase activity of FLAG-JARID1B on the bio-H3K4me3 peptide substrate upon titration of a-ketoglutarate for Kₘ determination.

FIG. 4, comprising FIGS. 4A-4E, illustrates the finding that PBIT is selective for JARID1 enzymes. JARID1B (FIG. 4A), JARID1A (FIG. 4B), and JARID1C (FIG. 4C) were assayed with 64 nM bio-H3K4me3 peptide and PBIT or 2,4-PDCA (10 μM). UTX (FIG. 4D) and JMJD3 (FIG. 4E) were assayed with 64 nM bio-H3K27me3 peptide and PBIT or 2,4-PDCA (10 μM), and demethylase activity was detected using anti-H3K27me2 antibody.

FIG. 5 is a series of illustrations that show that PBIT inhibits H3K4me3 demethylation in vivo. 3×HA-JARID1B was expressed in HeLa cells, and cells were incubated with 0.1% DMSO, or 10 μM or 30 μM PBIT for 24 hours. Cell nuclei were identified by DAPI staining (top panel, blue). 3×HA-JARID1B was identified by HA-immunofluorescence (second panel, red), and H3K4me3 was visualized by H3K4me3 immunofluorescence (third panel, green). The merged images of HA and H3K4me3 immunofluorescence are illustrated in the bottom panel. Triangles indicate transfected cells.

FIG. 6, comprising FIGS. 6A-6H, is a series of graphs illustrating the finding that PBIT inhibits cell proliferation in a JARID1B level-dependent manner. FIG. 6A: Western blot analysis of UACC-812, MCF7 and MCF10A cells with the indicated antibodies. FIGS. 6B-6D: WST-1 cell proliferation assays of UACC-812 (FIG. 6B), MCF7 (FIG. 6C) and MCF10A (FIG. 6D) cells in the presence of PBIT at the indicated concentrations. Illustrated are the ratio of absorbance at 440 nm of day 3/day 0 (D3/D0) with SEM. FIG. 6E: Real time RT-PCR analysis of JARID1B mRNA in stable cell lines with the indicated shRNA hairpins. Illustrated are mean values with SEM. FIGS. 6F-6H: WST-1 cell proliferation assays of UACC-812 (FIG. 6F), MCF7 (FIG. 6G) and MCF10A (FIG. 6H) cells with control or JARID1B shRNA hairpins. Illustrated are the ratio of absorbance at 440 nm of day 3 or 4/day 0 (D3 or D4/D0) with SEM.

FIG. 7, comprising FIGS. 7A-7C, is a series of graphs illustrating the dose response analysis of PBIT on JARID1A (FIG. 7A) and JARID1C (FIG. 7B), and of 2,4-PDCA on JARID1A (FIG. 7C).

FIG. 8 is a graph illustrating the antibody optimization of the AlphaScreen assay for UTX and JMJD3 demethylases. Bio-H3K27me3/2/1 peptides were titrated and subject to AlphaScreen detection with anti-H3K27me2 antibody. Significant signal was only observed for the bio-H3K27me2 peptide.

FIG. 9 is a series of gel images illustrating the finding that PBIT increases global H3K4me3 level in MCF7 cells. Histone extracts from MCF7 cells treated with 10 μM PBIT or DMSO (0.01%) for 72 h were analyzed by western blotting analysis with the indicated antibodies.

FIG. 10, comprising FIGS. 10A-10D, illustrates the finding that JARID1B is required for trastuzumab resistance. FIG. 10A: Schematic of the methods to generate trastuzumab resistant SKBR3 cells (SKBR3-R) from trastuzumab sensitive SKBR3 cells (SKBR3-S). FIG. 10B: WST-1 cell proliferation assays of SKBR3-S and SKBR3-R cells in the presence of 30 μM PBIT. Illustrated are the ratio of absorbance at 440 nm of day 3/day 0 (D3/D0). FIG. 10C: Real time RT-PCR analysis of JARID1B mRNA in stable cell lines with the indicated shRNA hairpins. Illustrated are mean values with SEM. FIG. 10D: WST-1 cell proliferation assays of SKBR3-S and SKBR3-R cells with control or JARID1B shRNA hairpins, in the presence or absence of 100 μg/ml trastuzumab. Illustrated are the ratio of absorbance at 440 nm of day 5/day 0 (D5/D0).

FIG. 11 is a bar graph illustrating the finding that melanoma cells are sensitive to PBIT treatment. WST-1 cell proliferation assays of 1445, YUAME, YULAC, YURIF melanoma cells in the presence of 0, 10 and 30 μM PBIT. Illustrated are the relative ratio of absorbance at 440 nm of day 3/day 0 (D3/D0) with SEM, normalized to DMSO mock treated cells.

FIG. 12, comprising FIGS. 12A-12C, illustrates the JARID1A demethylase assay. FIG. 12A: Schematic of the demethylase assay used for detection of JARID1A demethylase activity. Upon laser excitation, energy was transferred from the streptavidin-coated donor beads to the protein A coated acceptor beads. A luminescence signal was detected at 520-620 nm. FIG. 12B: AlphaScreen optimization for antibody specificity. Bio-H3K4me3/2/1 peptides were titrated in the absence of enzyme and detected by the H3K4me1 antibody/bead mix. FIG. 12C: Overview of the high throughput screen and validation for JARID1A inhibitors.

FIG. 13, comprising FIGS. 13A-13B, illustrates the analysis of recombinant FLAG-JARID1A by coomassie brilliant blue staining (FIG. 13A) and western blot analysis (FIG. 13B). FT, flow-through. FLAG-JARID1A appeared as a ˜200 kDa band.

FIG. 14, comprising FIGS. 14A-14M, illustrates selected active compounds that inhibit the demethylase activity of JARID1A. The figures comprise compound structures, dose response curves and IC₅₀ value from dose response curves performed at 50 μM Fe(II).

FIG. 15 is a graph illustrating RBP2 (at 19 nM) enzyme activity with 5 μM MIF inhibitors (MIF-143=2-(4-chlorophenyl)-5,6-difluorobenzo[d]isothiazol-3(2H)-one; MIF-110=2-(4-chlorophenyl)-5-(trifluoromethyl)benzo[d]isothiazol-3(2H)-one; MIF-112=2-(4-chlorophenyl)-6-isocyanobenzo[d]isothiazol-3 (2H)-one).

FIG. 16 is a graph illustrating RBP2 (at 19 nM) enzyme activity and PLU1 (at 25 nM) enzyme activity with 5 μM MIF inhibitors.

FIG. 17 is a graph illustrating RBP2 (at 19 nM) enzyme activity with MIF-110 and MIF-112.

FIG. 18 is a graph illustrating RBP2 (at 19 nM) titration with MIF-143.

FIG. 19, comprising FIGS. 19A-19D, illustrates the finding that high RBP2 expression level is associated with breast cancer metastasis. FIG. 19A: Correlation of the mRNA levels of histone modifying enzymes with breast cancer metastasis. The patients were divided into two groups with either higher or lower expression as compared to the median based on each probe. Plotted were hazard ratio (HR) with 95% confidence and Bonferroni multiple testing corrected p-value (MTCPV). FIG. 19B: Kaplan-Meier analysis of metastasis-free survival of lymph node negative patients with breast cancer, stratified by RBP2 expression level based on the 202040_s_at probe. FIG. 19C: Summary of Kaplan-Meier analysis of metastasis-free survival of all patients, ER⁺ or ER⁻ breast cancer patients in the EMC286 cohort. FIG. 19D: Western blot analysis of RBP2 and tubulin in MDA-MB-231 (231), LM2, 67NR, and 4T1 cells.

FIG. 20, comprising FIGS. 20A-20F, illustrates the finding that RBP2 regulates the expression of lung metastasis genes. FIG. 20A: Gene-set enrichment analysis showing decreased enrichment of the lung metastasis gene signature in MDA-MB-231 cells transfected with RBP2 siRNA compared with those with control siRNA. RBP2 KD, RBP2 siRNA knockdown; Ctrl KD, control siRNA knockdown. FIG. 20B: Real time RT-PCR analysis of RBP2 and TNC in MDA-MB-231 (231) or LM2 cells transfected with control or RBP2 siRNA. Scr si, scrambled control siRNA; RBP2 si-1, RBP2 siRNA-1; RBP2 si-2, RBP2 siRNA-2. FIG. 20C: Western blot analysis of the indicated proteins in whole cell lysates or culture media of MDA-MB-231 (231) and LM2 cells transfected with the indicated siRNAs. FIG. 20D: Western blot analysis of the indicated proteins in whole cell lysates or culture media of LM2 cells transfected with the indicated siRNAs and/or HA-RBP2 plasmid. FIG. 20E: Box plots showing TNC expression level in ER⁺ or ER⁻ tumors expressing high, or medium and low RBP2 in the EMC286 clinical dataset. RBP2 high, M (medium) and L (low) were defined using k-means clustering. FIG. 20F: Scatter plot showing the positive correlation between RBP2 and TNC expression in ER⁻ breast tumors in the EMC286 clinical dataset. Pearson correlation test was performed to assess statistical significance.

FIG. 21, comprising FIGS. 21A-21D, illustrates the finding that knockdown of RBP2 reduces cell invasion in vitro. (FIG. 21A) Representative image of DAPI staining and (FIG. 21B) left panel quantification of LM2 cells invaded through Matrigel coated membrane inserts after treatment with the indicated siRNA. FIG. 21B: Right panel, western blot analysis of the indicated proteins. Luc, luciferase siRNA; Scr, scamble siRNA, RBP2 si-1, RBP2 siRNA-1; RBP2 si-2, RBP2 siRNA-2. FIG. 21C: Left panel, quantification of MDA-MB-231 and LM2 cells invaded through Matrigel coated membrane inserts after transfection with the indicated siRNAs and plasmids. FIG. 21C: Right panel, western blot analysis of the indicated proteins. EV plasmid, empty vector plasmid; RBP2 plasmid, HA-RBP2 plasmid; scr siRNA, scrambled siRNA; RBP2 siRNA, RBP2 siRNA-1; 231, MDA-MB-231 cells. FIG. 21D: Left panel, quantification of LM2 cells invaded through Matrigel coated membrane inserts after transfection with the indicated siRNAs and treatment with the indicated concentration of recombinant TNC protein. FIG. 21D: Right panel, western blot analysis of the indicated proteins. Luc siRNA, luciferase siRNA. FIGS. 21B-D: 4 random fields of each insert were quantified. Error bars represent s.e.m. of three inserts. **, p<0.01; ***, p<0.001

FIG. 22, comprising FIGS. 22A-22F, illustrates the finding that knockdown of RBP2 decreases tumor metastasis in vivo. (FIG. 22A,C) Normalized bioluminescence signals of lung metastasis of mice injected intravenously with LM2 cells stably expressing control or RBP2 shRNA. Ctrl sh, control shRNA. The data represent average±s.e.m. *, p<0.05; ***, p<0.001. (FIG. 22B, D) Representative bioluminescence images of mice in each experiment group at Day 35 (FIG. 22B) or Day 49 (FIG. 22D). (FIG. 22E) Representative H&E-stained lung sections. Mice injected with LM2 cells carrying control shRNA (Control sh) have more tumors [1 *(not all tumor foci marked), 3] than mice with LM2 cells carrying RBP2 shRNA (RBP2 sh-1) (2 arrowheads, 4) visible at low power. Vascular invasion (3, arrow) and small foci of metastatic nodules (4 arrows) are observed at increased magnification. Scale bars a, b=500 μm; c, d=100 μm. (FIG. 22F) The average weight of primary tumors at the endpoint in mice implanted in mammary fat pads with LM2 cells stably expressing control or RBP2 shRNA. Ctrl, control shRNA. Sh-1, RBP2 sh-1.

FIG. 23, comprising FIGS. 23A-23D, illustrates the finding that loss of RBP2 decreases tumor progression and metastasis in the MMTV-neu transgenic mice. (FIG. 23A) Kaplan-Meier tumor-free survival curves of the MMTV-neu transgenic mice with the indicated genotypes of Rbp2. N, animal number in each group. M represents days of medium survival in each group. p<0.0001 based on Log-rank (Mental-Cox) test. (FIG. 23B) Scatter plot showing the number of lung metastatic nodules in the MMTV-neu transgenic mice with the indicated Rbp2 genotypes. (FIG. 23C) Incidence of lung metastasis of the MMTV-neu transgenic mice with the indicated Rbp2 genotypes. (FIG. 23D) Representative H&E-stained lung sections. Rbp2^{+/+}:MMTV-neu mice (1, 3, 5) showed greater numbers of tumors in the lung than Rbp2^{-/-}:MMTV-neu mice (2, 4, 6), and the morphology of the tumor cells are similar (5, 6). Scale bars for panels 1-4=500 μm and for panels 5−6=100 μm.

FIG. 24, comprising FIGS. 24A-24D, illustrates the finding that high RBP2 expression level is associated with breast cancer metastasis. (FIG. 24A) Kaplan-Meier analysis of metastasis-free survival of lymph node negative patients with breast cancer, stratified by RBP2 expression level based on the probe 215698_at. (FIGS. 24B-D) Kaplan-Meier analyses of metastasis-free survival of patients from the EMC286 cohort with the indicated ER status. High and low RBP2 levels were defined by top 25% and bottom 25%, respectively.

FIG. 25, comprising FIGS. 25A-24B, illustrates the finding that knockdown of RBP2 in MDA-MB-231 cells affects histone H3 methylation status globally. (FIG. 25A) Real time RT-PCR analysis of RBP2 expression in MDA-MB-231 cells transfected with the indicated siRNA. RBP2 mRNA level was normalized to GAPDH. (FIG. 25B) Western blot analysis of histone or histone modifications in MDA-MB-231 cells transfected with the indicated siRNA. Scr, scrambled siRNA.

FIG. 26 is a set of graphs illustrating real time RT-PCR analysis of the indicated mRNAs in MDA-MB-231 (231) or LM2 cells transfected with control or RBP2 siRNA. Scr si, scrambled control siRNA; RBP2 si-1, RBP2 siRNA-1; RBP2 si-2, RBP2 siRNA-2.

FIG. 27 illustrates the finding that stable knockdown of RBP2 in LM2 cells decreases TNC secretion. Western blot analysis of the indicated proteins in LM2 cells stably expressing the indicated shRNA. Ctrl sh, control shRNA.

FIG. 28 illustrates the finding that box plot showing the average weight of primary tumors from the MMTV-neu mice with the indicated genotypes examined in FIG. 5.

FIG. 29, comprising FIGS. 29A-29C, is a table illustrates the association of expression levels of histone modifying enzymes with metastasis-free survival.

FIG. 30 is a table illustrating the Cox multivariate analysis of RBP2, ER, PR, HER2 levels and stage for metastasis free survival in the EMC286 cohort.

FIG. 31 is a table illustrating gene-set enrichment analyses of MDA-MB-231 cells with RBP2 or control shRNA using organ-specific metastasis gene signatures. Shown are normalized enrichment scores (NES), nominal p-value (NOM p-val), and false discovery rate q-value (FDR q-val) comparing cells with RBP2 shRNA versus those with control shRNA. LMS, lung metastasis signature; BoMS, bone metastasis signature; BrMS, brain metastasis signature; Up, upregulated genes; Down, downregulated genes.

### DETAILED DESCRIPTION OF THE INVENTION

The invention relates to the unexpected discovery of a novel high-throughput screen to identify small molecule inhibitors of full length JARID1A or JARID1B using the AlphaScreen platform. By implementing AlphaScreen technology, a very sensitive assay for detecting demethylation of a biotinylated H3K4me3 peptide in vitro was developed.

In one aspect, JARID1B was assayed against a diverse library consisting of 15,134 molecules, and several compounds that yielded low μM IC₅₀ values were identified. In a non-limiting example, 2-4(4-methylphenyl)-1,2-benzisothiazol-3(2H)-one (PBIT) inhibits JARID1B up to 95%, with an IC₅₀ value of about 3 μM. This compound may also inhibit other members of the JARID1 family, but did not inhibit the H3K27me3 demethylases UTX or JMJD3, suggesting that PBIT may be specific for the JARID1 enzymes. Furthermore, PBIT modulates H3K4me3 levels in cells and attenuate proliferation of UACC-812 breast cancer cells and melanoma cells. Taken together, these studies reveal the identification of novel inhibitors of JARID1B in vitro with therapeutic implications for cancer, such as but not limited to breast cancer and melanoma.

In another aspect, JARID1A was assayed against a diverse library consisting of 9,600 molecules, and several compounds that yielded high nM IC₅₀ values were identified. Most of these compounds did not inhibit the other member of the JARID1 family JARID1B. Taken together, these studies reveal the identification of novel inhibitors of JARID1A in vitro with therapeutic implications for cancer, such as but not limited to breast cancer and lung cancer.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “MIF-143” refers to 2-(4-chlorophenyl)-5,6-difluorobenzo[d]isothiazol-3(2H)-one, or a salt or solvate thereof.

As used herein, the term “MIF-110” refers to 2-(4-chlorophenyl)-5-(trifluoromethyl)benzo[d]isothiazol-3(2H)-one, or a salt or solvate thereof.

As used herein, the term “MIF-112” refers to 2-(4-chlorophenyl)-6-isocyanobenzo[d]isothiazol-3(2H)-one), or a salt or solvate thereof.

As used herein, the term “caffeic acid” refers to (E)-3-(3,4-dihydroxyphenyl)acrylic acid, or a salt or solvate thereof.

As used herein, the term “esculetin” refers to 6,7-dihydroxy-2H-chromen-2-one, or a salt or solvate thereof.

As used herein, the term “2,4-PDCA” refers to 2,4-pyridinedicarboxylic acid monohydrate, or a salt or solvate thereof.

As used herein, the term “α-KG” refers to alpha-ketoglutarate, or a salt or solvate thereof.

As used herein, the term “bio” refers to biotin or biotinylated.

As used herein, the term “DAPI” refers to 4,6-diamidino-2-phenylindole dihydrochloride, or a salt or solvate thereof.

As used herein, the term “DMSO” refers to dimethyl sulfoxide.

As used herein, the term “EDTA,” refers to ethylenediamine tetraacetic acid, or a salt or solvate thereof.

As used herein, the term “EGF” refers to epidermal growth factor.

As used herein, the term “FBS” refers to fetal bovine serum.

As used herein, the term “H3K4me1” refers to monomethylated lysine 4 in histone H3.

As used herein, the term “H3K4me2” refers to dimethylated lysine 4 in histone H3.

As used herein, the term “H3K4me3” refers to trimethylated lysine 4 in histone H3.

As used herein, the term “H3K27me2” refers to dimethylated lysine 27 in histone H3.

As used herein, the term “H3K27me1” refers to monomethylated lysine 27 in histone H3.

As used herein, the term “H3K27me3” refers to trimethylated lysine 27 in histone H3.

As used herein, the term “HER2+” refers to HER2 positive.

As used herein, the term “IC₅₀” refers to half maximal inhibitory concentration.

As used herein, the term “JARID1” refers to Jumonji AT-Rich Interactive Domain 1.

As used herein, the term “KDMS” refers to Lysine Demethylase 5.

As used herein, the term “JmjC” refers to jumonji.

As used herein, the term “PBIT” refers to 2-4(4-methylphenyl)-1,2-benzisothiazol-3(2H)-one, or a salt or solvate thereof.

As used herein, the term “p/s” refers to penicillin/streptomycin.

As used herein, the term “RT-PCR” refers to reverse transcription PCR.

As used herein, the term “trastuzumab” refers to a monoclonal antibody that interferes with the HER2/neu receptor (tradenames Herclon, Herceptin) (Hudis, 2007, N. Engl. J. Med. 3577(1):39-51).

As used herein, a “solvate” of a molecule refers to a complex between the molecule and a finite number of solvent molecules. In one embodiment, the solvate is a solid isolated from solution by precipitation or crystallization. In another embodiment, the solvate is a hydrate.

As used herein, a “subject” may be a human or non-human mammal or a bird. Non-human mammals include, for example, livestock and pets, such as ovine, bovine, porcine, canine, feline and murine mammals. Preferably, the subject is human.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “non-cancer control sample” as relating to a subject's tissue refers to a sample from the same tissue type, obtained from the patient, wherein the sample is known or found not to be afflicted with cancer. For example, a non-cancer control sample for a subject's lung tissue refers to a lung tissue sample obtained from the subject, wherein the sample is known or found not to be afflicted with cancer. “Non-cancer control sample” for a subject's tissue also refers to a reference sample from the same tissue type, obtained from another subject, wherein the sample is known or found not to be afflicted with cancer. “Non-cancer control sample” for a subject's tissue also refers to a standardized set of data (such as, but not limited to, identity and levels of gene expression, protein levels, pathways activated or deactivated etc.), originally obtained from a sample of the same tissue type and thought or considered to be a representative depiction of the non-cancer status of that tissue.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, an “effective amount”, “therapeutically effective amount” or “pharmaceutically effective amount” of a compound is that amount of compound that is sufficient to provide a beneficial effect to the subject to which the compound is administered.

The terms “treat” “treating” and “treatment,” as used herein, means reducing the frequency or severity with which symptoms of a disease or condition are experienced by a subject by virtue of administering an agent or compound to the subject.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “composition” or “pharmaceutical composition” refers to a mixture of at least one compound useful within the invention with a pharmaceutically acceptable carrier. The pharmaceutical composition facilitates administration of the compound to a subject.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

In one aspect, the terms “co-administered” and “co-administration” as relating to a subject refer to administering to the subject a compound useful within the invention, or salt thereof, along with a compound that may also treat any of the diseases contemplated within the invention. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

By the term “specifically bind” or “specifically binds,” as used herein, is meant that a first molecule preferentially binds to a second molecule (e.g., a particular receptor or enzyme), but does not necessarily bind only to that second molecule.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “substituted alkyl,” “substituted cycloalkyl,” “substituted alkenyl” or “substituted alkynyl” means alkyl, cycloalkyl, alkenyl or alkynyl, as defined above, substituted by one, two or three substituents selected from the group consisting of halogen, —OH, alkoxy, tetrahydro-2-H-pyranyl, —NH₂, —N(CH₃)₂, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain may be branched, straight or cyclic, with straight being preferred.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention relates to a high-throughput screen for inhibitors of the JARID1 family of demethylases. This screen allows for the rapid and reliable identification of inhibitors of JARID1 demethylase activity.

Very robust high throughput screens using the AlphaScreen platform are disclosed herein and facilitate searching for novel small molecule inhibitors of the histone lysine demethylase JARID1A and JARID1B. In one embodiment, the high-throughput screen of the invention utilizes full length JARID1A or JARID1B. In another embodiment, the substrate for the assay comprises bio-H3K4me3. The Kₘ for bio-H3K4me3 using full length JARID1B was found to be 15 nM, which is much lower than the reported Kₘ for the JARID1B catalytic core (Kristensen et al., 2012, FEBS J. 279:1905-1914). In one embodiment, domains of JARID1B contribute to folding of the protein or substrate recognition and can be targeted for inhibition.

The signal-to-noise ratio associated with the assay of the invention was high (˜17), even with only 4 nM enzyme, producing a Z′ factor of ˜0.8 (Table 5). This allowed the use of small amounts of enzymes and the identification of inhibitors with very low IC₅₀ values.

After screening over 15,000 small molecules, over 90 validated compounds that inhibit JARID1B activity (Table 3) were identified, many of which have IC₅₀ values in the low micromolar range. After screening 9,600 small molecules, 257 validated compounds that inhibit JARID1A activity were identified, many of which have IC₅₀ values in the high nanomolar or low micromolar range.

Some of these known JmjC demethylase inhibitors were identified in the present screens. For example, several of the hits in the screening assay disclosed herein were identified in the miniaturized screen for inhibitors of the H3K9 demethylase JMJD2E with similar IC₅₀ values (Table 1) (Sakurai et al., 2010, Molecular bioSystems 6:357-364), suggesting these are non-specific demethylase inhibitors. Some of these structures contain catechols, which are likely iron chelators and thus may be non-specific inhibitors (Baell & Holloway, 2010, J. Med. Chem. 53:2719-2740).

Another potent hit (2,4-PDCA) was identified as an inhibitor for multiple demethylases (King et al., 2010, PloS one 5:e15535; Rose et al., 2008, J. Med. Chem. 51:7053-7056; Thalhammer et al., 2011, Org. & Biomol. Chem. 9:127-135) and recently shown to inhibit the JARID1B catalytic domain (Kristensen et al., 2012, FEBS J. 279:1905-1914). The present studies indicate that 2,4-PDCA can also efficiently inhibit the JARID1 proteins, suggesting that it is a non-specific demethylase inhibitor.

The screening assay of the invention also identified several novel inhibitors. One such inhibitor, named PBIT, inhibited JARID1B at a low micromolar IC₅₀ value. Without wishing to be limited by theory, PBIT is unlikely to be an iron chelator as similar IC₅₀ values were obtained in experiments performed at both 15 μM and 50 μM Fe (II).

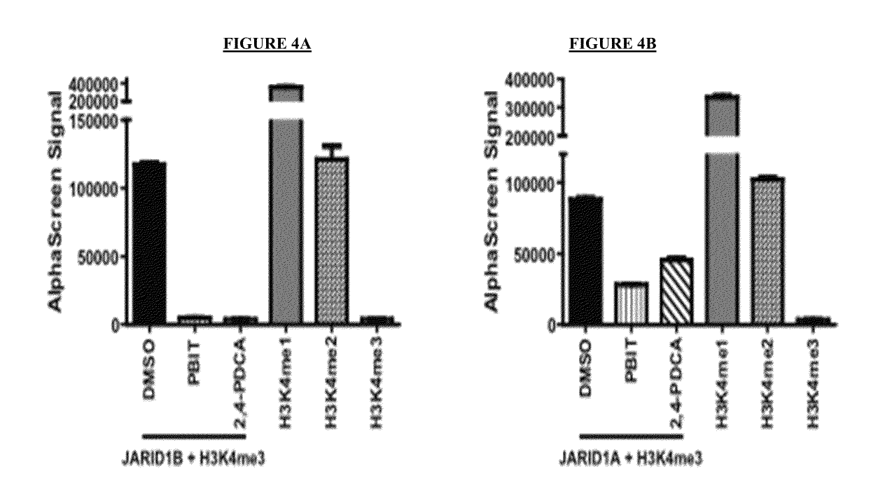

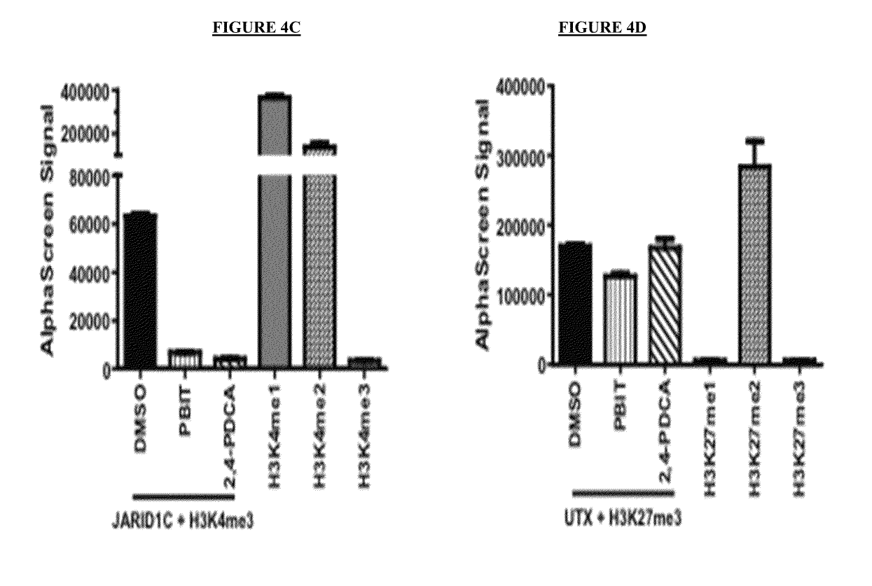

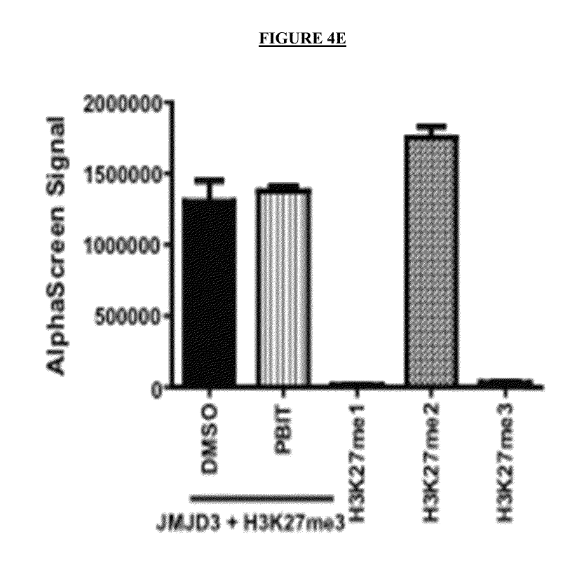

True iron chelators are more effective at lower iron concentrations by scavenging much of the available iron. PBIT potently inhibits JARID1A/B/C, suggesting that it can act as a pan-JARID1 inhibitor. 10 μM PBIT had a minimal effect on the H3K27 demethylases UTX and JMJD3 (FIGS. 4D-4E). In addition, the IC₅₀ value of PBIT for JMJD2E is 28 μM (King et al., 2010, PloS one 5:e15535). Without wishing to be bound by theory, although it may be possible that PBIT also inhibits other JmjC demethylases and hydroxylases, the results presented herein suggested that PBIT is specific for the JARID1 enzymes.

PBIT is a derivative of benzisothiazolinone (BIT), a widely used microbicide and fungicide used in home cleaning products (Dou et al., 2011, Bioorg. Med. Chem. 19:5782-5787). PBIT and its analogues were previously identified as inhibitors of salicylate synthase from Myocobacterium tuberculosis (Vasan et al., 2010, ChemMedChem 5:2079-2087). Derivatives of BIT are potential antiviral drugs, acting by inhibiting enzymes such as macrophage migration inhibitory factor (Jorgensen et al., 2011, Bioorg. Med. Chem. Lett. 21:4545-4549). The PBIT analogue ebselen exhibited an IC₅₀ of ˜6 μM against JARID1B (Table 1).

No crystal structure of the catalytic domains of the JARID1 enzymes has been published. Structure-guided virtual screen was used to identify potent UTX inhibitors (Kruidenier et al., 2012, Nature 488:404-408). In one aspect, structural studies of the JARID1B enzyme with its inhibitors may decipher their inhibitory mechanisms and to derive more potent inhibitors.

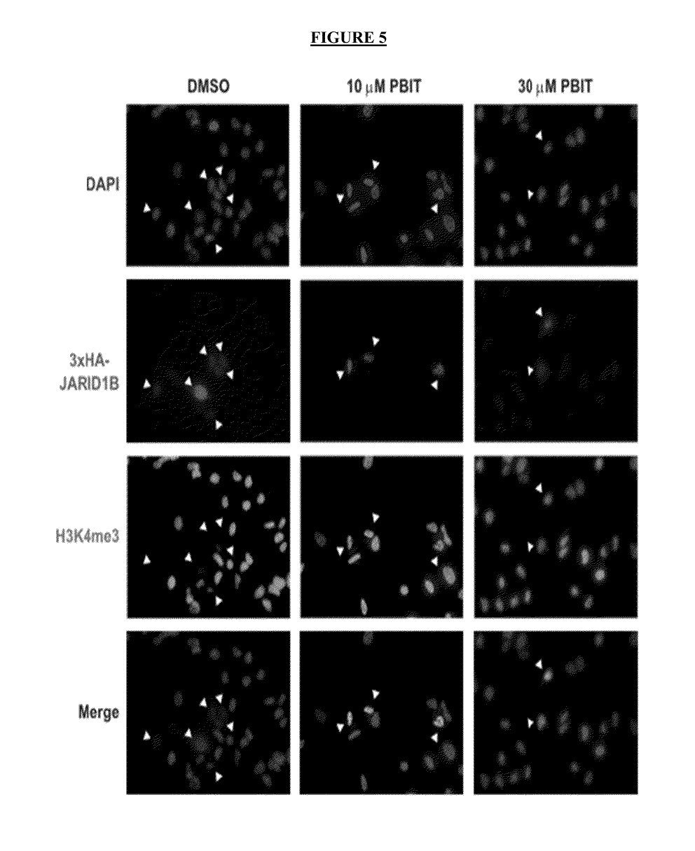

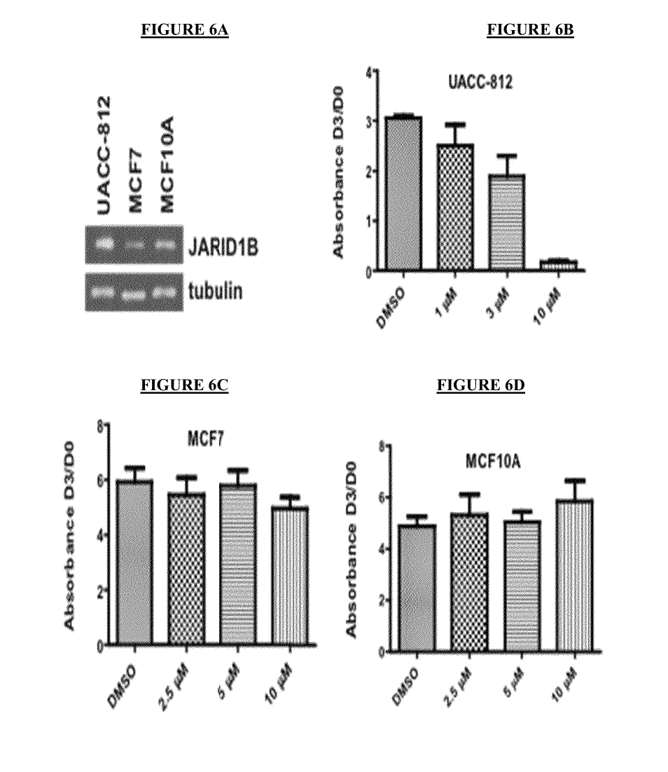

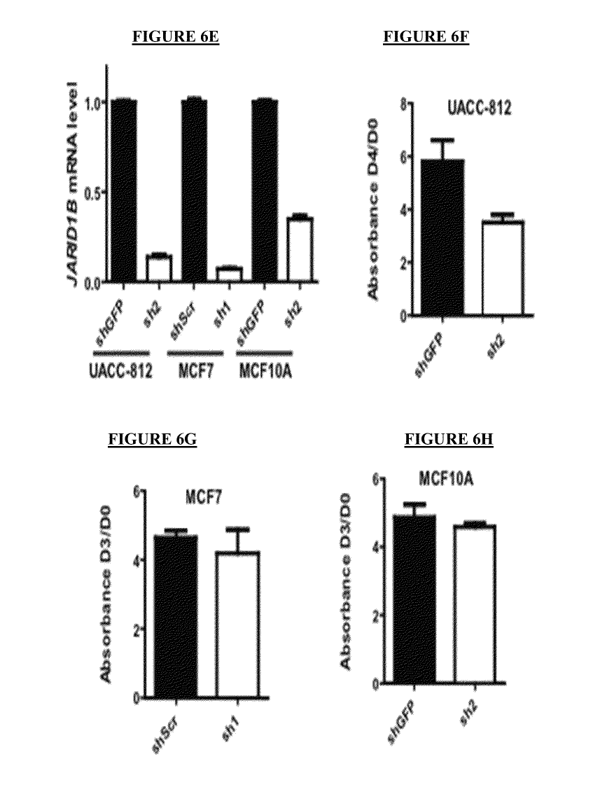

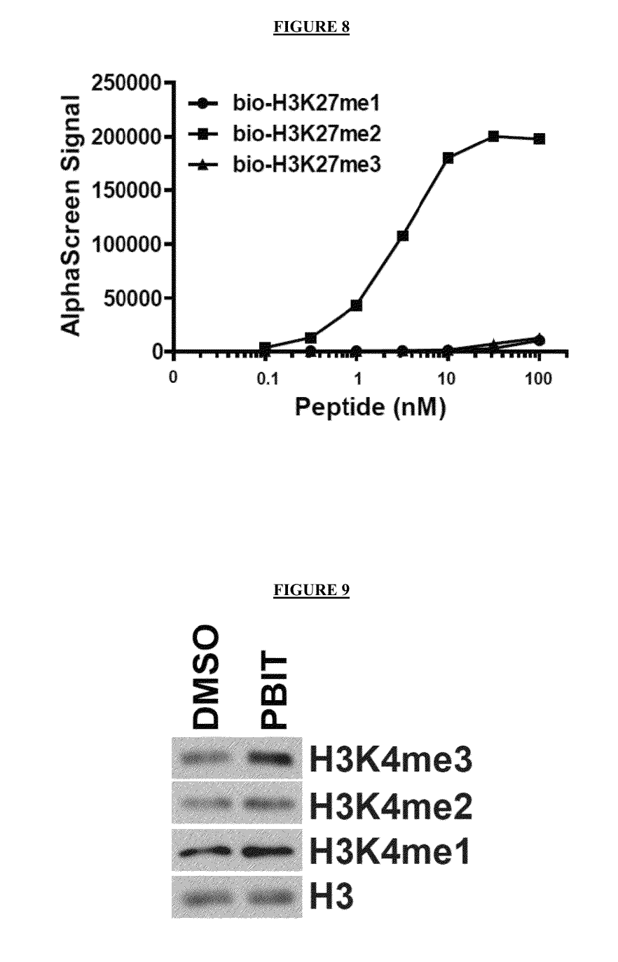

PBIT treatment prevented the JARID1B overexpression-induced decrease of H3K4me3 in HeLa cells (FIG. 5). In addition, treatment of MCF7 cells with PBIT increased global levels of H3K4me3 (FIG. 9), suggesting this compound entered the nucleus and inhibited JARID1 H3K4 demethylases. The cell based assays discussed herein showed that PBIT inhibited cell growth in a JARID1B level-dependent manner (FIGS. 6A-6D). Consistent with these experiments, JARID1B knockdown decreased the proliferation of UACC-812 cells, but not MCF7 or MCF10A cells (FIGS. 6E-6H). Without wishing to be limited by theory, the effect of JARID1B knockdown on UACC812 cells is not as dramatic as PBIT treatment, suggesting that either incomplete knockdown of JARID1B, or functional compensation of JARID1A contributes to proliferation and survival of HER2 positive (HER2+) UACC-812 cells.

JARID1B is overexpressed in HER2+ cells and human tumors, suggesting that PBIT may be used to treat the HER2+ subtype of breast cancer. Without wishing to be limited by theory, the fact that JARID1B knockdown did not affect the proliferation of MCF7 cells in the present studies may be due to the culture media used herein. Interestingly, PBIT treatment increased H3K4me3 level in MCF7 cells, but did not inhibit growth of these cells, suggesting that additional non-histone substrates of the JARID1 enzymes play critical roles in cell growth.

JARID1A and JARID1B knockout mouse are viable (Blair et al., 2011, Cancers 3:1383-1404; Klose et al., 2007, Cell 128:889-900; Schmitz et al., 2011, EMBO J. 30:4586-4600), suggesting that inhibition of JARID1A or JARID1B has minimal effects on normal cells in vivo. JARID1A loss inhibits tumorigenesis in two mouse endocrine cancer models (Lin et al., 2011, Proc. Natl. Acad. Sci. U.S.A. 108:13379-13386), suggesting that a JARID1A inhibitor may be used to treat these cancers. In addition, the tumors formed in the JARID1A knockout mice showed increased JARID1B expression, implying that inhibitors that block both JARID1A and JARID1B enzymes are more effective in preventing tumor formation (Lin et al., 2011, Proc. Natl. Acad. Sci. U.S.A. 108:13379-13386). The importance of JARID1 inhibitors may be confirmed in mouse models in which the endogenous JARID1 genes were replaced with the genes encoding catalytic inactive enzymes.

As illustrated herein, the JARID1 inhibitor PBIT has selective inhibitory activity on a HER2+ breast cancer cell line, and the efficacy of PBIT and its derivatives on breast cancer may be further investigated with additional cell lines and in xenograft or genetically engineered mouse cancer models. As the JARID1 enzymes contribute strongly to tumorigenesis and drug resistance in multiple cancer types (Blair et al., 2011, Cancers 3:1383-1404; Hou et al., 2012, Am. J. Trans1. Res. 4, 247-256), these inhibitors may also be effective for cancer therapy in those settings.

### Compositions

The invention includes a pharmaceutical composition comprising a compound, or a salt or solvate thereof, selected from the group consisting of: caffeic acid (also known as (E)-3-(3,4-dihydroxyphenyl)acrylic acid); esculetin (also known as 6,7-dihydroxy-2H-chromen-2-one);

a compound of formula (I):

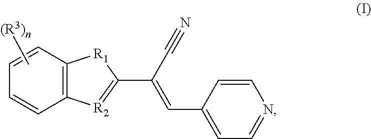

wherein in formula (I):
R¹ is S, O, NH or N(C₁-C₆ alkyl);
R² is N, CH or C—(C₁-C₆ alkyl); and
n is 0, 1, 2, 3 or 4, wherein each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy and carboxy; and,
   a compound of formula (II):

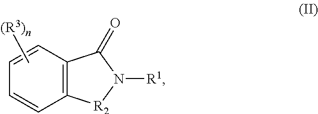

wherein in formula (II):
R¹ is C₁-C₆ alkyl, substituted C₁-C₆ alkyl, C₃-C₇ cycloalkyl, substituted C₃-C₇ cycloalkyl, aryl, substituted aryl, heteroaryl, substituted heteroaryl, heterocyclyl, substituted heretocyclyl, acyl, benzoyl, substituted benzoyl, or phenylacetyl;
R² is C(R₄)₂, O, S, C(O), S(O), S(O)₂ or Se;
n is 0, 1, 2, 3 or 4, wherein
   each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy, cyano and carboxy; and
   each occurrence of R⁴ is independently H, C₁-C₆ alkyl, or substituted C₁-C₆ alkyl.

In one embodiment, in formula (I) R¹ is S, NH or N(C₁-C₆ alkyl). In another embodiment, in formula (I) R¹ is S, NH or N(CH₃).

In one embodiment, in formula (I) R² is N.

In one embodiment, in formula (I) each occurrence of R³ is independently selected from the group consisting of C₁-C₆ alkyl, substituted C₁-C₆ alkyl, C₁-C₆ haloalkyl, C₃-C₆ cycloalkyl, heterocyclyl, substituted heterocyclyl, halogen, C₁-C₆ alkoxy, nitro, amino, acetamido, hydroxy and carboxy. In another embodiment, in formula (I) each occurrence of R³ is independently selected from the group consisting of C₁-C₆ alkyl, substituted C₁-C₆ alkyl, C₁-C₆ haloalkyl, halogen, C₁-C₆ alkoxy, nitro, amino, acetamido, hydroxy and carboxy. In yet another embodiment, in formula (I) R³ is CF₃ and n is 1.

In one embodiment, the compound of formula (I) is selected from the group consisting of (E)-3-(pyridin-4-yl)-2-(5-(trifluoromethyl)benzo[d]thiazol-2-yl)acrylonitrile; (E)-2-(1-methyl-1H-benzo[d]imidazol-2-yl)-3-(pyridin-4-yl)acrylonitrile;

or any combinations thereof.

In one embodiment, in formula (II) R¹ is aryl, substituted aryl, heteroaryl, or substituted heteroaryl. In another embodiment, in formula (II) R¹ is C₁-C₆ alkyl, aryl or substituted aryl. In yet another embodiment, the substituted aryl is substituted with at least one substituent selected from the group consisting of F, Cl, Br, methyl, ethyl, isopropyl, cyano and tert-butyl. In yet another embodiment, in formula (II) R¹ is phenyl, o-tolyl, m-tolyl, p-tolyl, o-fluorophenyl, m-fluorophenyl, p-fluorophenyl, o-chlorophenyl, m-chlorophenyl, p-chlorophenyl, o-isopropylphenyl, m-isopropylphenyl, p-isopropylphenyl or isopropyl.

In one embodiment, in formula (II) R² is S, SO₂, CH₂, C(O) or Se.

In one embodiment, in formula (II) each occurrence of R³ is independently selected from the group consisting of C₁-C₆ alkyl, substituted C₁-C₆ alkyl, C₁-C₆ haloalkyl, C₃-C₆ cycloalkyl, heterocyclyl, substituted heterocyclyl, halogen, C₁-C₆ alkoxy, nitro, amino, acetamido, hydroxy, cyano and carboxy. In another embodiment, in formula (II) each occurrence of R³ is independently selected from the group consisting of C₁-C₆ alkyl, C₁-C₆ haloalkyl, halogen, C₁-C₆ alkoxy, nitro, amino, cyano, acetamido, hydroxy and carboxy. In yet another embodiment, in formula (II) n is 0.

In one embodiment, the compound of formula (II) is selected from the group consisting of 2-(4-methylphenyl)-1,2-benzisothiazol-3(2H)-one; 2-phenylbenzo[d][1,2]selenazol-3(2H)-one, 2-(4-chlorophenyl)-5,6-difluorobenzo[d]isothiazol-3(2H)-one, 2-(4-chlorophenyl)-5-(trifluoromethyl)benzo[d]isothiazol-3(2H)-one, 2-(4-chlorophenyl)-6-isocyanobenzo[d]isothiazol-3(2H)-one or any combinations thereof.

In one embodiment, the compound of formula (II) is selected from the group consisting of:

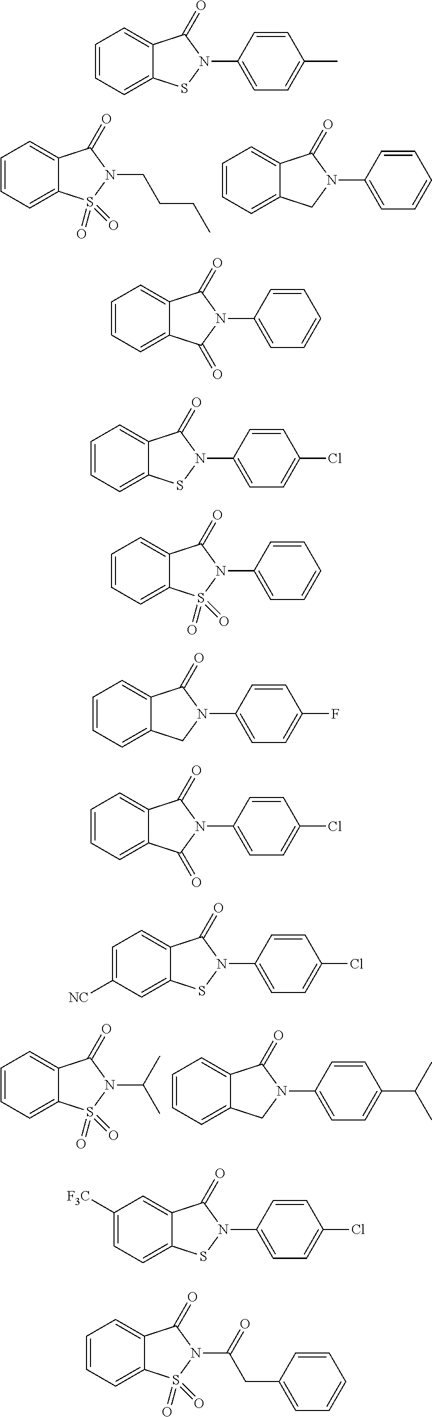

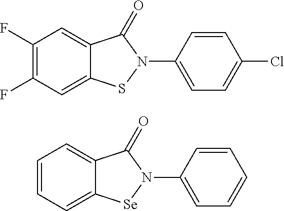

The compounds useful within the invention may be prepared according to the general methodology known to those skilled in the art, or purchased from commercial suppliers as appropriate.

### Salts

The compounds described herein may form salts with acids, and such salts are included in the present invention. In one embodiment, the salts are pharmaceutically acceptable salts. The term “salts” embraces addition salts of free acids or bases that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine.

All of these salts may be prepared from the corresponding compound by reacting, for example, the appropriate acid or base with the compound.

### Combination Therapies

In one embodiment, the compounds of the invention are useful in the methods of present invention in combination with at least one additional compound useful for preventing and/or treating cancer. These additional compounds may comprise compounds of the present invention or other compounds, such as commercially available compounds, known to treat, prevent, or reduce the symptoms of cancer. In one embodiment, the combination of at least one compound of the invention or a salt thereof and at least one additional compound useful for preventing and/or treating cancer has additive, complementary or synergistic effects in the prevention and/or treatment of cancer.

In one aspect, the present invention contemplates that a compound useful within the invention may be used in combination with a therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that are also useful in the invention.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In one embodiment, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In another embodiment, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

As used herein, combination of two or more compounds may refer to a composition wherein the individual compounds are physically mixed or wherein the individual compounds are physically separated. A combination therapy encompasses administering the components separately to produce the desired additive, complementary or synergistic effects. In one embodiment, the compound and the agent are physically mixed in the composition. In another embodiment, the compound and the agent are physically separated in the composition.

In one embodiment, the compound of the invention is co-administered with a compound that is used to treat cancer. The co-administered compound may be administered individually, or a combined composition as a mixture of solids and/or liquids in a solid, gel or liquid formulation or as a solution, according to methods known to those familiar with the art.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326), the median-effect equation (Chou & Talalay, 1984, Adv. Enzyme Regul. 22: 27-55), and through the use of isobolograms (Tallarida & Raffa, 1996, Life Sci. 58: 23-28). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Screening

The invention includes a high-throughput method of determining whether a compound inhibits JARID1B demethylase activity. The method comprises the step of providing tagged full length JARID1B enzyme. The method further comprises the step of incubating the tagged full length JARID1B enzyme with the compound and tagged H3K4Me3 peptide in a system at a determined temperature for a determined period of time. The method further comprises the step of determining whether any H3K4me2/1 peptide is formed in the system. If any H3K4me2/1 peptide is formed in the system, the compound is determined to inhibit JARID1B demethylase activity.

In one embodiment, the tagged full length JARID1B enzyme comprises FLAG-tagged full length JARID1B enzyme. In another embodiment, the tagged H3K4Me3 peptide comprises biotinylated H3K4Me3 peptide. In yet another embodiment, the system further comprises alpha-ketoglutarate, an iron (II) salt and ascorbate. In yet another embodiment, determining whether any H3K4me2/1 peptide is formed in the system comprises incubating an H3K4me2 antibody or H3K4me1 antibody with at least a portion of the system. In yet another embodiment, the system is heterogeneous. In yet another embodiment, the tagged H3K4Me3 peptide is immobilized on a solid support.

### Methods

The invention includes a method of treating or preventing cancer in a subject. The method comprises administering to the subject a therapeutically effective amount of a pharmaceutical composition comprising a compound selected from the group consisting of:

caffeic acid (also known as (E)-3-(3,4-dihydroxyphenyl)acrylic acid);
esculetin (also known as 6,7-dihydroxy-2H-chromen-2-one);

a compound of formula (I):

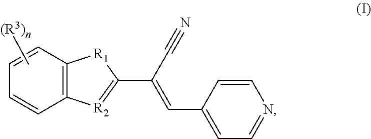

wherein in formula (I):
R¹ is S, O, NH or N(C₁-C₆ alkyl);
R² is N, CH or C—(C₁-C₆ alkyl); and
n is 0, 1, 2, 3 or 4, wherein each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy and carboxy; and,

a compound of formula (II):

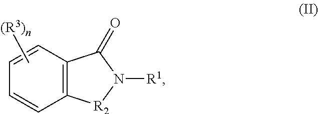

wherein in formula (II):
R¹ is C₁-C₆ alkyl, substituted C₁-C₆ alkyl, C₃-C₇ cycloalkyl, substituted C₃-C₇ cycloalkyl, aryl, substituted aryl, heteroaryl, substituted heteroaryl, heterocyclyl, substituted heretocyclyl, acyl, benzoyl, substituted benzoyl, or phenylacetyl;
R² is C(R₄)₂, O, S, C(O), S(O), S(O)₂ or Se;
n is 0, 1, 2, 3 or 4, wherein:
   each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy, cyano and carboxy; and
   each occurrence of R⁴ is independently H, C₁-C₆ alkyl or substituted C₁-C₆ alkyl.

In one embodiment, administration of the pharmaceutical composition to the subject inhibits at least one JARID1 enzyme in the subject. In another embodiment, the at least one JARID1 enzyme comprises JARID1B. In yet another embodiment, the at least one JARID1 enzyme comprises JARID1A. In yet another embodiment, the at least one JARID1 enzyme comprises JARID1A and JARID1B.

In one embodiment, the cancer comprises a solid cancer. In another embodiment, the solid cancer is selected from the group consisting of breast cancer, prostate cancer, melanoma, and any combinations thereof. In yet another embodiment, the breast cancer comprises HER2-positive breast cancer. In yet another embodiment, the HER2-positive breast cancer is resistant to trastuzumab.

In one embodiment, the subject is further administered an additional compound selected from the group consisting of a chemotherapeutic agent, an anti-cell proliferation agent and any combination thereof. In another embodiment, the chemotherapeutic agent comprises an alkylating agent, nitrosourea, antimetabolite, antitumor antibiotic, plant alkyloid, taxane, hormonal agent, bleomycin, hydroxyurea, L-asparaginase, or procarbazine. In yet another embodiment, the anti-cell proliferation agent comprises granzyme, a Bcl-2 family member, cytochrome C, or a caspase.

In one embodiment, the pharmaceutical composition and the additional compound are co-administered to the subject. In another embodiment, the pharmaceutical composition and the additional compound are co-formulated and co-administered to the subject. In yet another embodiment, the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combinations thereof. In yet another embodiment, the subject is a mammal. In yet another embodiment, the mammal is a human.

### Kits

The invention includes a kit comprising an applicator, an instructional material for use thereof, and a compound selected from the group consisting of:

caffeic acid (also known as (E)-3-(3,4-dihydroxyphenyl)acrylic acid);
esculetin (also known as 6,7-dihydroxy-2H-chromen-2-one);
a compound of formula (I):

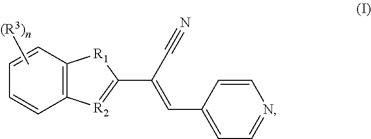

wherein in formula (I):
R¹ is S, O, NH or N(C₁-C₆ alkyl);
R² is N, CH or C—(C₁-C₆ alkyl); and
n is 0, 1, 2, 3 or 4, wherein each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy and carboxy; and,
   a compound of formula (II):

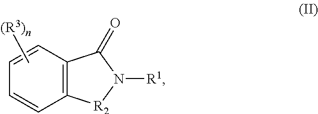

wherein in formula (II):
R¹ is C₁-C₆ alkyl, substituted C₁-C₆ alkyl, C₃-C₇ cycloalkyl, substituted C₃-C₇ cycloalkyl, aryl, substituted aryl, heteroaryl, substituted heteroaryl, heterocyclyl, substituted heretocyclyl, acyl, benzoyl, substituted benzoyl or phenylacetyl;
R² is C(R₄)₂, O, S, C(O), S(O), S(O)₂ or Se;
n is 0, 1, 2, 3 or 4, wherein:
   each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy, cyano and carboxy; and
   each occurrence of R⁴ is independently H, C₁-C₆ alkyl, or substituted C₁-C₆ alkyl.

The instructional material included in the kit comprises instructions for preventing or treating cancer in a subject. The instructional material recites that the subject is administered a therapeutically effective amount of a pharmaceutical composition comprising the compound contained in the kit. In one embodiment, the cancer comprises breast cancer, prostate cancer, melanoma, and any combinations thereof.

### Pharmaceutical Compositions and Formulations

The invention includes the use of pharmaceutical compositions of at least one compound of the invention or a salt thereof to practice the methods of the invention.

Such a pharmaceutical composition may consist of at least one compound of the invention or a salt thereof, in a form suitable for administration to a subject, or the pharmaceutical composition may comprise at least one compound of the invention or a salt thereof, and one or more pharmaceutically acceptable carriers, one or more additional ingredients, or some combination of these. The at least one compound of the invention may be present in the pharmaceutical composition in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, liposomal preparations, coated particles, resealed erythrocytes containing the active ingredient, and immunologically-based formulations. The route(s) of administration are readily apparent to the skilled artisan and depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions which are suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 100 mg/kg of body weight/per day. One of ordinary skill in the art is able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments thereinbetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of cancer in a patient.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing cancer in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically- or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 24 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials

Unless otherwise noted, all remaining starting materials were obtained from commercial suppliers and used without purification.

### Histone Peptides and Antibodies

C-terminal biotinylated (bio-) peptides used in assays were as follows. H3K4me3 [ART-K(Me3)-GTARKSTGGKAPRKQLA-GGK(Biotin); SEQ ID NO:1], H3K4me2 [ART-K(Me2)-GTARKSTGGKAPRKQLA-GGK(Biotin); SEQ ID NO:2], H3K4me1 [ART-K(Me1)-QTARKSTGGKAPRKQLA-GGK(Biotin); SEQ ID NO:3], H3K27me3 (ATKAAR-K(Me3)-SAPATGGVKKPHRYRPG-GK(Biotin); SEQ ID NO:4], H3K27me2 (ATKAAR-K(Me2)-SAPATGGVKKPHRYPG-GK(Biotin); SEQ ID NO:5], and H3K27me1(ATKAAR-K(Me1)-SAPATGGVKKPHRYRPG-GK(Biotin); SEQ ID NO:6] were obtained from AnaSpec.

Anti-H3K4me3 polyclonal antibody (ab8580), anti-H3K4me2 polyclonal antibody (ab7766), anti-H3K4me1 polyclonal antibody (ab8895), and anti-H3 polyclonal antibody (ab1791) were purchased from Abcam, and anti-H3K27me2 polyclonal antibody (07-452) was obtained from Upstate. Anti-JARID1A monoclonal antibody (3876S) was purchased from Cell Signaling, anti-JARID1B polyclonal antibody (A301-813A) and anti-JARID1C polyclonal antibody (A301-035A) were obtained from Bethyl Laboratories, anti-UTX antibody (M30076) was from Abmart, and anti-HA antibody (MMS-101P) was from Covance.

### Cell Lines

Sf21 insect cells were cultured in Grace's medium with 10% fetal bovine serum (FBS) and 1% penicillin/streptomycin (p/s). MCF7, UACC-812 and SKBR3 cells were cultured in RPMI 1640 with 10% FBS and 1% p/s. MCF10A cells were cultured in Dulbecco's modified Eagle's medium:Ham's F12 medium (1:1), 5% horse serum, 0.1 μg/ml cholera toxin, 10 ng/ml insulin, 0.5 μg/ml hydrocortisone, 20 ng/ml epidermal growth factor (EGF) and 1% p/s. SKBR3-R cells were generated by treating SKBR3-S cells with trastuzumab at each indicated concentration for about two weeks. SKBR3-R cells were maintained in RPMI 1640 with 10% FBS, 1% p/s and 100 μg/ml trastuzumab. 1445, YUAME, YULAC, and YURIF cells were cultured in OPTI-MEM with 5% FBS and 1% p/s.

### Enzyme Production

Sf21 cells infected with baculoviruses expressing FLAG-JARID1A (Klose et al., 2007, Cell 128:889-900), FLAG-JARID1B (Yamane et al., 2007, Mol. Cell 25:801-812), FLAG-JARID1C (Iwase et al., 2007, Cell 128:1077-1088), or His-FLAG-UTX (Agger et al., 2007, Nature 449:731-734) were cultured at 27° C. for three days, and the FLAG-tagged enzymes were purified via anti-FLAG M2 beads (Sigma) (FLAG; SEQ ID NO:7). Purification of these histone demethylases was confirmed by coomassie staining and western blot analysis using the specific antibodies against these enzymes.

### Histone Demethylase Assay

Histone demethylase assays were performed in 384 well white plates (Corning 3574). Demethylase buffer conditions for FLAG-JARID1B were as follows: 10 μM α-KG, 100 μM ascorbate, 50 μM (NH₄)₂Fe(SO₄)₂, 50 mM Hepes (pH 7.5), 0.01% (v/v) Tween 20, and 0.1% (w/v) bovine serum albumin. The demethylase reactions included 64 nM bio-H3K4me3 peptide alone or in the presence of 4 nM FLAG-JARID1B enzyme in a 10 μl reaction at 25° C. for 30 minutes. As a positive control, 64 nM bio-H3K4me2 peptide was assayed in the absence of enzyme. Assay conditions for FLAG-JARID1C is the same as for FLAG-JARID1B except that 20 nM enzyme was used. For FLAG-JARID1A, the demethylase buffer was similar to FLAG-JARID1B, except that 125 μM α-KG and 13 nM FLAG-JARID1A enzyme were used. The His-FLAG-UTX and FLAG-JMJD3 demethylase assays also employed the same buffer conditions as for FLAG-JARID1B, with 64 nM bio-H3K27me3 peptide assayed with or without 25 nM His-FLAG-UTX enzyme or 50 nM FLAG-JMJD3 (BPS Bioscience, 50115), and 64 nM bio-H3K27me2 peptide as a positive control. JARID1A, JARID1C, UTX, and JMJD3 histone demethylase assays proceeded at 37° C. for 1 hour.

### AlphaScreen Assay

The AlphaScreen General IgG (Protein A) detection kit was obtained from PerkinElmer. Demethylated H3K4 products were detected using AlphaScreen antibody/bead mix containing 7.5 mM ethylenediaminetetraacetic acid (EDTA) and 0.15 μg/ml anti-H3K4me1 antibody in a final volume of 20 μl. For detection of demethylated H3K27 products, the AlphaScreen antibody/bead mix containing 7.5 mM EDTA and 0.15 μg/ml anti-H3K27me2 antibody in a final volume of 20 μl. The luminescence signal was measured using the Envision (PerkinElmer) or Pherastar (BMG Labtech) Platereaders.

### Drug Screening Libraries and Conditions for JARID1B

FLAG-JARID1B was screened against 15,134 compounds. These compound libraries were from the Yale Center for Molecular Discovery. The libraries screened include the MicroSource Gen-Plus, MicroSource Pure Natural Products, NIH Clinical Collection, Enzo Epigenetics, Yale Compound, and ChemBridge MW-Set libraries, plus selected plates from the Maybridge Diversity, ChemBridge MicroFormats, DIVERSet and ChemDiv libraries containing 8-hydroxyquinolone analogs.

The first five libraries were screened twice, once under the standard demethylase assay conditions and once under similar conditions except that 1 mM α-KG was included. Compounds dissolved in dimethyl sulfoxide (DMSO) were pintooled into a 384 well plate containing bio-H3K4me3 peptide in demethylase buffer to a final concentration of 20 μM. The reactions were initiated by the addition of 4 nM FLAG-JARID1B and detected as described elsewhere herein. To eliminate the false positive hits, a counterscreen was performed against bio-H3K4me2 in the absence of enzyme. IC₅₀ values were generated from dose response curves using 0.1 μM to 11 μM of compound and 15 μM or 50 μM Fe (II).

### Drug Screening Libraries and Conditions for JARID1B

FLAG-JARID1A was screened against 9,600 compounds. These compound libraries were from the Yale Center for Molecular Discovery. The libraries screened include the MicroSource Gen-Plus, MicroSource Pure Natural Products, NIH Clinical Collection, Enzo Epigenetics, and ChemBridge MW-Set libraries. Compounds dissolved in dimethyl sulfoxide (DMSO) were pintooled into a 384 well plate containing bio-H3K4me3 peptide in demethylase buffer to a final concentration of 20 μM. The reactions were initiated by the addition of 13 nM FLAG-JARID1A, and detected as described above. To eliminate the false positive hits, a counterscreen was performed against bio-H3K4me2 in the absence of enzyme. IC₅₀ values were generated from dose response curves using 0.1 μM to 11 μM of compound and 50 μM Fe (II).

### Chemicals

2-4(4-methylphenyl)-1,2-benzisothiazol-3(2H)-one (PBIT) (PH009215) and 2,4-pyridinedicarboxylic acid monohydrate (2,4-PDCA) (P63395) were purchased from Sigma Aldrich. DMSO (9224-01) was purchased from J.T. Baker.

### Immunostaining

pcDNA3.1(−)-3×HA-JARID1B construct was generated by inserting 3×HA-JARID1B between BamHI and XbaI of pcDNA3.1(−) vector. MCF7 cells were plated on 12 mm circle coverslips in 24-well plates and transfected with pcDNA3.1(−)-3×HA-JARID1B in the presence of 0, 10 or 30 μM PBIT. After incubation for 24 hours, the cells were fixed, permeabilized, and stained with antibodies against HA and H3K4me3 for 2 hours. The coverslips were then incubated with anti-mouse Alexa-546 (Invitrogen, A-11003) and goat anti-rabbit Envision (Dako, K4002) for 1 hour. Cy5-Tyramide (Perkin Elmer, NEL775001KT) and 4,6-diamidino-2-phenylindole dihydrochloride (DAPI) (Biotium, 40011) were used to visualize 3×HA-tagged JARID1B and nuclei, respectively. The slides were sealed and analyzed under an Olympus fluorescence microscope.

### Histone Extraction and Western Blot

MCF7 cells treated with PBIT (10 μM) or DMSO (0.1%) for 72 hours were harvested and lysed with PBS containing 0.5% Triton X-100. Nuclei were spun down by centrifugation at 6,500×g for 10 minutes, and the pellets were re-suspended in 0.2 N HCl. The histones were extracted overnight, and cellular debris was removed by centrifugation. The samples were loaded onto 16% SDS-PAGE gels and probed with antibodies against H3K4me3, H3K4me2, H3K4me1 and H3.

### Cell Proliferation Assay

The colorimetric assay (WST-1 reagent) from Roche (11644807001) was performed in 96 well white clear bottom plates (Costar, 3610). 1,000 cells were seeded per well (in quadruplicate) overnight, and PBIT was added to the cells to the indicated concentration for 72 hrs. 0.01% DMSO was included as the control. The WST-1 reagent was added (5 μl per well) for 4 hrs, and OD 440 nm absorbance (which reflects the number of viable cells) was measured with the BioTek Synergy Mx Platereader.

### Generation of JARID1B Knockdown Cell Lines

Stable knockdown of JARID1B in UACC-812, MCF7, MCF10A and SKBR3 cells were performed as described previously (Yang et al., 2007, Mol. Cell 28:15-27) using two lentiviral shRNAs, pLK0.1-JARID1B sh1 (targeting CGAGATGGAATTAACAGTCTT; SEQ ID NO:8) and pLK0.1-JARID1B sh2 (targeting AGGGAGATGCACTTCGATATA; SEQ ID NO:9). pLKO.1-shScr (scramble shRNA control) was described previously (Yang et al., 2007, Mol. Cell 28:15-27; Niu et al., 2012, Oncogene 31:776-786). pLKO.1-shGFP control shRNA was gift from William Hahn (Dana-Farber Cancer Institute, Boston, Mass.). Knockdown cells were selected and maintained in medium containing 2 μg/ml puromycin.

### Real Time Reverse-Transcription (RT) PCR

Real time RT-PCR experiments were performed as described in Lin et al., 2011, Proc. Natl. Acad. Sci. U.S.A. 108:13379-13386. Values were normalized to the level of GAPDH or ACTIN mRNA. Primers specific for JARID1B were hPLU1F2 (CCATAGCCGAGCAGACTGG; SEQ ID NO:10) and hPLU1R2 (GGATACGTGGCGTAAAATGAAGT; SEQ ID NO:11). Primers specific for GAPDH were hGAPDHF (CGAGATCCCTCCAAAATCAA; SEQ ID NO:12) and hGAPDHR (GTCTTCTGGGTGGCAGTGAT; SEQ ID NO:13). Primers specific for ACTIN were described in Yan et al., 2007, Mol. Cell. Biol. 27:2092-2102.

### Example 1

### AlphaScreen Assay Setup

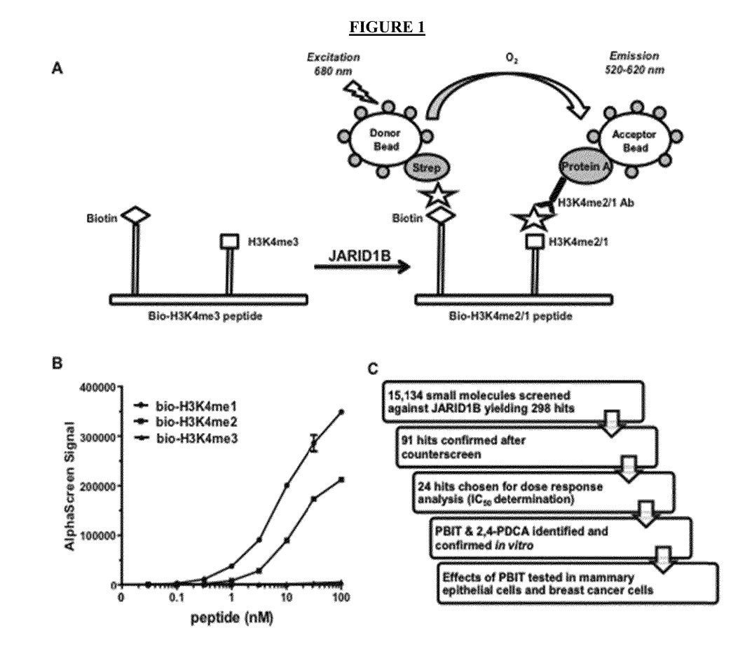

To identify small molecule inhibitors of the JARID1B enzyme, AlphaScreen technology was employed to monitor JARID1B activity (FIG. 1A) (Kawamura et al., 2010, Anal. Biochem. 404:86-93). In the demethylase assays, a biotinylated H3K4me3 peptide substrate underwent demethylation by JARID1B. The demethylated products (bio-H3K4me2/1) were detected by interaction with both streptavidin coated donor beads (via biotin label) and Protein A coated acceptor beads (via interaction with the H3K4me2/1 antibody). Laser excitation leads to a luminescence signal that corresponds to the amount of bio-H3K4me2/1 and thus demethylase activity. Antibody optimization for the AlphaScreen assay in the absence of enzyme was performed using various antibodies against H3K4me2 and H3K4me1. Among these antibodies, the H3K4me1 antibody can generate homogenous luminescence signals for both the bio-H3K4me1 and bio-H3K4me2 peptides (FIG. 1B). More importantly, the signal for the bio-H3K4me1 peptide is about twice that of the bio-H3K4me2 peptide. Therefore, the AlphaScreen signal can also indicate the degree of demethylation.

### Example 2

### Characterization of JARID1B

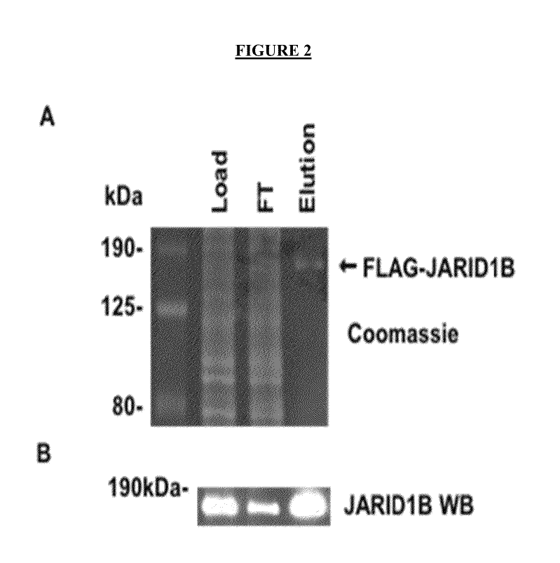

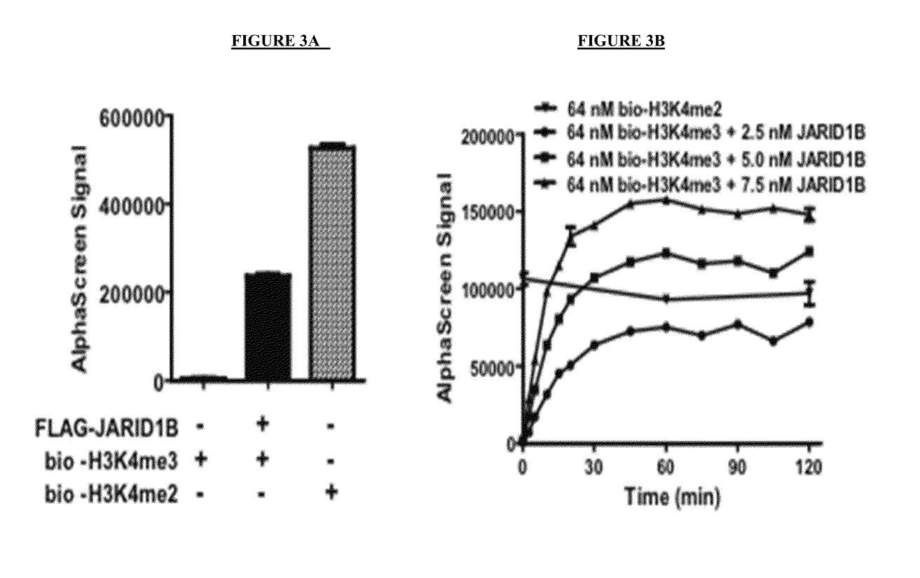

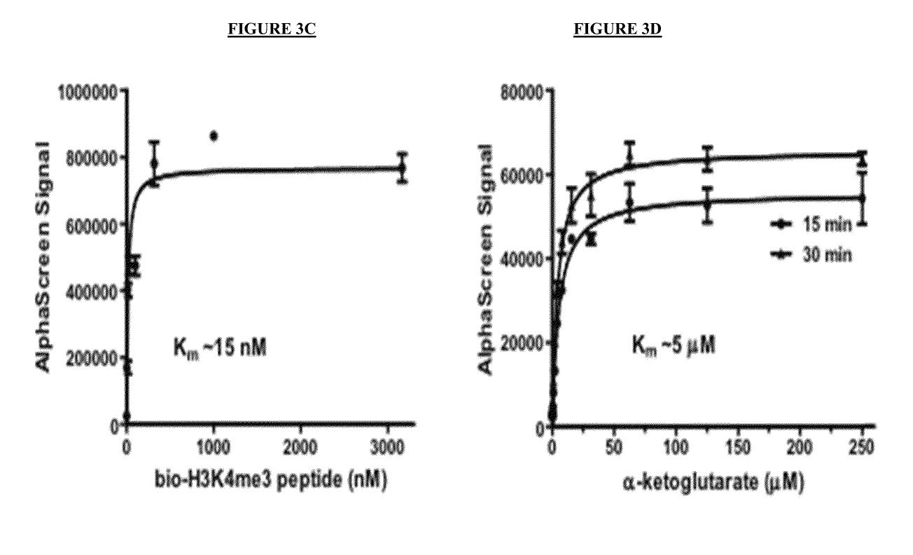

The FLAG tagged full length JARID1B enzyme was expressed in Sf21 insect cells using FLAG-JARID1B baculoviruses and affinity purified using anti-FLAG antibody. FLAG-JARID1B was analyzed by SDS-PAGE for purity (FIG. 2A), and by western blot for JARID1B expression (FIG. 2B). To assess the activity of FLAG-JARID1B, demethylase assays were performed in triplicate using AlphaScreen platform in the presence and absence of JARID1B (FIG. 3A). AlphaScreen signal was detected in demethylase assays performed in the presence of both the bio-H3K4me3 peptide and FLAG-JARID1B. Assays performed using only the bio-H3K4me2 peptide served as a positive control.

To optimize screening conditions, FLAG-JARID1B activity was further investigated in a time course and enzyme titration experiment (FIG. 3B). Robust AlphaScreen signal was observed using only 5 nM FLAG-JARID1B, and the demethylase reaction was essentially complete after 30 min at room temperature. Further optimization of the FLAG-JARID1B demethylase reaction included titration of the bio-H3K4me3 peptide (FIG. 3C), α-KG (FIG. 3D), Fe (II) and ascorbate. These results showed that the Kₘ for bio-H3K4me3 is ˜15 nM and for α-KG is −5 μM. Final screening conditions for JARID1B were 4 nM enzyme, 64 nM bio-H3K4me3 peptide, 50 μM Fe (II), 10 μM α-KG, and 100 μM ascorbate, and demethylase reactions proceeded for 30 min at room temperature.

### Example 3

### High-Throughput Screening for JARID1B Inhibitors

FLAG-JARID1B was screened against 15,134 compounds from several small molecule libraries. At a threshold of inhibition more than 3 standard deviations (about 30-40% inhibition), 298 hits were identified (FIG. 1C and Table 2). Among these hits, 91 compounds were validated after a counter-screen using the bio-H3K4me2 peptide, which eliminates the compounds that have non-specific effect on AlphaScreen assays (FIG. 1C and Table 3). Of these confirmed hits, 24 compounds were selected based on their inhibition efficiency and structure for further dose response analysis. As iron chelators tend to inhibit more efficiently at lower iron concentrations, dose response analysis was performed in the presence of 15 μM and 50 μM Fe (II) to eliminate potential iron chelators. Many of these 24 compounds yielded low micromolar IC₅₀ values (Table 1 and Table 4), including several known demethylase inhibitors, such as 2,4-PDCA and catechols. As 2,4-PDCA was recently shown to inhibit the JARID1B catalytic core (23), these results validated the present screening method. Consistent with this previous study, 2,4-PDCA inhibited JARID1B with an IC₅₀ value of about 5 μM (Table 4).

Among the top inhibitors, ChemBridge compounds 7812482 and 6339039 have very similar structures. Caffeic acid and esculetin are catechols, which are potential iron chelators (Sakurai et al., 2010, Molecular bioSystems 6:357-364; Baell & Holloway, 2010, J. Med. Chem. 53:2719-2740). Consistent with this, lower IC₅₀ values for these catechols were observed in the presence of 15 μM Fe (II) than in the presence of 50 μM Fe (II). Furthermore, caffeic acid was identified as an inhibitor of JMJD2C/KDM4C and UTX/KDM6A (Nielsen et al., 2012, FEBS Lett. 586:1190-1194).

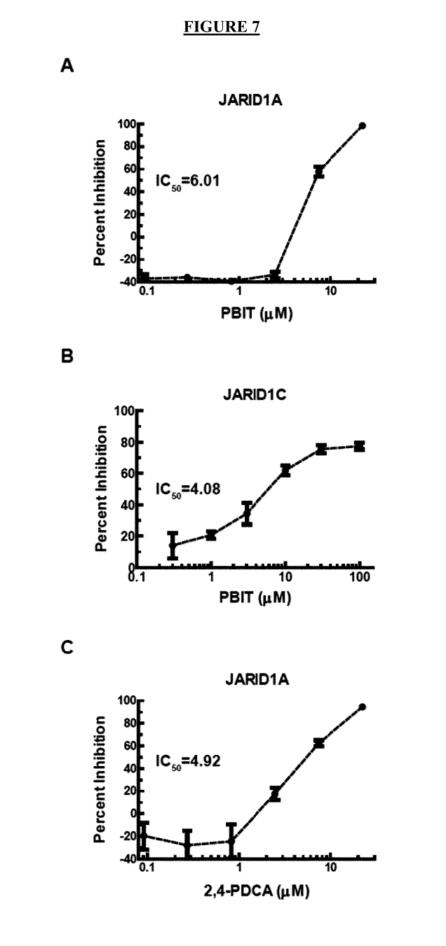

A novel demethylase inhibitor, 2-4(4-methylphenyl)-1,2-benzisothiazol-3(2H)-one (PBIT), was also identified as a potent inhibitor of JARID1B, with an IC₅₀ value of about 3 μM at both 15 μM and 50 μM Fe (II) (Table 4). To address the inhibitory specificity of PBIT and 2,4-PDCA against other JARID1 demethylases, these two compounds were tested against JARID1B (FIG. 4A), JARID1A (FIG. 4B), and JARID1C (FIG. 4C). 10 μM PBIT inhibited the activities of all the JARID1 enzymes tested (FIG. 4A-C). Dose response analysis showed that PBIT is also a potent inhibitor of JARID1A and JARID1C, with the IC₅₀ values of 6 μM and 4.9 μM, respectively (FIGS. 7A-7B). Similarly, 2,4-PDCA inhibited all the JARID1 proteins tested, with an IC₅₀ of 4.3 μM for JARID1B and 4.1 μM for JARID1A (FIG. 4A-C, FIG. 7C and Table 3).

The specificity of PBIT and 2,4-PDCA for other JmjC domain containing histone lysine demethylases was examined After initial optimization of the AlphaScreen assay for antibody specificity in the absence of enzyme (FIG. 8), analysis of the H3K27 demethylases UTX/KDM6A and JMJD3/KDM6B revealed that PBIT did not inhibit the activity of UTX or JMJD3 at 10 μM (FIGS. 4D-4E). Likewise, 2,4-PDCA did not inhibit UTX at 10 μM (FIG. 4D). These results suggest that PBIT is a specific inhibitor of the JARID1 enzymes.

### Example 4

### In Vivo Validation of Inhibitors

To determine whether JARID1B could be inhibited by PBIT in cells, HeLa cells overexpressing full-length JARID1B were treated with 10 μM or 30 μM PBIT. As expected, in JARID1B transfected cells, the levels of H3K4me3 decreased dramatically compared with untransfected cells (FIG. 5). In contrast, in PBIT treated cells, this decrease was blocked (FIG. 5).

To determine whether PBIT affects H3K4 methylation globally in vivo, H3K4 methylation levels were analyzed in histone extracts prepared from MCF7 cells after exposure to PBIT. Treatment of MCF7 cells with 10 μM PBIT for 72 hours led to a dramatic increase of H3K4me3 levels, while H3K4me2 and H3K4me1 levels did not change significantly (FIG. 9). Similar results were obtained from MCF10A cells and 1445 mouse melanoma cells, indicating that PBIT acts to inhibit the JARID1 H3K4 demethylases in vivo.

PBIT inhibits cell proliferation in a JARID1B level-dependent manner. JARID1B is over-expressed in human breast tumors (Lu et al., 1999, J. Biol. Chem. 274:15633-15645). To evaluate whether inhibition of JARID1B activity has any growth inhibitory effect on breast cancer cells, the expression levels of JARID1B in immortalized human mammary epithelial cells (MCF10A) and human breast cancer cell lines (MCF7 and UACC-812) were analyzed. UACC-812 cells expressed a higher level of JARID1B than MCF7 or MCF10A cells (FIG. 6A). These cells were then treated with PBIT and analyzed for cell proliferation. Consistent with the higher expression levels of JARID1B in UACC-812 cells, exposure to 10 μM PBIT killed most of the UACC-812 cells (FIG. 6B), but showed minimal toxicity to MCF7 cells (FIG. 6C) and MCF10A cells (FIG. 6D). Similar results were obtained when JARID1B was downregulated by shRNA (FIG. 6E), where JARID1B shRNA inhibited proliferation of UACC-812 cells (FIG. 6F), but not MCF7 cells (FIG. 6G) or MCF10A cells (FIG. 6H).

### Example 5

### PBIT Inhibited Proliferation of Trastuzumab Resistant Cells

JARID1B was previously identified as a gene that is down-regulated by 4D5 antibody (humanized version of 4D5 is trastuzumab/Herceptin) (Tan et al., 2003, J. Biol. Chem. 278:20507-20513). In one embodiment, in a subset of trastuzumab resistant cells, JARID1B expression is not affected by trastuzumab treatment and JARID1B activation contributes to trastuzumab resistance. To determine the mechanisms by which patients become resistant to trastuzumab treatment, a cell based model was set up to mimic the in vivo situation.

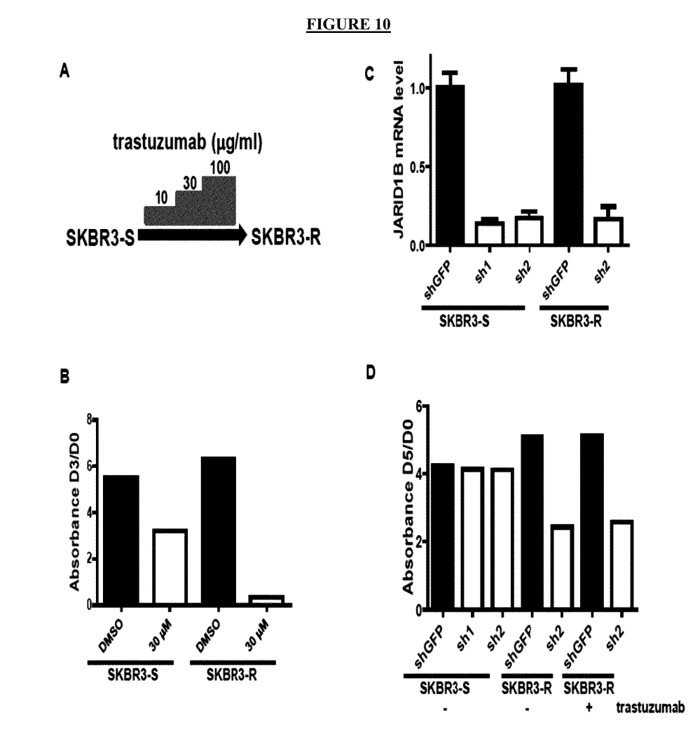

SKBR3 HER2+ cells (herein referred to as “SKBR3-S cells”), which are normally trastuzumab sensitive, were selected. By treating these cells with increasing concentrations of trastuzumab, trastuzumab resistant SKBR3 (SKBR3-R) cells were generated (FIG. 10A). IC₅₀s of trastuzumab for these trastuzumab cells are more than 300 μg/ml. Treatment with 30 μM PBIT killed most of the SKBR3-R cells, but only had small inhibitory effects on the growth of SKBR3-S cells (FIG. 10B). Similar results were obtained when JARID1B was downregulated by shRNA (FIG. 10C), where knockdown of JARID1B decreased proliferation of SKBR3-R cells in the presence or absence of trastuzumab, while it did not affect proliferation of SKBR3-S cells (FIG. 10D).

### Example 6

### PBIT Inhibited Proliferation of Melanoma Cells

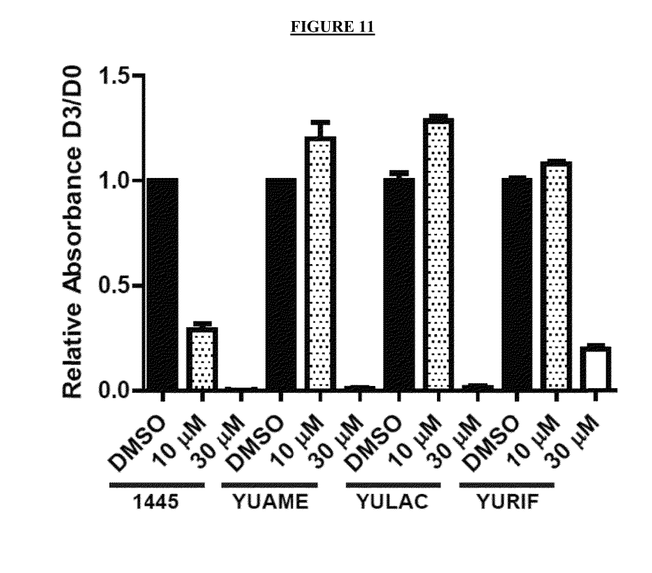

A panel of mouse and human melanoma cells was treated with 0, 10 and 30 μM PBIT and their proliferation rates were assessed using WST-1 assays. All the melanoma cell lines examined were sensitive to PBIT treatment, and 1445 mouse melanoma cells were most sensitive, while YURIF melanoma cells were least sensitive to PBIT treatment (FIG. 11).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Example 7

### Characterization of JARID1A

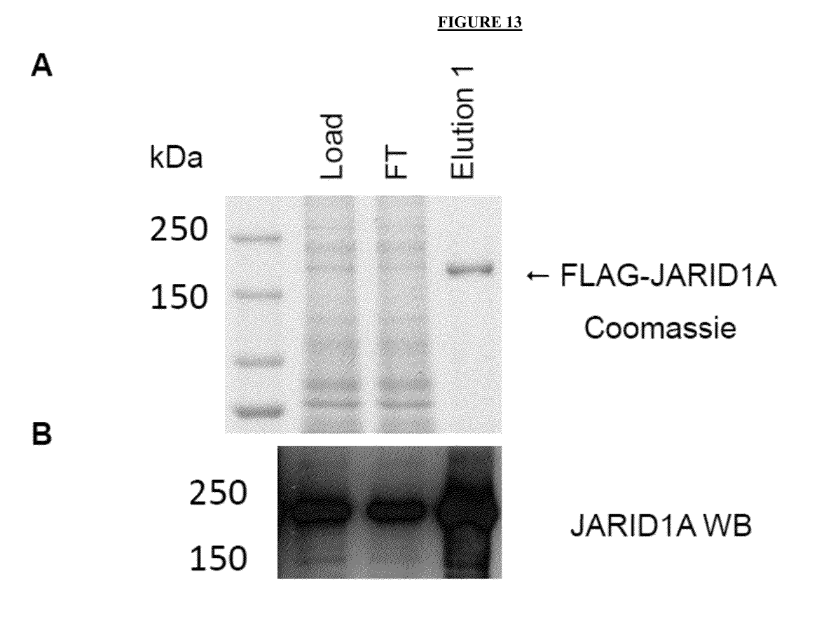

The FLAG tagged JARID1A enzyme was expressed in Sf21 insect cells using FLAG-JARID1A baculoviruses, and affinity purified using anti-FLAG antibody. FLAG-JARID1A was analyzed by SDS-PAGE for purity (FIG. 13A), and by western blot for JARID1A expression (FIG. 13B).

### Example 8

### High-Throughput Screening for JARID1A Inhibitors

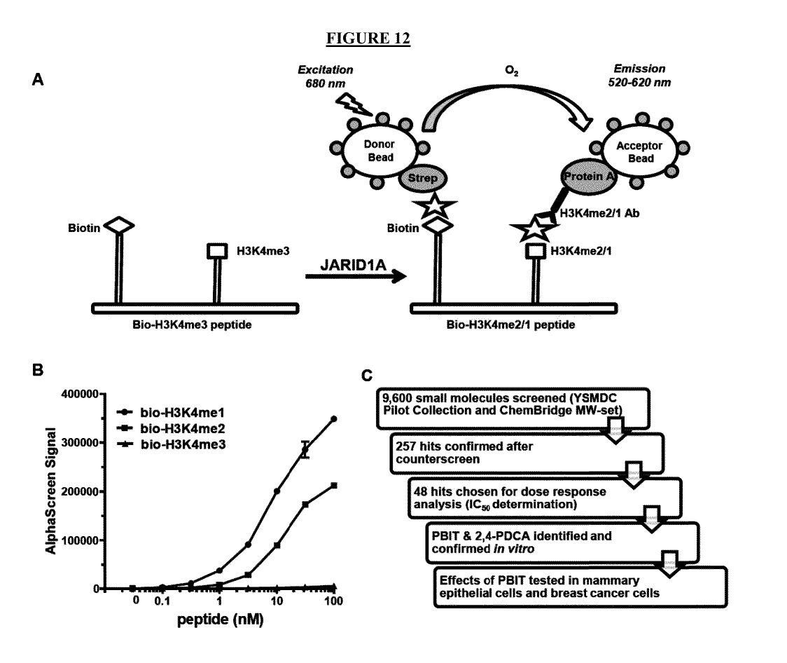

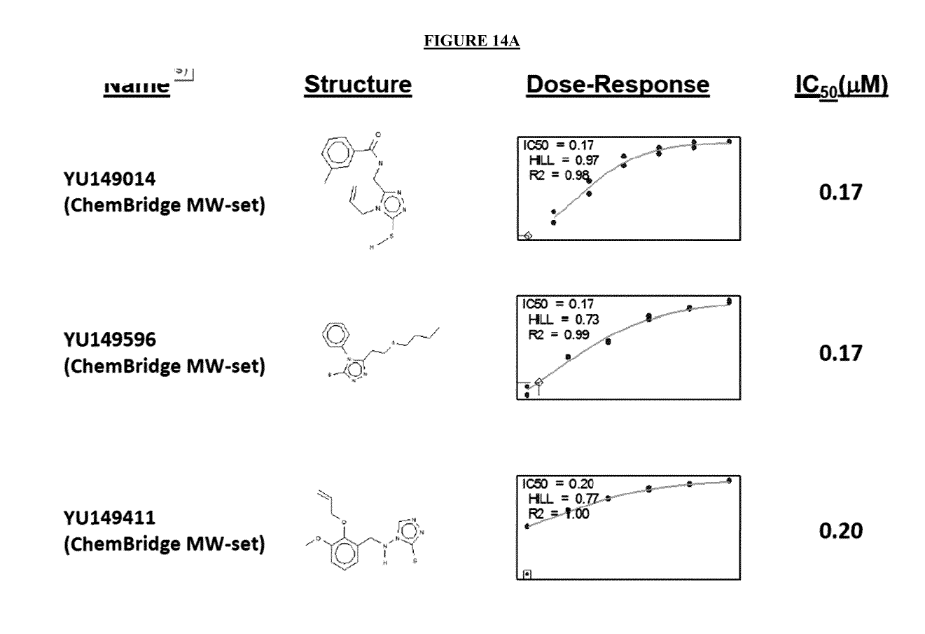

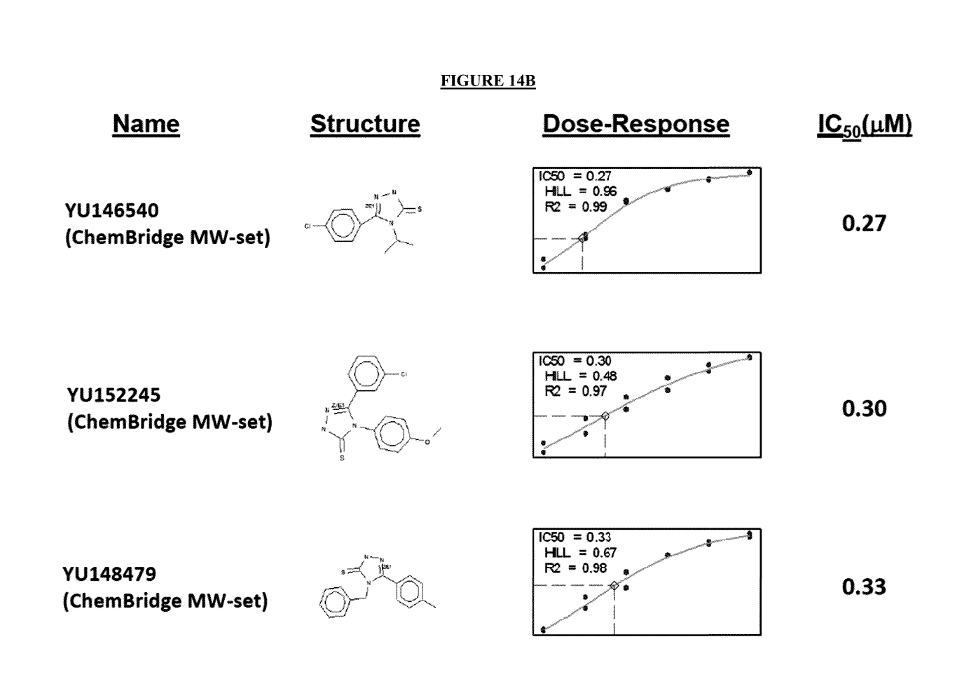

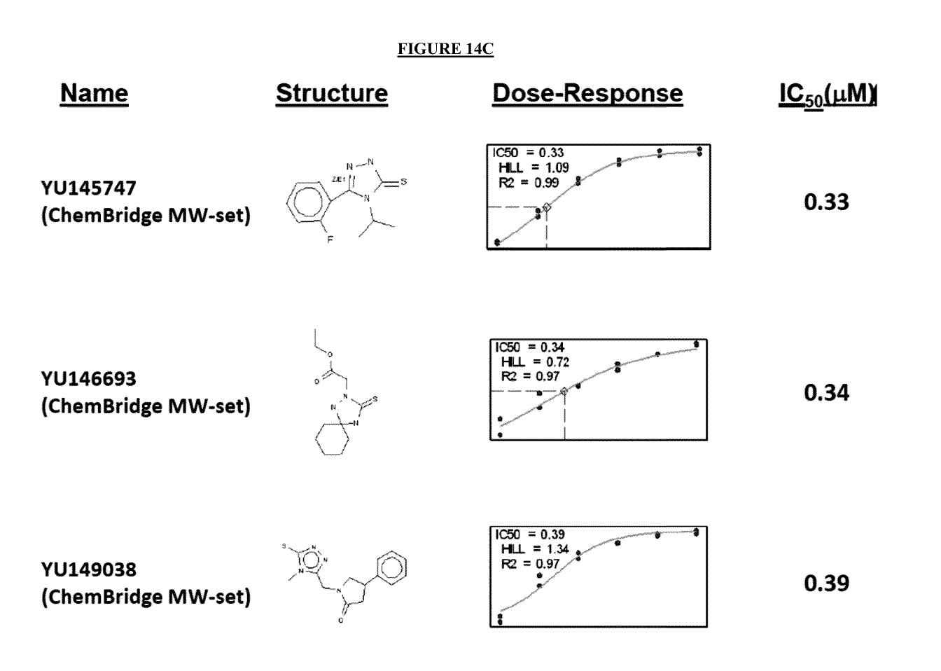

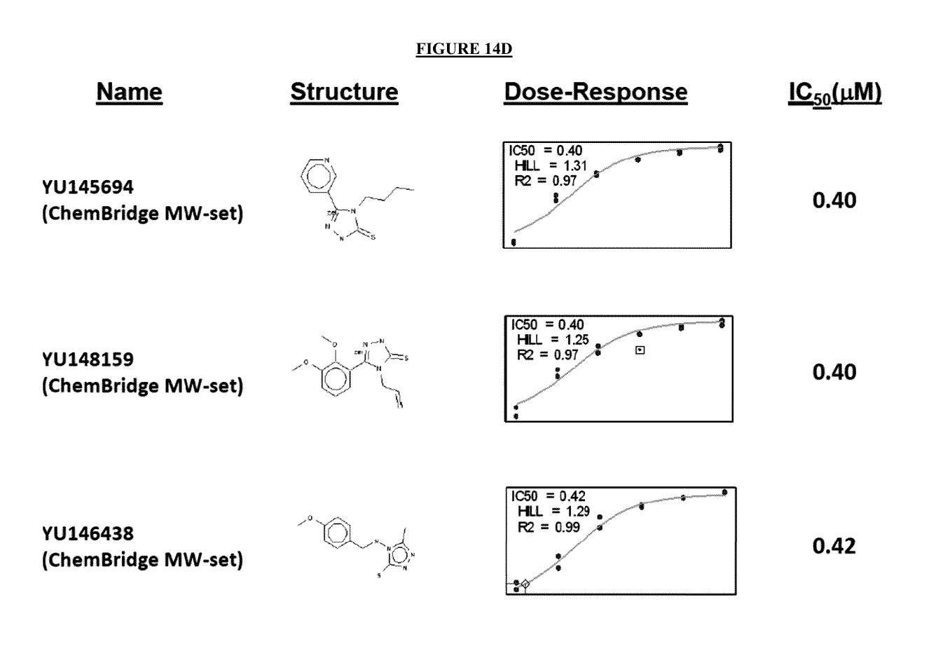

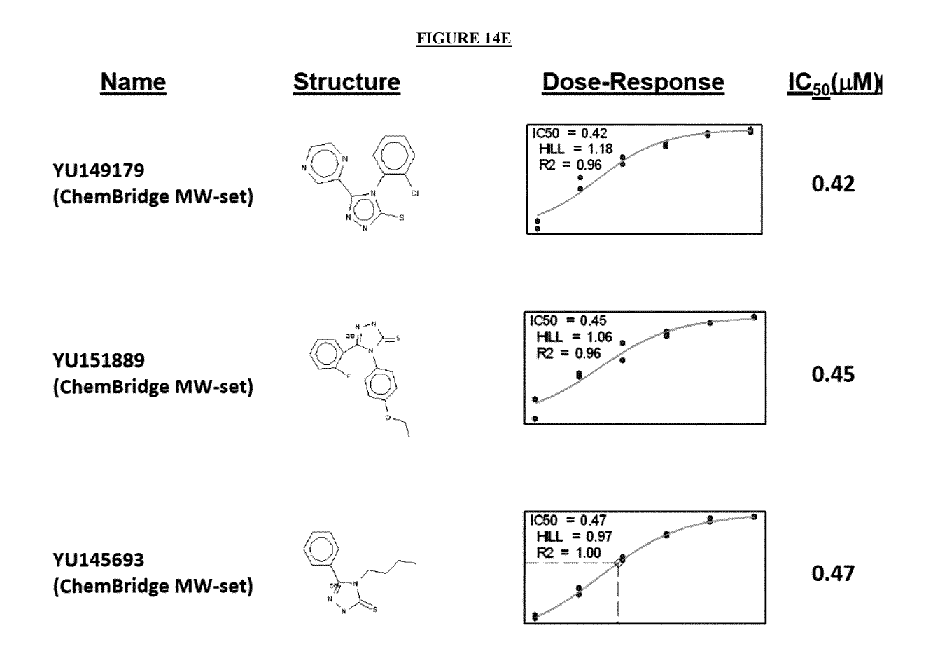

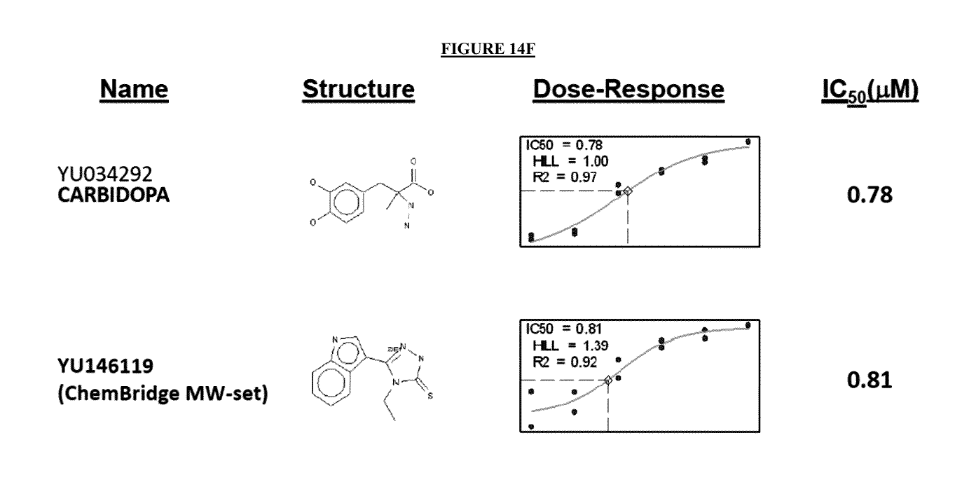

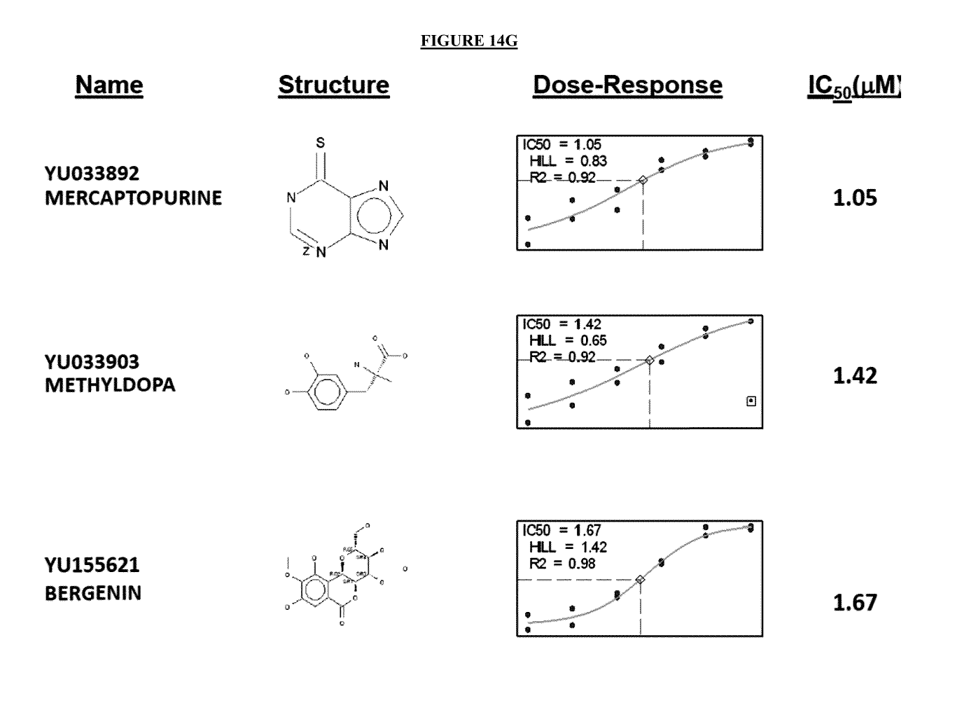

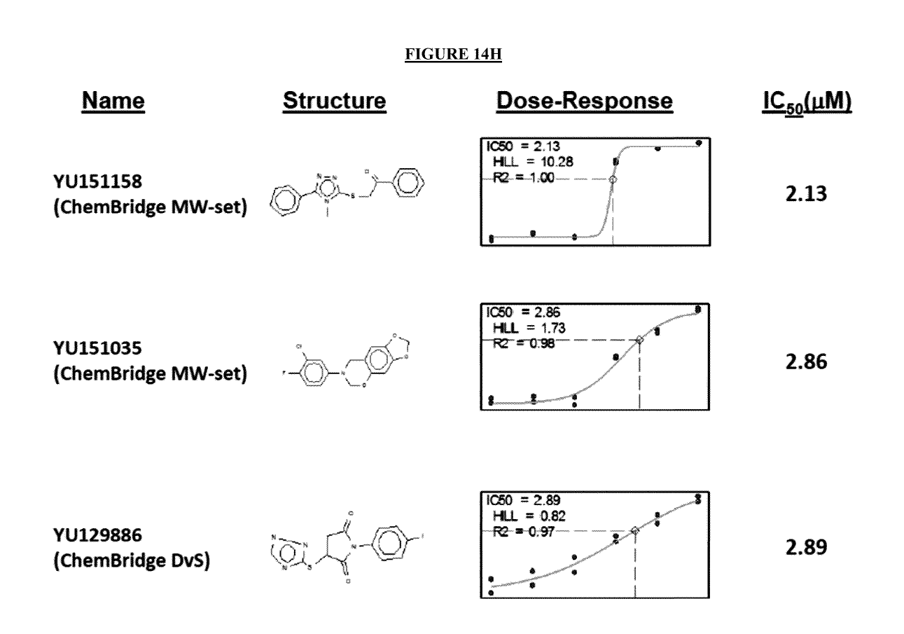

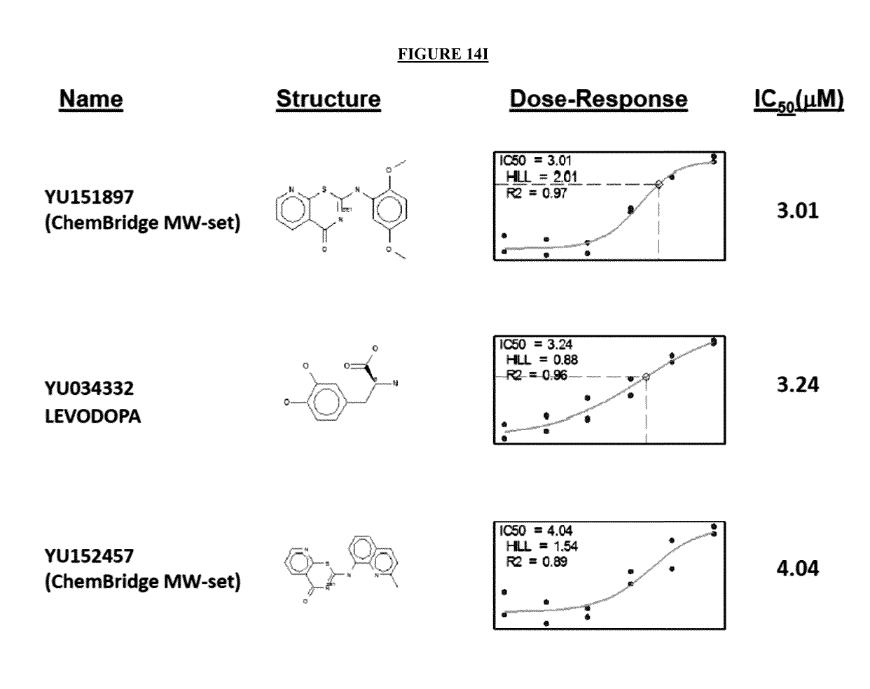

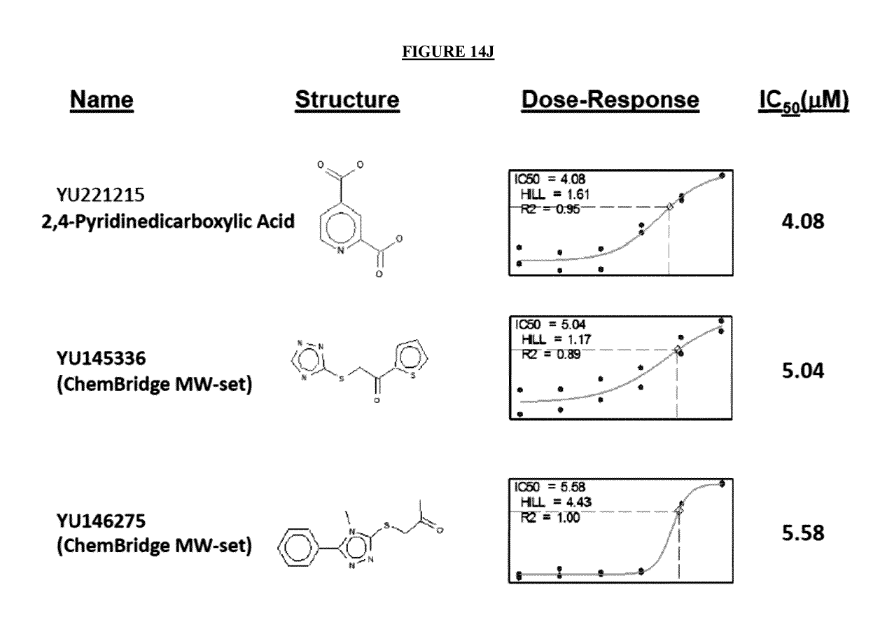

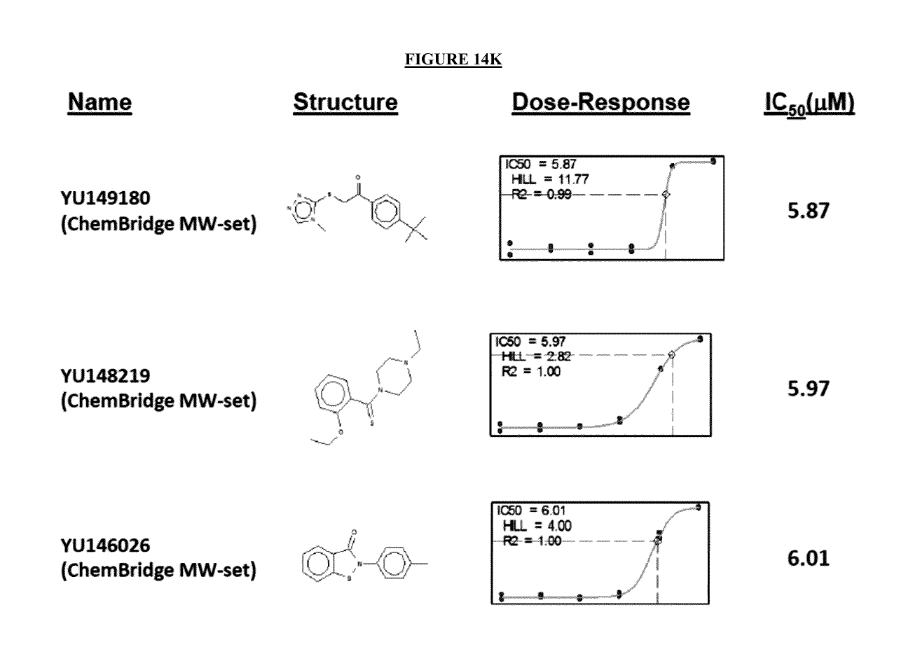

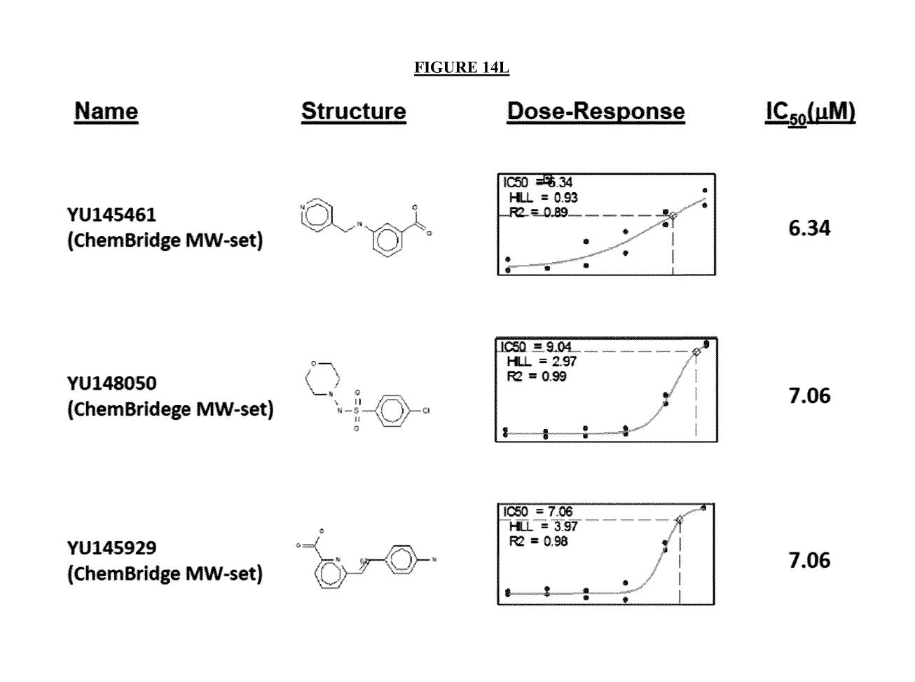

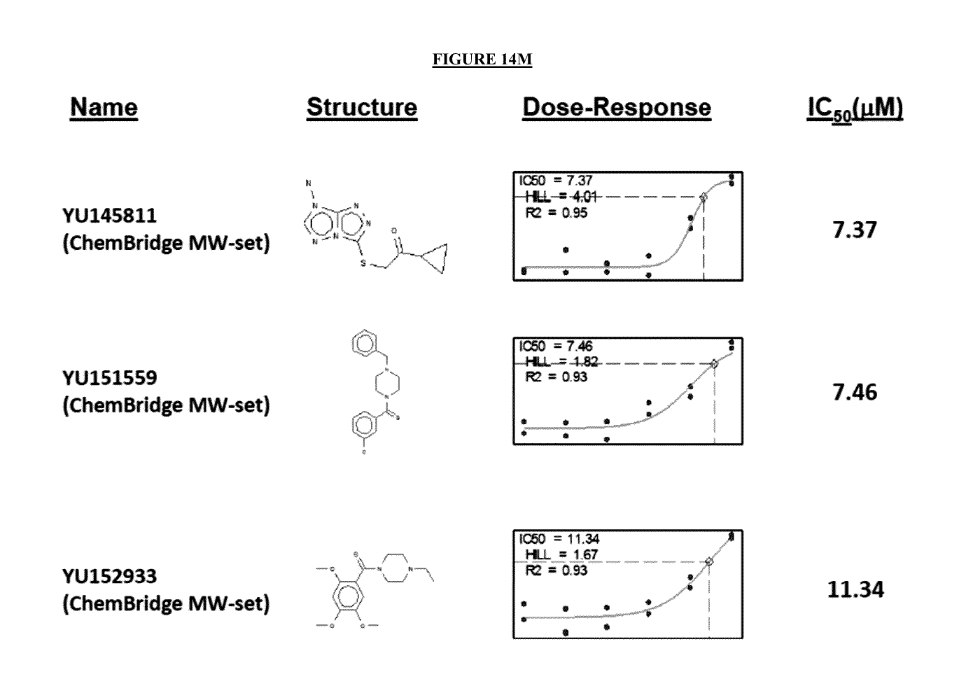

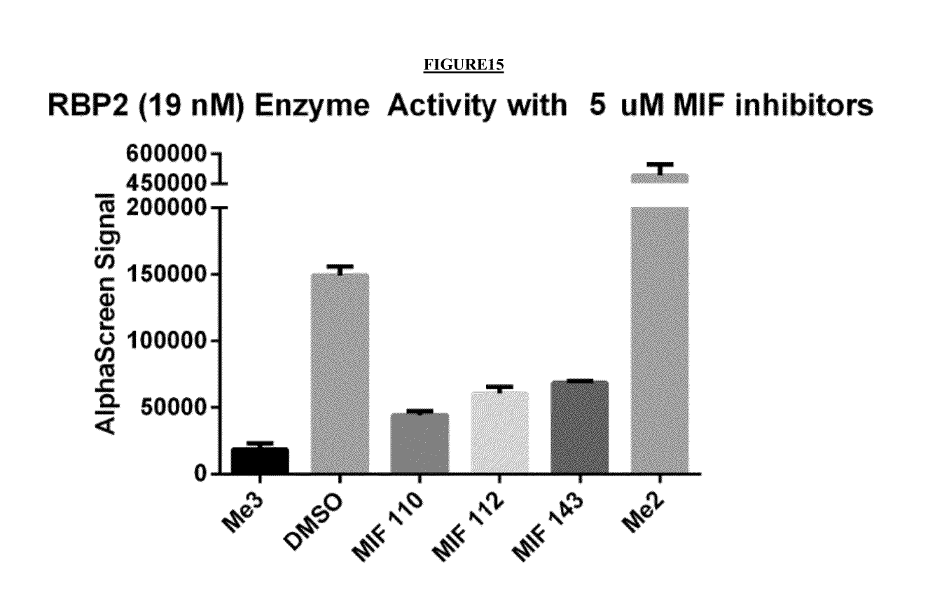

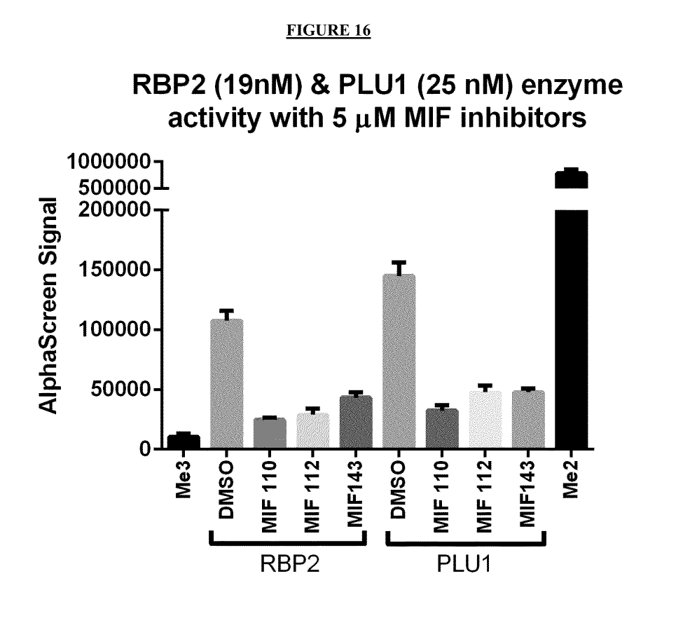

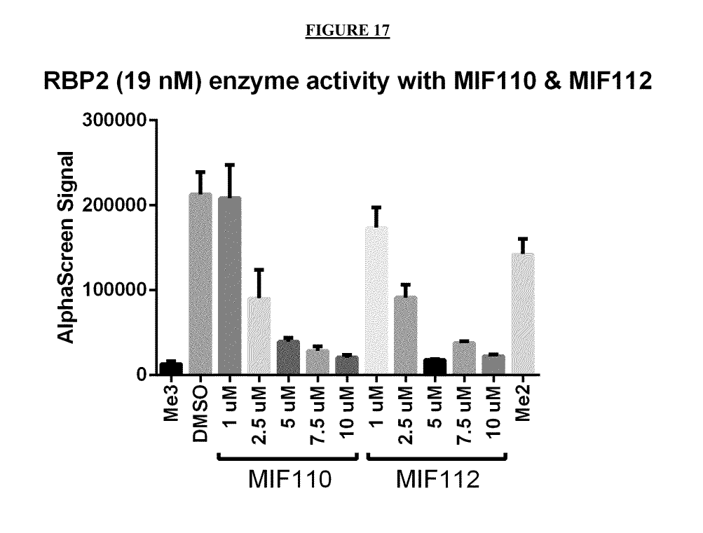

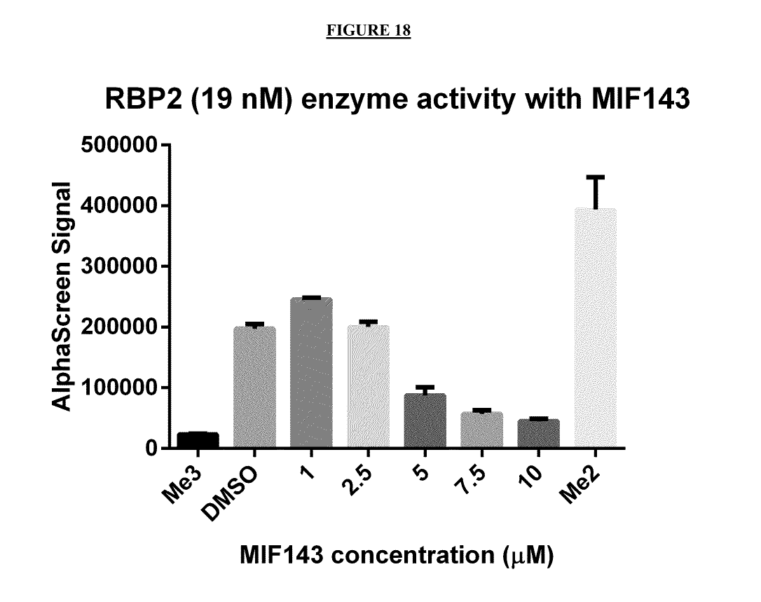

FLAG-JARID1A was screened against 9,600 compounds from several small molecule libraries (FIG. 12C). Among these hits, 257 compounds were validated after a counter-screen using the bio-H3K4me2 peptide, which eliminates the compounds that have non-specific effect on AlphaScreen assays (FIG. 12C). At the 30% threshold limit, 170 hits were identified. Of these, 48 compounds were selected based on their inhibition efficiency and structure for further dose response analysis. Dose response analysis was performed in the presence of 50 μM Fe (II). 16 of the 48 compounds chosen for dose response analysis yielded nM IC₅₀ values, and 18 compounds yielded IC₅₀ values under 5 μM (FIG. 14). These inhibitors included several known demethylase inhibitors, such as PBIT and 2,4-PDCA and PBIT. PBIT inhibited JARID1A with an IC₅₀ value of about 6 μM and 2,4-PDCA inhibited JARID1A with an IC₅₀ value of about 4 μM (FIG. 14).

Among the top inhibitors, several compounds were identified in the screen that inhibited JARID1A in the high nanomolar range (FIG. 14). Mercaptopurine (used to treat leukemia), inhibited JARID1A with an IC₅₀ value of about 1 μM (FIG. 14). Methyldopa, a known inhibitor of JMJD2E, was also identified as a potent inhibitor of JARID1A in our screen, also with an IC₅₀ value of just over 1 μM (FIG. 14). ChemBridge MW-set and ChemDiv compounds YU151035, YU129886, and YU151897 inhibited JARID1A with IC₅₀ values at or below 3 μM (FIG. 14).

### Example 9

### Epigenetic Regulator RBP2 is Critical for Breast Cancer Progression and Metastasis

To identify novel epigenetic regulators that can be targeted in breast cancer metastasis, unbiased bioinformatic analysis of human breast cancer datasets were conducted. Histone demethylase RBP2 was identified as a strong predictor of breast cancer metastasis. RBP2 is a JARID1 family histone demethylase, which catalyzes the removal of methyl-groups from tri- or di-methylated lysine 4 in histone H3. RBP2 positively regulates many metastasis related genes, including TNC, which is required for formation of the metastatic niche in the lung. Further, RBP2 is critical in invasion and metastasis using in vitro invasion and in vivo metastasis assays. These findings are further validated in the MMTV-neu transgenic mouse model. In a non-limiting embodiment, the findings suggest that RBP2 is a critical epigenetic switch that sets the stage for tumor metastasis and can be targeted to block breast cancer metastasis.

### Materials and Methods

### Cell Culture.

MDA-MB-231, LM2, 67NR, and 4T1 breast cancer cells were cultured in Dulbecco's Modified Eagle Medium (DMEM) supplemented with 10% fetal bovine serum (FBS) and 1% penicillin and streptomycin. Retroviruses were generated by co-transfection of pLKO.1 plasmids carrying the indicated shRNAs with packaging plasmids into 293T cells (Klose et al., 2007, Cell 128:889-900). To generate RBP2 stable knockdown cell lines, LM2 cells were infected with the indicated viruses and selected with 1 μg/ml puromycin for two weeks. siRNA transfections were performed using RNAiMAX (Invitrogen). Plasmid transfections and plasmid/siRNA cotransfections were performed using Lipofectamine 2000 (Invitrogen). The shRNA sequences targeting RBP2 were: sh-1, ccagacttacagggacactta (SEQ ID NO: 14); sh-2, ccttgaaagaagccttacaaa (SEQ ID NO: 15). Scrambled control shRNA was described previously (Yang et al., 2007, Mol. Cell 28:15-27). The siRNA targeting sequences of RBP2 were: siRNA-1, gctgtacgagagtatacac (SEQ ID NO: 16); siRNA-2: cttctgtactgctgactgg (SEQ ID NO: 17); siRNA-3: gccaagaacattccagcct (SEQ ID NO: 18). Scrambled siRNA was described previously (Beshiri et al., 2012, PNAS 109:18499-18504) and luciferase siRNA was obtained from Dhamacon.

### Immunoblot and Real-Time RT-PCR Analysis.

Immunoblotting of cellular proteins was performed as described previously (Klose et al., 2007, Cell 128:889-900). For immunoblotting of secreted proteins, cells were grown in Opti-MEM (Invitrogen) for 24 hours, and media were harvested and concentrated using acetone precipitation. Antibodies for immunoblotting were GAPDH (G9545, Sigma), RBP2 (mAB3876, Cell signaling), TNC (MAB1918, Millipore), IGFBP7 (SC-13095, Santa Cruz), HA (MMS101P, Covance), tubulin (T5168, Sigma), H3 (ab1791, Abeam), H3K4me (ab8895, Abeam), H3K4me3 (ab8580, Abeam).

Total RNA was isolated and reverse transcription was performed, followed by real-time PCR. Primers for real-time PCR were GAPDH-F: tgcaccaccaactgcttagc (SEQ ID NO: 19); GAPDH-R: ggcatggactgtggtcatgag (SEQ ID NO: 20); RBP2-F: ccgtctttgagccgagttg (SEQ ID NO: 21), RBP2-R: ggactcttggagtgaaacgaaa (SEQ ID NO: 22); TNC-F: gtcaccgtgtcaacctgatg (SEQ ID NO: 23), TNC-R: gcctgccttcaagatttctg (SEQ ID NO: 24); Sox4-F: aagcttcagcaaccagcatt (SEQ ID NO: 25), Sox4-R: ccctctctctcgctctctca (SEQ ID NO: 26); FSCN1-F: aggggactcagagctcttcc (SEQ ID NO: 27), FSCN1-R: tgcctgtggagtctttgatg (SEQ ID NO: 28); Co11A1-F: cctggatgccatcaaagtct (SEQ ID NO: 29), Co11A1-R: aatccatcggtcatgctctc (SEQ ID NO: 30); PDGFA-F: caagaccaggacggtcattt (SEQ ID NO: 31), PDGFA-R: cctgacgtattccaccttgg (SEQ ID NO: 32); SEPRINE2-F: ctttgaggatccagcctctg (SEQ ID NO: 33), SEPRINE2-R: tgcgtttctttgtgttctcg (SEQ ID NO: 34); PLCB1-F: cgtggctttccaagaagaag (SEQ ID NO: 35); PLCB1-R: gcttccgatctgctgaaaac (SEQ ID NO: 36).

### Trans-Well Invasion Assays.

Breast cancer cells with 60-70% confluence were serum starved in DMEM media supplemented with 0.2% FBS for 6 hours. After starvation, the cells were trypsinized and resuspended in DMEM media supplemented with 0.2% FBS, and seeded at 5×10⁴ cells per well into the insert of growth factor reduced Matrigel invasion chambers (BD Biosciences). DMEM media supplemented with 10% FBS was added to the bottom well as chemo-attractant. In TNC rescue experiment, BSA and/or recombinant TNC protein (CC065, Millipore) were pre-incubated with the insert for 2 hours and added to the media when seeding cells at the final concentration of 100 ng/ml. The inserts were washed with PBS and fixed with 4% paraformaldehyde after 16-hour incubation at 37° C. with 5% CO₂. The cells on the apical side of the insert were scrubbed off, and the cells invaded to the basal side of the membrane were visualized with DAPI staining. Pictures of four random fields from each well were taken under the microscope at 10× magnification, and the number of cells on the basal side was counted.

### Animal Studies

6-8 weeks old NOD/SCID female mice were used for lung metastasis and mammary fat pad tumor growth experiments. For lung metastasis assay, 2×10⁵ viable cells were re-suspended in 0.1 ml saline, and injected into the lateral tail vein. Lung metastatic colonization was monitored and quantified immediately after the injection and at the indicated time points using non-invasive bioluminescence. All values of luminescence photon flux were normalized to the value of the same mouse obtained immediately after the tail vein injection. For mammary fat pad tumor growth assay, 1×10⁶ viable cells were re-suspended in 0.1 ml of 1:1 mixture of saline and Matrigel, and injected into the fat pads of 4^{th} mammary glands of the mouse. Tumor weight was determined at the end point.

The MMTV-neu (FVB/N-Tg(MMTVneu)202Mul/J) mice were obtained from The Jackson Laboratory. Rbp2^{-/-} mice (Klose et al., 2007, Cell 128:889-900) were backcrossed to FVB strain for at least eight generations, and then crossed with the MMTV-neu transgenic mice for two generations. The copy numbers of the MMTV-neu transgene were determined by genotyping at least 12 off-springs from the crossing of Rbp2^{+/-} mice carrying the MMTV-neu transgene with wild type mice. Rbp2^{+/-} mice with two copies of the MMTV-neu transgenes were selected and intercrossed. Breast tumor formation of Rbp2^{+/+}:MMTV-neu and Rbp2^{-/-}:MMTV-neu mice were monitored weekly and mice were euthanized when primary tumors reached approximately 1 cm³.

### Histopathology

Mice were euthanized by CO₂ asphyxiation and lungs harvested and fixed in 10% neutral buffered formalin, processed, sectioned and stained by hematoxylin and eosin (H&E) by routine methods by Research Histology (Department of Pathology) or Yale Mouse Research Pathology (Section of Comparative Medicine), Yale University School of Medicine. Tissues were evaluated blind to experimental manipulation (by CJB) for the presence and number of tumor metastatic foci and percentage of lung effaced by tumor. Digital light microscopic images were recorded using a Axio Imager.A.1 microscope and an AxioCam MRc5 camera and AxioVision 4.7 imaging software (Zeiss) and optimized in Adobe Photoshop CS5 (Adobe Systems Incorporated, USA). All procedures involving animals were approved by the Yale University Institutional Animal Care and Use Committee.

### Bioinformatic and Statistical Analysis.

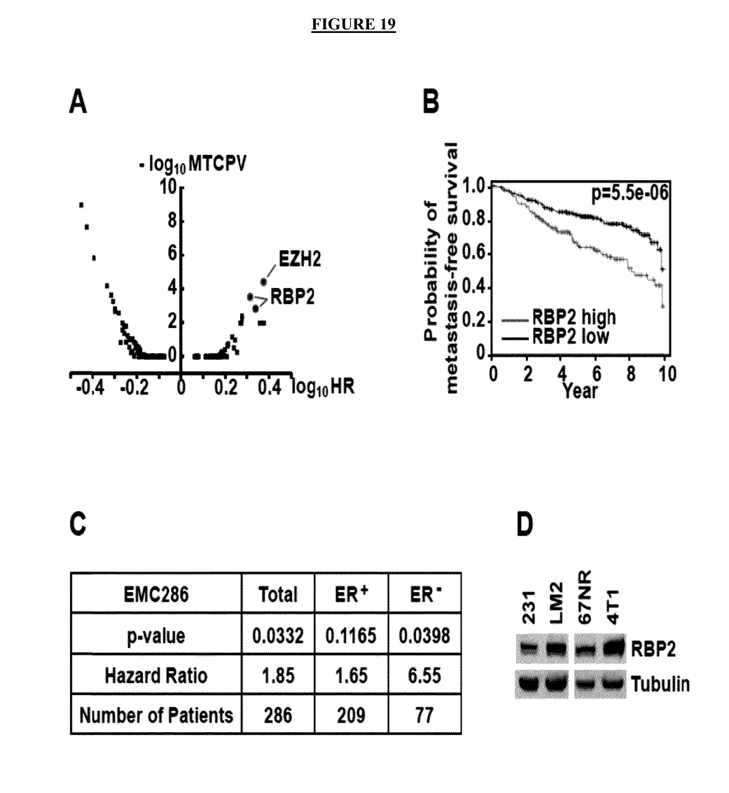

Kaplan Meier-plotter analysis of histone modifying enzymes in metastasis-free survival of breast cancer patients were performed by using the tool generated by the Szallasi group (Gyorffy et al., 2010, Breast Cancer Res. Treat. 123:725-731). The settings for the analysis were: DMFS, auto select best cutoff, 10 years follow up threshold, ER status all, PR status all, lymph node negative, grade all, and molecular subtype all. Multiple testing corrected p-values of each probe were calculated by timing original p-value with the number of genes analyzed. The results of these experiments were described in FIGS. 19A-C and 24, and Table 29.

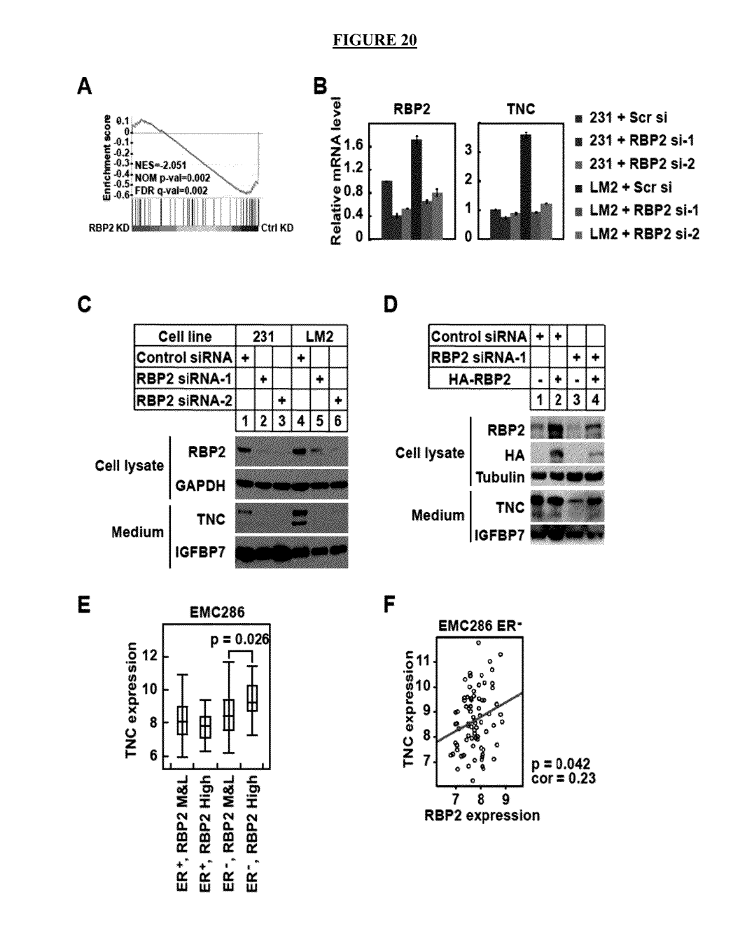

EMC286 cohort gene expression data was downloaded from GSE2034 (Wang et al 2005, Lancet 365:671-679) and normalized using robust multi-array average (RMA) method within R Affy package. For this cohort, RBP2 probe 202040_s_at was selected for Kaplan Meier metastasis-free survival analysis, and correlation analysis. Samples with high, medium and low RBP2 expression levels were clustered using quartile or k-means as indicated in the figure legends. The metastasis-free survival plots, Cox univariate and multivariate metastasis-free survival analyses were performed via R survival package. Pearson correlation test was performed using the cor.test R function. The results of these experiments were described in FIGS. 20E-F, 24B-D and 30.

Gene expression profiles of the control knockdown (scrambled siRNA) and the average of two RBP2 knockdown (RBP2 siRNAs si-1 and si-2) cells were used for gene set enrichment analysis using GSEA v2.0 software. Gene sets were generated from published gene signatures. Statistical significance was assessed by comparing the enrichment score to enrichment results generated from 10,000 random permutations of the gene set. Log-rank (Mantel-Cox) test were used for analysis of tumor-free survival curves of the MMTV-neu transgenic mice. Comparison of luminescence signals of LM2 cells with control or RBP2 knockdown hairpins in lungs was performed using Wilcoxon rank-sum test. Comparison of number of metastatic nodules in lungs from wild type or RBP2 knockout mice was performed using negative binomial model. T-test was used for other statistical analysis.

### RBP2 Expression Predicts Breast Cancer Metastasis

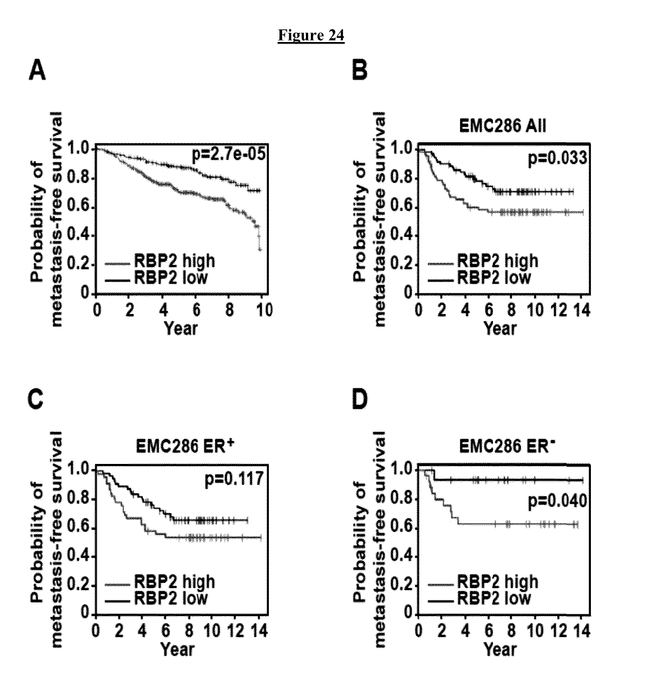

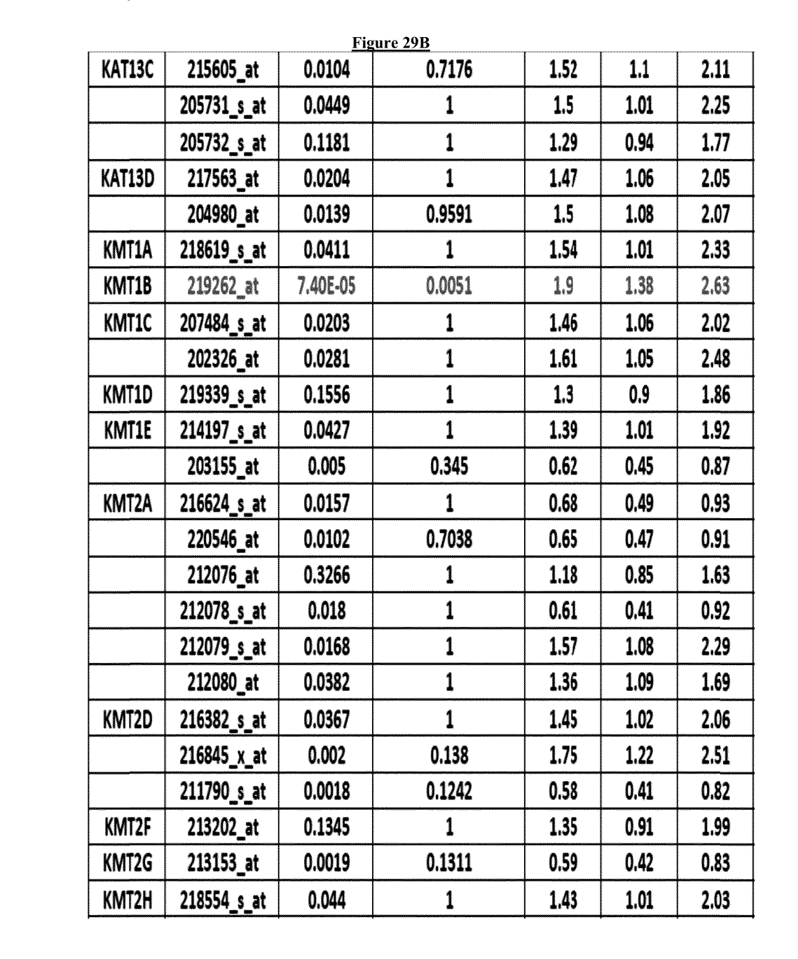

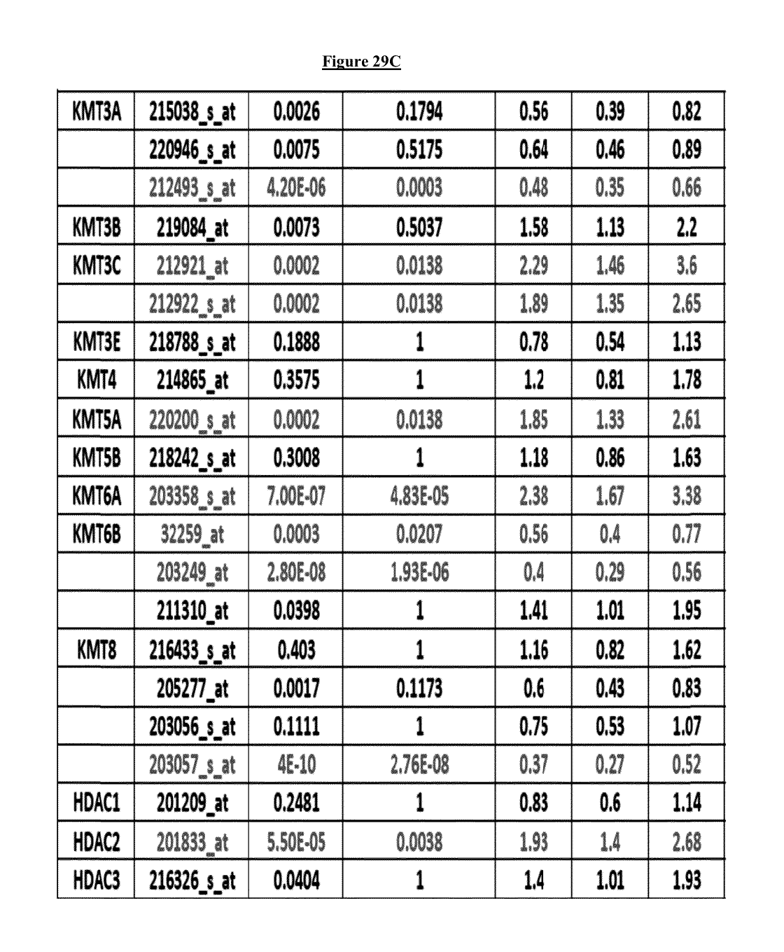

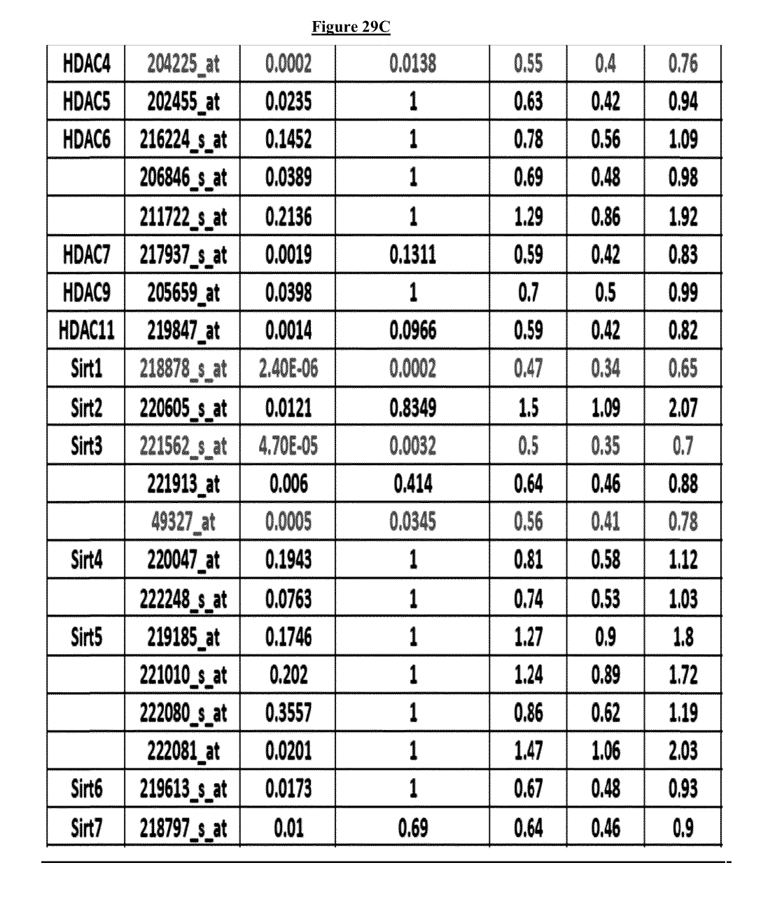

To identify novel epigenetic regulators of breast cancer metastasis, unbiased bioinformatic analysis of gene expression profiles of mammary tumors from 533 breast cancer patients were conducted using Kaplan-Meier Plotter, a meta-analysis based biomarker assessment tool (Gyorffy et al., 2010, Breast Cancer Res. Treat. 123:725-731). This analysis tool utilizes Affymetrix gene expression profiling data, which have multiple probe sets for most genes. Correlations were examined between increased incidence of distant tumor metastasis with the gene expression levels of selected histone modifying enzymes, including histone lysine methyltransferases (KMTs), histone lysine demethylases (KDMs), histone acetyltransferases (KATs), and histone deacetylases (HDACs). This analysis revealed that high mRNA levels of two enzymes, EZH2 and RBP2, correlated significantly with early and high incidence of tumor metastasis (FIGS. 19A and 29). The approach was validated by the fact that EZH2 was previously shown to promote breast cancer metastasis. Two probes of RBP2 are present on the microarray platform, and both probes showed similar results (FIGS. 19B and 24A). RBP2 was found to contribute substantially to tumorigenesis and drug resistance. However, the role of RBP2 in tumor metastasis has not been determined.

The association of RBP2 expression with breast cancer metastasis was validated using a large lymph node negative clinical dataset, EMC286 (FIG. 24B). To determine whether the association of RBP2 expression and metastasis is subtype specific, estrogen receptor (ER) positive (ER⁺) and negative (ER⁻) patients were separated in the EMC286 cohort. This revealed that RBP2 predicts metastasis in ER⁻patients, but not ER⁺ patients (FIGS. 19C and 24C-D). To further determine whether the correlation of RBP2 with metastasis was dependent on other clinical parameters, a Cox multivariate analysis of EMC286 dataset was conducted and it was found that RBP2 predicted tumor metastasis independent of ER, PR, and HER2 status (FIG. 30).

The analysis was then extended to two well-established matched breast cancer cell line pairs, including LM2 and MDA-MB-231, and 4T1 and 67NR. LM2 cells were derived from MDA-MB-231 human breast cancer cells by in vivo selection, and have increased metastatic activity to the lung when compared to the parental MDA-MB-231 cells. Mouse poorly metastatic 67NR cells and highly metastatic 4T1 cells were isolated from a single spontaneous mammary tumor. Western blot analysis showed that RBP2 protein was expressed at higher levels in the more metastatic LM2 and 4T1 cells, compared to their poorly metastatic counterparts, respectively (FIG. 19D). These results are consistent with the observation in patient samples that RBP2 expression correlates with increased metastatic potential.

### RBP2 is Critical for Metastasis Gene Expression

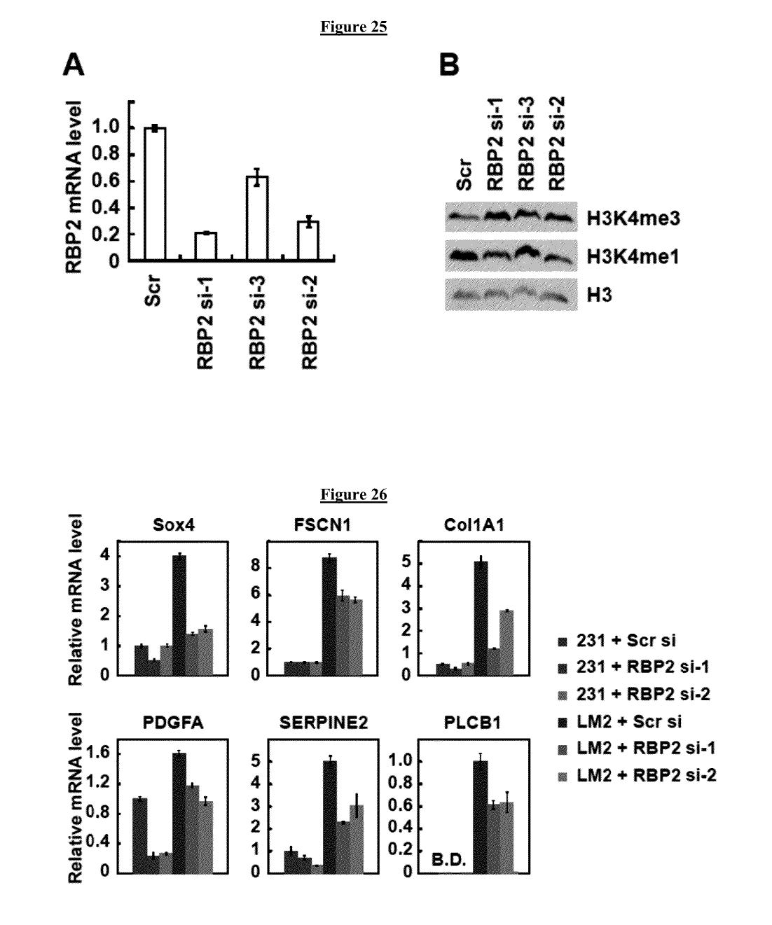

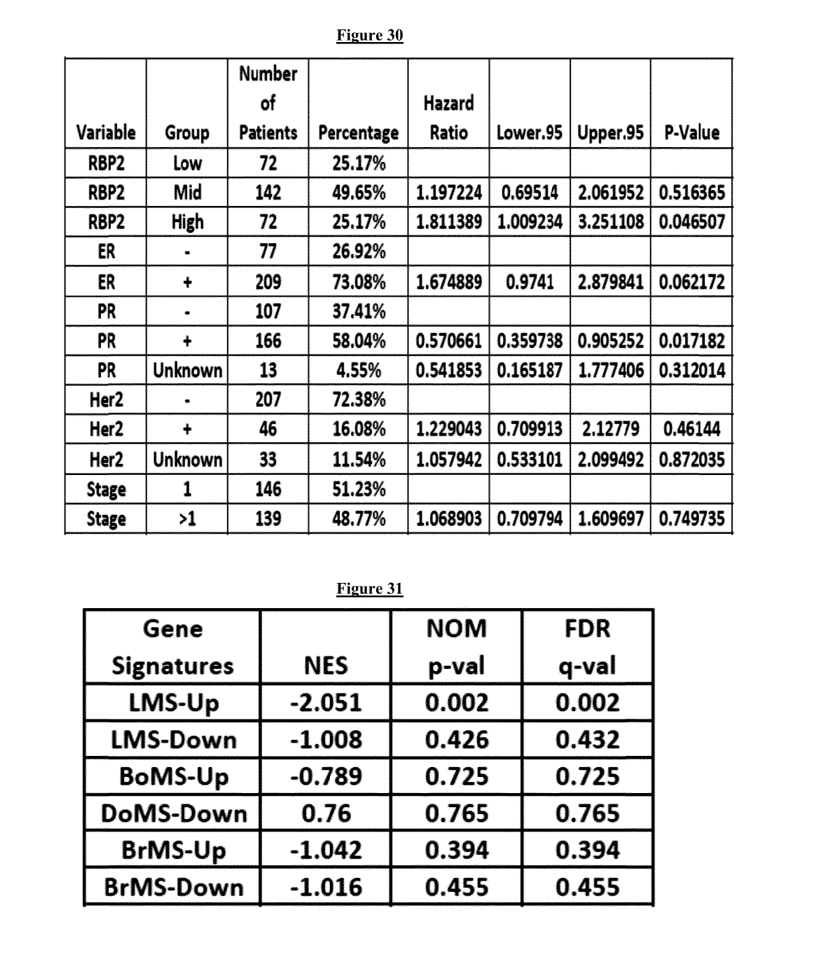

To determine the roles of RBP2 in breast cancer, MDA-MD-231 cells were transfected with siRNAs against RBP2 or luciferase control. Knockdown of RBP2 led to global increase of H3K4me3 level, suggesting that RBP2 is the major H3K4me3 demethylase in MDA-MB-231 cells (FIG. 25A-B). To determine the broad transcriptional effects of RBP2 depletion in breast cancer cells, microarray analysis of these knockdown cells was conducted. In the analysis the focus was on the potential regulation by RBP2 of known organotropic metastasis gene expression programs including a lung metastasis signature (LMS), bone metastasis signature (BoMS), and brain metastasis signature (BrMS). These analyses revealed that RBP2 knockdown significantly decreased expression of genes linked to breast cancer metastasis to lung (FIG. 20A). In contrast, there were no significant changes of genes involved in breast cancer metastasis to bone or brain in RBP2 knockdown cells (FIG. 31). Several genes, including TNC, SOX4, FSCN1, COL1A1, PDGFA, SERPINE2, and PLCB1, were selected, all of which were implicated in breast cancer metastasis to the lung, for real time RT-PCR analysis. As expected, these genes were expressed at higher levels in LM2 cell than in MDA-MB-231 cells (FIGS. 20B and 26). Consistent with the GSEA results, these genes were significantly down-regulated in RBP2 knockdown cells (FIGS. 20B and 26).

Among these genes, the TNC gene encoding tenascin C [an extracellular matrix protein that promotes colonization of breast cancer cells to the lung] showed the most significant decrease in both MDA-MB-231 and LM2 cells with RBP2 knockdown. Since TNC is a secreted protein, the proteins in the growth media of these cells were analyzed and confirmed the decrease of TNC protein production by RBP2 knockdown cells (FIG. 20C). To exclude off-target effects of the siRNAs, an siRNA-resistant form of RBP2 was re-introduced into RBP2 knockdown cells. Consistent with the idea that RBP2 is critical for TNC expression, restoration of RBP2 expression rescued the decrease of TNC expression in RBP2 knockdown cells (FIG. 20D). Next, the correlation of expression of RBP2 and TNC in primary breast tumors was examined Consistent with the data from the cell lines, ER⁻ tumors with high levels of RBP2 expressed high levels of TNC (FIG. 20E), and RBP2 expression positively correlated with TNC expression in ER⁻ tumors (FIG. 20F).

### Knockdown of RBP2 Suppresses Invasion

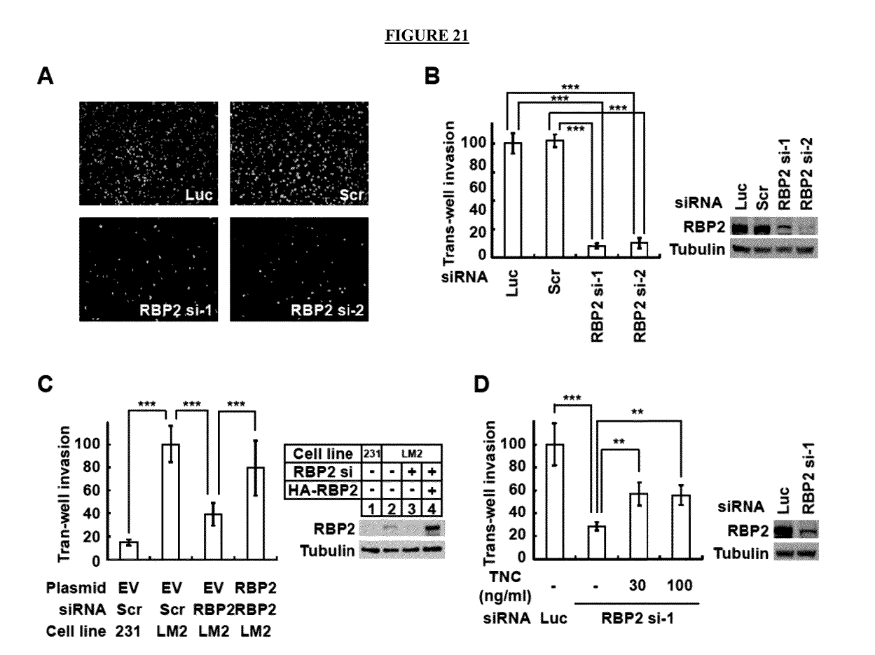

To dissect the roles of RBP2 in tumor metastasis, its role in invasion was examined using trans-well invasion assays. The LM2 cells transfected with siRNAs against RBP2 showed a dramatic decrease of their ability to invade through Matrigel, compared with the cells transfected with the control siRNAs (FIGS. 21A-B). Restoration of RBP2 expression in LM2 cells rescued the diminished cell invasion induced by RBP2 knockdown (FIG. 21C). TNC is an extracellular matrix protein and promotes cancer cell migration and invasion. Since TNC expression was regulated by RBP2 in LM2 cells (FIGS. 20B-C) the question of whether TNC mediates the regulation of invasion by RBP2 was addressed. To this end, recombinant TNC protein was added into the chambers in trans-well assays. The addition of TNC partially rescued the decreased invasion phenotype in RBP2 knockdown cells (FIG. 21D), suggesting that regulation of invasion by RBP2 was at least partially due to the decrease of TNC expression in RBP2 knockdown cells.

### RBP2 is Critical for Breast Cancer Metastasis to the Lung In Vivo.

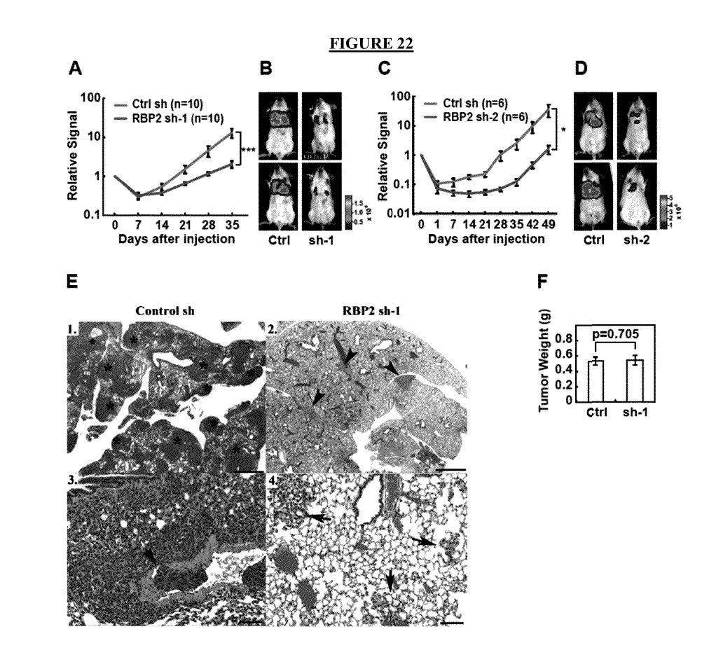

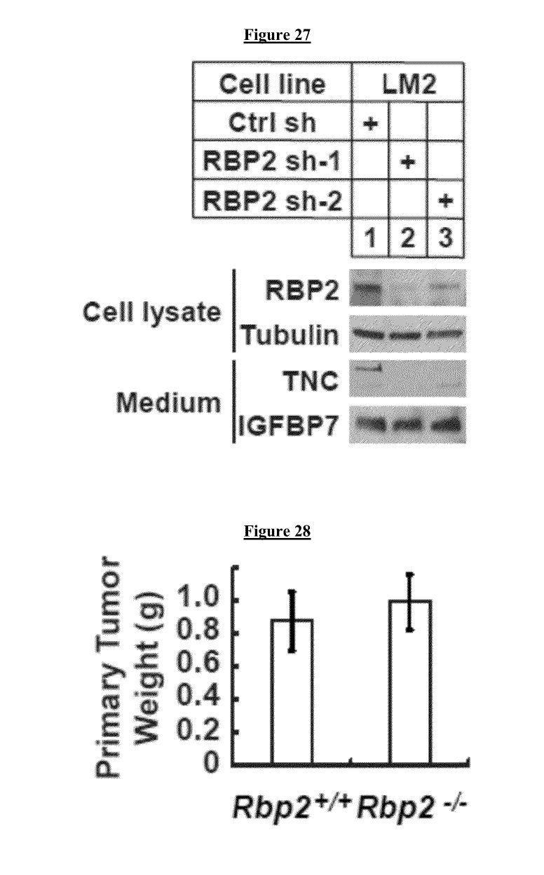

To investigate the roles of RBP2 in metastasis using in vivo lung metastasis assays, LM2 cells with stable knockdown of RBP2 were generated using two independent shRNA hairpins or with scrambled shRNA control hairpin. LM2 cells were engineered to express firefly luciferase, which allows for live imaging to monitor metastasis in vivo. Similar to the results from siRNA mediated knockdown experiments, LM2 cells with RBP2 stable knockdown secreted lower levels of TNC to the growth media compared to the control cells (FIG. 27). These cells were then injected into the tail vein of SCID mice, and lung metastatic activity was assayed by bioluminescence imaging weekly, as well as by examination of the lungs at necropsy. RBP2 knockdown hairpins led to approximately 10-fold decrease of lung colonization abilities of LM2 cells (FIGS. 22A-D). The effect was observed even at the early time points (FIGS. 22A-D), suggesting that RBP2 controls extravasation and/or early seeding of lung metastasis. Histological analysis of the lungs isolated at necropsy confirmed that mice injected with RBP2 knockdown cells had fewer and smaller lesions in the lungs (FIG. 22E). In contrast, knockdown of RBP2 had no effect on mammary tumor formation in orthotopic xenograft experiments (FIG. 22F).

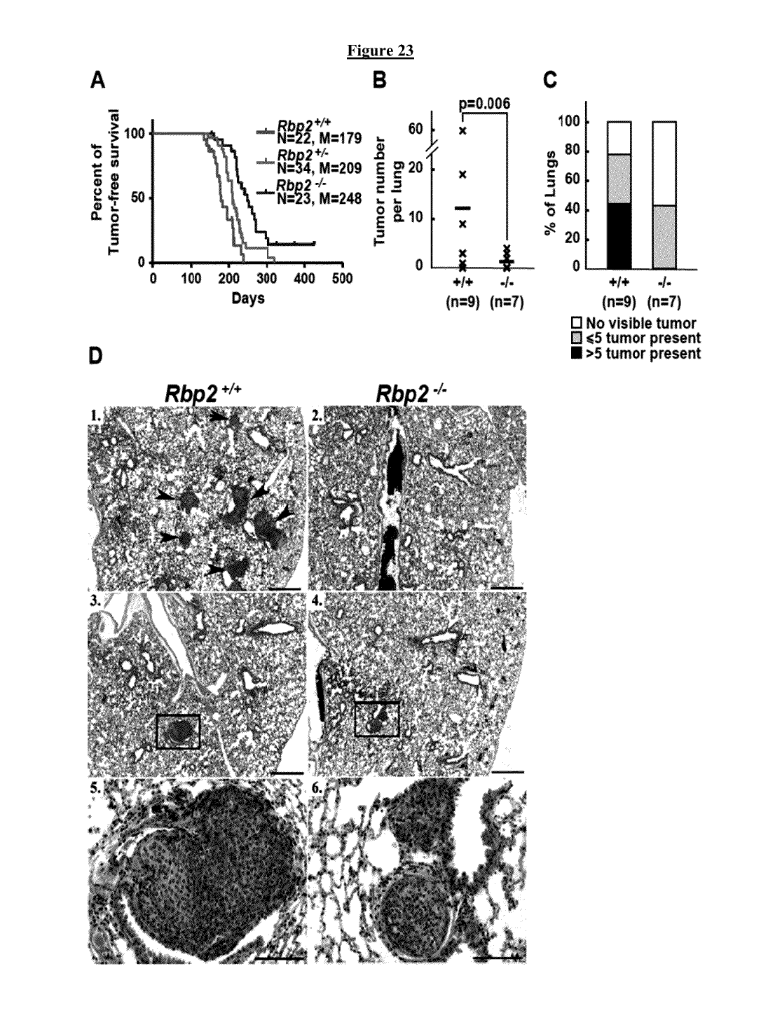

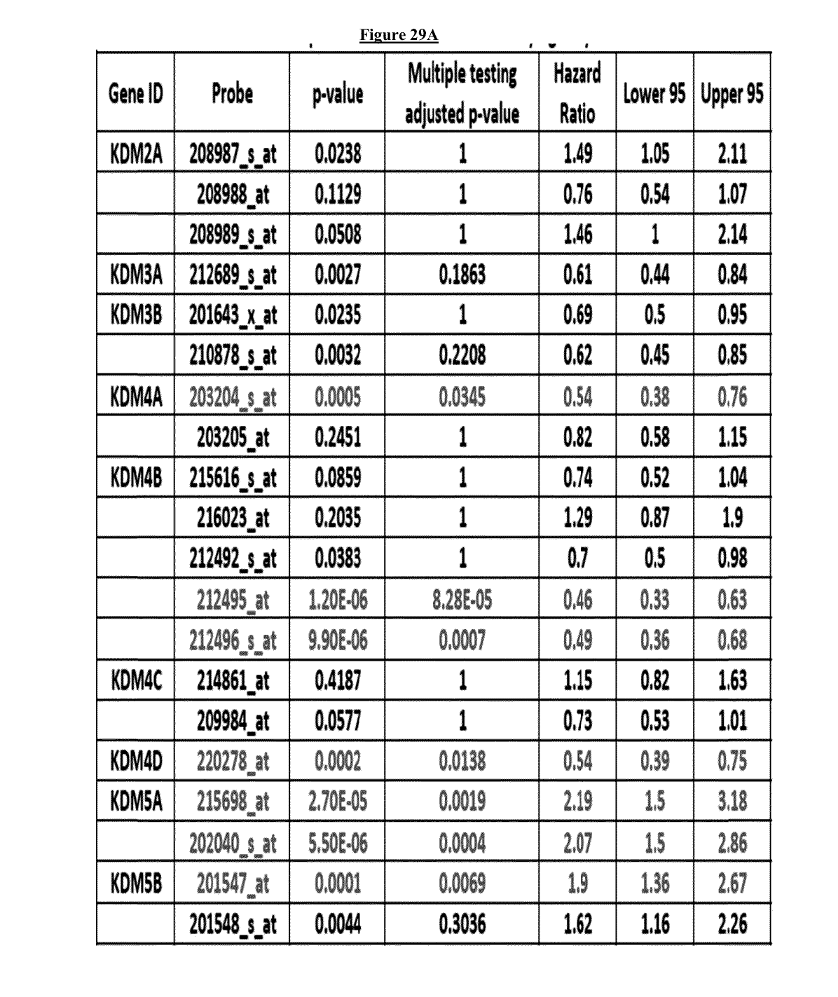

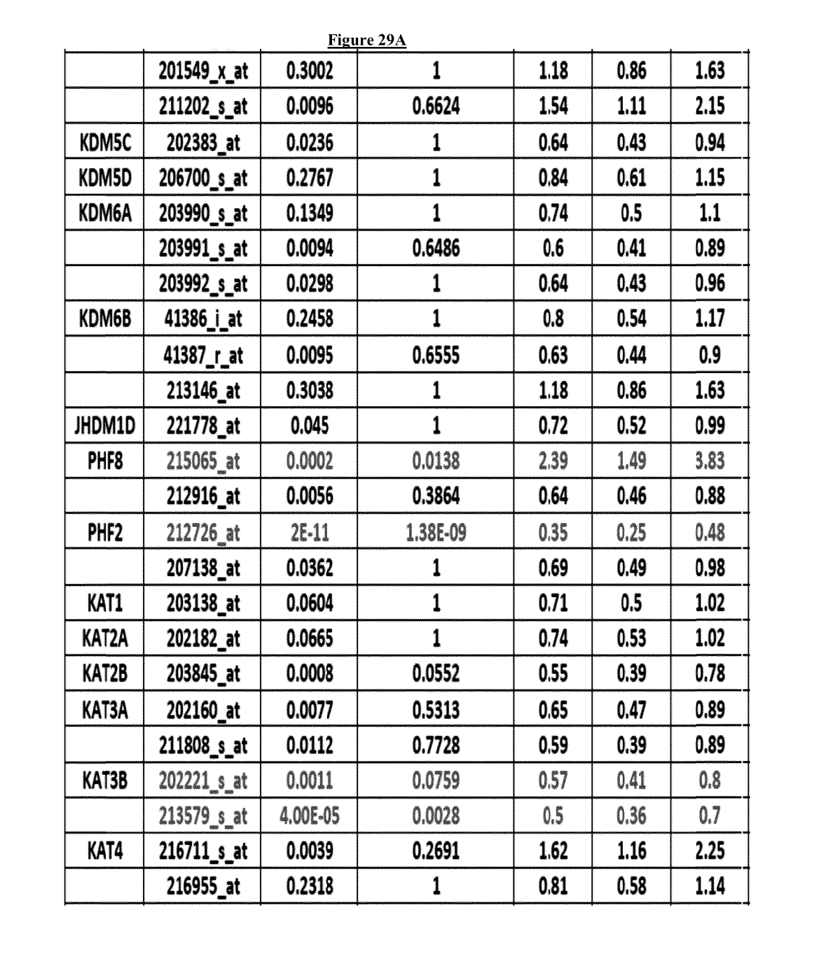

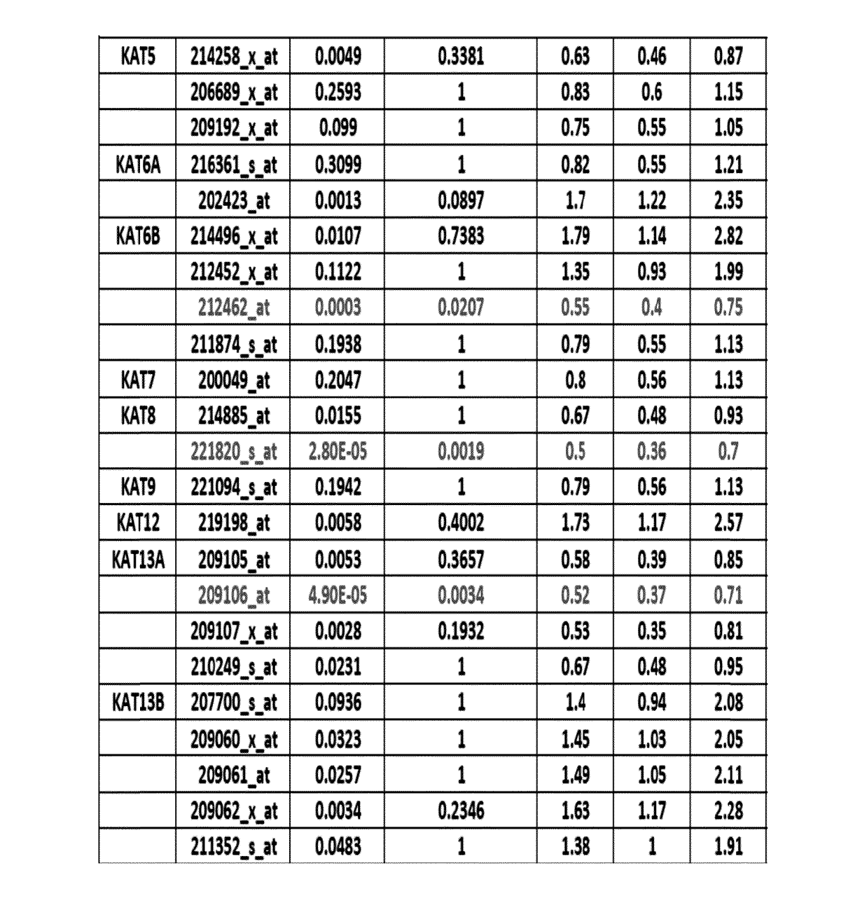

To further validate the findings in a genetically engineered mouse model, the effects of RBP2 loss on breast cancer progression and metastasis was examined using the MMTV-neu transgenic mice, a breast cancer mouse model wherein more than 70% of mice with mammary tumors developed lung metastases. These mice carry wild type neu (the rat HER2 gene) under the control of the MMTV promoter. The RBP2 knockout mice was crossed with the MMTV-neu transgenic mice, and mammary tumor formation and lung metastasis were monitored. RBP2 knockout delayed mammary tumor formation in the MMTV-neu mice (FIG. 23A), suggesting that RBP2 can contribute to mammary tumor development. Lungs were obtained from mice when their primary mammary tumors were approximately 1 cm³ and of similar size (FIG. 28). Similar to the results obtained from the experimental lung colonization model, RBP2 knockout mice showed dramatic decrease of the number of lung metastasis nodules and the incidence of lung metastasis (FIG. 23B-D). Interestingly, in this model, most of the lung metastasis nodules were located inside the blood vessels, suggesting that RBP2 can also mediate the survival and proliferation of breast cancer cells in the pulmonary vasculature. Taken together, the findings from the cell line models, preclinical mouse models and clinical tumor samples strongly support a role for RBP2 as an epigenetic driver of metastasis.

In summary, through mining the gene expression profiles of human mammary tumors, RBP2 was identified as a strong predictor of breast cancer metastasis. Consistent with this finding, RBP2 was overexpressed in highly metastatic breast cancer cell lines. RBP2 have pleiotropic roles in invasion and metastasis and RBP2 function is linked to regulation of several genes known to be involved in metastasis. Although recent studies highlighted the connection of several epigenetic regulators to tumor metastasis, these studies were mostly limited to experiments using cancer cell lines or mouse xenograft models. The present study is bolstered by both clinical and functional data, which identify and functionally demonstrate RBP2 as a critical epigenetic mediator of metastasis and a promising cancer target. These results provide a strong rationale to target RBP2 in the treatment of invasive and metastatic breast cancer.

Triple negative (for ER, PR and HER2) breast cancer is the most aggressive subtype of breast cancer and is often associated with increased and early incidence of tumor metastasis and poor outcome. However, only a limited number of targeted therapeutic methods for these patients are currently under investigation in the clinic. In this study, it was demonstrated that RBP2 is critical for invasion and metastasis of triple negative breast cancer cells (FIGS. 21-22). Suppression of tumor formation and metastasis by RBP2 loss in the MMTV-neu model (FIG. 23) suggests that RBP2 also promotes breast cancer metastasis of HER2 positive (HER2⁺) patients, the majority of whom are also ER⁻. These findings are consistent with the results from human patient samples, where RBP2 is associated with increased metastasis in ER⁻ patients (FIGS. 19C and 24D). Taken together, these results support the specific requirement of RBP2 in ER⁻ breast cancer and provide the rationale for novel treatment methods targeting RBP2 in patients with aggressive subtypes of breast cancer.

A bioinformatics analysis tool indicated that RBP2 level correlates with increased incidence of breast cancer metastasis to distant organs in a total of 2,977 non-redundant breast tumors. The functional studies clearly indicate that RBP2 can enhance metastasis and that it is a pleiotropic positive regulator of many metastasis genes. Thus, RBP2 may be a critical epigenetic switch that enables tumor cells to metastasize through activating a constellation of metastasis genes.

Several possible mechanisms could mediate the activation of these genes by RBP2. Contrary to the prevailing notion that RBP2 can only repress gene expression through histone demethylation at promoters, RBP2 can also directly activate gene expression through several possible mechanisms. The carboxyl-terminal PHD domain of RBP2 specifically recognizes the active H3K4me3/2 marks and is involved in active gene transcription. JARID1C (also known as SMCX or KDM5C) promotes enhancer function by removing spurious H3K4me3/2 at enhancers. It is therefore possible that RBP2 activates enhancers and ultimately gene expression through analogous mechanism. RBP2 was shown to interact with other transcription factors, including c-Myc, which could also lead to gene activation. Lastly, RBP2 might activate gene expression indirectly through repressing the negative regulators of these metastasis genes.

RBP2 has been implicated as an oncoprotein in various cancer types. For example, RBP2 amplification was reported in approximately 15% of breast cancers. In this study, it was showed that knockdown or loss of RBP2 inhibits breast cancer metastasis using two mouse metastasis models. The requirement for RBP2 demethylase activity in cancer cell proliferation suggests that RBP2 demethylase can be therapeutically targeted. Based on the required demethylase activity of RBP2 for breast cancer progression and metastasis, JARID1/2 demethylase inhibitors may be further developed into agents to treat metastatic breast cancer.

Sequences

| | |
|---|---|
| SEQ ID NO: 1 | H3K4me3[ART-K(Me3)- |
| | GTARKSTGGKAPRKQLA-GGK(Biotin)] |
| SEQ ID NO: 2 | H3K4me2[ART-K(Me2)- |
| | GTARKSTGGKAPRKQLA-GGK(Biotin)] |
| SEQ ID NO: 3 | H3K4me1[ART-K(Me1)- |
| | QTARKSTGGKAPRKQLA-GGK(Biotin)] |
| SEQ ID NO: 4 | H3K27me3(ATKAAR-K(Me3)- |
| | SAPATGGVKKPHRYRPG-GK(Biotin)] |
| SEQ ID NO: 5 | H3K27me2(ATKAAR-K(Me2)- |
| | SAPATGGVKKPHRYPG-GK(Biotin)] |
| SEQ ID NO: 6 | H3K27me1(ATKAAR-K(Me1)- |
| | SAPATGGVKKPHRYRPG-GK(Biotin)] |
| SEQ ID NO: 7 | FLAG-tag (DYKDDDDK) |
| SEQ ID NO: 8 | CGAGATGGAATTAACAGTCTT |
| SEQ ID NO: 9 | AGGGAGATGCACTTCGATATA |
| SEQ ID NO: 10 | CCATAGCCGAGCAGACTGG |
| SEQ ID NO: 11 | GGATACGTGGCGTAAAATGAAGT |
| SEQ ID NO: 12 | CGAGATCCCTCCAAAATCAA |
| SEQ ID NO: 13 | GTCTTCTGGGTGGCAGTGAT |
| SEQ ID NO: 14 | CCAGACTTACAGGGACACTTA |
| SEQ ID NO: 15 | CCTTGAAAGAAGCCTTACAAA |
| SEQ ID NO: 16 | GCTGTACGAGAGTATACAC |
| SEQ ID NO: 17 | CTTCTGTACTGCTGACTGG |
| SEQ ID NO: 18 | GCCAAGAACATTCCAGCCT |
| SEQ ID NO: 19 | TGCACCACCAACTGCTTAGC |
| SEQ ID NO: 20 | GGCATGGACTGTGGTCATGAG |
| SEQ ID NO: 21 | CCGTCTTTGAGCCGAGTTG |
| SEQ ID NO: 22 | GGACTCTTGGAGTGAAACGAAA |
| SEQ ID NO: 23 | GTCACCGTGTCAACCTGATG |
| SEQ ID NO: 24 | GCCTGCCTTCAAGATTTCTG |
| SEQ ID NO: 25 | AAGCTTCAGCAACCAGCATT |
| SEQ ID NO: 26 | CCCTCTCTCTCGCTCTCTCA |
| SEQ ID NO: 27 | AGGGGACTCAGAGCTCTTCC |
| SEQ ID NO: 28 | TGCCTGTGGAGTCTTTGATG |
| SEQ ID NO: 29 | CCTGGATGCCATCAAAGTCT |
| SEQ ID NO: 30 | AATCCATCGGTCATGCTCTC |
| SEQ ID NO: 31 | CAAGACCAGGACGGTCATTT |
| SEQ ID NO: 32 | CCTGACGTATTCCACCTTGG |
| SEQ ID NO: 33 | CTTTGAGGATCCAGCCTCTG |
| SEQ ID NO: 34 | TGCGTTTCTTTGTGTTCTCG |
| SEQ ID NO: 35 | CGTGGCTTTCCAAGAAGAAG |
| SEQ ID NO: 36 | GCTTCCGATCTGCTGAAAAC |

TABLE 1
Selected active compounds that inhibit the JARID1B demethylase activity.

| Compound structure | Supplier ID/name | IC₅₀ (μM) high/low Fe (II) |
|---|---|---|
| 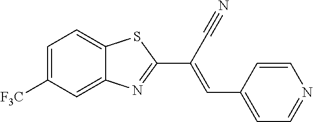 [SMILES: N#C/C(=C\c1ccncc1)c1nc2cc(C(F)(F)F)ccc2s1] | ChemBridge 7812182 | 1.15/1.66 |
| 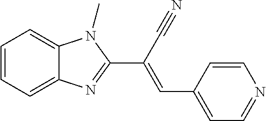 [SMILES: Cn1c(/C(C#N)=C/c2ccncc2)nc2ccccc21] | ChemBridge 6339039 | 1.31/1.80 |
| 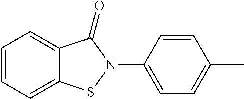 [SMILES: Cc1ccc(-n2sc3ccccc3c2=O)cc1] | 2-(4-methylphenyl)-1,2- benzisothiazol-3(2H)-one (PBIT) | 2.78/3.17 |
| 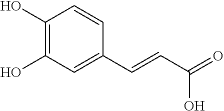 [SMILES: O=C(O)/C=C/c1ccc(O)c(O)c1] | Caffeic acid | 2.88/1.71 |
| 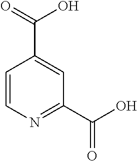 [SMILES: O=C(O)c1ccnc(C(=O)O)c1] | 2,4-pyridinedicarboxylic acid (2,4-PDCA) | 4.47/4.07 |
| 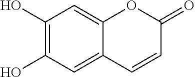 [SMILES: O=c1ccc2cc(O)c(O)cc2o1] | Esculetin | 4.60/2.57 |
| 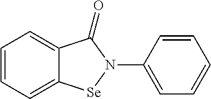 [SMILES: O=c1c2ccccc2[se]n1-c1ccccc1] | Ebselen | 5.17/7.63 |

The compounds are listed by structure, supplier ID or name (if available), and IC₅₀ value from dose response curves performed at 50 μM (high) and 15 μM (low) Fe (II).

TABLE 2
Active inhibitory compounds against JARID1B from a high-throughput screen of 15,134 small molecules.

| | | | | | Inhibitory |
|---|---|---|---|---|---|
| Compound | | | | | effect |
| ID | Structure | Supplier | Supplier ID | Drug Name | (percent) |
| YU033841 | 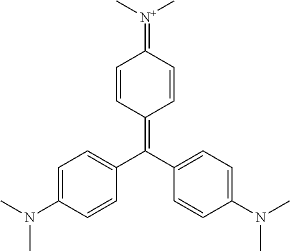 | Microsource | 01500315 | GENTIAN VIOLET | 105.84 |
| YU155507 | 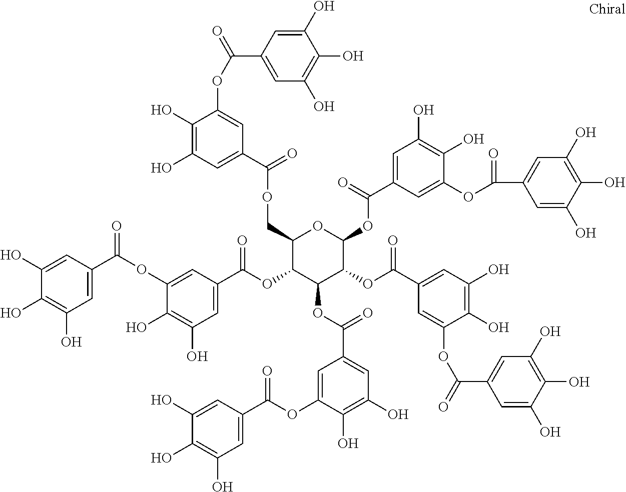 [SMILES: O=C(OC[C@H]1O[C@@H](OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1] | Microsource | 01504105 | TANNIC ACID | 105.46 |
| YU155257 | 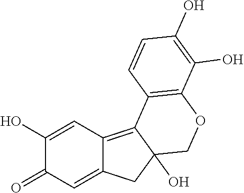 [SMILES: O=C1C=C2CC3(O)COc4c(ccc(O)c4O)C3=C2C=C1O] | Microsource | 01502253 | HEMATEIN | 105.45 |
| YU034398 | 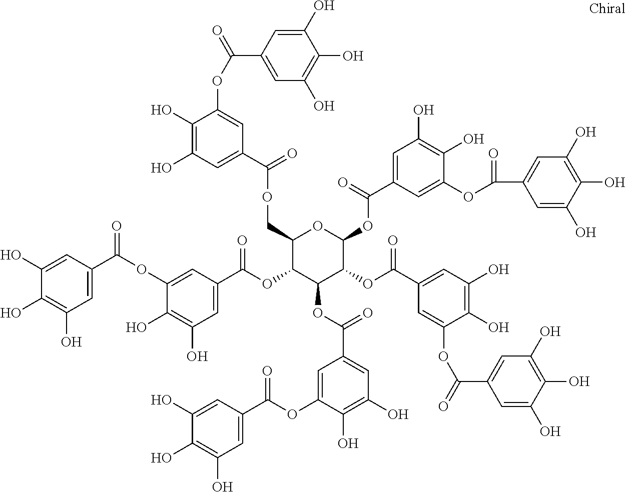 [SMILES: O=C(OC[C@H]1O[C@@H](OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1] | Microsource | 01504105 | TANNIC ACID | 105.34 |
| YU039791 | 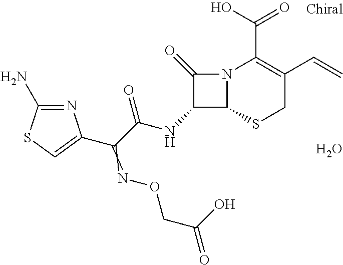 [SMILES: C=CC1=C(C(=O)O)N2C(=O)[C@@H](NC(=O)C(=NOCC(=O)O)c3csc(N)n3)[C@H]2SC1 ; O] | NCC | SAM001246816 | CEFIXIME TRIHYDRATE | 105.16 |
| YU155100 | 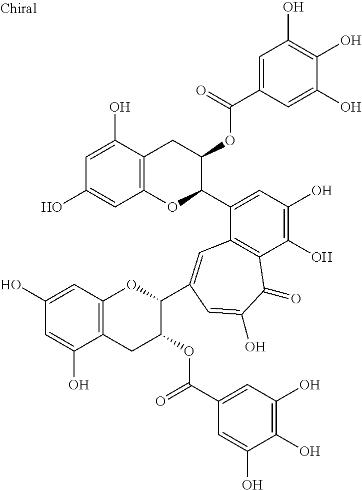 [SMILES: O=C(O[C@@H]1Cc2c(O)cc(O)cc2O[C@@H]1c1cc(O)c(=O)c2c(O)c(O)cc([C@H]3Oc4cc(O)cc(O)c4C[C@H]3OC(=O)c3cc(O)c(O)c(O)c3)c2c1)c1cc(O)c(O)c(O)c1] | Microsource | 00201515 | THEAFLAVIN DIGALLATE | 105.15 |
| YU155305 | 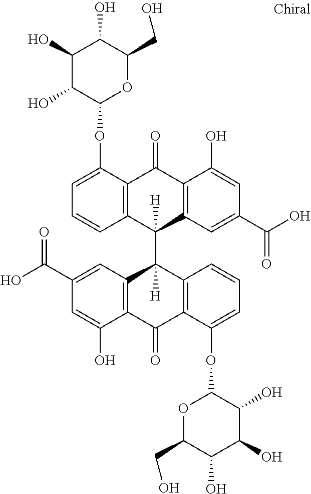 | Microsource | 01504080 | SENNOSIDE B | 105.06 |
| YU016748 | 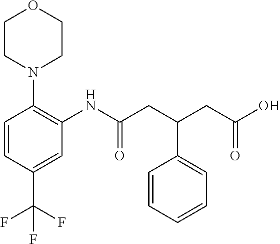 [SMILES: O=C(O)CC(CC(=O)Nc1cc(C(F)(F)F)ccc1N1CCOCC1)c1ccccc1] | MayBridge | HTS 06033 | | 104.42 |
| YU034331 | 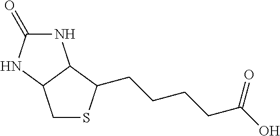 [SMILES: O=C(O)CCCCC1SCC2NC(=O)NC21] | Microsource | 01503009 | BIOTIN | 104.13 |
| YU155347 | 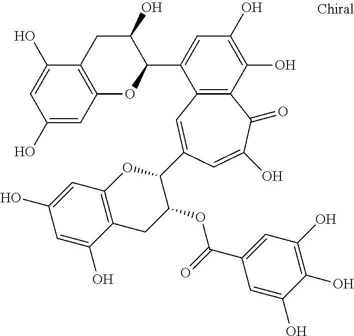 [SMILES: O=C(O[C@@H]1Cc2c(O)cc(O)cc2O[C@@H]1c1cc(O)c(=O)c2c(O)c(O)cc([C@H]3Oc4cc(O)cc(O)c4C[C@H]3O)c2c1)c1cc(O)c(O)c(O)c1] | Microsource | 00210242 | THEAFLAVIN MONOGALLATES | 103.69 |
| YU155360 | 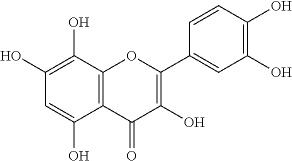 [SMILES: O=c1c(O)c(-c2ccc(O)c(O)c2)oc2c(O)c(O)cc(O)c12] | Microsource | 01505143 | GOSSYPETIN | 103.49 |
| YU154887 | 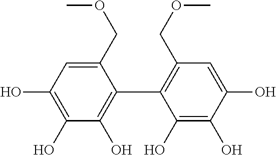 [SMILES: COCc1cc(O)c(O)c(O)c1-c1c(COC)cc(O)c(O)c1O] | Enzo | EI-273 | 2,2,3,3,4,4- HEXAHYDROXY-1,1- BIPHENYL-6,6- DIMETHANOL DIMETHYL ETHER | 103.22 |
| YU153074 | 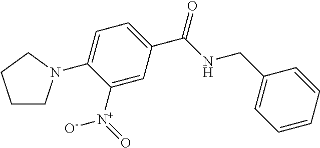 [SMILES: O=C(NCc1ccccc1)c1ccc(N2CCCC2)c([N+](=O)[O-])c1] | ChemBridge | 7672253 | | 101.87 |
| YU034556 | 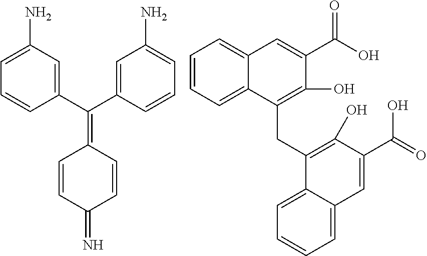 | Microsource | 01503223 | PARAROSANILINE PAMOATE | 101.78 |
| YU155084 | 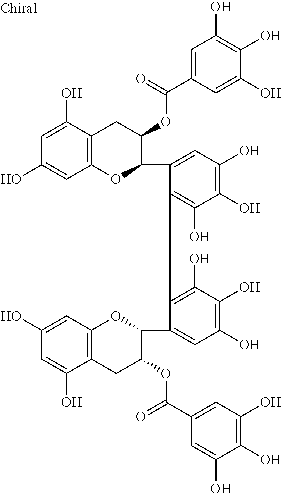 [SMILES: O=C(O[C@@H]1Cc2c(O)cc(O)cc2O[C@@H]1c1cc(O)c(O)c(O)c1-c1c([C@H]2Oc3cc(O)cc(O)c3C[C@H]2OC(=O)c2cc(O)c(O)c(O)c2)cc(O)c(O)c1O)c1cc(O)c(O)c(O)c1] | Microsource | 00201507 | 2,2-BISEPIGALLO- CATECHIN DIGALLATE | 101.51 |
| YU034420 | 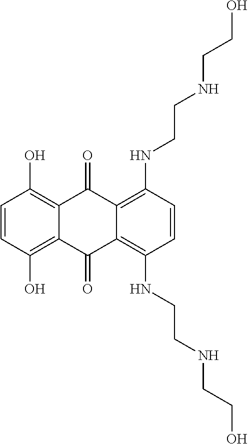 [SMILES: O=C1c2c(O)ccc(O)c2C(=O)c2c(NCCNCCO)ccc(NCCNCCO)c21] | Microsource | 01503278 | MITOXANTHRONE HYDROCHLORIDE | 101.44 |
| YU155005 | 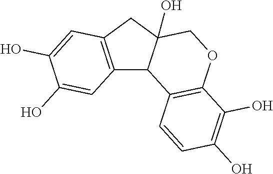 [SMILES: Oc1cc2c(cc1O)C1c3ccc(O)c(O)c3OCC1(O)C2] | Microsource | 00200010 | HAEMATOXYLIN | 100.83 |
| YU153128 | 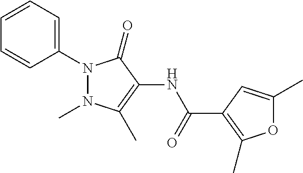 [SMILES: Cc1cc(C(=O)Nc2c(C)n(C)n(-c3ccccc3)c2=O)c(C)o1] | ChemBridge | 7944562 | | 100.64 |
| YU153545 | 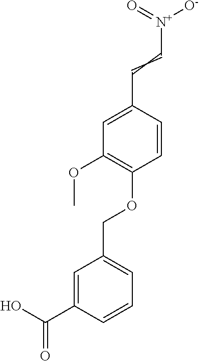 [SMILES: COc1cc(C=C[N+](=O)[O-])ccc1OCc1cccc(C(=O)O)c1] | ChemBridge | 6395104 | | 100.57 |
| YU034292 | 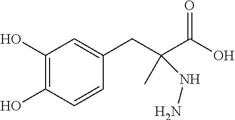 [SMILES: CC(Cc1ccc(O)c(O)c1)(NN)C(=O)O] | Microsource | 01502150 | CARBIDOPA | 100.30 |
| YU040338 | 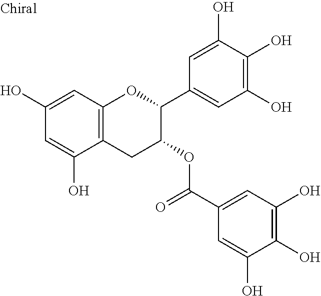 [SMILES: O=C(O[C@@H]1Cc2c(O)cc(O)cc2O[C@@H]1c1cc(O)c(O)c(O)c1)c1cc(O)c(O)c(O)c1] | NCC | SAM001247031 | Epigallocatechin gallate | 100.16 |
| YU152564 | 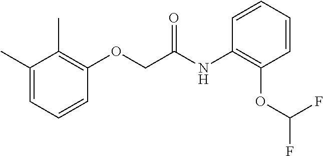 [SMILES: Cc1cccc(OCC(=O)Nc2ccccc2OC(F)F)c1C] | ChemBridge | 7930515 | | 99.63 |
| YU155085 | 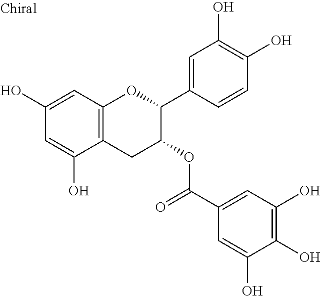 [SMILES: O=C(O[C@@H]1Cc2c(O)cc(O)cc2O[C@@H]1c1ccc(O)c(O)c1)c1cc(O)c(O)c(O)c1] | Microsource | 00210238 | EPICATECHIN MONOGALLATE | 99.55 |
| YU034068 | 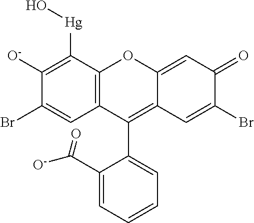 | Microsource | 01500637 | MERBROMIN | 99.27 |
| YU034020* | 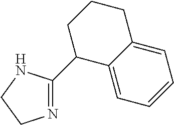 [SMILES: c1ccc2c(c1)CCCC2C1=NCCN1] | Microsource | 01500567 | TETRAHYDROZOLINE HYDROCHLORIDE | 99.25 |
| YU033800 | 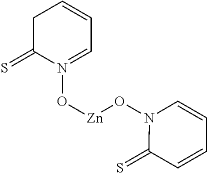 | Microsource | 01500260 | PYRITHIONE ZINC | 99.02 |
| YU034023 | 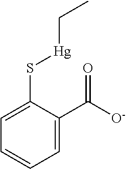 | Microsource | 01500572 | THIMEROSAL | 99.00 |
| YU152649 | 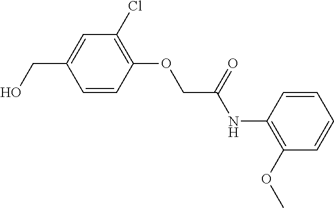 [SMILES: COc1ccccc1NC(=O)COc1ccc(CO)cc1Cl] | ChemBridge | 7937631 | | 98.99 |
| YU034506 | 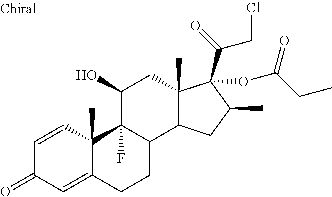 | Microsource | 01503918 | CLOBETASOL PROPIONATE | 98.92 |
| YU155356 | 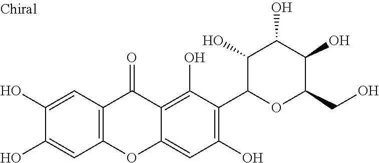 [SMILES: O=c1c2cc(O)c(O)cc2oc2cc(O)c(C3O[C@H](CO)[C@H](O)[C@@H](O)[C@H]3O)c(O)c12] | Microsource | 01505134 | MANGIFERIN | 98.82 |
| YU034074 | 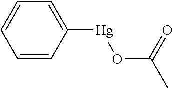 | Microsource | 01500644 | PHENYLMERCURIC ACETATE | 98.73 |
| YU155090 | 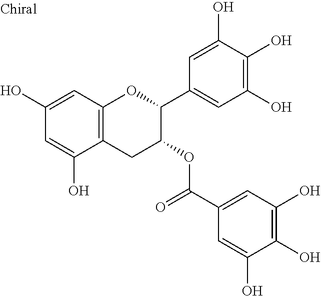 [SMILES: O=C(O[C@@H]1Cc2c(O)cc(O)cc2O[C@@H]1c1cc(O)c(O)c(O)c1)c1cc(O)c(O)c(O)c1] | Microsource | 00210239 | EPIGALLOCATECHIN-3- MONOGALLATE | 98.42 |
| YU152587 | 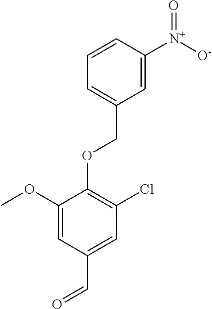 [SMILES: COc1cc(C=O)cc(Cl)c1OCc1cccc([N+](=O)[O-])c1] | ChemBridge | 6498914 | | 98.19 |
| YU155193 | 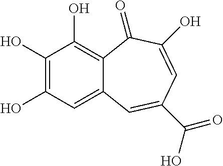 [SMILES: O=C(O)c1cc(O)c(=O)c2c(O)c(O)c(O)cc2c1] | Microsource | 00240826 | PURPUROGALLIN-4- CARBOXYLIC ACID | 97.52 |
| YU150403 | 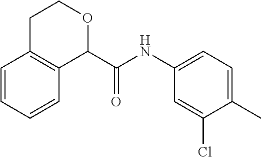 [SMILES: Cc1ccc(NC(=O)C2OCCc3ccccc32)cc1Cl] | ChemBridge | 7933837 | | 97.47 |
| YU153031 | 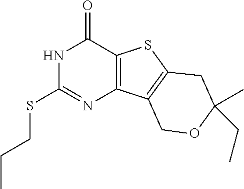 [SMILES: CCCSc1nc2c3c(sc2c(=O)[nH]1)CC(C)(CC)OC3] | ChemBridge | 7919640 | | 97.05 |
| YU155621 | 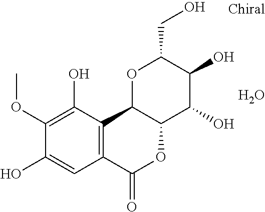 | Microsource | 01500819 | BERGENIN | 96.80 |
| YU152920 | 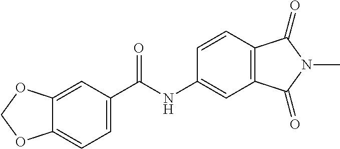 [SMILES: CN1C(=O)c2ccc(NC(=O)c3ccc4c(c3)OCO4)cc2C1=O] | ChemBridge | 7799774 | | 96.74 |
| YU172999 | 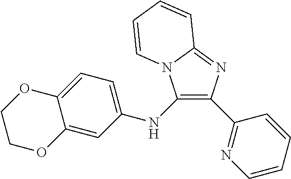 [SMILES: c1ccc(-c2nc3ccccn3c2Nc2ccc3c(c2)OCCO3)nc1] | ChemDiv | C177-0098 | | 96.73 |
| YU151071 | 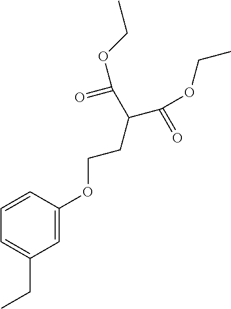 [SMILES: CCOC(=O)C(CCOc1cccc(CC)c1)C(=O)OCC] | ChemBridge | 7005264 | | 96.65 |
| YU035082* | 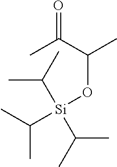 [SMILES: CC(=O)C(C)O[Si](C(C)C)(C(C)C)C(C)C] | Yale University | JS24 | | 95.85 |
| YU034263 | 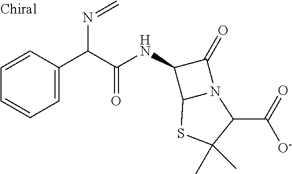 [SMILES: C=NC(C(=O)N[C@H]1C(=O)N2C1SC(C)(C)C2C(=O)[O-])c1ccccc1] | Microsource | 01502034 | METAMPICILLIN SODIUM | 95.63 |
| YU155411 | 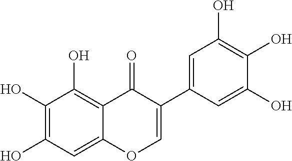 [SMILES: O=c1c(-c2cc(O)c(O)c(O)c2)coc2cc(O)c(O)c(O)c12] | Microsource | 00201182 | IRIGENOL | 94.87 |
| YU145461 | 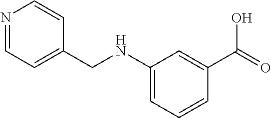 [SMILES: O=C(O)c1cccc(NCc2ccncc2)c1] | ChemBridge | 7935634 | | 94.61 |
| YU034377 | 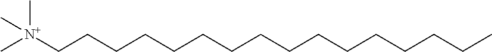 [SMILES: CCCCCCCCCCCCCCCC[N+](C)(C)C] | Microsource | 01503200 | CETRIMONIUM BROMIDE | 94.25 |
| YU173004 | 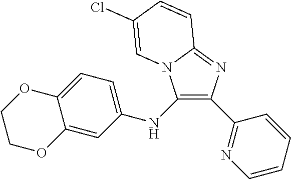 [SMILES: Clc1ccc2nc(-c3ccccn3)c(Nc3ccc4c(c3)OCCO4)n2c1] | ChemDiv | C177-0168 | | 93.95 |
| YU154882 | 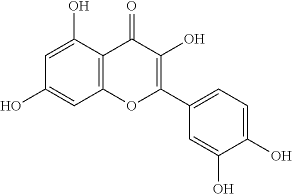 [SMILES: O=c1c(O)c(-c2ccc(O)c(O)c2)oc2cc(O)cc(O)c12] | Microsource | 01500672 | QUERCETIN | 93.51 |
| YU033898 | 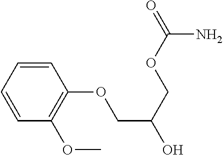 [SMILES: COc1ccccc1OCC(O)COC(N)=O] | Microsource | 01500397 | METHOCARBAMOL | 93.18 |
| YU172089 | 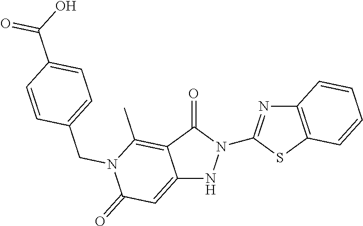 [SMILES: Cc1c2c(=O)n(-c3nc4ccccc4s3)[nH]c2cc(=O)n1Cc1ccc(C(=O)O)cc1] | ChemDiv | 8249-3642 | | 92.32 |
| YU040321 | 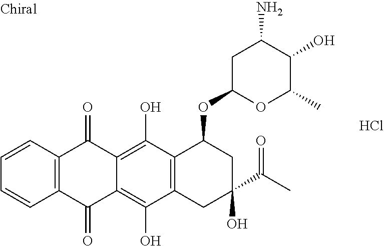 [SMILES: CC(=O)[C@]1(O)Cc2c(O)c3c(c(O)c2[C@@H](O[C@H]2C[C@H](N)[C@H](O)[C@H](C)O2)C1)C(=O)c1ccccc1C3=O ; Cl] | NCC | SAM001246676 | IDARUBICIN HCl | 92.30 |
| YU155091 | 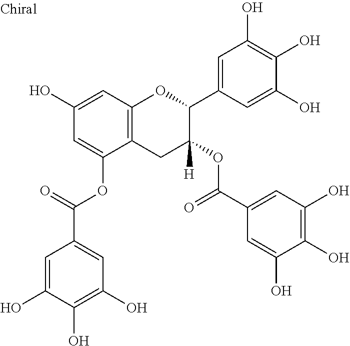 [SMILES: O=C(Oc1cc(O)cc2c1C[C@@H](OC(=O)c1cc(O)c(O)c(O)c1)[C@@H](c1cc(O)c(O)c(O)c1)O2)c1cc(O)c(O)c(O)c1] | Microsource | 00201513 | EPIGALLOCATECHIN 3,5-DIGALLATE | 92.18 |
| YU145132 | 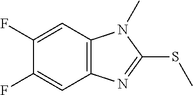 | ChemBridge | 7862194 | | 91.46 |
| YU155407 | 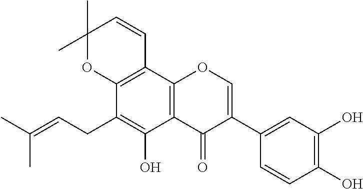 [SMILES: CC(C)=CCc1c2c(c3occ(-c4ccc(O)c(O)c4)c(=O)c3c1O)C=CC(C)(C)O2] | Microsource | 00201580 | POMIFERIN | 91.09 |
| YU153803 | 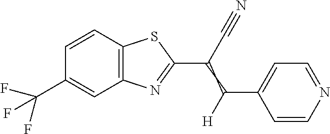 [SMILES: N#CC(=Cc1ccncc1)c1nc2cc(C(F)(F)F)ccc2s1] | ChemBridge | 7812182 | | 90.81 |
| YU146842 | 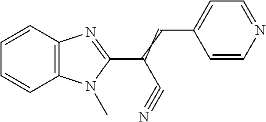 [SMILES: Cn1c(C(C#N)=Cc2ccncc2)nc2ccccc21] | ChemBridge | 6339039 | | 90.80 |
| YU144896 | 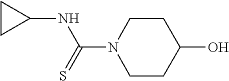 [SMILES: OC1CCN(C(=S)NC2CC2)CC1] | ChemBridge | 7853261 | | 90.56 |
| YU033743* | 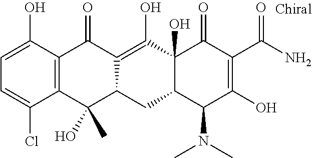 [SMILES: CN(C)[C@@H]1C(O)=C(C(N)=O)C(=O)[C@@]2(O)C(O)=C3C(=O)c4c(O)ccc(Cl)c4[C@@](C)(O)[C@H]3C[C@@H]12] | Microsource | 01500186 | AUREOMYCIN | 89.95 |
| YU033696 | 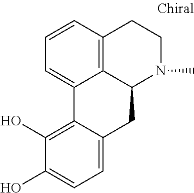 | Microsource | 01500129 | APOMORPHINE HYDROCHLORIDE | 89.43 |
| YU155353 | 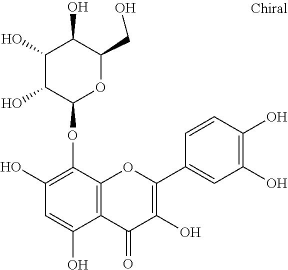 [SMILES: O=c1c(O)c(-c2ccc(O)c(O)c2)oc2c(O[C@@H]3O[C@H](CO)[C@H](O)[C@@H](O)[C@H]3O)c(O)cc(O)c12] | Microsource | 01505127 | GOSSYPIN | 89.03 |
| YU033698 | 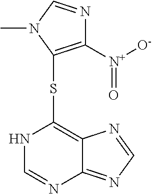 [SMILES: Cn1cnc([N+](=O)[O-])c1Sc1[nH]cnc2ncnc1-2] | Microsource | 01500133 | AZATHIOPRINE | 88.78 |
| YU129955 | 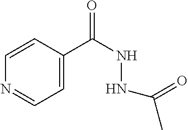 | ChemBridge | 5131356 | | 88.72 |
| YU035100 | 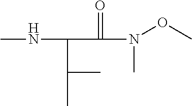 | Yale University | JS46 | | 87.99 |
| YU146026 | 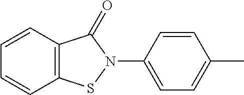 [SMILES: Cc1ccc(-n2sc3ccccc3c2=O)cc1] | ChemBridge | 7938963 | | 87.75 |
| YU145649 | 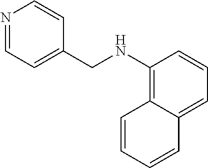 [SMILES: c1ccc2c(NCc3ccncc3)cccc2c1] | ChemBridge | 5224440 | | 87.71 |
| YU033662 | 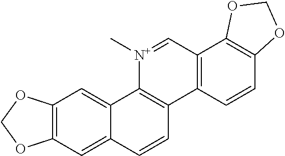 [SMILES: C[n+]1cc2c3c(ccc2c2ccc4cc5c(cc4c21)OCO5)OCO3] | Microsource | 00310035 | SANGUINARINE SULFATE | 86.66 |
| YU147750 | 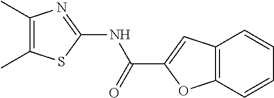 [SMILES: Cc1nc(NC(=O)c2cc3ccccc3o2)sc1C] | ChemBridge | 7911930 | | 86.41 |
| YU154882 | 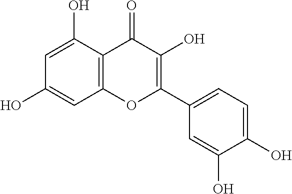 [SMILES: O=c1c(O)c(-c2ccc(O)c(O)c2)oc2cc(O)cc(O)c12] | Enzo | AC-1142 | QUERCETIN | 85.99 |
| YU151614 | 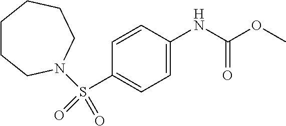 [SMILES: COC(=O)Nc1ccc(S(=O)(=O)N2CCCCCC2)cc1] | ChemBridge | 7944568 | | 85.50 |
| YU148374 | 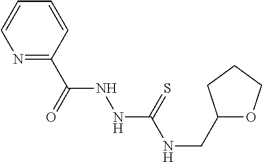 [SMILES: O=C(NNC(=S)NCC1CCCO1)c1ccccn1] | ChemBridge | 7813798 | | 84.83 |
| YU149951 | 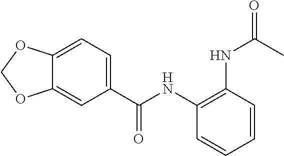 [SMILES: CC(=O)Nc1ccccc1NC(=O)c1ccc2c(c1)OCO2] | ChemBridge | 7498349 | | 84.69 |
| YU152893 | 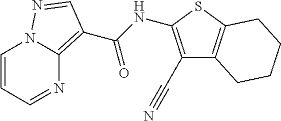 [SMILES: N#Cc1c(NC(=O)c2cnn3cccnc23)sc2c1CCCC2] | ChemBridge | 7961326 | | 84.55 |
| YU151859 | 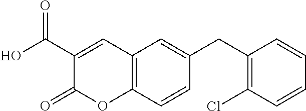 [SMILES: O=C(O)c1cc2cc(Cc3ccccc3Cl)ccc2oc1=O] | ChemBridge | 7960076 | | 84.36 |
| YU173003 | 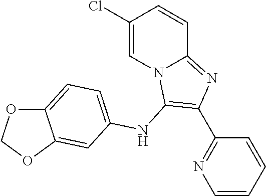 [SMILES: Clc1ccc2nc(-c3ccccn3)c(Nc3ccc4c(c3)OCO4)n2c1] | ChemDiv | C177-0167 | | 83.13 |
| YU039604 | 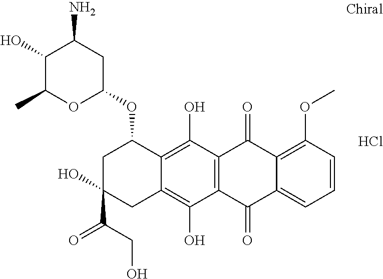 [SMILES: COc1cccc2c1C(=O)c1c(O)c3c(c(O)c1C2=O)C[C@@](O)(C(=O)CO)C[C@@H]3O[C@H]1C[C@H](N)[C@@H](O)[C@H](C)O1 ; Cl] | NCC | SAM001246559 | EPIRUBICIN HYDROCHLORIDE | 81.87 |
| YU034347 | 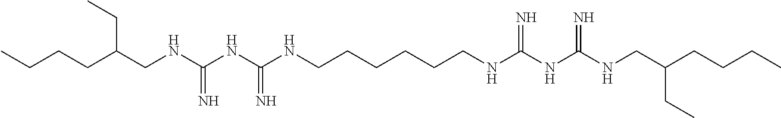 [SMILES: CCCCC(CC)CNC(=N)NC(=N)NCCCCCCNC(=N)NC(=N)NCC(CC)CCCC] | Microsource | 01503074 | ALEXIDINE HYDROCHLORIDE | 80.56 |
| YU039629 | 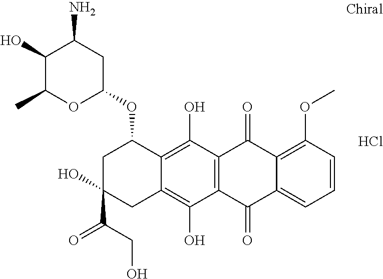 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; Cl] | NCC | SAM001246768 | DOXORUBICIN HYDROCHLORIDE | 80.31 |
| YU034090 | 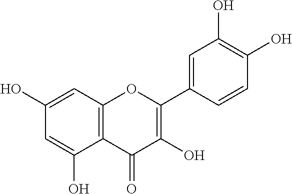 [SMILES: O=c1c(O)c(-c2ccc(O)c(O)c2)oc2cc(O)cc(O)c12] | Microsource | 01500672 | QUERCETIN | 80.17 |
| YU034518 | 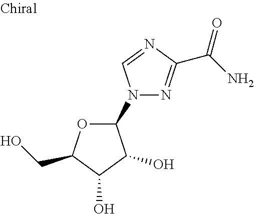 [SMILES: NC(=O)c1ncn([C@@H]2O[C@H](CO)[C@@H](O)[C@H]2O)n1] | Microsource | 0150398 | RIBAVIRIN | 79.69 |
| YU155025 | 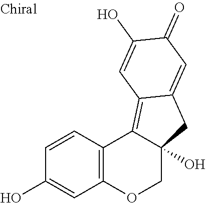 [SMILES: O=C1C=C2C[C@@]3(O)COc4cc(O)ccc4C3=C2C=C1O] | Microsource | 00200463 | BRAZILEIN | 78.77 |
| YU145089 | 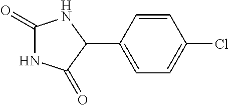 [SMILES: O=C1NC(=O)C(c2ccc(Cl)cc2)N1] | ChemBridge | 7990751 | | 78.33 |
| YU034280 | 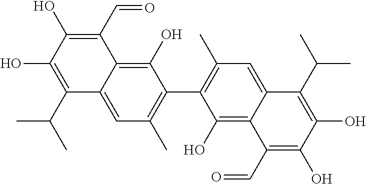 [SMILES: Cc1cc2c(C(C)C)c(O)c(O)c(C=O)c2c(O)c1-c1c(C)cc2c(C(C)C)c(O)c(O)c(C=O)c2c1O] | Microsource | 01502099 | GOSSYPOL-ACETIC ACID COMPLEX | 78.24 |
| YU154833 | 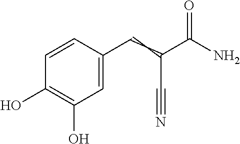 [SMILES: N#CC(=Cc1ccc(O)c(O)c1)C(N)=O] | Enzo | EI-257 | TYRPHOSTIN 46 | 76.83 |
| YU145853 | 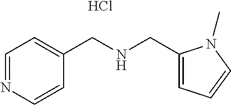 | ChemBridge | 7939195 | | 76.60 |
| YU155075 | 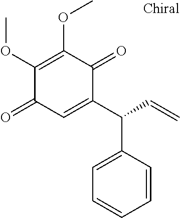 [SMILES: C=C[C@@H](C1=CC(=O)C(OC)=C(OC)C1=O)c1ccccc1] | Microsource | 00240828 | 3,4-DIMETHOXY- DALBERGIONE | 75.94 |
| YU034066 | 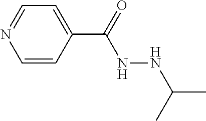 | Microsource | 01500634 | IPRONIAZID SULFATE | 75.93 |
| YU155188 | 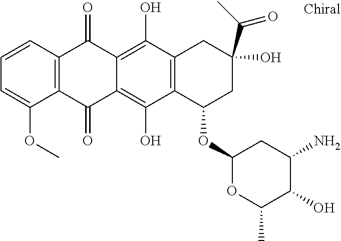 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1] | Microsource | 01500223 | DAUNORUBICIN | 74.66 |
| YU152451 | 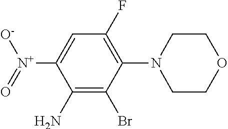 [SMILES: Nc1c([N+](=O)[O-])cc(F)c(N2CCOCC2)c1Br] | ChemBridge | 7846193 | | 74.58 |
| YU129874 | 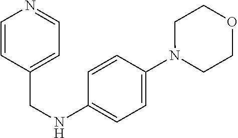 [SMILES: c1cc(CNc2ccc(N3CCOCC3)cc2)ccn1] | ChemBridge | 6104953 | | 74.48 |
| YU033699 | 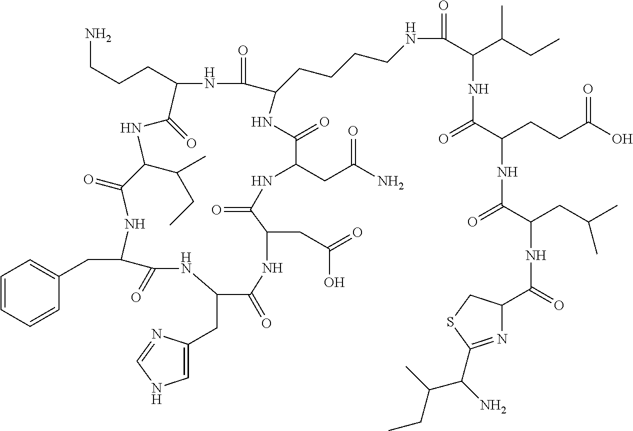 [SMILES: CCC(C)C(N)C1=NC(C(=O)NC(CC(C)C)C(=O)NC(CCC(=O)O)C(=O)NC(C(=O)NCCCCC2NC(=O)C(CC(N)=O)NC(=O)C(CC(=O)O)NC(=O)C(Cc3c[nH]cn3)NC(=O)C(Cc3ccccc3)NC(=O)C(C(C)CC)NC(=O)C(CCCN)NC2=O)C(C)CC)CS1] | Microsource | 01500134 | BACITRACIN | 74.07 |
| YU155528 | 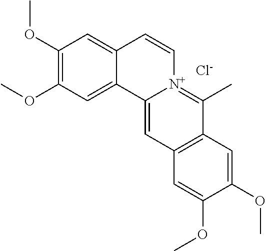 | Microsource | 01500861 | CORALYNE CHLORIDE | 73.15 |
| YU172169 | 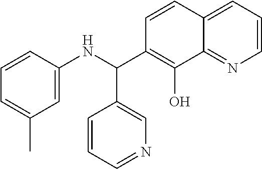 [SMILES: Cc1cccc(NC(c2cccnc2)c2ccc3cccnc3c2O)c1] | ChemDiv | 8397-0180 | | 72.70 |
| YU172178 | 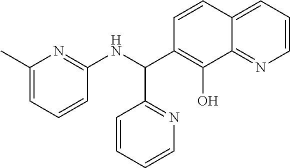 [SMILES: Cc1cccc(NC(c2ccccn2)c2ccc3cccnc3c2O)n1] | ChemDiv | 8397-0664 | | 72.27 |
| YU155300 | 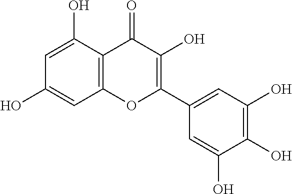 [SMILES: O=c1c(O)c(-c2cc(O)c(O)c(O)c2)oc2cc(O)cc(O)c12] | Microsource | 01504065 | MYRICETIN | 72.12 |
| YU150981 | 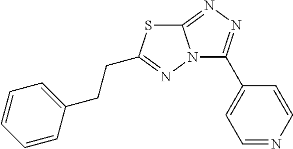 [SMILES: c1ccc(CCc2nn3c(-c4ccncc4)nnc3s2)cc1] | ChemBridge | 7958378 | | 71.24 |
| YU033702 | 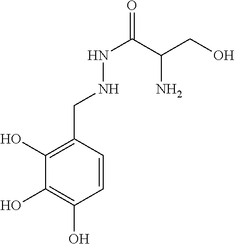 [SMILES: NC(CO)C(=O)NNCc1ccc(O)c(O)c1O] | Microsource | 01500137 | BENSERAZIDE HYDROCHLORIDE | 70.58 |
| YU147704 | 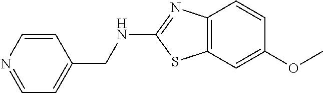 [SMILES: COc1ccc2nc(NCc3ccncc3)sc2c1] | ChemBridge | 7955825 | | 70.43 |
| YU154998 | 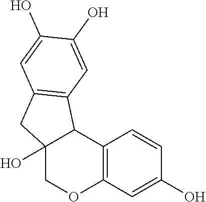 [SMILES: Oc1ccc2c(c1)OCC1(O)Cc3cc(O)c(O)cc3C21] | Microsource | 00200012 | BR AZILIN | 70.33 |
| YU172170 | 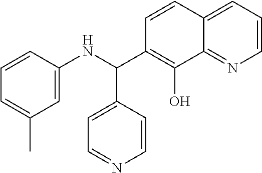 [SMILES: Cc1cccc(NC(c2ccncc2)c2ccc3cccnc3c2O)c1] | ChemDiv | 8397-0181 | | 69.97 |
| YU154835 | 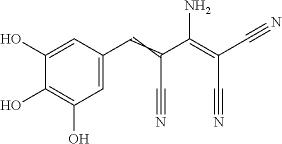 [SMILES: N#CC(=Cc1cc(O)c(O)c(O)c1)C(N)=C(C#N)C#N] | Enzo | EI-189 | TYRPHOSTIN 51 | 69.71 |
| YU033903 | 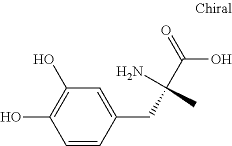 | Microsource | 01500403 | METHYLDOPA | 69.09 |
| YU105079 | 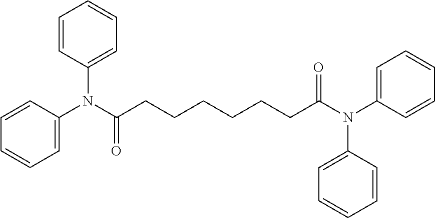 [SMILES: O=C(CCCCCCC(=O)N(c1ccccc1)c1ccccc1)N(c1ccccc1)c1ccccc1] | ChemBridge | 5152461 | | 68.91 |
| YU034332 | 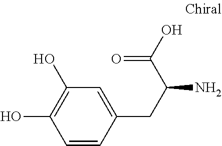 [SMILES: N[C@@H](Cc1ccc(O)c(O)c1)C(=O)O] | Microsource | 02300205 | LEVODOPA | 68.81 |
| YU034048 | 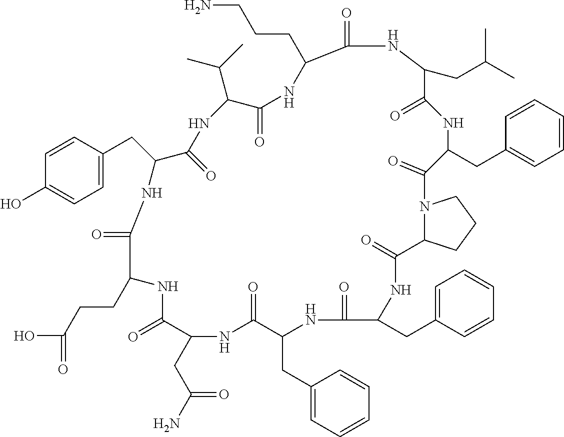 [SMILES: CC(C)CC1NC(=O)C(CCCN)NC(=O)C(C(C)C)NC(=O)C(Cc2ccc(O)cc2)NC(=O)C(CCC(=O)O)NC(=O)C(CC(N)=O)NC(=O)C(Cc2ccccc2)NC(=O)C(Cc2ccccc2)NC(=O)C2CCCN2C(=O)C(Cc2ccccc2)NC1=O] | Microsource | 01500603 | TYROTHRICIN | 68.66 |
| YU147266 | 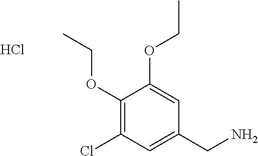 | ChemBridge | 7864151 | | 68.58 |
| YU033628 | 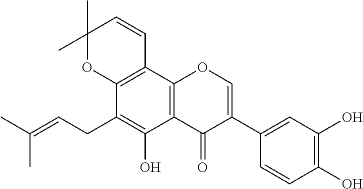 [SMILES: CC(C)=CCc1c2c(c3occ(-c4ccc(O)c(O)c4)c(=O)c3c1O)C=CC(C)(C)O2] | Microsource | 00201580 | POMIFERIN | 68.25 |
| YU145521 | 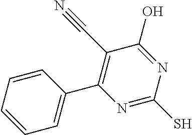 [SMILES: N#Cc1c(O)nc(S)nc1-c1ccccc1] | ChemBridge | 7694782 | | 68.15 |
| YU155280 | 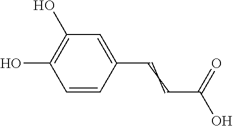 [SMILES: O=C(O)C=Cc1ccc(O)c(O)c1] | Microsource | 01503987 | CAFFEIC ACID | 67.29 |
| YU145037 | 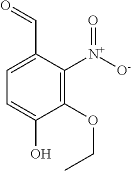 | ChemBridge | 7390437 | | 66.07 |
| YU034370 | 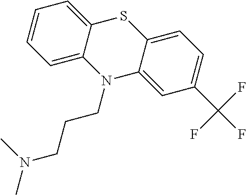 [SMILES: CN(C)CCCN1c2ccccc2Sc2ccc(C(F)(F)F)cc21] | Microsource | 01503118 | TRIFLUPROMAZINE HYDROCHLORIDE | 66.04 |
| YU155618 | 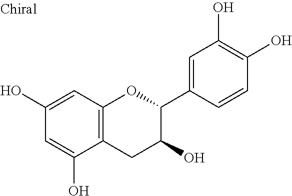 [SMILES: Oc1cc(O)c2c(c1)O[C@H](c1ccc(O)c(O)c1)[C@@H](O)C2] | Microsource | 00210206 | EPICATECHIN | 65.98 |
| YU033654 | 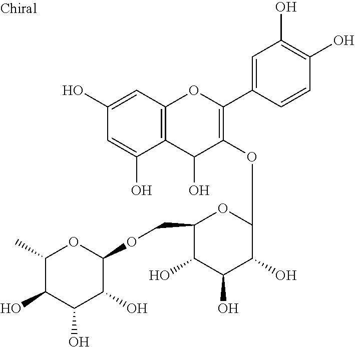 [SMILES: C[C@@H]1O[C@@H](OC[C@H]2OC(OC3=C(c4ccc(O)c(O)c4)Oc4cc(O)cc(O)c4C3O)[C@H](O)[C@@H](O)[C@@H]2O)[C@H](O)[C@H](O)[C@H]1O] | Microsource | 00300607 | RUTOSIDE (rutin) | 65.76 |
| YU153003 | 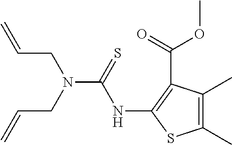 [SMILES: C=CCN(CC=C)C(=S)Nc1sc(C)c(C)c1C(=O)OC] | ChemBridge | 7817806 | | 65.56 |
| YU155214 | 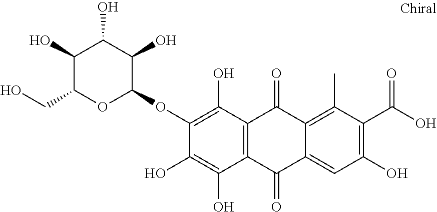 [SMILES: Cc1c(C(=O)O)c(O)cc2c1C(=O)c1c(O)c(O[C@H]3O[C@H](CO)[C@@H](O)[C@H](O)[C@H]3O)c(O)c(O)c1C2=O] | Microsource | 01500817 | CARMINIC ACID | 65.23 |
| YU155285 | 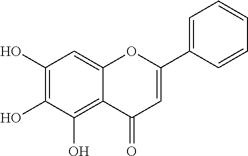 [SMILES: O=c1cc(-c2ccccc2)oc2cc(O)c(O)c(O)c12] | Microsource | 01504002 | BAICALEIN | 65.19 |
| YU034547 | 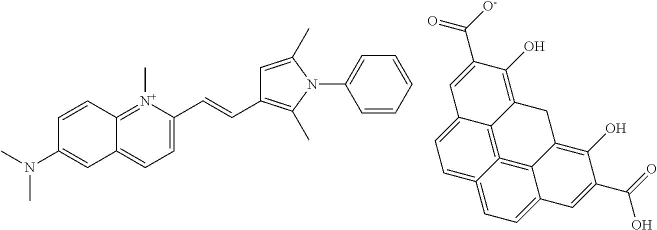 | Microsource | 01500521 | PYRVINIUM PAMOATE | 64.38 |
| YU034426 | 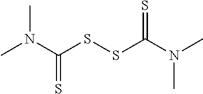 | Microsource | 01503322 | THIRAM | 64.22 |
| YU148680 | 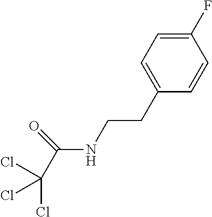 [SMILES: O=C(NCCc1ccc(F)cc1)C(Cl)(Cl)Cl] | ChemBridge | 6818678 | | 63.92 |
| YU034137 | 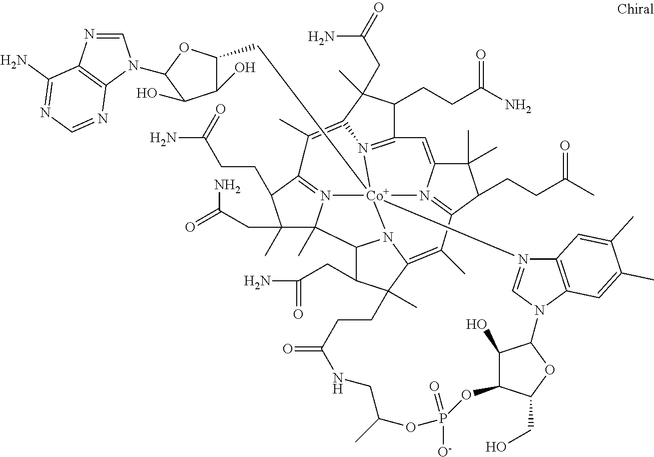 | Microsource | 01500844 | COBALAMINE | 63.90 |
| YU155562 | 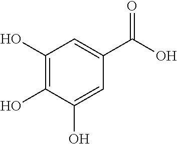 [SMILES: O=C(O)c1cc(O)c(O)c(O)c1] | Microsource | 00210369 | GALLIC ACID | 63.86 |
| YU208194 | 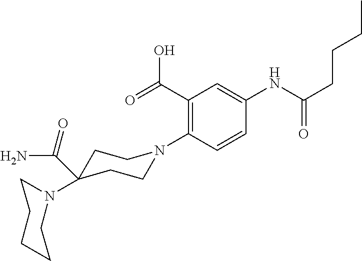 [SMILES: CCCCC(=O)Nc1ccc(N2CCC(C(N)=O)(N3CCCCC3)CC2)c(C(=O)O)c1] | ChemDiv | G889-0171 | | 63.83 |
| YU208199 | 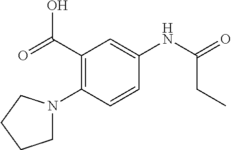 [SMILES: CCC(=O)Nc1ccc(N2CCCC2)c(C(=O)O)c1] | ChemDiv | G889-0409 | | 63.40 |
| YU033837 | 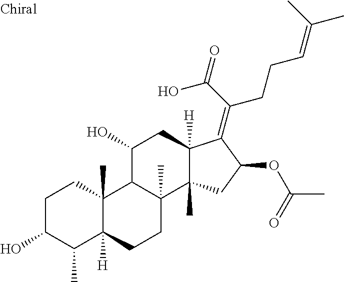 | Microsource | 01500311 | FUSIDIC ACID | 63.06 |
| YU155443 | 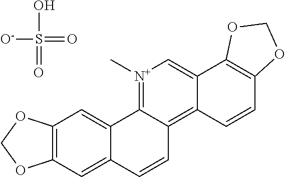 [SMILES: C[n+]1cc2c3c(ccc2c2ccc4cc5c(cc4c21)OCO5)OCO3 ; O=S(=O)([O-])O] | Microsource | 00310035 | SANGUINARINE SULFATE | 62.80 |
| YU172199 | 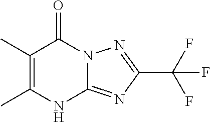 [SMILES: Cc1[nH]c2nc(C(F)(F)F)nn2c(=O)c1C] | ChemDiv | 8407-0795 | | 62.38 |
| YU145029 | 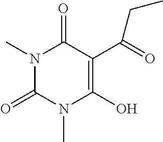 | ChemBridge | 6628987 | | 61.64 |
| YU185529 | 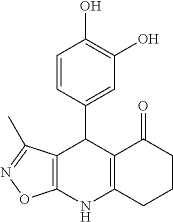 [SMILES: Cc1noc2c1C(c1ccc(O)c(O)c1)C1=C(CCCC1=O)N2] | ChemDiv | D588-0191 | | 61.45 |
| YU034320 | 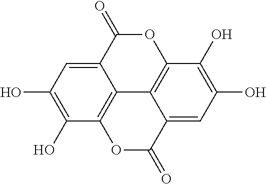 [SMILES: O=c1oc2c(O)c(O)cc3c(=O)oc4c(O)c(O)cc1c4c23] | Microsource | 01502245 | ELLAGIC ACID | 61.25 |
| YU034124 | 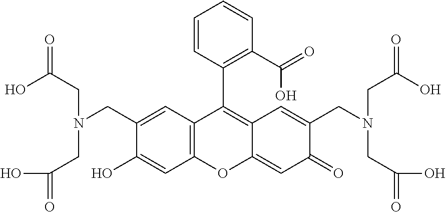 [SMILES: O=C(O)CN(CC(=O)O)Cc1cc2c(-c3ccccc3C(=O)O)c3cc(CN(CC(=O)O)CC(=O)O)c(=O)cc-3oc2cc1O] | Microsource | 01500763 | CALCEIN | 60.48 |
| YU155003 | 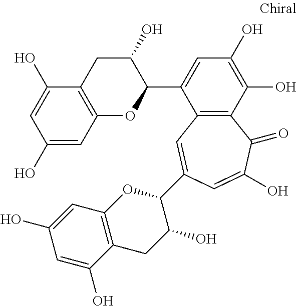 [SMILES: O=c1c(O)cc([C@H]2Oc3cc(O)cc(O)c3C[C@H]2O)cc2c([C@H]3Oc4cc(O)cc(O)c4C[C@@H]3O)cc(O)c(O)c12] | Microsource | 00200111 | THEAFLAVIN | 60.42 |
| YU155312 | 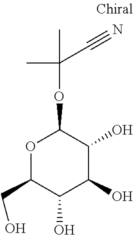 [SMILES: CC(C)(C#N)O[C@@H]1O[C@H](CO)[C@@H](O)[C@H](O)[C@H]1O] | Microsource | 01504124 | LINAMARIN | 60.30 |
| YU146590 | 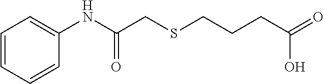 [SMILES: O=C(O)CCCSCC(=O)Nc1ccccc1] | ChemBridge | 7957074 | | 60.22 |
| YU172172 | 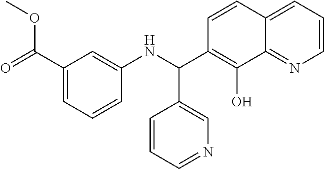 [SMILES: COC(=O)c1cccc(NC(c2cccnc2)c2ccc3cccnc3c2O)c1] | ChemDiv | 8397-0271 | | 60.11 |
| YU152226 | 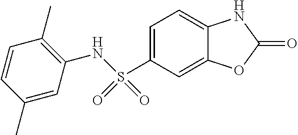 [SMILES: Cc1ccc(C)c(NS(=O)(=O)c2ccc3[nH]c(=O)oc3c2)c1] | ChemBridge | 7910527 | | 60.10 |
| YU155534 | 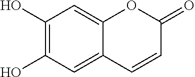 [SMILES: O=c1ccc2cc(O)c(O)cc2o1] | Microsource | 01500899 | ESCULETIN | 59.95 |
| YU155201 | 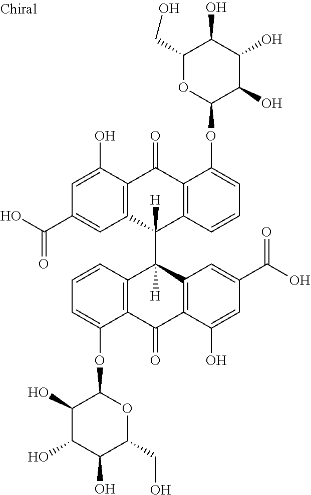 | Microsource | 01504078 | SENNOSIDE A | 59.95 |
| YU151128 | 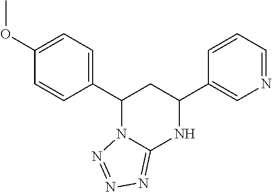 [SMILES: COc1ccc(C2CC(c3cccnc3)Nc3nnnn32)cc1] | ChemBridge | 7939491 | | 59.72 |
| YU121632 | 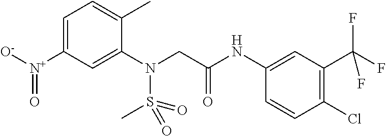 [SMILES: Cc1ccc([N+](=O)[O-])cc1N(CC(=O)Nc1ccc(Cl)c(C(F)(F)F)c1)S(C)(=O)=O] | ChemBridge | 7716211 | | 59.25 |
| YU221139 | 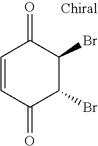 | Yale University | Crews04 | | 58.70 |
| YU033927 | 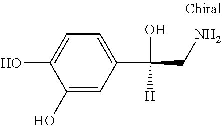 [SMILES: NC[C@H](O)c1ccc(O)c(O)c1] | Microsource | 01500436 | NOREPINEPHRINE | 58.47 |
| YU172179 | 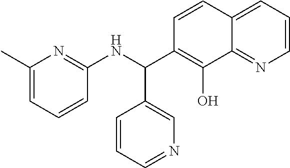 [SMILES: Cc1cccc(NC(c2cccnc2)c2ccc3cccnc3c2O)n1] | ChemDiv | 8397-0665 | | 58.12 |
| YU033971 | 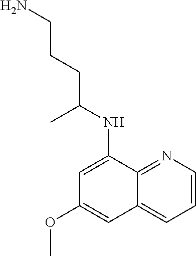 [SMILES: COc1cc(NC(C)CCCN)c2ncccc2c1] | Microsource | 01500500 | PRIMAQUINE DIPHOSPHATE | 57.47 |
| YU147801 | 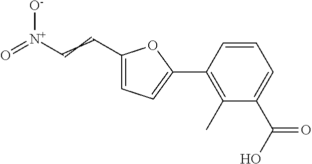 [SMILES: Cc1c(C(=O)O)cccc1-c1ccc(C=C[N+](=O)[O-])o1] | ChemBridge | 7220012 | | 56.87 |
| YU149922 | 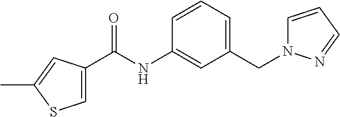 [SMILES: Cc1cc(C(=O)Nc2cccc(Cn3cccn3)c2)cs1] | ChemBridge | 7943809 | | 56.69 |
| YU034226* | 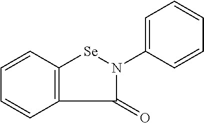 [SMILES: O=c1c2ccccc2[se]n1-c1ccccc1] | Microsource | 01501188 | EBSELEN | 56.49 |
| YU155465 | 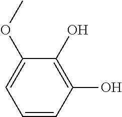 | Microsource | 01600919 | 3-METHOXYCATECHOL | 56.46 |
| YU172087 | 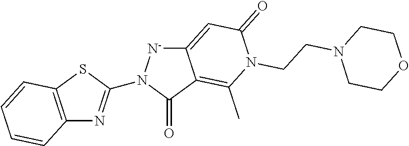 [SMILES: Cc1c2c(=O)n(-c3nc4ccccc4s3)[n-]c2cc(=O)n1CCN1CCOCC1] | ChemDiv | 8249-3507 | | 56.42 |
| YU033809 | 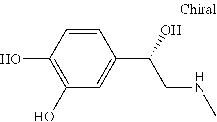 | Microsource | 01500274 | ADRENALINE BITARTRATE | 56.28 |
| YU033892* | 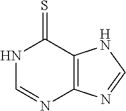 | Microsource | 01500387 | MERCAPTOPURINE | 55.98 |
| YU172164 | 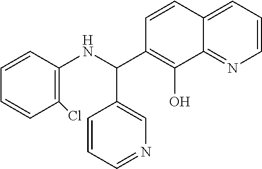 [SMILES: Oc1c(C(Nc2ccccc2Cl)c2cccnc2)ccc2cccnc12] | ChemDiv | 8297-0127 | | 55.97 |
| YU155087 | 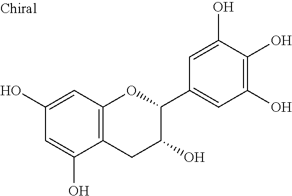 [SMILES: Oc1cc(O)c2c(c1)O[C@H](c1cc(O)c(O)c(O)c1)[C@H](O)C2] | Microsource | 00205113 | EPIGALLOCATECHIN | 55.49 |
| YU153281 | 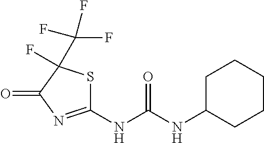 [SMILES: O=C(NC1=NC(=O)C(F)(C(F)(F)F)S1)NC1CCCCC1] | ChemBridge | 7934812 | | 55.33 |
| YU104987 | 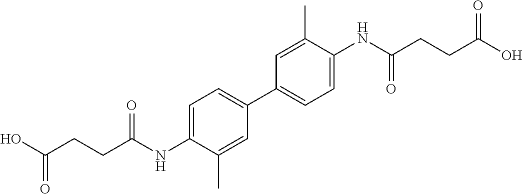 [SMILES: Cc1cc(-c2ccc(NC(=O)CCC(=O)O)c(C)c2)ccc1NC(=O)CCC(=O)O] | ChemBridge | 5105131 | | 55.24 |
| YU145655 | 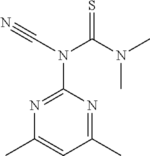 | ChemBridge | 6638931 | | 54.68 |
| YU033872 | 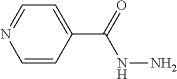 | Microsource | 01500355 | ISONIAZID | 54.49 |
| YU153287 | 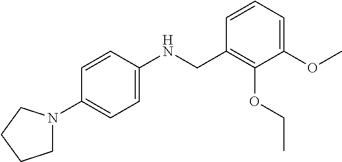 [SMILES: CCOc1c(CNc2ccc(N3CCCC3)cc2)cccc1OC] | ChemBridge | 7943091 | | 54.18 |
| YU154832 | 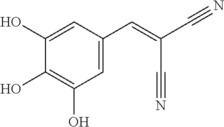 [SMILES: N#CC(C#N)=Cc1cc(O)c(O)c(O)c1] | Enzo | EI-187 | TYRPHOSTIN 25 | 53.71 |
| YU208200 | 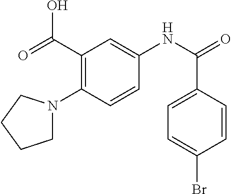 [SMILES: O=C(Nc1ccc(N2CCCC2)c(C(=O)O)c1)c1ccc(Br)cc1] | ChemDiv | G889-0412 | | 52.97 |
| YU208195 | 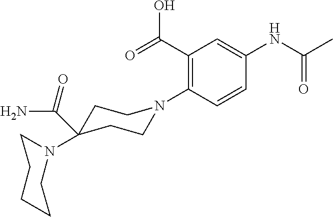 [SMILES: CC(=O)Nc1ccc(N2CCC(C(N)=O)(N3CCCCC3)CC2)c(C(=O)O)c1] | ChemDiv | G889-0172 | | 52.91 |
| YU146698 | 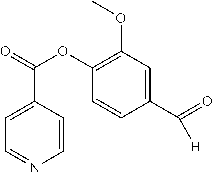 [SMILES: COc1cc(C=O)ccc1OC(=O)c1ccncc1] | ChemBridge | 7377697 | | 52.55 |
| YU034024* | 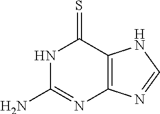 [SMILES: Nc1nc2nc[nH]c2c(=S)[nH]1] | Microsource | 01500573 | THIOGUANINE | 52.25 |
| YU154484 | 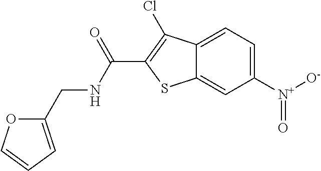 [SMILES: O=C(NCc1ccco1)c1sc2cc([N+](=O)[O-])ccc2c1Cl] | ChemBridge | 7947354 | | 52.19 |
| YU033694* | 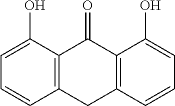 [SMILES: O=C1c2c(O)cccc2Cc2cccc(O)c21] | Microsource | 01500127 | ANTHRALIN | 51.68 |
| YU152236 | 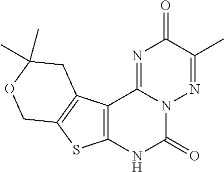 | ChemBridge | 7919641 | | 51.63 |
| YU208247 | 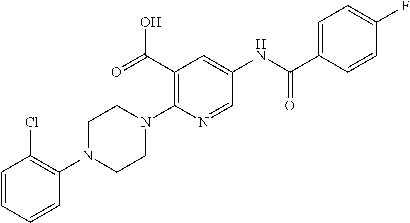 [SMILES: O=C(Nc1cnc(N2CCN(c3ccccc3Cl)CC2)c(C(=O)O)c1)c1ccc(F)cc1] | ChemDiv | G890-0098 | | 51.53 |
| YU034167 | 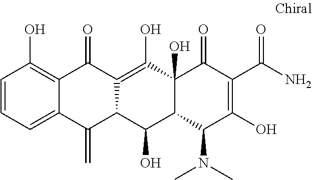 [SMILES: C=C1c2cccc(O)c2C(=O)C2=C(O)[C@]3(O)C(=O)C(C(N)=O)=C(O)[C@@H](N(C)C)[C@@H]3[C@@H](O)[C@H]12] | Microsource | 01501104 | METHACYCLINE HYDROCHLORIDE | 51.46 |
| YU148087 | 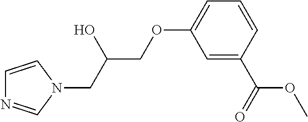 [SMILES: COC(=O)c1cccc(OCC(O)Cn2ccnc2)c1] | ChemBridge | 7917906 | | 51.38 |
| YU033802 | | Microsource | 01505155 | 3-HYDROXYTYRAMINE | 51.28 |
| YU149941 | 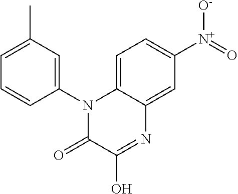 [SMILES: Cc1cccc(-n2c(=O)c(O)nc3cc([N+](=O)[O-])ccc32)c1] | ChemBridge | 7963683 | | 51.07 |
| YU208232 | 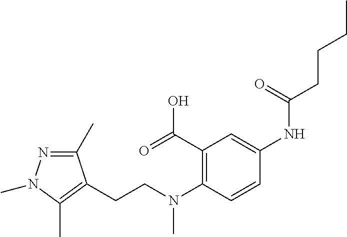 [SMILES: CCCCC(=O)Nc1ccc(N(C)CCc2c(C)nn(C)c2C)c(C(=O)O)c1] | ChemDiv | G889-1299 | | 50.49 |
| YU154829 | 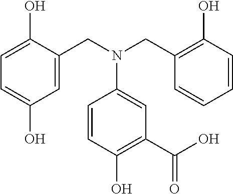 [SMILES: O=C(O)c1cc(N(Cc2ccccc2O)Cc2cc(O)ccc2O)ccc1O] | Enzo | EI-185 | LAVENDUSTIN A | 50.28 |
| YU208239 | 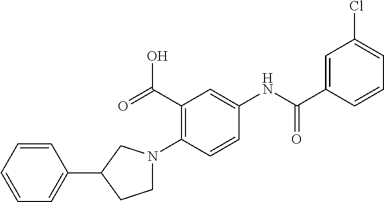 [SMILES: O=C(Nc1ccc(N2CCC(c3ccccc3)C2)c(C(=O)O)c1)c1cccc(Cl)c1] | ChemDiv | G889-1346 | | 50.24 |
| YU034463 | 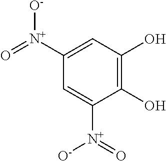 [SMILES: O=[N+]([O-])c1cc(O)c(O)c([N+](=O)[O-])c1] | Microsource | 01503631 | 3,5-DINITROCATECHOL (OR-486) | 49.87 |
| YU152899 | 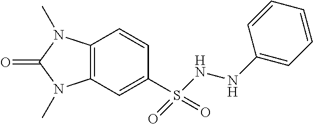 [SMILES: Cn1c(=O)n(C)c2cc(S(=O)(=O)NNc3ccccc3)ccc21] | ChemBridge | 7910497 | | 49.65 |
| YU035165 | 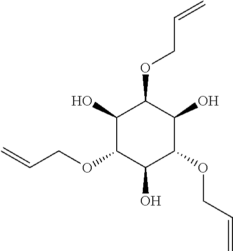 [SMILES: C=CCO[C@H]1[C@H](O)[C@H](OCC=C)[C@H](O)[C@@H](OCC=C)[C@@H]1O] | Yale University | SK_6 | | 49.52 |
| YU221065 | 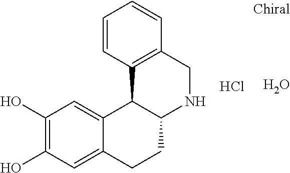 [SMILES: Oc1cc2c(cc1O)[C@H]1c3ccccc3CN[C@@H]1CC2 ; Cl ; O] | NCC | SAM001247083 | Benzo[a]phenanthridine- 10,11-diol, 5,6,6a,7,8,12b- hexahydro-, trans- [CAS] | 49.09 |
| YU146041 | 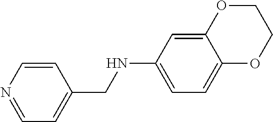 [SMILES: c1cc(CNc2ccc3c(c2)OCCO3)ccn1] | ChemBridge | 7986284 | | 48.73 |
| YU155007 | 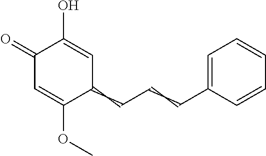 [SMILES: COC1=CC(=O)C(O)=CC1=CC=Cc1ccccc1] | Microsource | 00200090 | OBTUSAQUINONE | 48.70 |
| YU172175 | 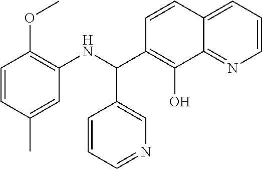 [SMILES: COc1ccc(C)cc1NC(c1cccnc1)c1ccc2cccnc2c1O] | ChemDiv | 8397-0559 | | 48.48 |
| YU149514 | 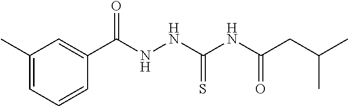 [SMILES: Cc1cccc(C(=O)NNC(=S)NC(=O)CC(C)C)c1] | ChemBridge | 7915263 | | 48.35 |
| YU149834 | 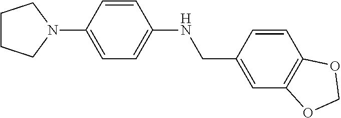 [SMILES: c1cc2c(cc1CNc1ccc(N3CCCC3)cc1)OCO2] | ChemBridge | 7927434 | | 48.09 |
| YU033938 | 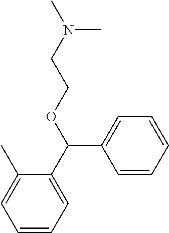 [SMILES: Cc1ccccc1C(OCCN(C)C)c1ccccc1] | Microsource | 01500447 | ORPHENADRINE CITRATE | 47.93 |
| YU033874 | 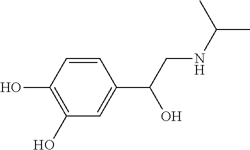 | Microsource | 01500357 | ISOPROTERENOL HYDROCHLORIDE | 47.67 |
| YU151897 | 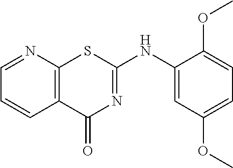 [SMILES: COc1ccc(OC)c(Nc2nc(=O)c3cccnc3s2)c1] | ChemBridge | 7905968 | | 47.60 |
| YU033712* | 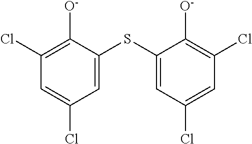 [SMILES: [O-]c1c(Cl)cc(Cl)cc1Sc1cc(Cl)cc(Cl)c1[O-]] | Microsource | 01500148 | BITHIONOL | 47.58 |
| YU149839 | 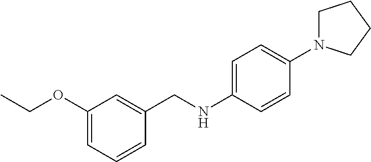 [SMILES: CCOc1cccc(CNc2ccc(N3CCCC3)cc2)c1] | ChemBridge | 7932017 | | 47.48 |
| YU035181* | 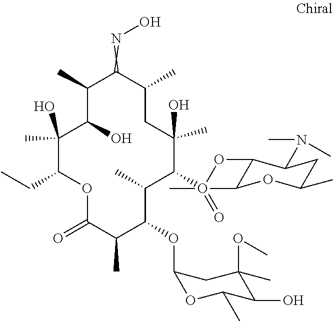 | Yale University | CAL_oxime | | 47.14 |
| YU033752 | 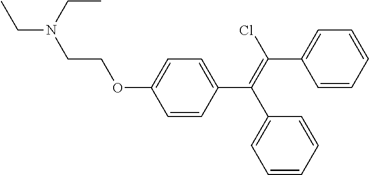 [SMILES: CCN(CC)CCOc1ccc(/C(=C(\Cl)c2ccccc2)c2ccccc2)cc1] | Microsource | 01500196 | CLOMIPHENE CITRATE | 47.01 |
| YU033619 | 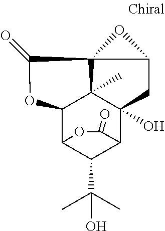 | Microsource | 00100346 | PICROTIN | 46.86 |
| YU033802 | 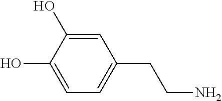 [SMILES: NCCc1ccc(O)c(O)c1] | Microsource | 01500263 | DOPAMINE HYDROCHLORIDE | 46.86 |
| YU185530 | 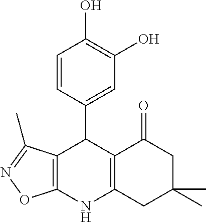 [SMILES: Cc1noc2c1C(c1ccc(O)c(O)c1)C1=C(CC(C)(C)CC1=O)N2] | ChemDiv | D588-0192 | | 46.52 |
| YU154834 | 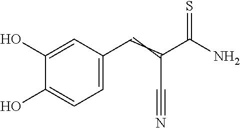 [SMILES: N#CC(=Cc1ccc(O)c(O)c1)C(N)=S] | Enzo | EI-188 | TYRPHOSTIN 47 | 46.51 |
| YU154859 | 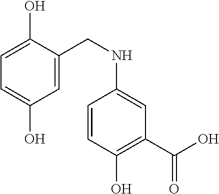 [SMILES: O=C(O)c1cc(NCc2cc(O)ccc2O)ccc1O] | Enzo | EI-232 | 2-HYDROXY-5-(2,5- DIHYDROXYBENZYL- AMINO)BENZOIC ACID | 46.44 |
| YU034557 | 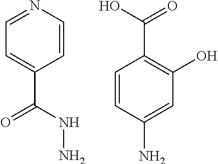 [SMILES: NNC(=O)c1ccncc1 ; Nc1ccc(C(=O)O)c(O)c1] | Microsource | 01503381 | PASINIAZID | 46.37 |
| YU208193 | 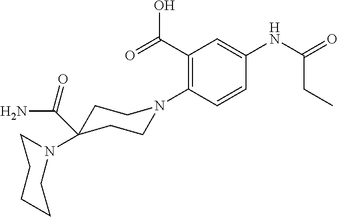 [SMILES: CCC(=O)Nc1ccc(N2CCC(C(N)=O)(N3CCCCC3)CC2)c(C(=O)O)c1] | ChemDiv | G889-0167 | | 46.12 |
| YU152583 | 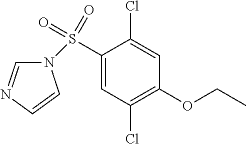 [SMILES: CCOc1cc(Cl)c(S(=O)(=O)n2ccnc2)cc1Cl] | ChemBridge | 7973763 | | 45.85 |
| YU155255 | 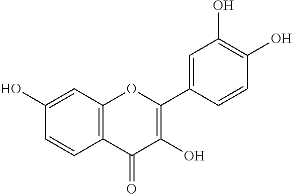 [SMILES: O=c1c(O)c(-c2ccc(O)c(O)c2)oc2cc(O)ccc12] | Microsource | 01502247 | FISETIN | 45.84 |
| YU145266 | 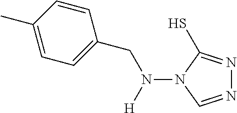 | ChemBridge | 7490877 | | 45.71 |
| YU153110 | 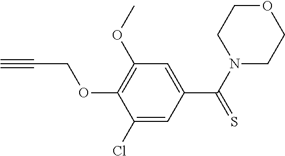 [SMILES: C#CCOc1c(Cl)cc(C(=S)N2CCOCC2)cc1OC] | ChemBridge | 7916412 | | 45.18 |
| YU155369 | 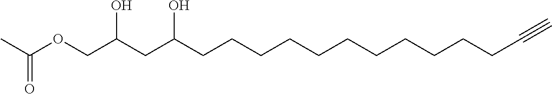 [SMILES: C#CCCCCCCCCCCCC(O)CC(O)COC(C)=O] | Microsource | 00240929 | AVOCADYNE ACETATE | 45.13 |
| YU151409 | 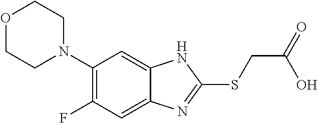 [SMILES: O=C(O)CSc1nc2cc(F)c(N3CCOCC3)cc2[nH]1] | ChemBridge | 7849329 | | 45.07 |
| YU221011 | 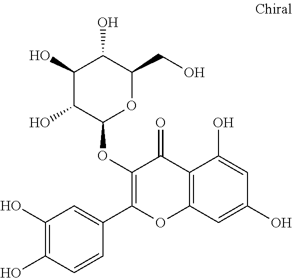 [SMILES: O=c1c(O[C@@H]2O[C@H](CO)[C@@H](O)[C@H](O)[C@H]2O)c(-c2ccc(O)c(O)c2)oc2cc(O)cc(O)c12] | NCC | SAM001246767 | Isoquercitrin | 45.06 |
| YU154737 | 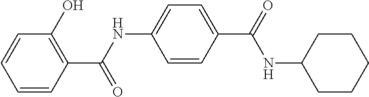 [SMILES: O=C(NC1CCCCC1)c1ccc(NC(=O)c2ccccc2O)cc1] | ChemBridge | 7954771 | | 44.99 |
| YU221013 | 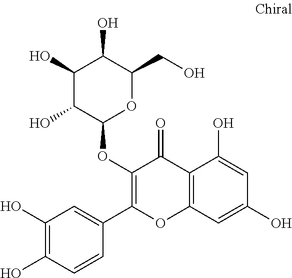 [SMILES: O=c1c(O[C@@H]2O[C@H](CO)[C@H](O)[C@H](O)[C@H]2O)c(-c2ccc(O)c(O)c2)oc2cc(O)cc(O)c12] | NCC | SAM001246776 | HYPEROSIDE | 44.91 |
| YU144893 | 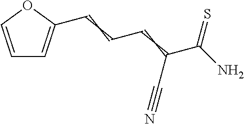 [SMILES: N#CC(=CC=Cc1ccco1)C(N)=S] | ChemBridge | 7850219 | | 44.60 |
| YU208215 | 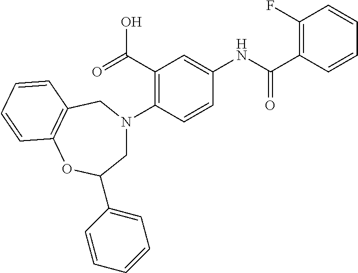 [SMILES: O=C(Nc1ccc(N2Cc3ccccc3OC(c3ccccc3)C2)c(C(=O)O)c1)c1ccccc1F] | ChemDiv | G889-1039 | | 44.59 |
| YU148889 | 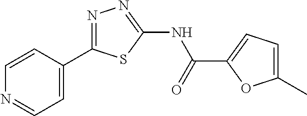 [SMILES: Cc1ccc(C(=O)Nc2nnc(-c3ccncc3)s2)o1] | ChemBridge | 7945627 | | 44.57 |
| YU208246 | 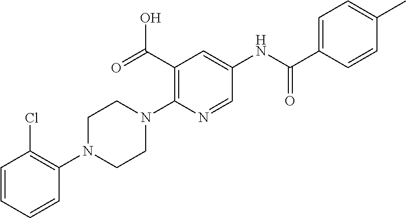 [SMILES: Cc1ccc(C(=O)Nc2cnc(N3CCN(c4ccccc4Cl)CC3)c(C(=O)O)c2)cc1] | ChemDiv | G890-0096 | | 44.53 |
| YU208250 | 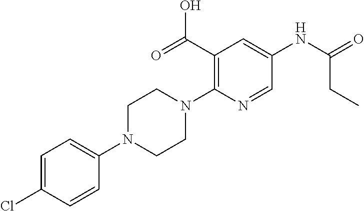 [SMILES: CCC(=O)Nc1cnc(N2CCN(c3ccc(Cl)cc3)CC2)c(C(=O)O)c1] | ChemDiv | G890-0200 | | 44.46 |
| YU221071* | 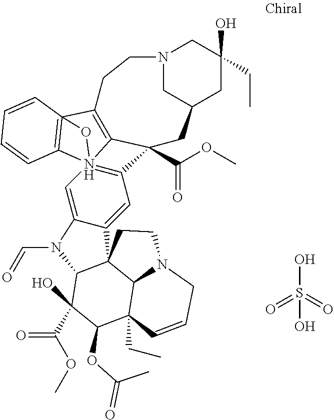 | NCC | SAM001246570 | VINCRISTINE SULFATE | 44.20 |
| YU154853 | 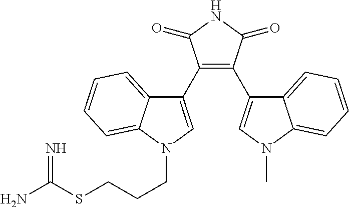 [SMILES: Cn1cc(C2=C(c3cn(CCCSC(=N)N)c4ccccc34)C(=O)NC2=O)c2ccccc21] | Enzo | EI-283 | Ro 31-8220 | 44.01 |
| YU154885 | 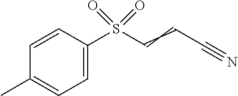 | Enzo | EI-278 | BAY 11-7082 | 43.57 |
| YU033863* | 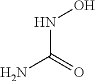 | Microsource | 01500344 | HYDROXYUREA | 43.49 |
| YU208228 | 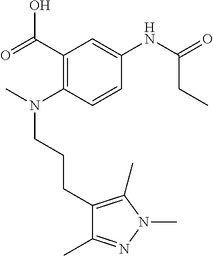 [SMILES: CCC(=O)Nc1ccc(N(C)CCCc2c(C)nn(C)c2C)c(C(=O)O)c1] | ChemDiv | G889-1199 | | 43.43 |
| YU155086* | 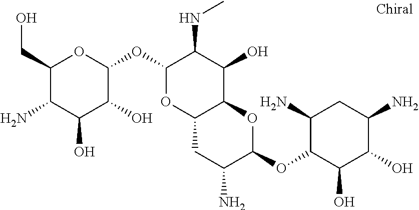 | Microsource | 01505249 | APRAMYCIN | 43.18 |
| YU149607 | 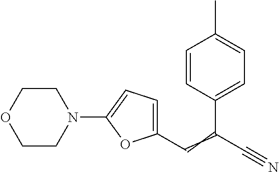 [SMILES: Cc1ccc(C(C#N)=Cc2ccc(N3CCOCC3)o2)cc1] | ChemBridge | 7937853 | | 43.14 |
| YU145407 | 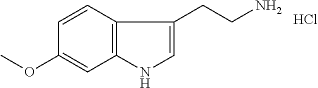 | ChemBridge | 5107324 | | 43.05 |
| YU155440* | 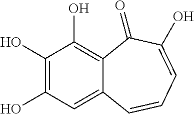 [SMILES: O=c1c(O)cccc2cc(O)c(O)c(O)c12] | Microsource | 00210505 | PURPUROGALLIN | 42.52 |
| YU145239 | 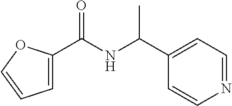 [SMILES: CC(NC(=O)c1ccco1)c1ccncc1] | ChemBridge | 7947845 | | 42.38 |
| YU155016* | 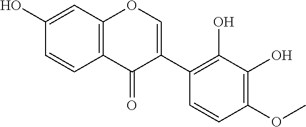 [SMILES: COc1ccc(-c2coc3cc(O)ccc3c2=O)c(O)c1O] | Microsource | 00200422 | KOPARIN | 42.33 |
| YU172165 | 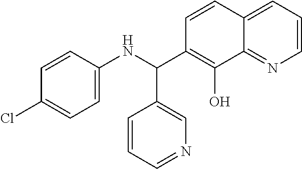 [SMILES: Oc1c(C(Nc2ccc(Cl)cc2)c2cccnc2)ccc2cccnc12] | ChemDiv | 8397-0140 | | 42.30 |
| YU146454 | 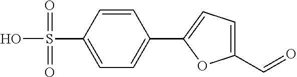 [SMILES: O=Cc1ccc(-c2ccc(S(=O)(=O)O)cc2)o1] | ChemBridge | 6914720 | | 42.30 |
| YU221030* | 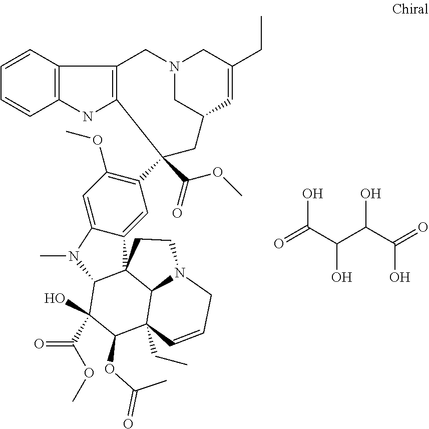 | NCC | SAM001246780 | VINORELBINE BITATRATE | 42.18 |
| YU019467* | 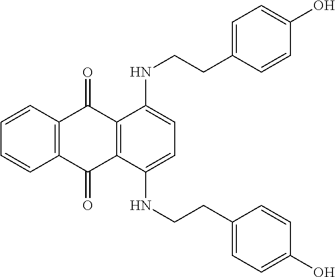 [SMILES: O=C1c2ccccc2C(=O)c2c(NCCc3ccc(O)cc3)ccc(NCCc3ccc(O)cc3)c21] | Enzo | GR-346 | BML-266 | 42.15 |
| YU034372 | 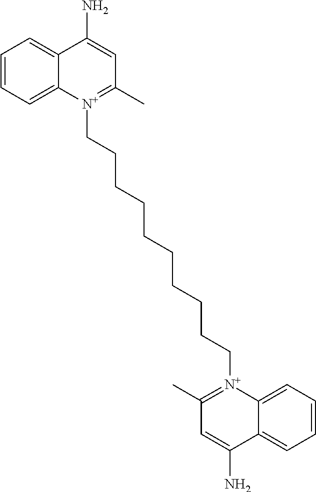 [SMILES: Cc1cc(N)c2ccccc2[n+]1CCCCCCCCCC[n+]1c(C)cc(N)c2ccccc21] | Microsource | 01503127 | DEQUALINIUM CHLORIDE | 42.11 |
| YU152765 | 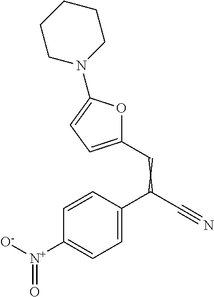 [SMILES: N#CC(=Cc1ccc(N2CCCCC2)o1)c1ccc([N+](=O)[O-])cc1] | ChemBridge | 7752357 | | 41.95 |
| YU033631* | 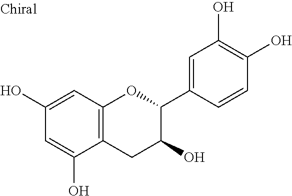 [SMILES: Oc1cc(O)c2c(c1)O[C@H](c1ccc(O)c(O)c1)[C@@H](O)C2] | Microsource | 00210205 | CIANIDANOL | 41.49 |
| YU152560 | 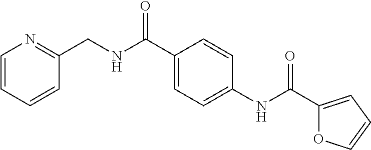 [SMILES: O=C(NCc1ccccn1)c1ccc(NC(=O)c2ccco2)cc1] | ChemBridge | 7926149 | | 41.45 |
| YU034226 | 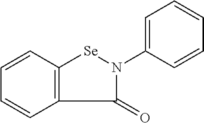 [SMILES: O=c1c2ccccc2[se]n1-c1ccccc1] | NCC | SAM001247071 | EBSELEN | 41.43 |
| YU153907 | 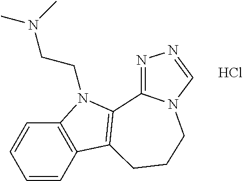 [SMILES: CN(C)CCn1c2c(c3ccccc31)CCCn1cnnc1-2 ; Cl] | ChemBridge | 7921224 | | 41.23 |
| YU033734 | 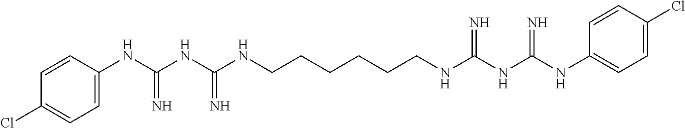 [SMILES: N=C(NCCCCCCNC(=N)NC(=N)Nc1ccc(Cl)cc1)NC(=N)Nc1ccc(Cl)cc1] | Microsource | 01500177 | CHLORHEXIDINE | 41.01 |
| YU033940 | 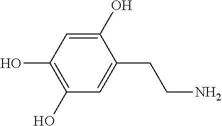 [SMILES: NCCc1cc(O)c(O)cc1O] | Microsource | 01500450 | OXIDOPAMINE HYDROCHLORIDE | 40.97 |
| YU221143 | 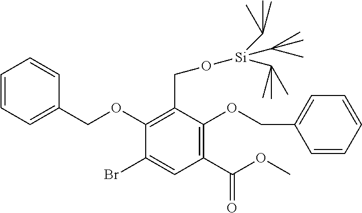 [SMILES: COC(=O)c1cc(Br)c(OCc2ccccc2)c(CO[Si](C(C)(C)C)(C(C)(C)C)C(C)(C)C)c1OCc1ccccc1] | Yale University | Crews08 | | 40.92 |
| YU034320 | 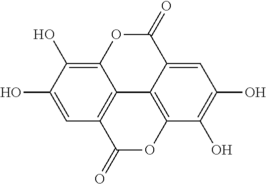 [SMILES: O=c1oc2c(O)c(O)cc3c(=O)oc4c(O)c(O)cc1c4c23] | Microsource | 01502245 | ELLAGIC ACID | 40.88 |
| YU221215* | 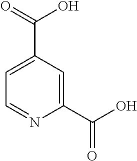 [SMILES: O=C(O)c1ccnc(C(=O)O)c1] | Enzo | A-280 | 2,4-Pyridinedicarboxylic Acid | 40.80 |
| YU155308 | 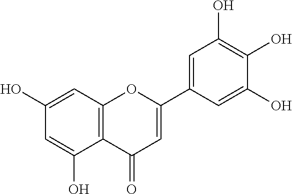 [SMILES: O=c1cc(-c2cc(O)c(O)c(O)c2)oc2cc(O)cc(O)c12] | Microsource | 01504115 | HIERACIN | 40.74 |
| YU154851 | 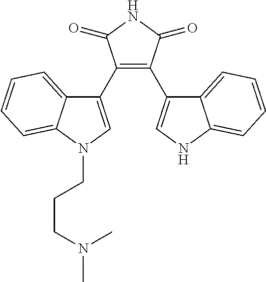 [SMILES: CN(C)CCCn1cc(C2=C(c3c[nH]c4ccccc34)C(=O)NC2=O)c2ccccc21] | Enzo | 01-246 | GF 109203X | 40.71 |
| YU153935 | 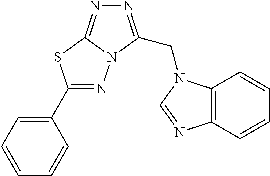 [SMILES: c1ccc(-c2nn3c(Cn4cnc5ccccc54)nnc3s2)cc1] | ChemBridge | 7951242 | | 40.54 |
| YU034265* | 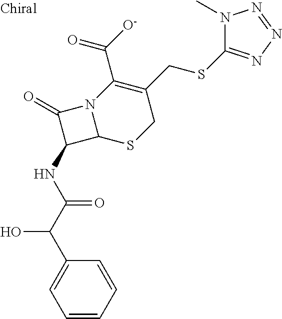 [SMILES: Cn1nnnc1SCC1=C(C(=O)[O-])N2C(=O)[C@H](NC(=O)C(O)c3ccccc3)C2SC1] | Microsource | 01502038 | CEFAMANDOLE SODIUM | 40.54 |
| YU034447 | 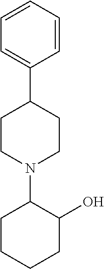 [SMILES: OC1CCCCC1N1CCC(c2ccccc2)CC1] | Microsource | 02300309 | VESAMICOL HYDROCHLORIDE | 40.49 |
| YU035331* | 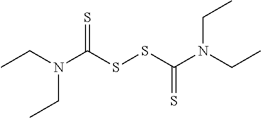 [SMILES: CCN(CC)C(=S)SSC(=S)N(CC)CC] | NCC | SAM001247028 | TETRAETHYLTHIURAM DISULFIDE | 40.45 |
| YU147615 | 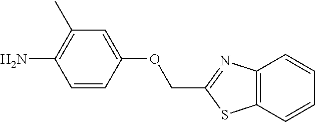 [SMILES: Cc1cc(OCc2nc3ccccc3s2)ccc1N] | ChemBridge | 7938899 | | 40.44 |
| YU034538 | 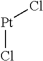 | Microsource | 01502107 | CISPLATIN | 40.06 |
| YU154091 | 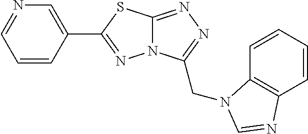 [SMILES: c1cncc(-c2nn3c(Cn4cnc5ccccc54)nnc3s2)c1] | ChemBridge | 7952897 | | 40.02 |
| YU172173 | 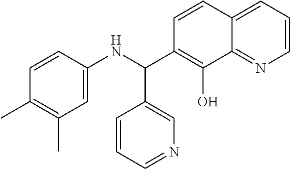 [SMILES: Cc1ccc(NC(c2cccnc2)c2ccc3cccnc3c2O)cc1C] | ChemDiv | 8397-0490 | | 39.98 |
| YU147088 | 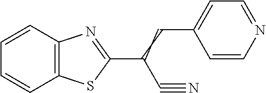 [SMILES: N#CC(=Cc1ccncc1)c1nc2ccccc2s1] | ChemBridge | 6145186 | | 39.81 |
| YU152562 | 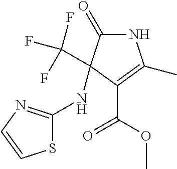 [SMILES: COC(=O)C1=C(C)NC(=O)C1(Nc1nccs1)C(F)(F)F] | ChemBridge | 7928138 | | 39.42 |
| YU152183 | 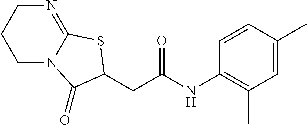 [SMILES: Cc1ccc(NC(=O)CC2SC3=NCCCN3C2=O)c(C)c1] | ChemBridge | 7980473 | | 39.38 |
| YU147640 | 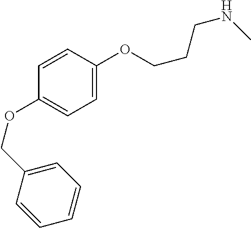 [SMILES: CNCCCOc1ccc(OCc2ccccc2)cc1] | ChemBridge | 7706494 | | 39.06 |
| YU105195 | 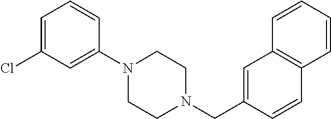 [SMILES: Clc1cccc(N2CCN(Cc3ccc4ccccc4c3)CC2)c1] | ChemBridge | 5268565 | | 38.88 |
| YU144949 | 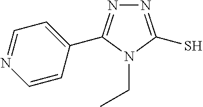 | ChemBridge | 7117164 | | 38.84 |
| YU208230 | 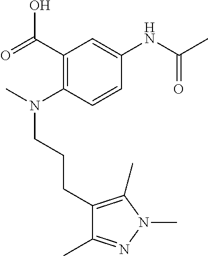 [SMILES: CC(=O)Nc1ccc(N(C)CCCc2c(C)nn(C)c2C)c(C(=O)O)c1] | ChemDiv | G889-1205 | | 38.49 |
| YU149002 | 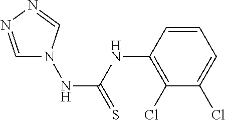 [SMILES: S=C(Nc1cccc(Cl)c1Cl)Nn1cnnc1] | ChemBridge | 7788977 | | 37.90 |
| YU152624 | 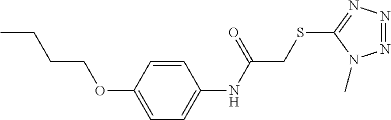 [SMILES: CCCCOc1ccc(NC(=O)CSc2nnnn2C)cc1] | ChemBridge | 7903590 | | 37.69 |
| YU172071 | 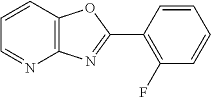 [SMILES: Fc1ccccc1-c1nc2ncccc2o1] | ChemDiv | 8188-2521 | | 37.33 |
| YU147019 | 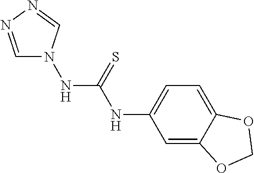 [SMILES: S=C(Nc1ccc2c(c1)OCO2)Nn1cnnc1] | ChemBridge | 7819221 | | 37.27 |
| YU172168 | 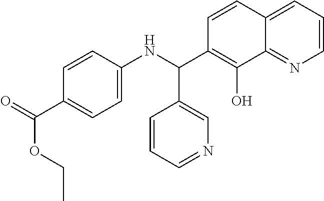 [SMILES: CCOC(=O)c1ccc(NC(c2cccnc2)c2ccc3cccnc3c2O)cc1] | ChemDiv | 8397-0166 | | 37.07 |
| YU151027 | 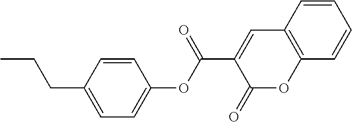 [SMILES: CCCc1ccc(OC(=O)c2cc3ccccc3oc2=O)cc1] | ChemBridge | 7911913 | | 36.99 |
| YU146438 | 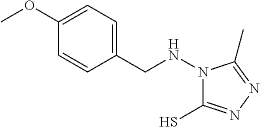 [SMILES: COc1ccc(CNn2c(C)nnc2S)cc1] | ChemBridge | 7985526 | | 36.92 |
| YU145520 | 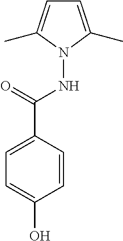 | ChemBridge | 7642641 | | 36.89 |
| YU151559 | 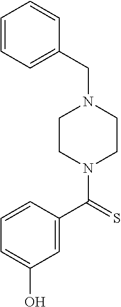 [SMILES: Oc1cccc(C(=S)N2CCN(Cc3ccccc3)CC2)c1] | ChemBridge | 7784869 | | 36.88 |
| YU147005 | 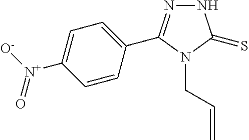 [SMILES: C=CCn1c(-c2ccc([N+](=O)[O-])cc2)n[nH]c1=S] | ChemBridge | 5954633 | | 36.81 |
| YU146294 | 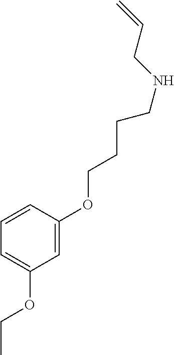 [SMILES: C=CCNCCCCOc1cccc(OCC)c1] | ChemBridge | 7008394 | | 36.52 |
| YU146471 | 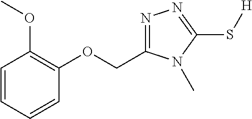 [SMILES: COc1ccccc1OCc1nnc(S)n1C] | ChemBridge | 7851437 | | 36.38 |
| YU148050 | 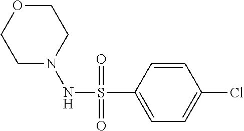 [SMILES: O=S(=O)(NN1CCOCC1)c1ccc(Cl)cc1] | ChemBridge | 7243257 | | 36.09 |
| YU145702 | 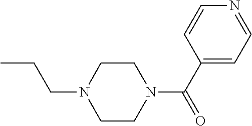 [SMILES: CCCN1CCN(C(=O)c2ccncc2)CC1] | ChemBridge | 7943502 | | 35.91 |
| YU149014 | 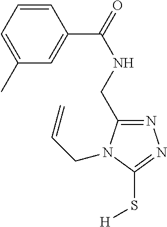 [SMILES: C=CCn1c(S)nnc1CNC(=O)c1cccc(C)c1] | ChemBridge | 7840569 | | 35.66 |
| YU105182 | 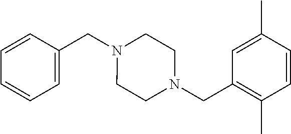 [SMILES: Cc1ccc(C)c(CN2CCN(Cc3ccccc3)CC2)c1] | ChemBridge | 5265368 | | 35.34 |
| YU151591 | 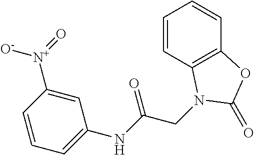 [SMILES: O=C(Cn1c(=O)oc2ccccc21)Nc1cccc([N+](=O)[O-])c1] | ChemBridge | 7914537 | | 34.96 |
| YU145796 | 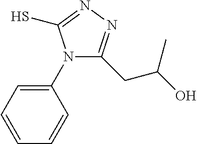 [SMILES: CC(O)Cc1nnc(S)n1-c1ccccc1] | ChemBridge | 7951575 | | 34.75 |
| YU148636 | 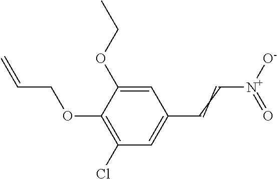 [SMILES: C=CCOc1c(Cl)cc(C=C[N+](=O)[O-])cc1OCC] | ChemBridge | 7916410 | | 34.68 |
| YU148611 | 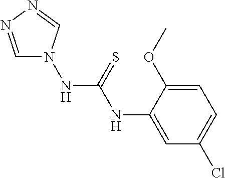 [SMILES: COc1ccc(Cl)cc1NC(=S)Nn1cnnc1] | ChemBridge | 7792444 | | 34.65 |
| YU150447 | 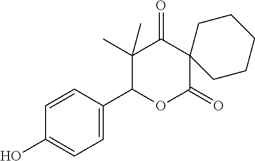 [SMILES: CC1(C)C(=O)C2(CCCCC2)C(=O)OC1c1ccc(O)cc1] | ChemBridge | 7823376 | | 34.34 |
| YU016812 | 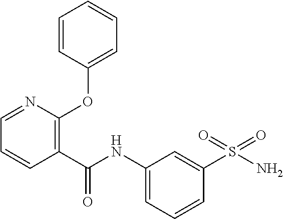 [SMILES: NS(=O)(=O)c1cccc(NC(=O)c2cccnc2Oc2ccccc2)c1] | Maybridge | HTS 06219 | | 34.19 |
| YU146768 | 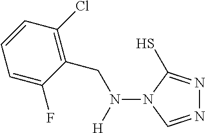 [SMILES: Fc1cccc(Cl)c1CNn1cnnc1S] | ChemBridge | 7507138 | | 34.04 |
| YU154843 | 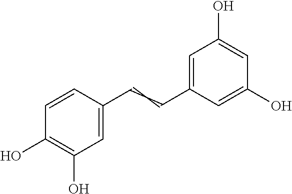 [SMILES: Oc1cc(O)cc(C=Cc2ccc(O)c(O)c2)c1] | Enzo | EI-271 | PICEATANNOL | 34.02 |
| YU149831 | 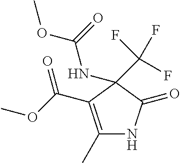 [SMILES: COC(=O)NC1(C(F)(F)F)C(=O)NC(C)=C1C(=O)OC] | ChemBridge | 7919340 | | 33.95 |
| YU147054 | 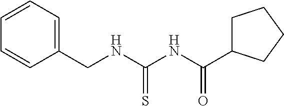 [SMILES: O=C(NC(=S)NCc1ccccc1)C1CCCC1] | ChemBridge | 7942455 | | 33.93 |
| YU153729 | 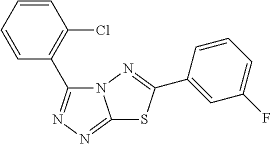 [SMILES: Fc1cccc(-c2nn3c(-c4ccccc4Cl)nnc3s2)c1] | ChemBridge | 7879885 | | 33.93 |
| YU151184 | 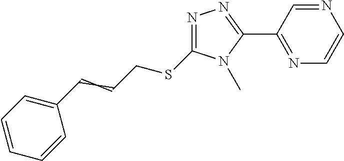 [SMILES: Cn1c(SCC=Cc2ccccc2)nnc1-c1cnccn1] | ChemBridge | 7911245 | | 33.84 |
| YU145432 | 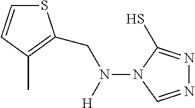 | ChemBridge | 7496439 | | 33.33 |
| YU146034 | 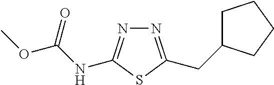 [SMILES: COC(=O)Nc1nnc(CC2CCCC2)s1] | ChemBridge | 7949611 | | 33.03 |
| YU147658 | 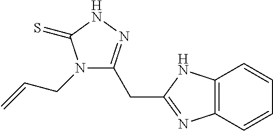 [SMILES: C=CCn1c(Cc2nc3ccccc3[nH]2)n[nH]c1=S] | ChemBridge | 7862976 | | 32.83 |
| YU147278 | 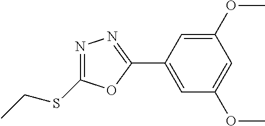 [SMILES: CCSc1nnc(-c2cc(OC)cc(OC)c2)o1] | ChemBridge | 7916510 | | 32.81 |
| YU146534 | 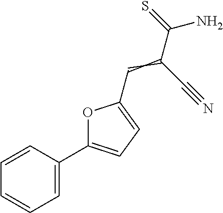 [SMILES: N#CC(=Cc1ccc(-c2ccccc2)o1)C(N)=S] | ChemBridge | 7494112 | | 32.78 |
| YU146037 | 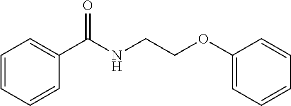 [SMILES: O=C(NCCOc1ccccc1)c1ccccc1] | ChemBridge | 7959412 | | 32.61 |
| YU151714 | 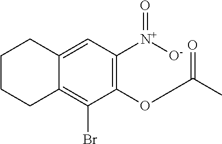 [SMILES: CC(=O)Oc1c([N+](=O)[O-])cc2c(c1Br)CCCC2] | ChemBridge | 7774023 | | 32.33 |
| YU145980 | 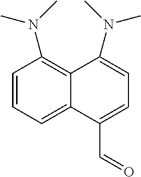 | ChemBridge | 6647257 | | 32.29 |
| YU150472 | 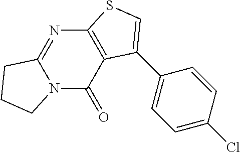 [SMILES: O=c1c2c(-c3ccc(Cl)cc3)csc2nc2n1CCC2] | ChemBridge | 7921066 | | 32.11 |
| YU149180 | 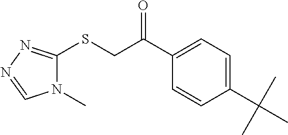 [SMILES: Cn1cnnc1SCC(=O)c1ccc(C(C)(C)C)cc1] | ChemBridge | 7907173 | | 32.02 |
| YU150371 | 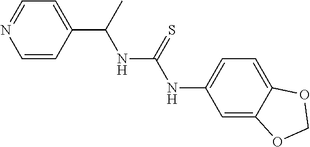 [SMILES: CC(NC(=S)Nc1ccc2c(c1)OCO2)c1ccncc1] | ChemBridge | 7842697 | | 31.77 |
| YU147024 | 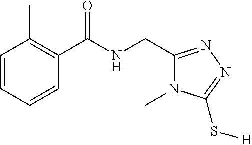 [SMILES: Cc1ccccc1C(=O)NCc1nnc(S)n1C] | ChemBridge | 7854533 | | 31.62 |
| YU147191 | 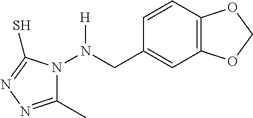 [SMILES: Cc1nnc(S)n1NCc1ccc2c(c1)OCO2] | ChemBridge | 7879054 | | 31.47 |
| YU145747 | 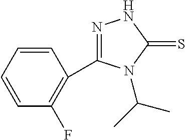 [SMILES: CC(C)n1c(-c2ccccc2F)n[nH]c1=S] | ChemBridge | 7784784 | | 31.29 |
| YU147498 | 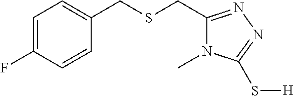 [SMILES: Cn1c(S)nnc1CSCc1ccc(F)cc1] | ChemBridge | 7832823 | | 31.15 |
| YU147245 | 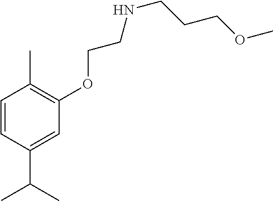 [SMILES: COCCCNCCOc1cc(C(C)C)ccc1C] | ChemBridge | 7015081 | | 30.89 |
| YU146020 | 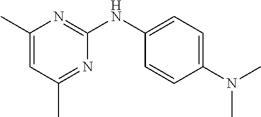 | ChemBridge | 7931802 | | 30.85 |
| YU151441 | 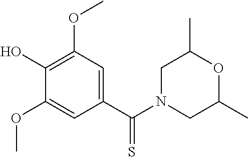 [SMILES: COc1cc(C(=S)N2CC(C)OC(C)C2)cc(OC)c1O] | ChemBridge | 7934320 | | 30.81 |
| YU148159 | 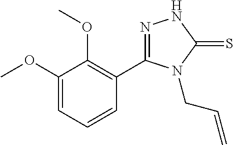 [SMILES: C=CCn1c(-c2cccc(OC)c2OC)n[nH]c1=S] | ChemBridge | 7915345 | | 30.35 |

Compounds with ID marked with “*” were the additional hits identified from screening of the MicroSource Gen-Plus, MicroSource Pure Natural Products, NIH Clinical Collection, Enzo Epigenetics, Yale Compound libraries under demethyalse reaction condition with 1 mM α-KG.

TABLE 3
Validation and counterscreen results of HTS actives.
Artifact signal is the effect of the compound when it is incubated with bio-H3K4me2 peptide.

| Compound | | | |
|---|---|---|---|
| ID | Structure | Supplier | Supplier Library |
| YU146842 | 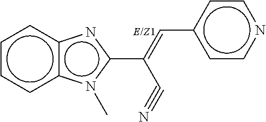 | ChemBridge | MW-Set |
| YU034226 | 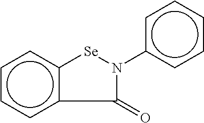 | NCC | NCC |
| YU155312 | 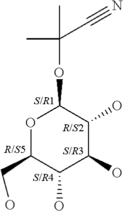 [SMILES: CC(C)(C#N)O[C@@H]1O[C@H](CO)[C@@H](O)[C@H](O)[C@H]1O] | Microsource | NaturalProducts |
| YU153803 | 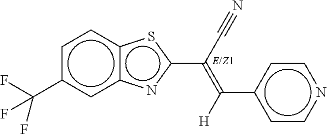 | ChemBridge | MW-Set |
| YU129955 | 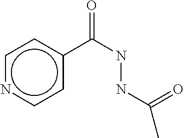 | ChemBridge | DvS |
| YU221215 | 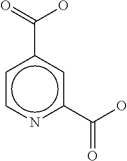 | Enzo | EpigensticsLib |
| YU145461 | 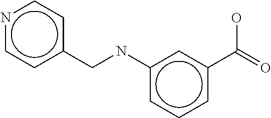 | ChemBridge | MW-Set |
| YU172999 | 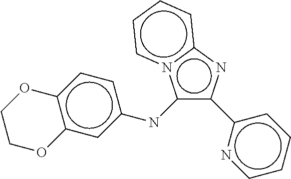 | ChemDiv | ChemDiv |
| YU145649 | 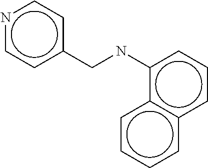 | ChemBridge | MW-Set |
| YU146026 | 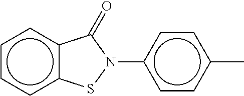 | ChemBridge | MW-Set |
| YU155621 | 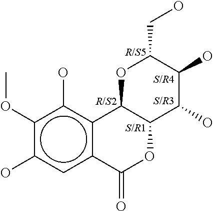 | Microsource | NaturalProducts |
| YU147266 | 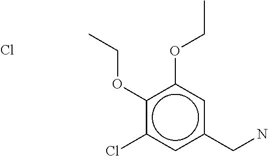 | ChemBridge | MW-Set |
| YU034066 | 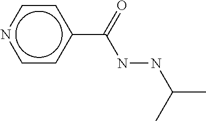 | Microsource | GenPlus |
| YU146454 | 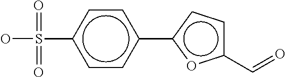 | ChemBridge | MW-Set |
| YU129874 | 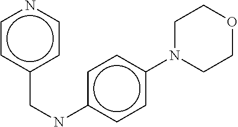 | ChemBridge | DvS |
| YU034347 | 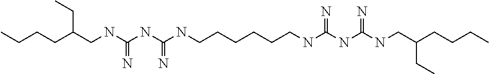 [SMILES: CCCCC(CC)CNC(=N)NC(=N)NCCCCCCNC(=N)NC(=N)NCC(CC)CCCC] | Microsource | GenPlus |
| YU033938 | 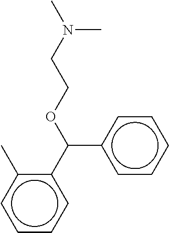 | Microsource | GenPlus |
| YU155005 | 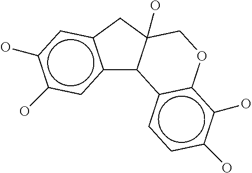 | Microsource | NaturalProducts |
| YU155280 | 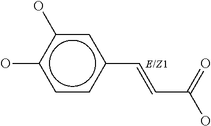 | Microsource | NaturalProducts |
| YU155534 | 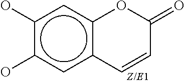 | Microsource | NaturalProducts |
| YU034426 | 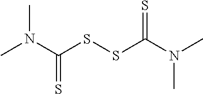 | Microsource | GenPlus |
| YU033662 | 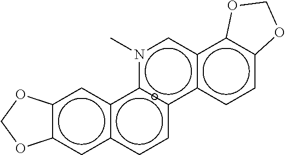 | Microsource | GenPlus |
| YU033696 | 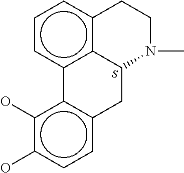 | Microsource | GenPlus |
| YU155618 | 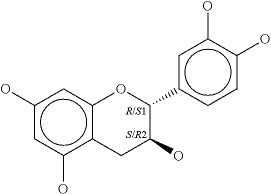 | Microsource | NaturalProducts |
| YU145853 | 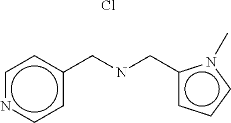 | ChemBridge | MW-Set |
| YU154887 | 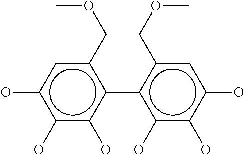 | Enzo | KinaseInhLib |
| YU221065 | 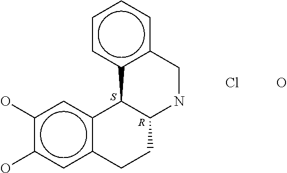 | NCC | NCC |
| YU148374 | 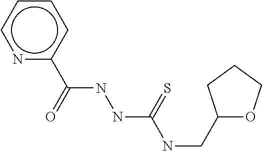 | ChemBridge | MW-Set |
| YU155214 | 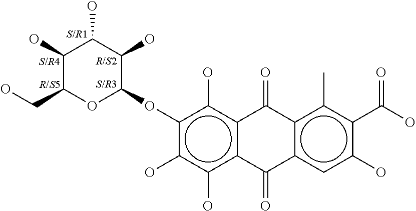 | Microsource | NaturalProducts |
| YU146041 | 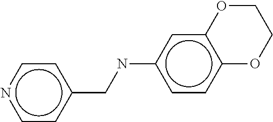 | ChemBridge | MW-Set |
| YU147704 | 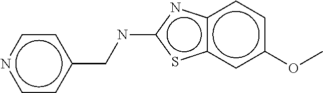 | ChemBridge | MW-Set |
| YU146698 | 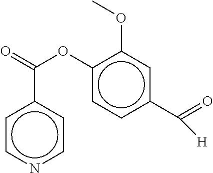 | ChemBridge | MW-Set |
| YU033654 | 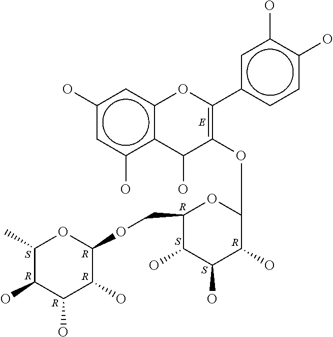 | Microsource | GenPlus |
| YU149180 | 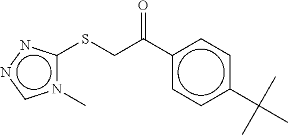 | ChemBridge | MW-Set |
| YU033702 | 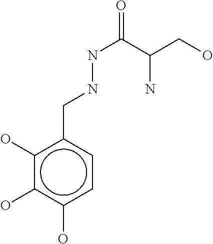 | Microsource | GenPlus |
| YU033872 | 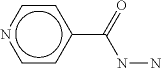 | Microsource | GenPlus |
| YU155562 | 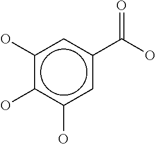 | Microsource | NaturalProducts |
| YU033903 | 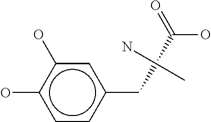 | Microsource | GenPlus |
| YU033971 | 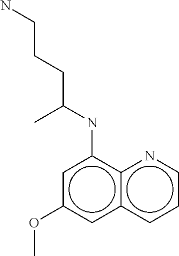 | Microsource | GenPlus |
| YU146768 | 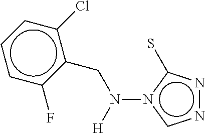 | ChemBridge | MW-Set |
| YU149014 | 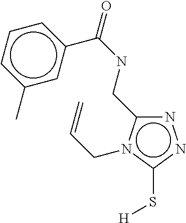 | ChemBridge | MW-Set |
| YU155465 | 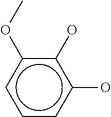 | Microsource | NaturalProducts |
| YU173004 | 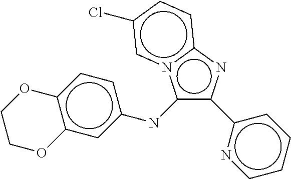 | ChemDiv | ChemDiv |
| YU150981 | 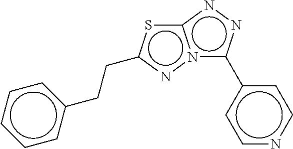 | ChemBridge | MW-Set |
| YU146471 | 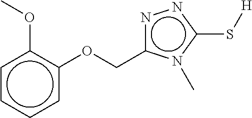 | ChemBridge | MW-Set |
| YU033809 | 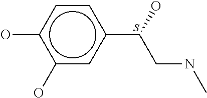 | Microsource | GenPlus |
| YU145432 | 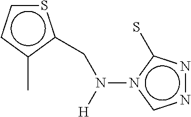 | ChemBridge | MW-Set |
| YU154833 | 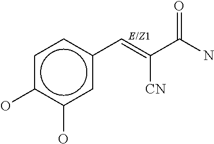 | Enzo | KinaseInhLib |
| YU145702 | 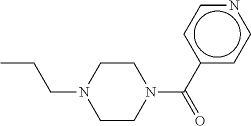 | ChemBridge | MW-Set |
| YU147088 | 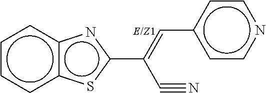 | ChemBridge | MW-Set |
| YU155488 | 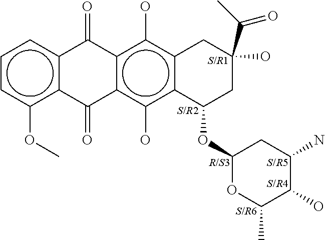 | Microsource | NaturalProducts |
| YU155369 | 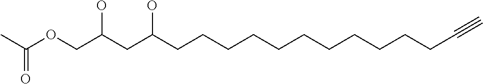 [SMILES: C#CCCCCCCCCCCCC(O)CC(O)COC(C)=O] | Microsource | NaturalProducts |
| YU145266 | 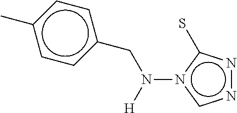 | ChemBridge | MW-Set |
| YU144949 | 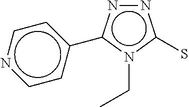 | ChemBridge | MW-Set |
| YU155007 | 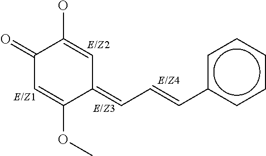 | Microsource | NaturalProducts |
| YU152236 | 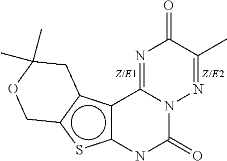 | ChemBridge | MW-Set |
| YU147191 | 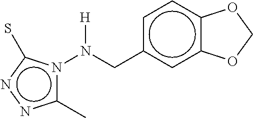 | ChemBridge | MW-Set |
| YU172199 | 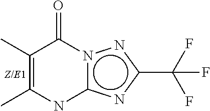 | ChemDiv | ChemDiv |
| YU034557 | 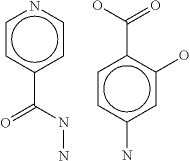 | Microsource | GenPlus |
| YU154859 | 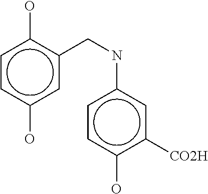 | Enzo | KinaseInhLib |
| YU151897 | 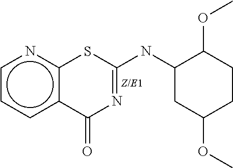 | ChemBridge | MW-Set |
| YU146438 | 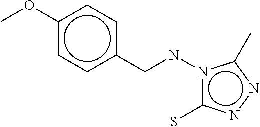 | ChemBridge | MW-Set |
| YU221013 | 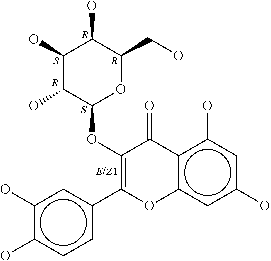 | NCC | NCC |
| YU034332 | 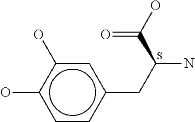 | Microsource | GenPlus |
| YU154885 | 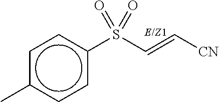 | Enzo | KinaseInhLib |
| YU145796 | 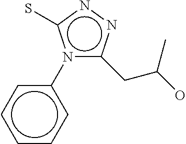 | ChemBridge | MW-Set |
| YU034538 | 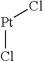 | Microsource | GenPlus |
| YU035331 | 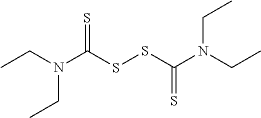 [SMILES: CCN(CC)C(=S)SSC(=S)N(CC)CC] | NCC | NCC |
| YU155075 | 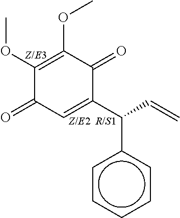 | Microsource | NaturalProducts |
| YU033927 | 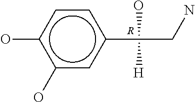 | Microsource | GenPlus |
| YU153110 | 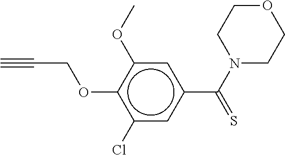 | ChemBridge | MW-Set |
| YU149607 | 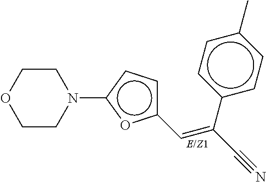 | ChemBridge | MW-Set |
| YU153353 | 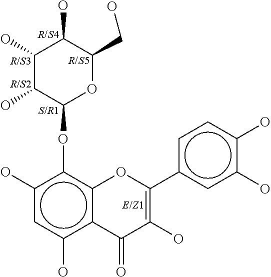 | Microsource | NaturalProducts |
| YU034226 | 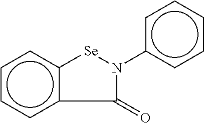 | Microsource | GenPlus |
| YU173003 | 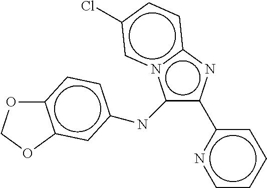 | ChemDiv | ChemDiv |
| YU221011 | 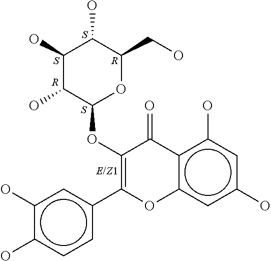 | NCC | NCC |
| YU034124 | 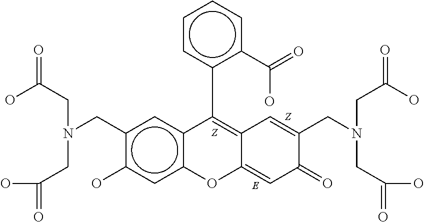 | Microsource | GenPlus |
| YU034024 | 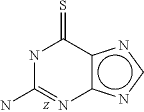 | Microsource | GenPlus |
| YU185530 | 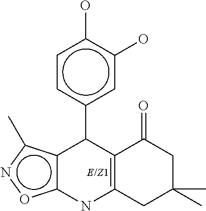 | ChemDiv | ChemDiv |
| YU183529 | 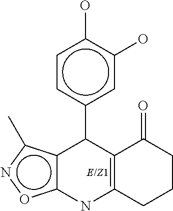 | ChemDiv | ChemDiv |
| YU033892 | 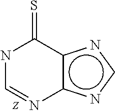 | Microsource | GenPlus |
| YU148159 | 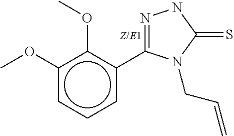 | ChemBridge | MW-Set |
| YU147024 | 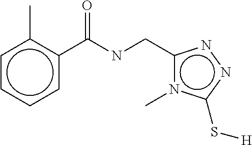 | ChemBridge | MW-Set |
| YU033874 | 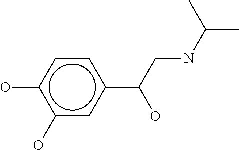 | Microsource | GenPlus |
| YU145029 | 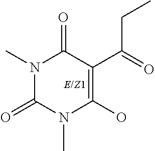 | ChemBridge | MW-Set |
| YU147005 | 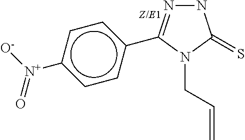 | ChemBridge | MW-Set |
| YU033802 | 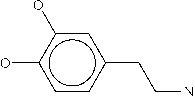 | Microsource | GenPlus |
| YU034372 | 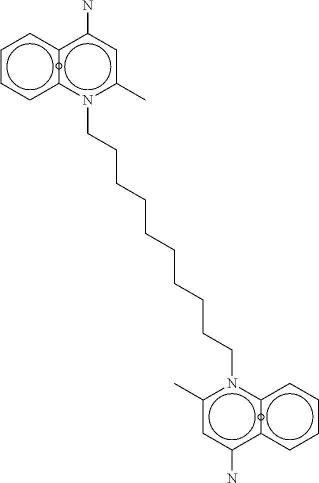 | Microsource | GenPlus |
| YU155411 | 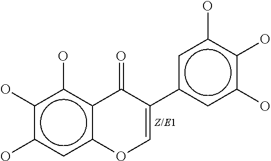 | Microsource | NaturalProducts |
| YU145747 | 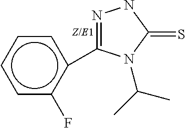 | ChemBridge | MW-Set |
| YU154829 | 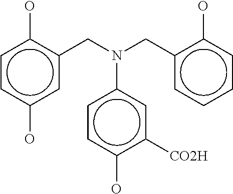 | Enzo | KinaseInhLib |
| YU034137 | 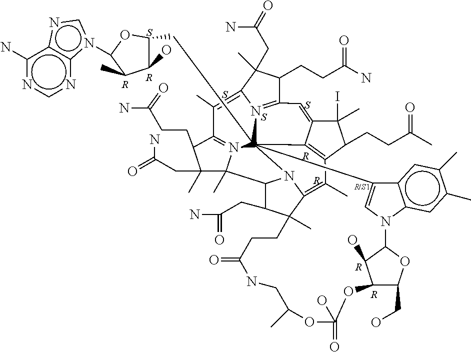 | Microsource | GenPlus |
| YU147658 | 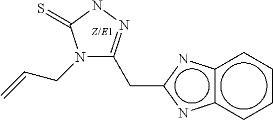 | ChemBridge | MW-Set |
| YU145980 | 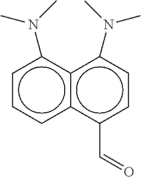 | ChemBridge | MW-Set |
| YU033743 | 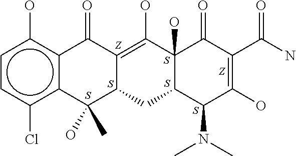 | Microsource | GenPlus |
| YU145653 | 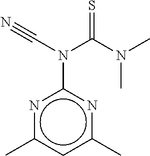 | ChemBridge | MW-Set |
| YU034463 | 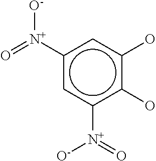 | Microsource | GenPlus |
| YU221143 | 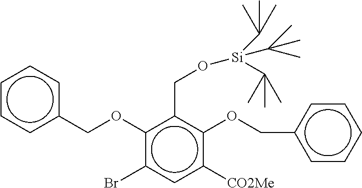 | Yale University | |
| YU034292 | 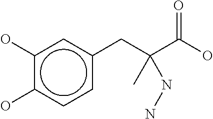 | Microsource | GenPlus |
| YU033631 | 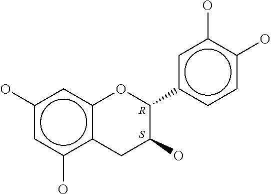 | Microsource | GenPlus |
| YU147054 | 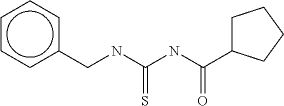 | ChemBridge | MW-Set |
| YU154091 | 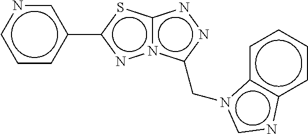 | ChemBridge | MW-Set |
| YU034265 | 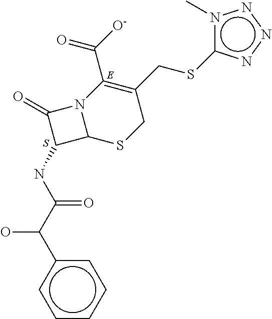 | Microsource | GenPlus |
| YU154737 | 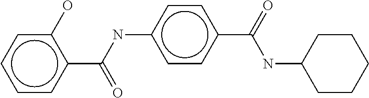 | ChemBridge | MW-Set |
| YU153281 | 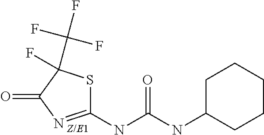 [SMILES: O=C(NC1=NC(=O)C(F)(C(F)(F)F)S1)NC1CCCCC1] | ChemBridge | MW-Set |
| YU221139 | 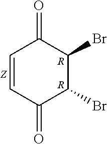 | Yale University | |
| YU147498 | 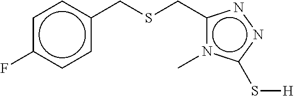 | ChemBridge | MW-Set |
| YU034518 | 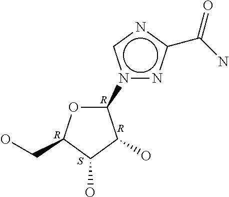 | Microsource | GenPlus |
| YU154835 | 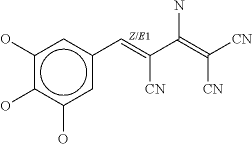 | Enzo | KinaseInhLib |
| YU144893 | 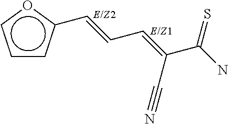 | ChemBridge | MW-Set |
| YU155255 | 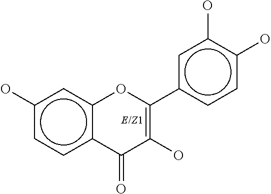 | Microsource | NaturalProducts |
| YU154882 | 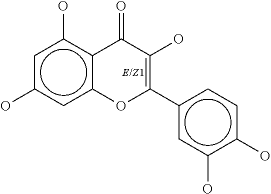 | Microsource | NaturalProducts |
| YU154834 | 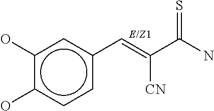 | Enzo | KinaseInhLib |
| YU153935 | 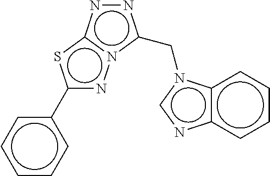 | ChemBridge | MW-Set |
| YU153356 | 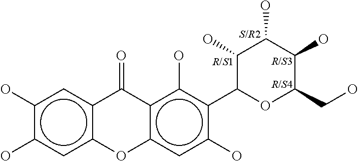 | Microsource | NaturalProducts |
| YU172087 | 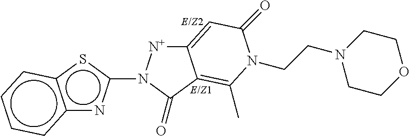 | ChemDiv | ChemDiv |
| YU033863 | 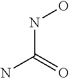 | Microsource | GenPlus |
| YU146034 | 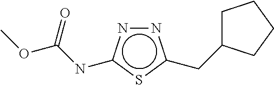 | ChemBridge | MW-Set |
| YU019467 | 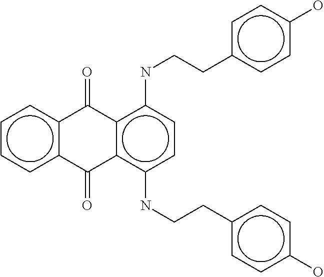 | Enzo | EpigensticsLib |
| YU221030 | 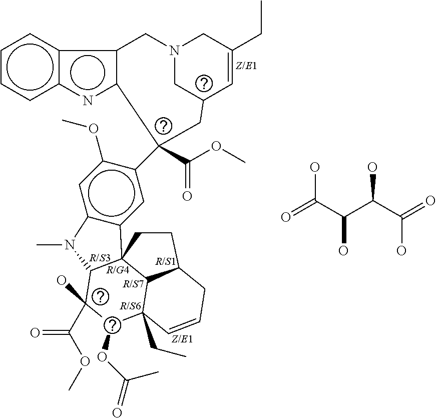 | NCC | NCC |
| YU033940 | 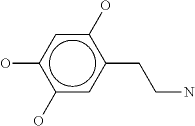 | Microsource | GenPlus |
| YU155087 | 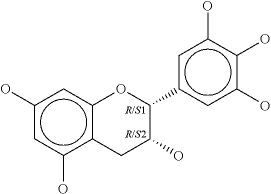 | Microsource | NaturalProducts |
| YU208232 | 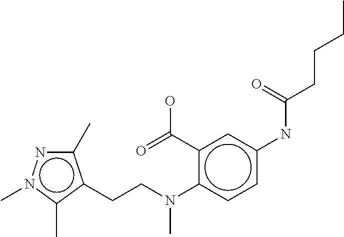 | ChemDiv | ChemDiv |
| YU155360 | 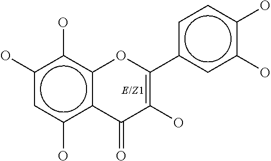 | Microsource | NaturalProducts |
| YU151559 | 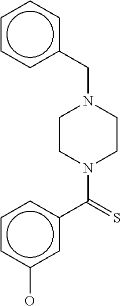 | ChemBridge | MW-Set |
| YU154484 | 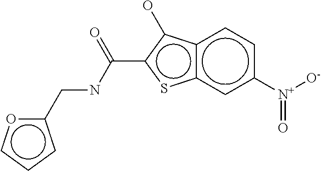 | ChemBridge | MW-Set |
| YU154843 | 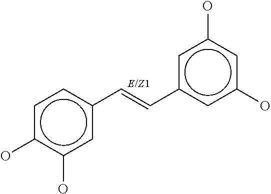 | Enzo | KinaseInhLib |
| YU154832 | 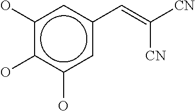 | Enzo | KinaseInhLib |
| YU148611 | 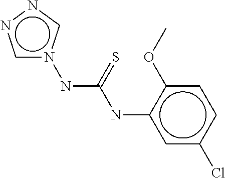 | ChemBridge | MW-Set |
| YU147019 | 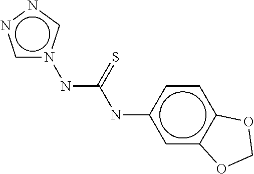 | ChemBridge | MW-Set |
| YU155443 | 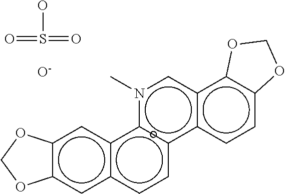 | Microsource | NaturalProducts |
| YU208199 | 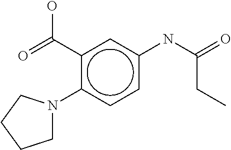 | ChemDiv | ChemDiv |
| YU148050 | 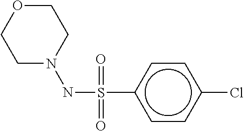 | ChemBridge | MW-Set |
| YU172170 | 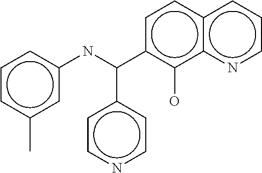 | ChemDiv | ChemDiv |
| YU151441 | 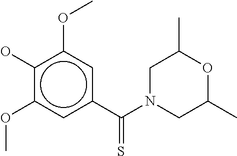 | ChemBridge | MW-Set |
| YU153003 | 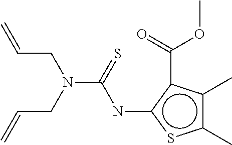 | ChemBridge | MW-Set |
| YU154998 | 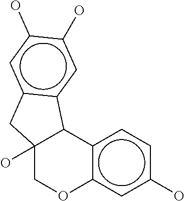 | Microsource | NaturalProducts |
| YU033752 | 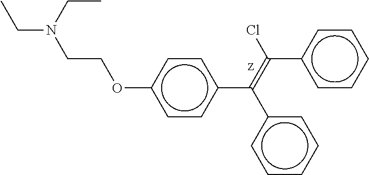 | Microsource | GenPlus |
| YU035082 | 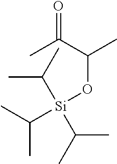 [SMILES: CC(=O)C(C)O[Si](C(C)C)(C(C)C)C(C)C] | Yale University | |
| YU040338 | 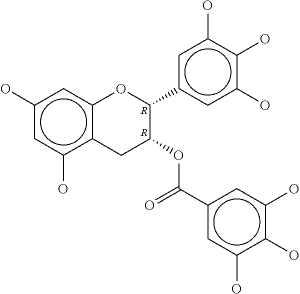 | NCC | NCC |
| YU147278 | 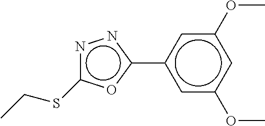 | ChemBridge | MW-Set |
| YU153729 | 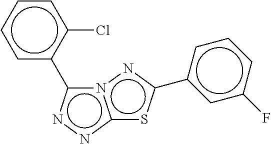 | ChemBridge | MW-Set |
| YU208250 | 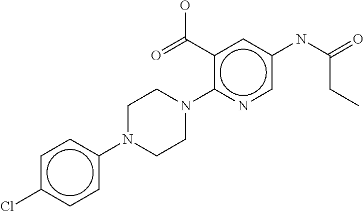 | ChemDiv | ChemDiv |
| YU151591 | 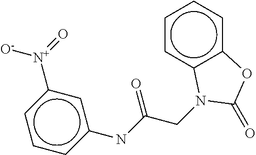 | ChemBridge | MW-Set |
| YU147801 | 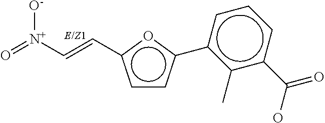 | ChemBridge | MW-Set |
| YU172165 | 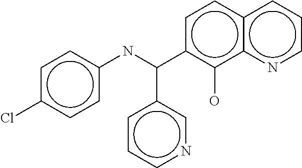 | ChemDiv | ChemDiv |
| YU147615 | 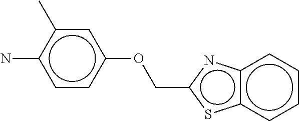 | ChemBridge | MW-Set |
| YU155016 | 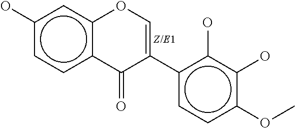 | Microsource | NaturalProducts |
| YU035181 | 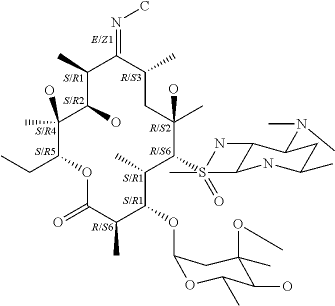 [SMILES: CC[C@H]1OC(=O)[C@H](C)[C@@H](OC2CC(C)(OC)C(O)C(C)O2)[C@H](C)[C@@H](S2(C)(=O)NC3C(N(C)C)CC(C)NC32)[C@](C)(O)C[C@@H](C)/C(=N\C)[C@H](C)[C@@H](O)[C@]1(C)O] | Yale University | |
| YU155003 | 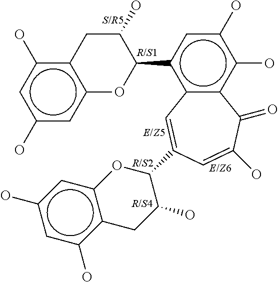 | Microsource | NaturalProducts |
| YU033694 | 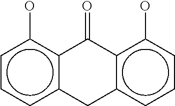 | Microsource | GenPlus |
| YU149839 | 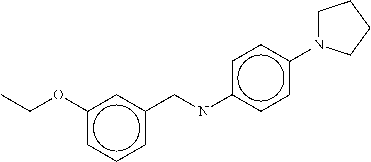 | ChemBridge | MW-Set |
| YU035165 | 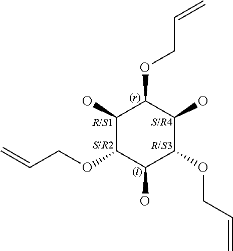 | Yale University | |
| YU149002 | 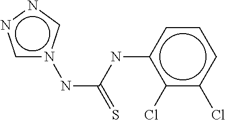 | ChemBridge | MW-Set |
| YU151714 | 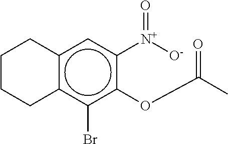 | ChemBridge | MW-Set |
| YU147245 | 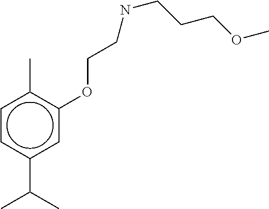 | ChemBridge | MW-Set |
| YU172169 | 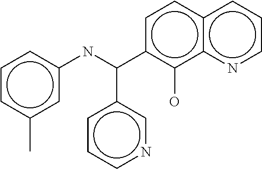 | ChemDiv | ChemDiv |
| YU035100 | 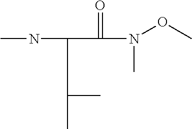 | Yale University | |
| YU208193 | 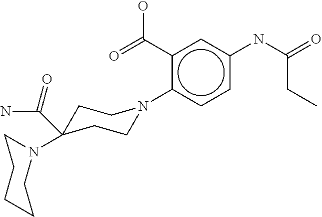 | ChemDiv | ChemDiv |
| YU149831 | 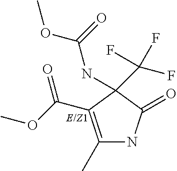 [SMILES: COC(=O)NC1(C(F)(F)F)C(=O)NC(C)=C1C(=O)OC] | ChemBridge | MW-Set |
| YU153074 | 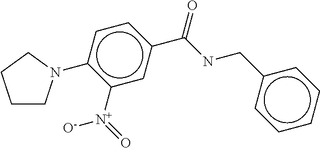 | ChemBridge | MW-Set |
| YU172089 | 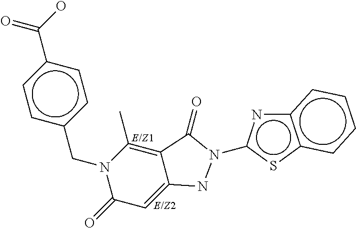 | ChemDiv | ChemDiv |
| YU152226 | 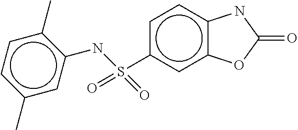 | ChemBridge | MW-Set |
| YU153287 | 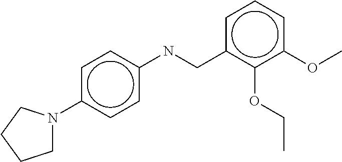 | ChemBridge | MW-Set |
| YU172175 | 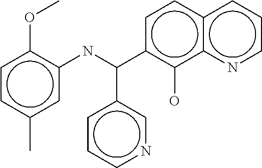 | ChemDiv | ChemDiv |
| YU172164 | 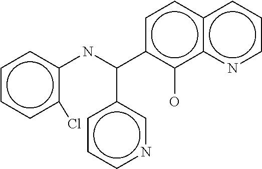 | ChemDiv | ChemDiv |
| YU148636 | 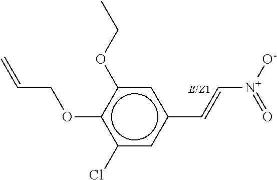 | ChemBridge | MW-Set |
| YU146534 | 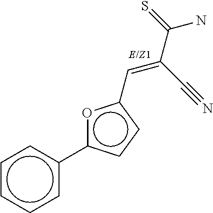 | ChemBridge | MW-Set |
| YU146590 | 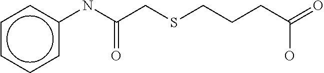 | ChemBridge | MW-Set |
| YU034420 | 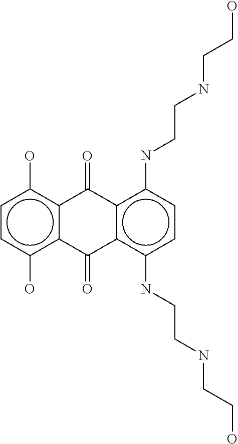 | Microsource | GenPlus |
| YU172179 | 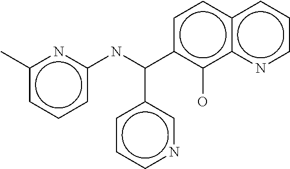 | ChemDiv | ChemDiv |
| YU208246 | 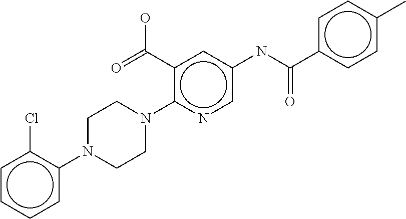 | ChemDiv | ChemDiv |
| YU154882 | 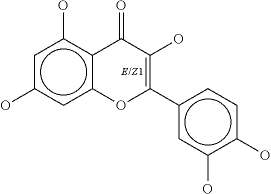 | Enzo | KinaseInhLib |
| YU145521 | 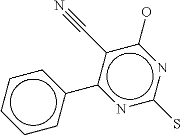 | ChemBridge | MW-Set |
| YU151071 | 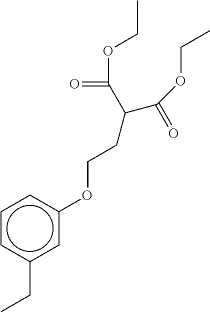 | ChemBridge | MW-Set |
| YU034320 | 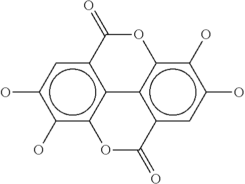 | Microsource | NaturalProducts |
| YU152765 | 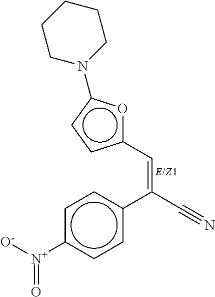 | ChemBridge | MW-Set |
| YU208239 | 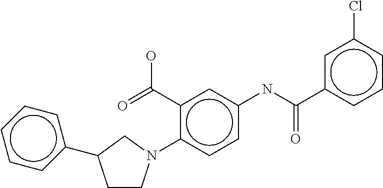 | ChemDiv | ChemDiv |
| YU148889 | 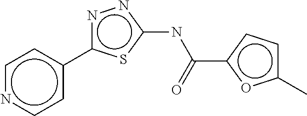 | ChemBridge | MW-Set |
| YU155025 | 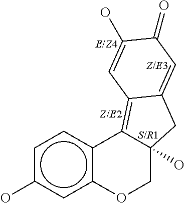 | Microsource | NaturalProducts |
| YU153031 | 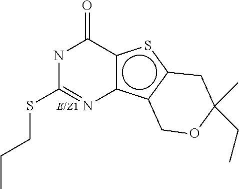 | ChemBridge | MW-Set |
| YU146020 | 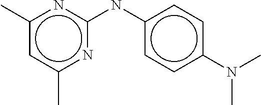 | ChemBridge | MW-Set |
| YU153545 | 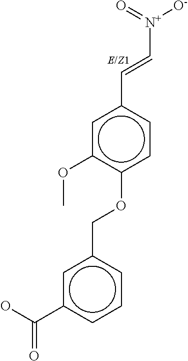 | ChemBridge | MW-Set |
| YU034447 | 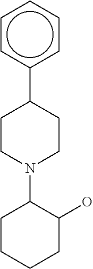 | Microsource | GenPlus |
| YU153085 | 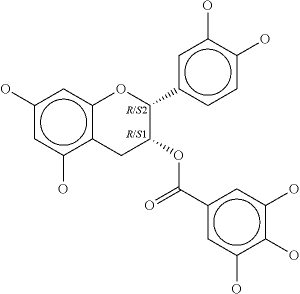 | Microsource | NaturalProducts |
| YU145407 | 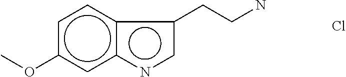 | ChemBridge | MW-Set |
| YU172178 | 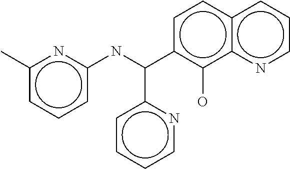 | ChemDiv | ChemDiv |
| YU148087 | 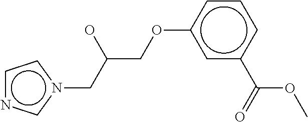 | ChemBridge | MW-Set |
| YU149951 | 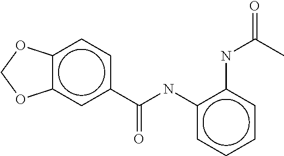 | ChemBridge | MW-Set |
| YU016748 | 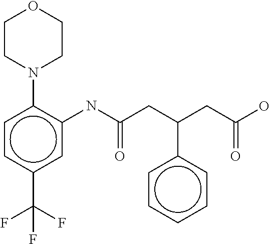 | Maybridge | |
| YU151128 | 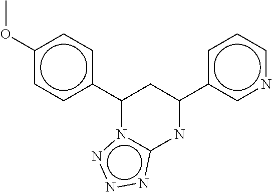 | ChemBridge | MW-Set |
| YU153128 | 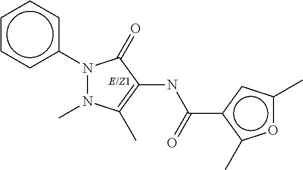 | ChemBridge | MW-Set |
| YU146294 | 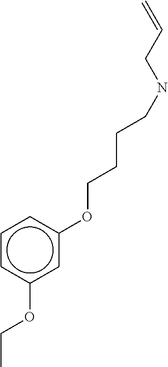 | ChemBridge | MW-Set |
| YU150371 | 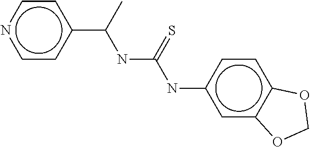 | ChemBridge | MW-Set |
| YU155347 | 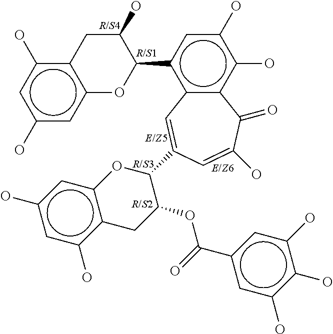 | Microsource | NaturalProducts |
| YU147640 | 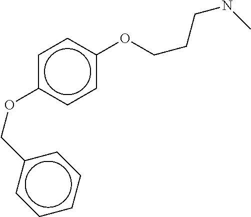 | ChemBridge | MW-Set |
| YU148680 | 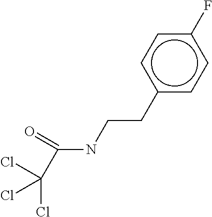 | ChemBridge | MW-Set |
| YU153899 | 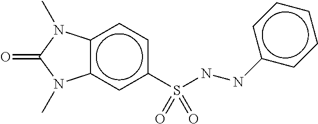 | ChemBridge | MW-Set |
| YU039791 | 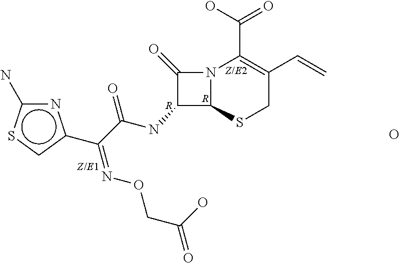 | NCC | NCC |
| YU033699 | 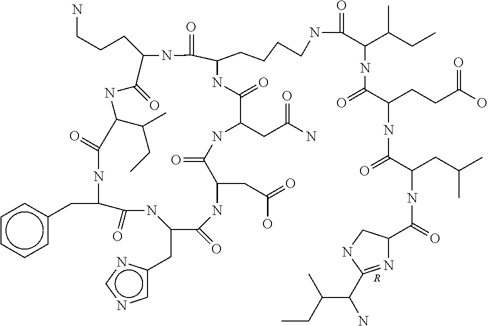 | Microsource | GenPlus |
| YU152893 | 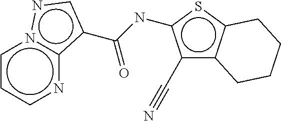 | ChemBridge | MW-Set |
| YU172168 | 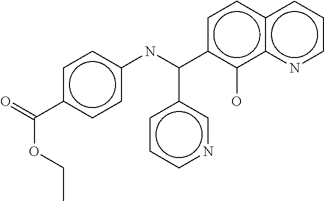 | ChemDiv | ChemDiv |
| YU172173 | 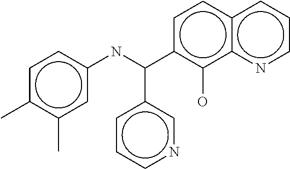 | ChemDiv | ChemDiv |
| YU145239 | 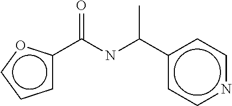 | ChemBridge | MW-Set |
| YU208247 | 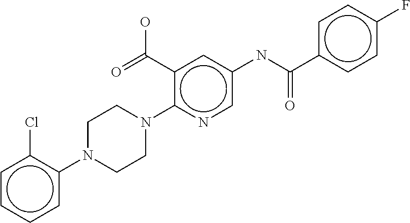 | ChemDiv | ChemDiv |
| YU151027 | 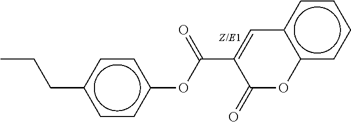 | ChemBridge | MW-Set |
| YU155084 | 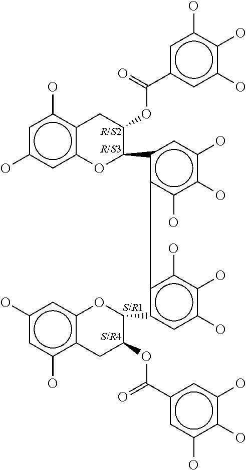 | Microsource | NaturalProducts |
| YU150472 | 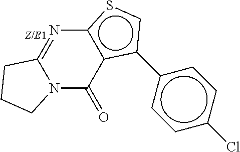 | ChemBridge | MW-Set |
| YU155440 | 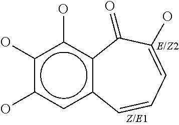 | Microsource | NaturalProducts |
| YU034048 | 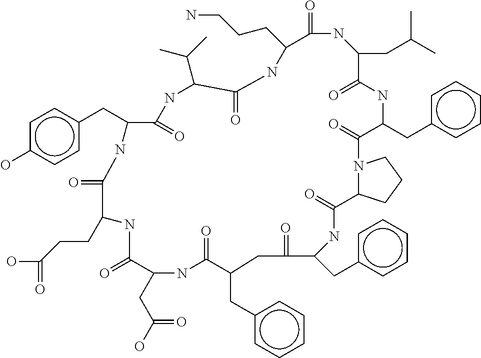 | Microsource | GenPlus |
| YU034370 | 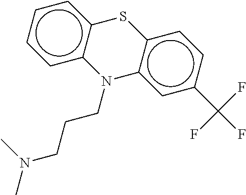 | Microsource | GenPlus |
| YU039604 | 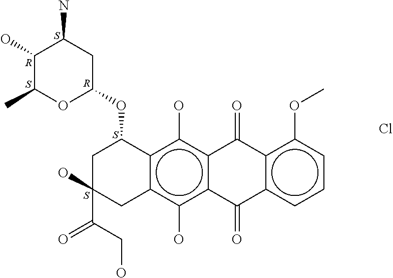 | NCC | NCC |
| YU152562 | 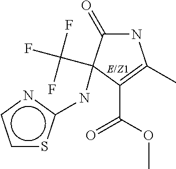 | ChemBridge | MW-Set |
| YU034506 | 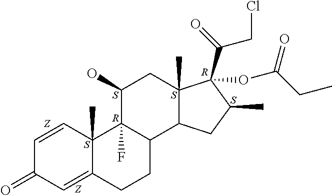 | Microsource | GenPlus |
| YU208230 | 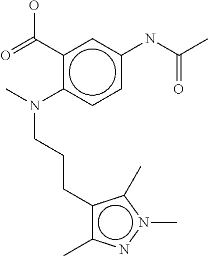 | ChemDiv | ChemDiv |
| YU172172 | 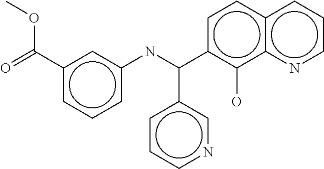 | ChemDiv | ChemDiv |
| YU151184 | 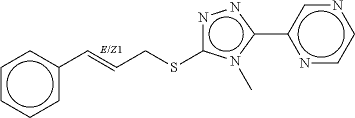 | ChemBridge | MW-Set |
| YU034320 | 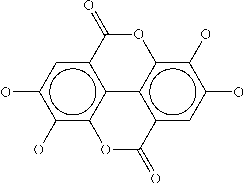 | Microsource | GenPlus |
| YU152583 | 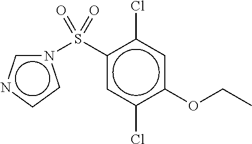 | ChemBridge | MW-Set |
| YU149514 | 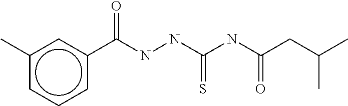 | ChemBridge | MW-Set |
| YU152451 | 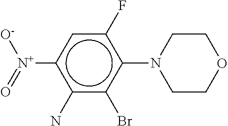 | ChemBridge | MW-Set |
| YU152649 | 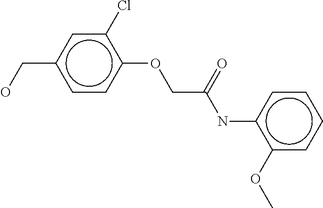 | ChemBridge | MW-Set |
| YU172071 | 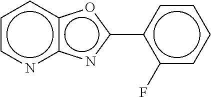 | ChemDiv | ChemDiv |
| YU155300 | 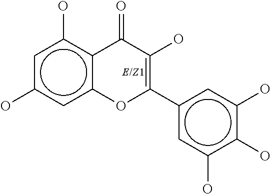 | Microsource | NaturalProducts |
| YU155257 | 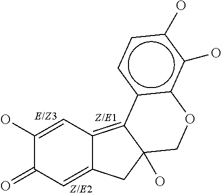 | Microsource | NaturalProducts |
| YU155407 | 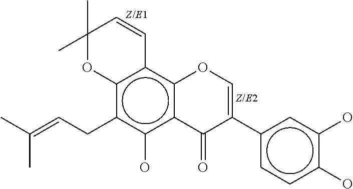 | Microsource | NaturalProducts |
| YU153090 | 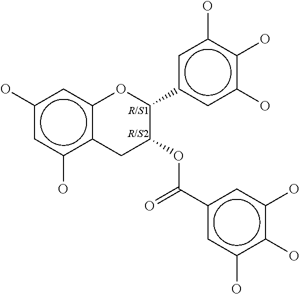 | Microsource | NaturalProducts |
| YU034167 | 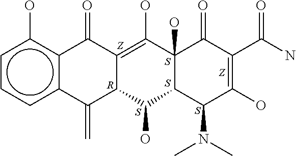 | Microsource | GenPlus |
| YU155100 | 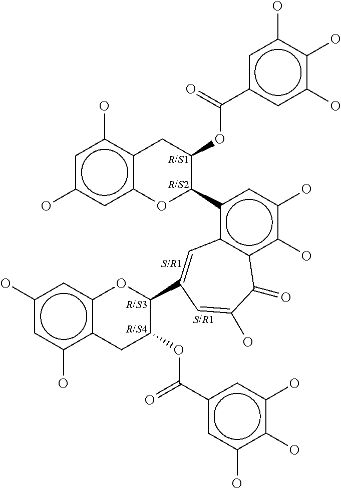 | Microsource | NaturalProducts |
| YU152183 | 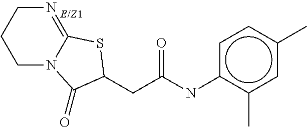 | ChemBridge | MW-Set |
| YU208194 | 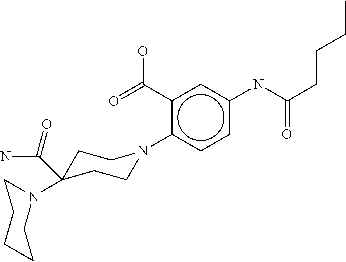 | ChemDiv | ChemDiv |
| YU208215 | 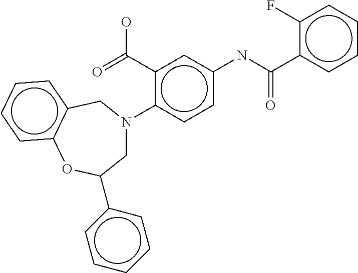 | ChemDiv | ChemDiv |
| YU208195 | 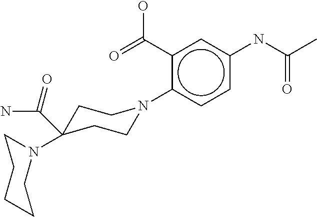 | ChemDiv | ChemDiv |
| YU153507 | 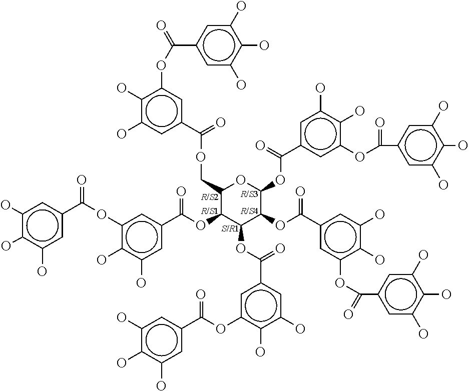 | Microsource | NaturalProducts |
| YU152560 | 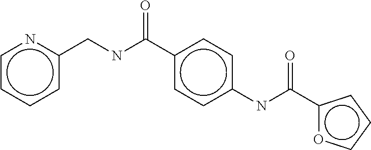 | ChemBridge | MW-Set |
| YU034398 | 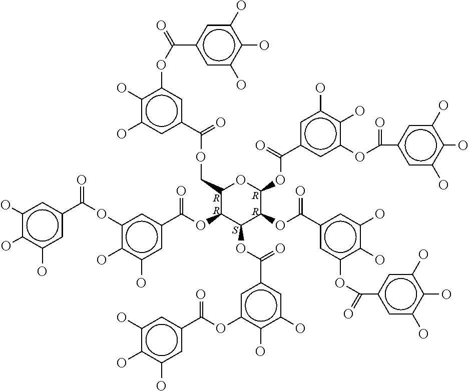 | Microsource | GenPlus |
| YU145089 | 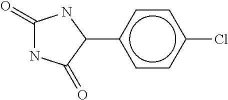 | ChemBridge | MW-Set |
| YU034068 | 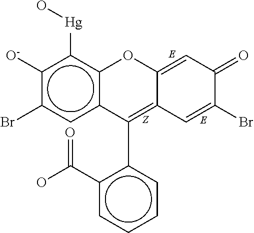 | Microsource | GenPlus |
| YU144896 | 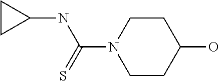 [SMILES: OC1CCN(C(=S)NC2CC2)CC1] | ChemBridge | MW-Set |
| YU033698 | 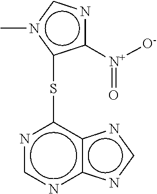 | Microsource | GenPlus |
| YU034331 | 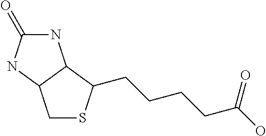 [SMILES: O=C(O)CCCCC1SCC2NC(=O)NC21] | Microsource | GenPlus |
| YU039629 | 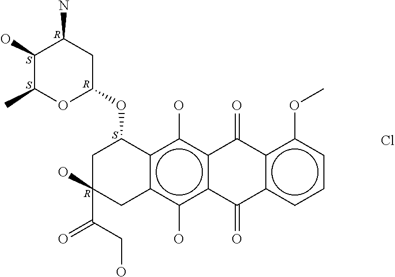 | NCC | NCC |
| YU105182 | 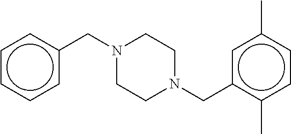 | ChemBridge | McF |
| YU152564 | 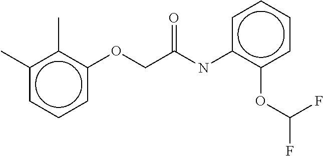 | ChemBridge | MW-Set |
| YU121632 | 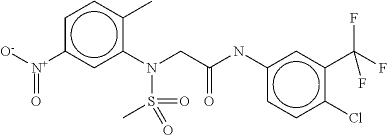 | ChemBridge | McF |
| YU034556 | 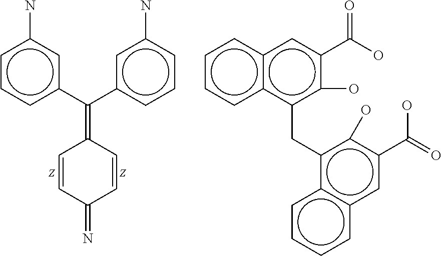 | Microsource | GenPlus |
| YU208200 | 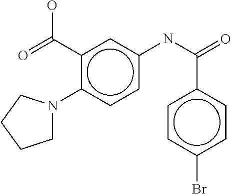 | ChemDiv | ChemDiv |
| YU034090 | 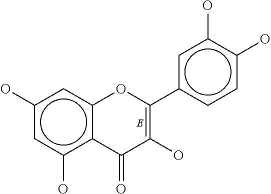 | Microsource | GenPlus |
| YU208228 | 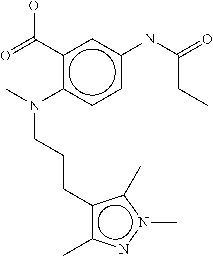 | ChemDiv | ChemDiv |
| YU155305 | 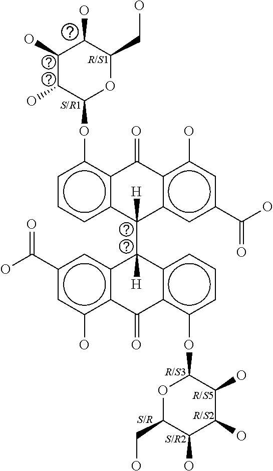 | Microsource | NaturalProducts |
| YU033712 | 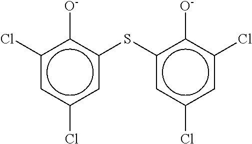 | Microsource | GenPlus |
| YU155528 | 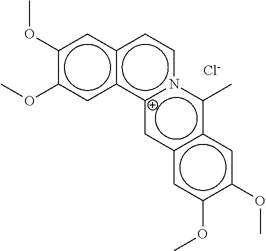 | Microsource | NaturalProducts |
| YU151409 | 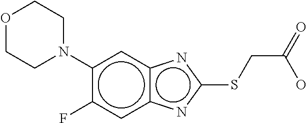 | ChemBridge | MW-Set |
| YU104987 | 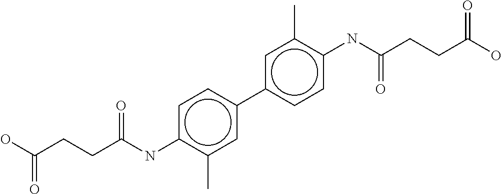 | ChemBridge | McF |
| YU151859 | 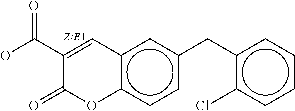 | ChemBridge | MW-Set |
| YU147750 | 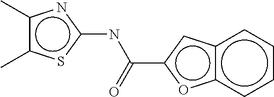 | ChemBridge | MW-Set |
| YU034074 | 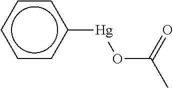 | Microsource | GenPlus |
| YU150447 | 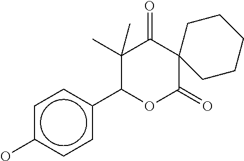 | ChemBridge | MW-Set |
| YU149941 | 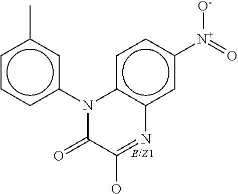 | ChemBridge | MW-Set |
| YU155201 | 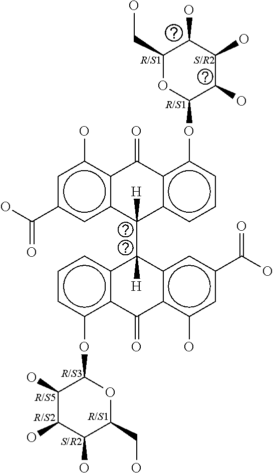 | Microsource | NaturalProducts |
| YU153907 | 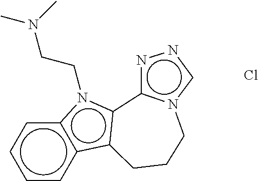 | ChemBridge | MW-Set |
| YU105195 | 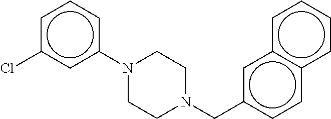 | ChemBridge | McF |
| YU150403 | 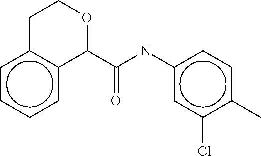 | ChemBridge | MW-Set |
| YU151614 | 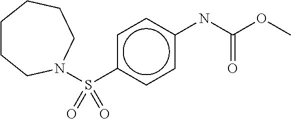 | ChemBridge | MW-Set |
| YU152920 | 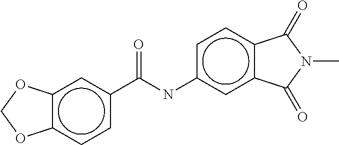 | ChemBridge | MW-Set |
| YU155091 | 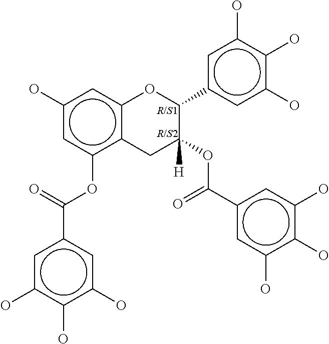 | Microsource | NaturalProducts |
| YU033837 | 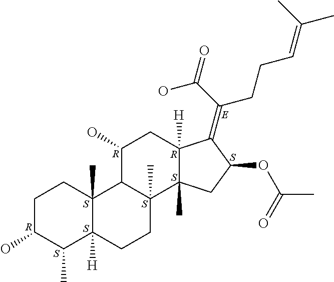 | Microsource | GenPlus |
| YU033841 | 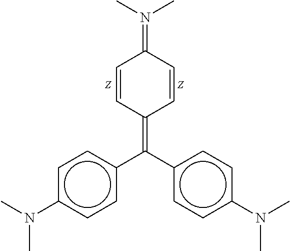 | Microsource | GenPlus |
| YU034263 | 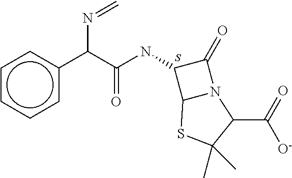 | Microsource | GenPlus |
| YU034023 | 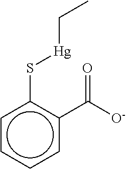 | Microsource | GenPlus |
| YU152624 | 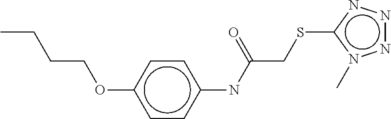 | ChemBridge | MW-Set |
| YU149922 | 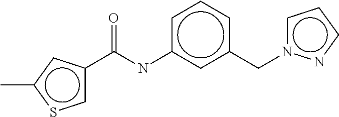 | ChemBridge | MW-Set |
| YU154853 | 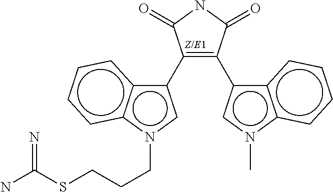 | Enzo | KinaseInhLib |
| YU145520 | 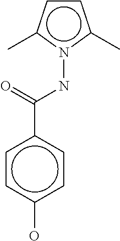 | ChemBridge | MW-Set |
| YU221071 | 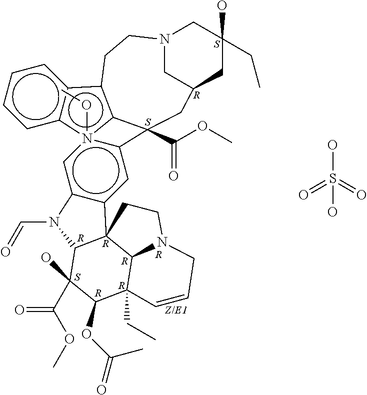 | NCC | NCC |
| YU155193 | 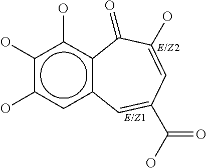 | Microsource | NaturalProducts |
| YU033800 | 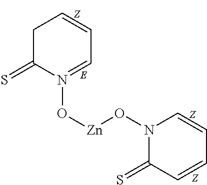 | Microsource | GenPlus |
| YU033802 | 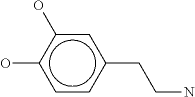 | Microsource | NaturalProducts |
| YU152587 | 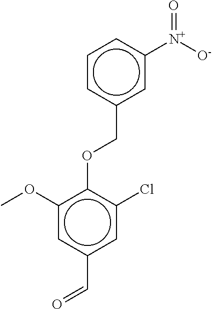 | ChemBridge | MW-Set |
| YU105079 | 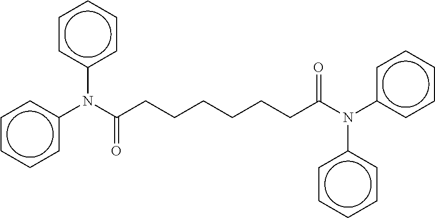 | ChemBridge | McF |
| YU146037 | 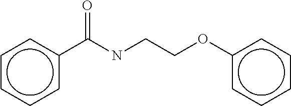 | ChemBridge | MW-Set |
| YU034547 | 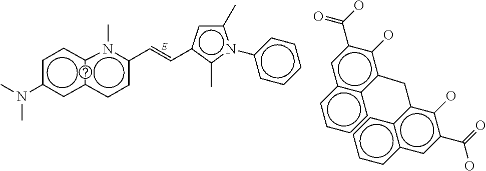 | Microsource | GenPlus |
| YU149834 | 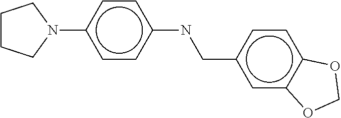 | ChemBridge | MW-Set |
| YU154851 | 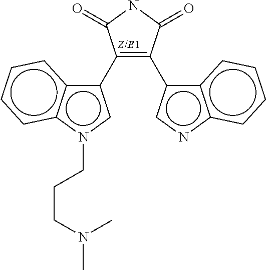 | Enzo | KinaseInhLib |
| YU145132 | 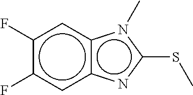 | ChemBridge | MW-Set |
| YU155285 | 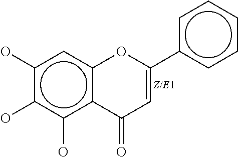 | Microsource | NaturalProducts |
| YU033619 | 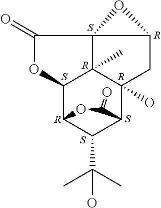 | Microsource | GenPlus |
| YU034377 | 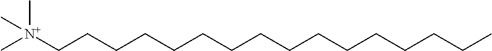 [SMILES: CCCCCCCCCCCCCCCC[N+](C)(C)C] | Microsource | GenPlus |
| YU040321 | 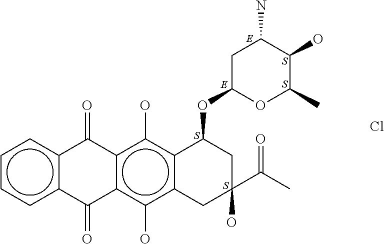 | NCC | NCC |
| YU145037 | 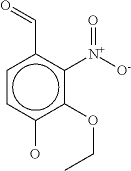 | ChemBridge | MW-Set |
| YU016812 | 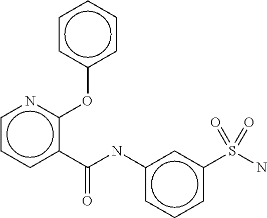 | Maybridge | |
| YU033734 | 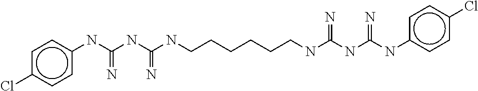 | Microsource | GenPlus |
| YU034020 | 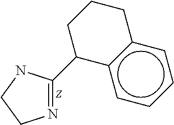 | Microsource | GenPlus |
| YU033628 | 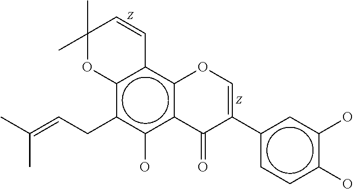 | Microsource | GenPlus |
| YU034280 | 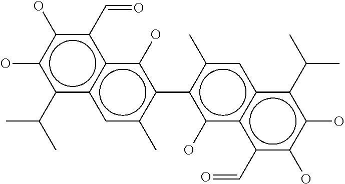 | Microsource | GenPlus |
| YU155086 | 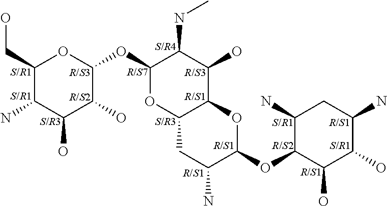 [SMILES: CN[C@@H]1[C@H](O[C@H]2O[C@H](CO)[C@@H](N)[C@H](O)[C@H]2O)O[C@H]2C[C@@H](N)[C@H](O[C@@H]3[C@H](O)[C@@H](O)[C@H](N)C[C@@H]3N)O[C@@H]2[C@@H]1O] | Microsource | NaturalProducts |
| YU033898 | 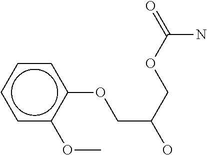 | Microsource | GenPlus |
| YU155308 | 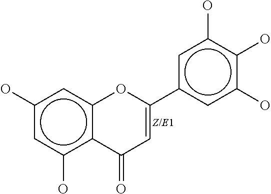 | Microsource | NaturalProducts |

| Compound | | | Assay Signal | Artifact Signal | Assay − Artifact |
|---|---|---|---|---|---|
| ID | Supplier ID | Drug Name | (percent) | (percent) | Signal (percent) |
| YU146842 | 6339039 | | 97.21 | −6.95 | 104.16 |
| YU034226 | SAM001247071 | EBSELEN | 92.26 | −7.26 | 99.53 |
| YU155312 | 01504124 | LINAMARIN | 92.68 | −6.55 | 99.23 |
| YU153803 | 7812182 | | 93.75 | −5.27 | 99.03 |
| YU129955 | 5131356 | | 92.06 | −4.84 | 96.89 |
| YU221215 | A-280 | 2,4-Pyridinedicarboxylic Acid | 99.69 | 3.00 | 96.69 |
| YU145461 | 7935634 | | 96.37 | 0.89 | 95.48 |
| YU172999 | C177-0098 | | 98.42 | 10.03 | 88.40 |
| YU145649 | 5224440 | | 89.97 | 2.10 | 87.87 |
| YU146026 | 7938963 | | 88.51 | 2.88 | 85.63 |
| YU155621 | 01500819 | BERGENIN | 93.44 | 20.42 | 73.02 |
| YU147266 | 7864151 | | 69.21 | 0.11 | 69.10 |
| YU034066 | 01500634 | IPRONIAZID SULFATE | 72.51 | 3.41 | 69.09 |
| YU146454 | 6914720 | | 64.68 | −4.14 | 68.83 |
| YU129874 | 6104953 | | 72.06 | 4.18 | 67.88 |
| YU034347 | 01503074 | [symbol] ALEXIDINE HYDROCHLORID [symbol] | 72.00 | 5.60 | 66.40 |
| YU033938 | 01500447 | ORPHENADRINE CITRATE | 66.35 | 0.79 | 56.56 |
| YU155005 | 00200010 | HAEMATOXYLIN | 98.90 | 34.32 | 64.59 |
| YU155280 | 01503987 | CAFFEIC ACID | 62.26 | −1.92 | 64.18 |
| YU155534 | 01500899 | ESCULETIN | 66.12 | 6.30 | 59.82 |
| YU034426 | 01503322 | THIRAM | 66.73 | 7.49 | 59.23 |
| YU033662 | 00310035 | SANGUINARINE SULFATE | 67.85 | 9.21 | 58.64 |
| YU033696 | 01500129 | [symbol] OMORPHINE HYDROCHLOR [symbol] | 66.78 | 8.29 | 58.49 |
| YU155618 | 00210206 | EPICATECHIN | 64.74 | 6.96 | 57.78 |
| YU145853 | 7939195 | | 66.64 | 8.99 | 57.65 |
| YU154887 | EJ-273 | [symbol] Y-1,1-BIPHENYL-6,6-DIMET [symbol] | 99.57 | 42.41 | 57.15 |
| YU221065 | SAM001247083 | [symbol] ne-10,11-diol, 5,6,6a,7,8,12b-hex [symbol] | 59.95 | 4.21 | 55.73 |
| YU148374 | 7813798 | | 77.28 | 22.77 | 54.50 |
| YU155214 | 91500817 | CARMINIC ACID | 78.58 | 24.32 | 54.26 |
| YU146041 | 7986284 | | 55.23 | 0.99 | 54.24 |
| YU147704 | 7955823 | | 55.47 | 1.42 | 54.05 |
| YU146698 | 7377697 | | 48.27 | −5.12 | 53.39 |
| YU033654 | 90300607 | RUTOSIDE (midn) | 58.08 | 5.37 | 52.71 |
| YU149180 | 7907173 | | 50.21 | −2.49 | 52.70 |
| YU033702 | 01500137 | [symbol] NSERAZIDE HYDROCHLOR [symbol] | 73.96 | 21.46 | 52.50 |
| YU033872 | 01500355 | ISONIAZID | 45.10 | −6.34 | 51.63 |
| YU155562 | 00210369 | GALLIC ACID | 59.88 | 8.44 | 51.44 |
| YU033903 | 01500403 | METHYLDOPA | 50.21 | −0.54 | 30.75 |
| YU033971 | 01500500 | PRIMAQUINE DIPHOSPHATE | 46.49 | −4.11 | 50.60 |
| YU146768 | 7507138 | | 48.97 | −0.52 | 49.49 |
| YU149014 | 7840569 | | 47.61 | −1.43 | 49.04 |
| YU155465 | 01600919 | 3-METHOXYCATECHOL | 56.84 | 7.97 | 48.87 |
| YU173004 | C177-0168 | | 49.67 | 0.87 | 48.80 |
| YU150981 | 7958378 | | 52.35 | 3.65 | 48.70 |
| YU146471 | 7851437 | | 46.29 | −1.64 | 47.93 |
| YU033809 | 01500274 | ADRENALINE BITARTRATE | 40.90 | −6.64 | 47.54 |
| YU145432 | 7496439 | | 48.50 | 1.67 | 46.83 |
| YU154833 | EI-257 | TYRPHOSTIN 46 | 50.42 | 4.32 | 46.11 |
| YU145702 | 7943502 | | 44.33 | −1.53 | 45.86 |
| YU147088 | 6145186 | | 46.00 | 0.34 | 45.66 |
| YU155488 | 01500223 | DAUNORUBICIN | 48.63 | 3.31 | 45.32 |
| YU155369 | 00240929 | AVOCADYNE ACETATE | 44.82 | −0.21 | 45.03 |
| YU145266 | 7490877 | | 45.11 | 1.04 | 44.07 |
| YU144949 | 7117164 | | 40.70 | −3.18 | 43.88 |
| YU155007 | 00200090 | OBTUSAQUINONE | 51.64 | 7.90 | 43.75 |
| YU152236 | 7919641 | | 49.40 | 5.71 | 43.69 |
| YU147191 | 7879054 | | 45.05 | 1.94 | 43.11 |
| YU172199 | 8407-0795 | | 38.73 | −4.00 | 42.72 |
| YU034557 | 0150338J | PASINIAZID | 44.38 | 1.76 | 42.63 |
| YU154859 | EI-232 | [symbol] 5-DIHYDROXYBENZYLAMI [symbol] | 46.26 | 3.65 | 42.61 |
| YU151897 | 7905968 | | 40.43 | −2.12 | 42.55 |
| YU146438 | 7985526 | | 50.33 | 8.17 | 42.15 |
| YU221013 | SAM001246776 | HYPEROSIDE | 51.29 | 9.46 | 41.83 |
| YU034332 | 02300203 | LEVODOPA | 55.74 | 14.27 | 41.47 |
| YU154885 | EI-278 | BAY 11-7082 | 35.57 | −5.88 | 41.45 |
| YU145796 | 7951573 | | 33.04 | −7.79 | 40.83 |
| YU034538 | 01502107 | CISPLATIN | 49.17 | 8.53 | 40.64 |
| YU035331 | SAM001247028 | [symbol] RAETHYLTHIURAMDISUL [symbol] | 36.77 | −3.39 | 40.15 |
| YU155075 | 00240828 | 4-DIMETHOXYDALBERGIO [symbol] | 49.97 | 11.14 | 38.83 |
| YU033927 | 01500436 | NOREPINEPHRINE | 39.80 | 1.83 | 37.97 |
| YU153110 | 7916412 | | 34.87 | −3.09 | 37.96 |
| YU149607 | 7937853 | | 40.44 | 2.50 | 37.94 |
| YU153353 | 01505127 | GOSSYPIN | 99.61 | 61.80 | 37.81 |
| YU034226 | 01501188 | EBSHLEN | 32.63 | −4.70 | 37.33 |
| YU173003 | C177-0167 | | 42.73 | 3.56 | 37.18 |
| YU221011 | SAM001246767 | Isoquercitrin | 52.16 | 15.17 | 36.99 |
| YU034124 | 01500763 | CALCEIN | 33.76 | −3.21 | 36.96 |
| YU034024 | 91500573 | THIOGUANINE | 36.89 | 0.37 | 36.52 |
| YU185530 | D588-0192 | | 38.23 | 1.72 | 36.51 |
| YU183529 | D588-0191 | | 40.22 | 4.16 | 36.06 |
| YU033892 | 01500387 | MERCAPTOPURINE | 33.79 | −2.15 | 35.94 |
| YU148159 | 7915345 | | 38.72 | 2.98 | 35.74 |
| YU147024 | 7854533 | | 29.44 | −5.84 | 35.27 |
| YU033874 | 01500357 | [symbol] PROTERENOL HYDROCHLO [symbol] | 32.81 | −2.38 | 35.20 |
| YU145029 | 6628987 | | 34.53 | 0.11 | 34.42 |
| YU147005 | 5954633 | | 37.42 | 3.36 | 34.06 |
| YU033802 | 01500263 | [symbol] OPAMINE HYDROCHLORID [symbol] | 42.68 | 10.00 | 32.68 |
| YU034372 | 01503127 | DEQUALILIUM CHLORIDE | 32.80 | 0.57 | 32.23 |
| YU155411 | 90201182 | TRIGENOL | 22.64 | −9.11 | 31.75 |
| YU145747 | 7784784 | | 36.22 | 4.74 | 31.49 |
| YU154829 | EI-185 | LAVENDUSTIN A | 38.57 | 8.32 | 30.24 |
| YU034137 | 01500844 | COBALAMINE | 27.06 | 1.27 | 28.34 |
| YU147658 | 7862976 | | 27.63 | −0.55 | 28.18 |
| YU145980 | 6647257 | | 35.60 | 7.56 | 28.03 |
| YU033743 | 01500186 | AUREOMYCIN | 28.24 | 0.43 | 27.81 |
| YU145653 | 6638931 | | 18.58 | −8.59 | 27.17 |
| YU034463 | 01503631 | [symbol] 5-DINITROCATECHOL (OR-4 [symbol] | 38.78 | 11.72 | 27.05 |
| YU221143 | Crews08 | | 22.60 | −3.99 | 26.59 |
| YU034292 | 01502150 | CARBIDOPA | 80.27 | 53.76 | 26.51 |
| YU033631 | 00210205 | CIANIDANOL | 30.96 | 4.84 | 26.12 |
| YU147054 | 7942453 | | 22.98 | −1.54 | 24.52 |
| YU154091 | 7952897 | | 29.87 | 5.50 | 24.37 |
| YU034263 | 01502038 | CEFAMANDOLE SODIUM | 24.27 | 0.13 | 24.14 |
| YU154737 | 7954771 | | 28.23 | 4.58 | 23.65 |
| YU153281 | 7934812 | | 25.46 | 1.95 | 23.51 |
| YU221139 | Crews04 | | 30.40 | 7.07 | 23.33 |
| YU147498 | 7832823 | | 19.36 | −3.87 | 23.24 |
| YU034518 | 01503938 | RIBAVIRIN | 17.20 | −5.97 | 23.17 |
| YU154835 | EI-189 | TYRPHOSTIN 51 | 46.05 | 23.01 | 23.04 |
| YU144893 | 7850219 | | 62.72 | 40.11 | 22.61 |
| YU155255 | 01502247 | FISETIN | 91.26 | 68.78 | 22.49 |
| YU154882 | 01500672 | QUERCETIN | 100.88 | 78.83 | 22.05 |
| YU154834 | Ef-188 | TYRPHOSTIN 47 | 40.23 | 18.48 | 21.75 |
| YU153935 | 7951242 | | 14.67 | −6.99 | 21.66 |
| YU153356 | 01505134 | MANGIFERIN | 98.21 | 76.59 | 21.62 |
| YU172087 | 8249-3507 | | 14.44 | −6.77 | 21.21 |
| YU033863 | 91300344 | HYDROXYUREA | 13.20 | −8.00 | 21.20 |
| YU146034 | 7949611 | | 18.33 | <2.79 | 21.12 |
| YU019467 | GR-346 | BML-266 | 76.09 | 55.18 | 20.91 |
| YU221030 | SAM001246780 | VINORELBINE BITATRATE | 38.77 | 18.13 | 20.64 |
| YU033940 | 01500450 | [symbol] IDOPAMINE HYDROCHLOR [symbol] | 36.84 | 16.96 | 19.88 |
| YU155087 | 00203113 | EPIGALLOCATECHIN | 44.50 | 24.76 | 19.74 |
| YU208232 | G889-1299 | | 37.92 | 18.67 | 19.26 |
| YU155360 | 01505143 | GOSSYPETIN | 100.77 | 81.81 | 18.96 |
| YU151559 | 7784869 | | 27.61 | 9.22 | 18.39 |
| YU154484 | 7947354 | | 32.51 | 14.38 | 18.13 |
| YU154843 | Ef-27J | PICEATANNOL | 13.28 | −4.85 | 18.12 |
| YU154832 | EI-187 | TYRPHOSTIN 25 | 23.08 | 5.21 | 17.87 |
| YU148611 | 7792444 | | 15.61 | −2.20 | 17.82 |
| YU147019 | 7819221 | | 16.46 | −1.29 | 17.76 |
| YU155443 | 00310035 | SANGUINARINE SULFATE | 5.51 | −11.85 | 17.36 |
| YU208199 | G889-0409 | | 50.16 | 32.96 | 17.19 |
| YU148050 | 7243257 | | 20.47 | 3.44 | 17.03 |
| YU172170 | 8397-0181 | | 65.09 | 48.19 | 16.90 |
| YU151441 | 7934320 | | 12.98 | −3.82 | 16.81 |
| YU153003 | 7817806 | | 23.56 | 6.76 | 16.80 |
| YU154998 | 00200012 | BRAZILIN | 15.51 | −0.48 | 15.98 |
| YU033752 | 01500196 | CLOMIPHENE CITRATE | 16.67 | 0.86 | 15.81 |
| YU035082 | JS24 | | 22.36 | 6.78 | 15.58 |
| YU040338 | SAM001247031 | Epigallocatechin gallate | 96.04 | 80.56 | 15.48 |
| YU147278 | 7916510 | | 9.43 | −5.71 | 15.14 |
| YU153729 | 7879885 | | 11.41 | −3.54 | 14.96 |
| YU208250 | G890-0200 | | 21.70 | 7.01 | 14.69 |
| YU151591 | 7914537 | | 16.26 | 1.65 | 14.61 |
| YU147801 | 7220012 | | 31.41 | 16.98 | 14.43 |
| YU172165 | 8397-0140 | | 43.01 | 28.76 | 14.24 |
| YU147615 | 7938899 | | 14.43 | 0.54 | 13.88 |
| YU155016 | 90200422 | KOPARIN | 28.09 | 14.33 | 13.76 |
| YU035181 | CAL_oxime | | 18.53 | 4.88 | 13.65 |
| YU155003 | 0020011J | THEAFLAVIN | 60.81 | 47.32 | 13.49 |
| YU033694 | 01500127 | ANTHRALIN | 22.65 | 9.35 | 13.30 |
| YU149839 | 7932017 | | 31.65 | 18.44 | 13.21 |
| YU035165 | SK_6 | | 15.19 | 2.11 | 13.07 |
| YU149002 | 7788977 | | 15.58 | 3.30 | 12.28 |
| YU151714 | 7774023 | | 13.44 | 1.24 | 12.20 |
| YU147245 | 7015081 | | 13.31 | 1.25 | 12.07 |
| YU172169 | 8397-0180 | | 73.22 | 61.44 | 11.78 |
| YU035100 | JS46 | | 13.14 | 1.39 | 11.75 |
| YU208193 | G829-0167 | | 27.63 | 16.13 | 11.50 |
| YU149831 | 7919340 | | 13.59 | 2.14 | 11.45 |
| YU153074 | 7672253 | | 16.52 | 5.15 | 11.37 |
| YU172089 | 8249-3642 | | 22.52 | 11.16 | 11.36 |
| YU152226 | 7910527 | | 5.25 | −6.03 | 11.28 |
| YU153287 | 7943091 | | 28.35 | 17.14 | 11.21 |
| YU172175 | 8397-0559 | | 48.09 | 36.93 | 11.16 |
| YU172164 | 8397-0127 | | 47.38 | 36.24 | 11.14 |
| YU148636 | 7916410 | | 10.64 | −0.47 | 11.11 |
| YU146534 | 7494112 | | 15.42 | 4.53 | 10.89 |
| YU146590 | 7957074 | | 13.35 | 2.50 | 10.85 |
| YU034420 | 01503278 | [symbol] OXANTHRONE HYDROCHLO [symbol] | 99.87 | 89.32 | 10.55 |
| YU172179 | 8397-0663 | | 63.74 | 53.31 | 10.43 |
| YU208246 | G890-0096 | | 10.32 | −0.08 | 10.41 |
| YU154882 | AC-1142 | QUERCETIN | 78.65 | 68.48 | 10.18 |
| YU145521 | 7694782 | | 6.99 | −2.92 | 9.91 |
| YU151071 | 7005264 | | 6.13 | −3.73 | 9.86 |
| YU034320 | 01502245 | ELLAGIC ACID | 12.08 | 2.37 | 9.70 |
| YU152765 | 7752357 | | 11.96 | 2.40 | 9.56 |
| YU208239 | G889-1346 | | 36.49 | 26.96 | 9.53 |
| YU148889 | 7945627 | | 16.16 | 6.66 | 9.49 |
| YU155025 | 90200463 | BRAZILEIN | 9.12 | −0.14 | 9.26 |
| YU153031 | 7919640 | | 9.27 | 0.09 | 9.19 |
| YU146020 | 7931802 | | 12.15 | 3.15 | 8.99 |
| YU153545 | 6395104 | | 100.32 | 91.35 | 8.98 |
| YU034447 | 02300309 | [symbol] ESAMICOL HYDROCHLORI [symbol] | 8.67 | −0.18 | 8.85 |
| YU153085 | 00210238 | [symbol] PICATECHIN MONOGALLA [symbol] | 94.59 | 85.87 | 8.72 |
| YU145407 | 5107324 | | 6.76 | −1.79 | 8.55 |
| YU172178 | 8397-0664 | | 46.45 | 37.93 | 8.53 |
| YU148087 | 7917906 | | 15.48 | 7.00 | 8.48 |
| YU149951 | 7498349 | | 6.61 | −1.85 | 8.46 |
| YU016748 | H′I′S 06033 | | 19.64 | 11.20 | 8.43 |
| YU151128 | 7939491 | | 6.20 | −2.19 | 8.40 |
| YU153128 | 7944562 | | 12.52 | 4.15 | 8.37 |
| YU146294 | 7008394 | | 14.08 | 5.94 | 8.13 |
| YU150371 | 7842697 | | 6.16 | −1.93 | 8.09 |
| YU155347 | 00210242 | [symbol] HEAFLAVIN MONOGALLAT [symbol] | 103.69 | 95.69 | 8.00 |
| YU147640 | 7706494 | | 5.15 | −2.82 | 7.97 |
| YU148680 | 6818678 | | −2.95 | −10.90 | 7.95 |
| YU153899 | 7910497 | | 8.44 | 0.54 | 7.90 |
| YU039791 | SAM001246816 | CEFIXIME TRIHYDRATE | 104.49 | 96.65 | 7.84 |
| YU033699 | 01500134 | BACITRACIN | 6.01 | −1.65 | 7.66 |
| YU152893 | 7961326 | | 9.21 | 1.62 | 7.59 |
| YU172168 | 8397-0166 | | 44.99 | 37.50 | 7.50 |
| YU172173 | 8397-0490 | | 44.79 | 37.31 | 7.48 |
| YU145239 | 7947845 | | 7.68 | 0.21 | 7.47 |
| YU208247 | G890-0098 | | 7.92 | 0.47 | 7.44 |
| YU151027 | 7911913 | | 2.93 | −4.49 | 7.42 |
| YU155084 | 00201507 | [symbol] SEPIGALLOCATECHIN DIGA [symbol] | 98.89 | 91.60 | 7.29 |
| YU150472 | 7921066 | | 4.75 | −2.52 | 7.27 |
| YU155440 | 00210505 | PURPUROGALLIN | 13.64 | 6.39 | 7.25 |
| YU034048 | 01500603 | TYROTHERICIN | 27.36 | 20.14 | 7.23 |
| YU034370 | 01503118 | LUPROMAZINE HYDROCHL | 4.23 | −2.95 | 7.18 |
| YU039604 | SAM001246559 | PIRUBICIN HYDROCHLORID | 10.74 | 3.76 | 6.98 |
| YU152562 | 7928138 | | −8.43 | −15.40 | 6.96 |
| YU034506 | 01503918 | CLOBETASOL PROPIONATE | 5.62 | −1.34 | 6.96 |
| YU208230 | G889-1205 | | 24.45 | 17.72 | 6.73 |
| YU172172 | 8397-0271 | | 63.48 | 56.78 | 6.69 |
| YU151184 | 7911245 | | 1.02 | −5.59 | 6.61 |
| YU034320 | 91502245 | ELJ_AGIC ACID | 37.80 | 31.44 | 6.37 |
| YU152583 | 7973763 | | 1.32 | −4.74 | 6.06 |
| YU149514 | 7915263 | | −2.24 | −8.22 | 5.98 |
| YU152451 | 7846193 | | 2.82 | −3.09 | 5.91 |
| YU152649 | 7937631 | | 6.08 | 0.22 | 5.86 |
| YU172071 | 8188-2521 | | 3.80 | −1.99 | 5.79 |
| YU155300 | 01504065 | MYRICETIN | 74.84 | 69.07 | 5.77 |
| YU155257 | 01502253 | HEMATEIN | 104.73 | 99.07 | 5.06 |
| YU155407 | 00201580 | POMIFERIN | 16.14 | 10.52 | 5.62 |
| YU153090 | 00210239 | [symbol] LLOCATECHIN-3-MONOGA [symbol] | 95.83 | 90.25 | 5.58 |
| YU034167 | 01501104 | [symbol] THACYCLINE HYDROCHLO [symbol] | 62.51 | 56.96 | 5.55 |
| YU155100 | 90201515 | THEAFLAVIN DIGALLATE | 104.28 | 98.76 | 5.52 |
| YU152183 | 7980473 | | 6.95 | 1.46 | 5.49 |
| YU208194 | G889-0171 | | 17.11 | 11.63 | 5.48 |
| YU208213 | G889-1039 | | 37.72 | 32.32 | 5.39 |
| YU208195 | G889-0172 | | 14.20 | 8.81 | 5.39 |
| YU153507 | 01504105 | TANNIC ACID | 105.15 | 99.80 | 5.35 |
| YU152560 | 7926149 | | −1.53 | −6.86 | 5.33 |
| YU034398 | 91504105 | TANNIC ACID | 104.83 | 99.54 | 5.29 |
| YU145089 | 7990751 | | 4.23 | −1.01 | 5.24 |
| YU034068 | 01500637 | MERBROMIN | 3.43 | −1.81 | 5.24 |
| YU144896 | 7853261 | | −9.38 | −14.49 | 5.11 |
| YU033698 | 01500133 | AZATHIOPRINE | 10.40 | 5.31 | 5.09 |
| YU034331 | 01503009 | BIOTIN | 104.67 | 99.61 | 5.06 |
| YU039629 | SAM001246768 | [symbol] XORUBICIN HYDROCHLOR [symbol] | 8.94 | 3.92 | 5.03 |
| YU105182 | 5265368 | | 3.14 | −1.74 | 4.87 |
| YU152564 | 7930515 | | 9.13 | 4.29 | 4.83 |
| YU121632 | 7716211 | | 2.95 | −1.84 | 4.79 |
| YU034556 | 01503223 | PARAROSANILINE PAMOAT [symbol] | 5.12 | 0.35 | 4.77 |
| YU208200 | G889-0412 | | 24.86 | 20.10 | 4.76 |
| YU034090 | 01500672 | QUERCETIN | 62.82 | 58.14 | 4.68 |
| YU208228 | G889-1199 | | 24.80 | 20.21 | 4.59 |
| YU155305 | 91504080 | SENNOSIDE B | 4.04 | −0.34 | 4.38 |
| YU033712 | 01500148 | BITHIONOL | 0.82 | −3.70 | 4.52 |
| YU155528 | 01500861 | CORALYNE CHLORIDE | −1.03 | −5.30 | 4.27 |
| YU151409 | 7849329 | | 4.03 | −0.23 | 4.26 |
| YU104987 | 5105131 | | 7.41 | 3.17 | 4.24 |
| YU151859 | 7960076 | | 9.33 | 5.15 | 4.19 |
| YU147750 | 7911930 | | −2.53 | −6.59 | 4.06 |
| YU034074 | 01500644 | PHENYLMERCURIC ACETAT [symbol] | −6.82 | −10.79 | 3.97 |
| YU150447 | 7823376 | | 4.56 | 0.80 | 3.76 |
| YU149941 | 7963683 | | −12.32 | −16.01 | 3.69 |
| YU155201 | 01504078 | SENNOSIDE A | 72.27 | 68.65 | 3.62 |
| YU153907 | 7921224 | | 1.57 | −2.05 | 3.62 |
| YU105195 | 5268563 | | 8.75 | 5.28 | 3.47 |
| YU150403 | 7933837 | | −3.10 | −6.36 | 3.26 |
| YU151614 | 7944468 | | −1.67 | −4.92 | 3.26 |
| YU152920 | 7799774 | | 0.53 | −2.69 | 3.22 |
| YU155091 | 00201513 | GALLOCATECHIN 3,5-DIGAL [symbol] | −0.05 | −3.13 | 3.08 |
| YU033837 | 91500311 | FUSIDIC ACID | 0.93 | −2.04 | 2.98 |
| YU033841 | 01500315 | GENTIAN VIOLET | 96.28 | 93.38 | 2.90 |
| YU034263 | 01502034 | METAMPICILLIN SODIUM | 3.14 | 0.44 | 2.70 |
| YU034023 | 01500572 | THIMEROSAL | −3.15 | −5.81 | 2.66 |
| YU152624 | 7903590 | | −1.90 | −4.45 | 2.54 |
| YU149922 | 7943809 | | −6.24 | −8.78 | 2.54 |
| YU154853 | EI-283 | Ro 31-8220 | 41.07 | 38.79 | 2.28 |
| YU145520 | 7642641 | | 3.79 | 1.80 | 1.98 |
| YU221071 | SAM001246570 | VINCRISTINE SULFATE | 0.59 | −1.27 | 1.86 |
| YU155193 | 00240826 | [symbol] UROGALTIN-4-CARBOXYLI [symbol] | −1.37 | −3.21 | 1.84 |
| YU033800 | 01500260 | PYRITHIONE ZINC | −18.35 | −19.95 | 1.60 |
| YU033802 | 01505153 | 3-HYDROXYTYRAMINE | −1.42 | −2.83 | 1.41 |
| YU152587 | 6498914 | | −1.32 | −2.67 | 1.35 |
| YU105079 | 5152461 | | 44.39 | 43.24 | 1.15 |
| YU146037 | 7959412 | | −0.56 | −1.42 | 0.87 |
| YU034547 | 01500521 | PYRVINIUM PAMOATE | −3.96 | −4.64 | 0.68 |
| YU149834 | 7929434 | | 28.52 | 27.95 | 0.57 |
| YU154851 | EI-246 | GF 109203X | 5.76 | 5.46 | 0.30 |
| YU145132 | 7862194 | | −6.93 | −7.06 | 0.13 |
| YU155285 | 01504002 | BAICALEIN | 0.59 | 1.01 | −0.42 |
| YU033619 | 00100346 | PICROTIN | −12.66 | −12.15 | −0.51 |
| YU034377 | 01503200 | CETRIMONIUM BROMIDE | −6.81 | −6.13 | −0.67 |
| YU040321 | SAM001246676 | IDARUBICIN HCl | −11.32 | −9.53 | −1.78 |
| YU145037 | 7390437 | | −4.02 | −1.09 | −2.93 |
| YU016812 | HTS 06219 | | 10.22 | 13.27 | −3.05 |
| YU033734 | 01500177 | CHLORHEXIDINE | −1.12 | 1.97 | −3.09 |
| YU034020 | 01500567 | [symbol] AHYDROZOLINE HYDROCH [symbol] | −5.13 | −0.11 | −5.03 |
| YU033628 | 00201580 | POMIFERIN | 6.60 | 13.55 | −6.95 |
| YU034280 | 01502099 | [symbol] SSYPOL-ACETIC ACID COM [symbol] | 54.61 | 64.46 | −9.86 |
| YU155086 | 01505249 | APRAMYCIN | −19.45 | −4.67 | −14.78 |
| YU033898 | 01500397 | METHOCARBAMOL | 0.74 | 18.66 | −17.93 |
| YU155308 | 01504115 | HIERACIN | 34.84 | 92.13 | −57.29 |

[symbol]

indicates data missing or illegible when filed

TABLE 5
Z′ scores and Signal to Background ratios of the JARID1B inhibitor screen.

| Plate number | Library | Z′ Score | Signal/Background |
|---|---|---|---|
| 1 | MicroSource GenPlus | 0.64 | 17.07 |
| 2 | MicroSource GenPlus | 0.76 | 18.96 |
| 3 | MicroSource GenPlus | 0.76 | 18.67 |
| 4 | MicroSource NatProd | 0.8 | 18.05 |
| 5 | MicroSource NatProd | 0.76 | 18.43 |
| 6 | MicroSource NatProd | 0.78 | 18.61 |
| 7 | NIH Clinical Collection | 0.78 | 19.41 |
| 8 | NIH Clinical Collection | 0.8 | 17.97 |
| 9 | Yale Compound | 0.81 | 17.79 |
| 10 | ChemBridge MW-Set | 0.81 | 16.4 |
| 11 | ChemBridge MW-Set | 0.72 | 16.6 |
| 12 | ChemBridge MW-Set | 0.8 | 17 |
| 13 | ChemBridge MW-Set | 0.79 | 16 |
| 14 | ChemBridge MW-Set | 0.77 | 17.4 |
| 15 | ChemBridge MW-Set | 0.83 | 19 |
| 16 | ChemBridge MW-Set | 0.82 | 18.8 |
| 17 | ChemBridge MW-Set | 0.8 | 18.4 |
| 18 | ChemBridge MW-Set | 0.79 | 18.5 |
| 19 | ChemBridge MW-Set | 0.83 | 18.7 |
| 20 | ChemBridge MW-Set | 0.81 | 18.4 |
| 21 | ChemBridge MW-Set | 0.82 | 18.5 |
| 22 | ChemBridge MW-Set | 0.87 | 19.63 |
| 23 | ChemBridge MW-Set | 0.86 | 20.16 |
| 24 | ChemBridge MW-Set | 0.85 | 20.04 |
| 25 | ChemBridge MW-Set | 0.89 | 19.98 |
| 26 | ChemBridge MW-Set | 0.86 | 19.74 |
| 27 | ChemBridge MW-Set | 0.87 | 19.56 |
| 28 | ChemBridge MW-Set | 0.88 | 19.84 |
| 29 | ChemBridge MW-Set | 0.8 | 18.17 |
| 30 | ChemBridge MW-Set | 0.83 | 17.67 |
| 31 | ChemBridge MW-Set | 0.74 | 17.53 |
| 32 | ChemBridge MW-Set | 0.82 | 17.45 |
| 33 | ChemBridge MW-Set | 0.82 | 16.66 |
| 34 | ChemBridge MW-Set | 0.75 | 14.3 |
| 35 | ChemBridge MW-Set | 0.78 | 14.4 |
| 36 | ChemBridge MW-Set | 0.81 | 12.9 |
| 37 | ChemBridge MW-Set | 0.8 | 14.7 |
| 38 | ChemBridge MW-Set | 0.78 | 14.4 |
| 39 | ChemBridge MW-Set | 0.8 | 14.4 |
| 40 | ChemBridge MW-Set | 0.79 | 13.9 |
| 41 | ChemBridge MW-Set | 0.81 | 14.6 |
| 42 | Maybridge | 0.75 | 12.9 |
| 43 | ChemBridge McF | 0.77 | 13.9 |
| 44 | ChemBridge DvS | 0.78 | 15 |
| 45 | ChemDiv | 0.81 | 14.4 |
| 46 | ChemBridge McF | 0.8 | 19.25 |
| 47 | ChernDiv | 0.81 | 17.46 |
| 48 | ChemDiv | 0.79 | 18.41 |
| 49 | ChemDiv | 0.78 | 17.92 |

1. A pharmaceutical composition comprising a compound, or a salt or solvate thereof, selected from the group consisting of:
caffeic acid;
esculetin; 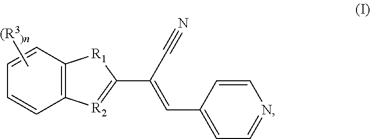
a compound of formula (I):
wherein in formula (I):
R¹ is S, O, NH or N(C₁-C₆ alkyl);
R² is N, CH or C—(C₁-C₆ alkyl); and
n is 0, 1, 2, 3 or 4, wherein each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy and carboxy;
a compound of formula (II): 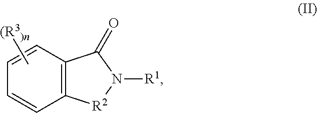
wherein in formula (II):
R¹ is C₁-C₆ alkyl, substituted C₁-C₆ alkyl, C₃-C₇ cycloalkyl, substituted C₃-C₇ cycloalkyl, aryl, substituted aryl, heteroaryl, substituted heteroaryl, heterocyclyl, substituted heretocyclyl, acyl, benzoyl, substituted benzoyl or phenylacetyl;
R² is C(R₄)₂, O, S, C(O), S(O), S(O)₂ or Se;
n is 0, 1, 2, 3 or 4, wherein:
each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy, cyano and carboxy; and
each occurrence of R⁴ is independently H, C₁-C₆ alkyl, or substituted C₁-C₆ alkyl;

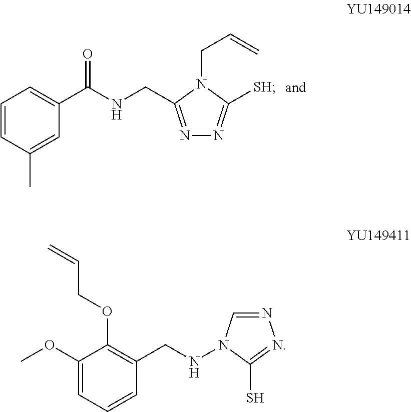

2. The composition of claim 1, wherein in formula (I) R¹ is S, NH or N(CH₃).

3. The composition of claim 1, wherein in formula (I) R² is N.

4.-5. (canceled)

6. The composition of claim 1, wherein the compound of formula (I) is selected from the group consisting of (E)-3-(pyridin-4-yl)-2-(5-(trifluoromethyl)benzo[d]thiazol-2-yl)acrylonitrile; (E)-2-(1-methyl-1H-benzo[d]imidazol-2-yl)-3-(pyridin-4-yl)acrylonitrile; and any combinations thereof.

7. The composition of claim 1, wherein in formula (II) R¹ is C₁-C₆ alkyl, phenylacetyl, aryl or substituted aryl selected from the group consisting of phenyl, o-tolyl, m-tolyl, p-tolyl, o-fluorophenyl, m-fluorophenyl, p-fluorophenyl, o-chlorophenyl, m-chlorophenyl, p-chlorophenyl, o-isopropylphenyl, m-isopropylphenyl, p-isopropylphenyl or isopropyl.

8. (canceled)

9. The composition of claim 1, wherein in formula (II) R² is C(O), S, SO₂, CH₂ or Se.

10.-11. (canceled)

12. The composition of claim 1, wherein the compound of formula (II) is selected from the group consisting of 2-(4-methylphenyl)-1,2-benzisothiazol-3(2H)-one; 2-phenylbenzo[d][1,2]selenazol-3(2H)-one, 2-(4-chlorophenyl)-5,6-difluorobenzo[d]isothiazol-3(2H)-one, 2-(4-chlorophenyl)-5-(trifluoromethyl)benzo[d]isothiazol-3(2H)-one, 2-(4-chlorophenyl)-6-isocyanobenzo[d]isothiazol-3(2H)-one, and any combinations thereof.

13. (canceled)

14. A method of treating or preventing cancer in a subject in need thereof, the method comprising administering to the subject a therapeutically effective amount of a pharmaceutical composition comprising a compound selected from the group consisting of:
caffeic acid;
esculetin;
a compound of formula (I): 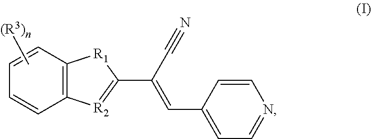
wherein in formula (I):
R¹ is S, O, NH or N(C₁-C₆ alkyl);
R² is N, CH or C—(C₁-C₆ alkyl); and
n is 0, 1, 2, 3 or 4, wherein each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy and carboxy;
a compound of formula (II): 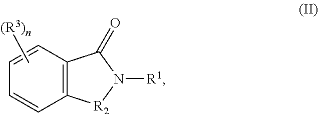
wherein in formula (II):
R¹ is C₁-C₆ alkyl, substituted C₁-C₆ alkyl, C₃-C₇ cycloalkyl, substituted C₃-C₇ cycloalkyl, aryl, substituted aryl, heteroaryl, substituted heteroaryl, heterocyclyl, substituted heretocyclyl, acyl, benzoyl, substituted benzoyl or phenylacetyl;
R² is C(R₄)₂, O, S, C(O), S(O), S(O)₂ or Se;
n is 0, 1, 2, 3 or 4, wherein:
each occurrence of R³ is independently selected from the group consisting of C₁-C₆ alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, halogen, C₁-C₆ alkoxy, nitro, amino, acetamido, hydroxy, cyano and carboxy; and
each occurrence of R⁴ is independently H, C₁-C₆ alkyl, or substituted C₁-C₆ alkyl;

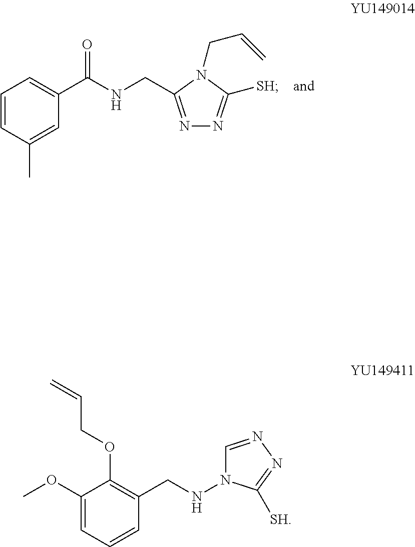

15. The method of claim 14, wherein administration of the pharmaceutical composition to the subject inhibits the activity of at least one JARID1 demethylase in the subject.

16. The method of claim 15, wherein the at least one JARID1 demethylase comprises JARID1B.

17. The method of claim 15, wherein the at least one JARID1 demethylase comprises JARID1A and JARID1B.

18. The method of claim 14, wherein the cancer comprises a solid cancer selected from the group consisting of breast cancer, prostate cancer, melanoma, lung cancer, and any combinations thereof.

19. (canceled)

20. The method of claim 19, wherein the breast cancer comprises at least one HER2-positive breast cancer cell that is resistant to trastuzumab.

21. (canceled)

22. The method of claim 14, wherein the subject is further administered an additional compound selected from the group consisting of a chemotherapeutic agent, an anti-cell proliferation agent, and any combinations thereof.

23.-33. (canceled)

34. A high-throughput method of determining whether a compound inhibits JARID1B or JARID1A demethylase activity, the method comprising the steps of:
providing tagged full length JARID1B enzyme or JARID1A enzyme;
incubating the tagged full length JARID1B enzyme or JARID1A enzyme with the compound and tagged H3K4Me3 peptide in a system at a determined temperature for a determined period of time; and
determining whether any H3K4me2/1 peptide is formed in the system, whereby, if any H3K4me2/1 peptide is formed in the system, the compound is determined to inhibit JARID1B or JARID1A demethylase activity.

35. The method of claim 34, wherein the tagged full length JARID1B or JARID1A enzyme comprises FLAG-tagged full length JARID1B or JARID1A enzyme.

36. The method of claim 34, wherein the tagged H3K4Me3 peptide comprises biotinylated H3K4Me3 peptide.

37. The method of claim 34, wherein the system further comprises alpha-ketoglutarate, an iron (II) salt and ascorbate.

38. The method of claim 34, wherein determining whether any H3K4me2/1 peptide is formed in the system comprises incubating an H3K4me2 antibody or an H3K4me1 antibody with at least a portion of the system.

39. The method of claim 34, wherein the system is heterogeneous.

40. The method of claim 39, wherein the tagged H3K4Me3 peptide is immobilized on a solid support.

41-47. (canceled)
